SEQUENCE LISTING (186 sequences)

SEQ ID NO: 1 - DNA, 23 bp, Sus scrofa
ggaaacccaggctggttggaggg

SEQ ID NO: 2 - DNA, 23 bp, Sus scrofa
ggaactacagtgcggcactgtgg

SEQ ID NO: 3 - DNA, 23 bp, Sus scrofa
cagtagcaccccgccctgacggg

SEQ ID NO: 4 - DNA, 23 bp, Sus scrofa
tgtagccacagcagggacgtcgg

SEQ ID NO: 5 - DNA, 23 bp, Sus scrofa
ccagcctcgcccagcgacatggg

SEQ ID NO: 6 - DNA, 23 bp, Sus scrofa
ctttcatttatctgaactcaggg

SEQ ID NO: 7 - DNA, 23 bp, Sus scrofa
ttatctgaactcagggtccccgg

SEQ ID NO: 8 - DNA, 23 bp, Sus scrofa
cagctgcagcatatatttaaggg

SEQ ID NO: 9 - DNA, 23 bp, Artificial sequence
  Synthetic oligonucleotide
ctcctcgcccttgctcaccatgg

SEQ ID NO: 10 - DNA, 23 bp, Artificial sequence
  Synthetic oligonucleotide
gaccaggatgggcaccaccccgg

SEQ ID NO: 11 - DNA, 23 bp, Artificial sequence
  Synthetic oligonucleotide
ctctccctcactctaacctactt

SEQ ID NO: 12 - DNA, 22 bp, Artificial sequence
  Synthetic oligonucleotide
tatttctctcacatggccagtc

SEQ ID NO: 13 - DNA, 23 bp, Artificial sequence
  Synthetic oligonucleotide
ctctccctcactctaacctactt

SEQ ID NO: 14 - DNA, 22 bp, Artificial sequence
  Synthetic oligonucleotide
gactggccatgtgagagaaata

SEQ ID NO: 15 - DNA, 27767 bp, Sus scrofa
atacaagtgccttttacagacaatctgcacaagttatttgttagacatatttgattatagaattaatattaaaaggggttataacaatcaagcattgataatttaattatgtttgcctattttactttagttttttgacataactgtgtaactattgcgatttttttattcctaatgtaattagttcaaaacaaagtgcagaaatttaaaatattcaattcaacaacagtatataagtcaatattccccccttaaatttttacaaatctttagggagtgtttctcaatttctcaatttctttggttgtttcatgtcccatatggaagaaaacatgggtgtgaaagggaagcttactcttttgattacttcccttttctggttgactccacctccattatgaagcctttctgtatttttgtggaagtgaaatgatttttagaattcttagtggttctcttcttcaggagaacatttctaggtaataatacaagaagatttaaatggcataaaaccttggaatggacaaactcagaatggtgctacatgaaaactctggatctgcaggtaaaatcttctcatttattctatatttaccttttaaatagagtgtagcaatattccgacagtcaatcaatctgatttaatagtgattggcatctggagaagaagtaacagggaaaaaggcaataagctttataaggggaacttttatcttccatagactcaaaattgaagacgtgactagaagattgctagatttggcatcagttttgtaaaattgctgaggtgaaattaagtaagggatgaaaattaactaaattgtgttgagtatgaaactagtagttgttagaaaagatagaacatgaaggaatgaatattgattgaaagttgatgacctagaggacatttagactaacacctctgagtgtcaaagtctaatttatgatttacatcgatgcgttaaactcatttaacattcttacttttttcccctcaagcatttaagctgaagtataacatttcacatgaaagcctggattataaatgcacagttcagtgacctatctcagaggagtgactgccatagcattttttttgtctttttgccttcagagccacagcaacgcgggatccgaagccgcgtctgcgacccacaccacagctcacggcaatgccggatctttaacccactgagcgaggccggggatcgaacccgcagtctcatggttcctagtaggattcgttaaccactgcgccacgacgggaactcctaccatagcatttttacttttaagttactgttggtttagagtaagaaggagaaatgagagtgatggagcgtttgctatatttggagacaaggtcctatattggaggttctcaaatataaattttgtcgctttttcctccaatgtattgttcaactactatttagcaggccactgtgccaggtactggtgaaactggtgaacatgatagatgtaattcattccctcatggaactttccatctaacaatgtggatcaggtaggcttggagatgagaatgccagtggttgactatgactctgtggctgaagggagagctactcacttcgtagtttcatcaatgtctttttggttttccaggttttaagccctgctcttgcaattcttttcccttctccaactttcttctaatttctcacccctaggatgcctataaacatgagtattttcaaagctacttcactgaggttatatgatcctggtgtgaatttttcctgcctgacttgccatttagaaggaagtgtttcctggaatttccattgtggcttggtggttaaagaccctgcattgtctctgtgaggatgtgggttcaatctctggcctcattcagtgagtgggttaaggatctggtgtcgctgcaagctgtggctaagatcccacattgccatgtctgtggtgtagactggcacctggagctctgatttgaccacaatcttaggaacttcagatgtggccataaaaaggaaaaaaaagttaggaagggttttctgtcttgtttggaccttcgttaatctcaaacctttggaaccatctctcctccaaaacctcctttgggtaagactgtatgtttgccctctctcttcttttcgcagactttagaagatgttctgcccatttaagttccttcactttggctgtagtcgctgttctcagtgcctgcttggtcactagttctcttggtgagtactttgacaaatttacttgtaaccgagcccaactgtgacaagaaacactgaaaagcaaataattgctcctgaagtctagatagcatctaaaaacatgcttcatggtttcaaggatcatatattgaaaccccagggatcctctagagtcgacctgcagcatgcaggggggggggggggggggggggggggggggggggggggggggggggggggggggggggggggggggggggggggggggggggggggggggggggggggggggggtgcataaggaaagactatctcaacgtcttattcctcagcttacattagatttgaaactctagtcacctaaaatgcaaatctcatttacttaccatcagagatattaatgacctatagaattcagcataaataaagtttcatgtatggatattagcttatggttctagtcactgctaattgaaacctgtgatattgctgtttgttttgactcctatgaaataacattctcccattgtaccatggatgggtccagaaacatttctcaaatcctggcttgaaaaaataaataagtaatctaaagaataataattctctacttgctctttgaatcttgaccaattgctgcatttacctattgttacaggaggaaaagacaaggagctgaggctaacgggtggtgaaaacaagtgctctggaagagtggaggtgaaagtgcaggaggagtggggaactgtgtgtaataatggctgggacatggatgtggtctctgttgtttgtaggcagctgggatgtccaactgctatcaaagccactggatgggctaattttagtgcaggttctggacgcatttggatggatcatgtttcttgtcgagggaatgagtcagctctctgggactgcaaacatgatggatggggaaagcataactgtactcaccaacaggatgctggagtaacctgctcaggtaagacatacacaaataagtcaagcctatacatgaaatgctttgtgggaaaaaatgtatagatgagttaaaaacaaaaaggaaccagttttctataagtcatctagtccatgtataaaattacccaatccattactaaaagaccacttctggtattttacacatgacaaagcccatattaaaaaaaaaaaattcagaagagattctgaatgctataataaatgagcaagtgactagcttcaattttatattaggtcattctaccttctacttctacatgaaaatatcataatgtctaagttaattccttgtcccctttcccaataaagcactgctttcatgcactggcctatgaatcatgaactttttgccctttaactgatgatcaacttaccaaatcaagaaataaatattcttagcactgatccttttttgttgttgttggaggaagaatgttttgcaaagtagaattgcttttttctgtttaacagtgctattcatttcatttacatggtcgttttaatttataaaacatttcataagtttcacctcatatgcccttacaataactcaggaagttatatgttagacctttctgctgacaaatcccagagtcatgtttctgacccagttcagattccttggcttcccatttctctttgctcatgtcattgacctttatgcagccctcttacctcccacctttctattacagaccatctcctccataggactggtgttagaaagtactaatctctacccaggcattgtggtgcaatgtgggcagcacaggctggtatctagaaaaatgctgaagtgaattccagctcagctgctcgttaatactatcgttttaagtaagctgttcaatcctttgaaattcactttctgagcactcagtgatataataaatgtagagctactggtacactgtctggtatgtaataggtgttaccaattaaccttagtttcctcatgggtcactggttctcattacctagacaactcatttctctttcttcctctttctctttctccattctcctcctccttcttcctcttcttcttgtctgttattgttatatcattttgctgagaaagttaagaaataacaactctaacctctacatcgaccacctagagcaaagttaaaaataataataaaccttgccagactcttactataattgttgctgtctatagagttgactgtttaagttaagacatcagtatatatttttaatttttgtgttttttttttcatacttttacatgaggatcctttatataaggatgagttaaacaaacttgatttttgaagtttatacccctgaggctcaactgcataataatagaaagggatccatagcctctcaaggacttaactagtttcatgagttttcagaatctgaatttctgagattctccaccccaattaaagctcaagcctcagaacatatatccttctcttggtaaattctattcttatcacatgcgtaataataaaaaagagagatgttggagacagatttttttcctcacattctgtctctactgttttctaggtgtttgattctgtgttatttaacctcagtttgcttatctgtgaagtagggattatggtaataacatataatgctttatgttgtaaagactaaagaagatagcatatgtaacacatttggaacagggaatgcatattttgattgtgagctcttattattattaccattcagccctaataaaaatcttggtaagtggaaggctttggatttcagaacttttaaaatctaattactttttcaaaaaagaacttcttagggttttttttttttaaccacaaagtgtttctattttttaggtgtcccaaaatttcgttccaaatatctttttctcagatattttagtcctcatagaacacctagggatagtggatagagaaaattttctttattaaaaagctgttctttgctaaaaattgtagcaggtacttttgggaggggggaaaactttgattcagaaactgctaagacatggagtgttttgactaatttttcctcaatttttaatgttttttataccatagggtacttttgcaaactattatgcatacttatatatttttacttttttcctgtcttttaacttccaaattcaacttcagacaattattcatgcactaaactgtttgtagtaagaaagattaaaattaaaaaattaaccattcaacaaatgactggtttgccatttttactactttgttgtatgaacaatttttttttctacaaatgaatactttgagtctgatttatccattcctacataaaagtttttactatatcttagtattggaaggaaacaaaacaaaacacaatgtaaattttaatctataaattttgggggggtaaatatacatagatgaaagtcttaaccattaattagagtcaaaagattaaaattctccaatatgtgaacttaggctgcatccaaaatgaagcatcatttttaaggacagcatcaaaagtgaccagaggaattttactttctttcttttttttttttttgaattttagtttctaaactcacttctgaataaatacaacttctaaattctcgtcttttctctactctagatggatctgatttagagatgaggctggtgaatggaggaaaccggtgcttaggaagaatagaagtcaaatttcaaggaacgtggggaacagtgtgtgatgatcacttcaacataaatcatgcttctgtggtttgtaaacaacttgaatgtggaagtgctgtcagtttctctggttcagctaattttggagaaggttctggaccaatctggtttgatgatcttgtatgcaatggaaatgagtcagctctctggaactgcaaacatgaaggatggggaaagcacaattgcgatcatgctgaggatgctggagtgatttgcttaagtaaggactgacctgggtttgttctgttctccatgagagggcaaaaaaaggggagtaaaagtcttaaaagctcaaactgttaaaaacataatgatgattgcttcttttatcatcttattattatctaatttcaggtcgaaattctagtacctgtgcagttttttaccttaactgaaattaagataaataggatagggaggaaggatgagcagtgacatttaggtccaagtcatgaggttagaaggaaatgttcagagaatagcccattccctcagccctcaaagaaagaaagaaagaaaaagaaaaaaaaaaagaaagcttaactagaaaattttgttctctggatgttttagaggcaaaccatccctttatcattcctacctacaaagccttctcttaatcacattacccaccctttcctactatagtcaggggggggggggggggggggggggggggggggggggggggggggggggggggggggggggggggggggggggggggggggggggggggggggggggggggtgaaaaaagaaccaaacaatttcaacaaaaaaccaaacaattccaacaaaattggtccaataagcaaacctctagataaatttcagtgccctggatgttttgttaggaactcttcctacaatgcgtgctttccattctgaaaagtcctatctacttgcctgatccacttctccttccatcctaaacgattttcagtggtagtatattactgttgtctctgtctctacttatatatcttccccttttcactcactcctctcaggtacagctcttcagtttgcccttattcttgtttccttgtcaatgacttgttttgtgtccctcttacagatggagcagacctgaaactgagagtggtagatggagtcactgaatgttcaggaagattggaagtgaaattccaaggagaatggggaacaatctgtgatgatggctgggatagtgatgatgccgctgtggcatgtaagcaactgggaggtccaactgctgtcactgccattgtcgagttaacgccagtgagggactggacacattggctcacacacatacagccatgacacgatctgctctatggtccgatgattaaaggggggggggggggggggggggggggggggggggggggggggggggggggggggggggggggggggggggggggggggggggggggggggggggggggggagaagagctggtggacatttctggaaaggaaccaaaacccggaagggccttgttcttcaggatttgggatggattggggagggagaaaattgtttctaatatttcttggtgggaattcttttacagttgtgacaaatctttcacatattcttcatttgagtagtttggagggttgtctgactgttttctataataaatgtcccaagtgctatgaggtaccacatttcaaattctaattctacctgaagctccaaaaagacaaaatgttataggtcttttctttatatctaatttgcttatggtttttagccattgacaatttttttttcttaactcttgaaactataaccctatttctaaccaaattcatgttctatactggctcttcaaaaacccaggagatgggaaagccagaatctccagtgtttcagcttctgggaaggagcaagtttttaaaaataccctctgggagctaaattccacatgtatctatggcctaagtgtatgtttattttgcagatggatcagatctggaactgagacttaaaggtggaggcagccactgtgctgggacagtggaggtggaaattcagaaactggtaggaaaagtgtgtgatagaagctggggactgaaagaagctgatgtggtttgcaggcagctgggatgtggatctgcactcaaaacatcatatcaagtttattccaaaaccaaggcaacaaacacatggctgtttgtaagcagctgtaatggaaatgaaacttctctttgggactgcaagaattggcagtggggtggacttagttgtgatcactatgacgaagccaaaattacctgctcaggtaagaatttcaatcaatgtgttaggaaattgcattctactttcttttacatgtagctgtccagttttcccagcaccacttgttgaagagactgtcttttcttcatcatatagtcctacatcctttgtcataaattaattgaccataggtgtgtgggtttatatctgggctctctattctgttcctttgatctatatgtctgtttttatgccagcaccatgctgttttgattactatagctttgtagtatcatctgaagtcaggaaacatgattcctccagctttgttcttctttctcaagattgttttgtctattcagagtttatgttcccatgcagatttaatttttaaatttatttaatttttattttttatttttaatttaaattaatttaaattttttatttcccaacgtacagccaagggggccagggtaacctttacatgtatacattaaaaatttcaggtttttcccccacccatttctttctgttggcaagtaaatttttgaacaaagtttcccaatgctttttaaggggaattcccttggggggggggggggggggggggggggggggggggggggggggggggggggggggggggggggggggggggggggggggggggggggggggggggggggggggggggggggggggagacgaaattgactatattttctttgttgggaatcttttacagttgtgacaaatctttcacatattcttcatttgagtagtttggagggttgtctgactgttttctataataaatgtcccaagtgctatgaggtaccacatttcaaattctaattctacctgaagctccaaaaagacaaaatgttataggtcttttctttatatctaatttgcttatggtttttagccattgacaatttttttttcttaactcttgaaactataaccctatttctaaccaaattcatgttctatactggctcttcaaaaacccaggagatgggaaagccagaatctccagtgtttcagcttctgggaaggagcaagtttttaaaaataccctctgggagctaaattccacatgtatctatggcctaagtgtatgtttattttgcagatggatcagatctggaactgagacttaaaggtggaggcagccactgtgctgggacagtggaggtggaaattcagaaactggtaggaaaagtgtgtgatagaagctggggactgaaagaagctgatgtggtttgcaggcagctgggatgtggatctgcactcaaaacatcatatcaagtttattccaaaaccaaggcaacaaacacatggctgtttgtaagcagctgtaatggaaatgaaacttctctttgggactgcaagaattggcagtggggtggacttagttgtgatcactatgacgaaaccaaaattacctgctcaggtaagaatttcaatcaatgtgttaggaaaattgcattctactttcttttacatgtagctgtccagttttcccagcaccacttgttgaaaaaactgtctttttcttcatcatatagtcctacatcccttggccataaattaattgaccataaggggtgtgggtttaatatccggggctcctcaattcgggtcccttggatcctaaaagccggttttataacccgacacatggcctgtttttgactaaataaaacctttggaaaacaatcccgaaggtcgaggaacatggaatccccccaacaaaggaccttctttccccaaaaatgcggctcagccaactcaaaaagattttatgaatcacaaaccgcacattatcttcctaaaattactattcctatgttttaatttgcaaagtcattccgatatagttggcgcagagtaactcatttagatatccaccccaccagttcctcactcaagtaaggggggggggggggggggggggggggggggggggggggggggggggggggggggggggggggggggggggggggggggggggggggggggggggggggggcccccatgtgagattttgtgtgtcctttaagagtggagtctctatttcccactgctctctggttctccccaaagtaagccctgctggctttcaaaacttctgggagcttgccttcttggtataggactcctgggctagggagtctaatgtttggcttagaccccttactgcttgggaagaatctctgcaactgtaatgaattatcttcctatttgtgggttgctgaggatatggtcttaactgttctgtgttctacccctcctatccatcttgttgtggttccttctttatatctttagttgtagaaaagtttttcttatcaacagttgctctgtaaattgtaacttgggtgtacacctagtaggaggtgagctcagggtcttcctactctgccatcttggccatgtcctctaaacattttggtgtatttcactgcaacctttttaaaaatctcaaaagtgagctgtgattggctagtcttgtggataatctctagcatttgatgctaatcatatttatacaaatactttgttgaaaagtgatgcctttttaactattattaaaaaacgtattgacataactattgctattatactgaaaagaaagaccttagagaaaatagcataagagcaaaaccattaaacatggagacatctagtcatagggtggaaattttatgtggtccatatcccctaaccagtggctttacaccaggcacatcctaactaagatctgctcccaagtgtcttccctgatgctttaaattgtgttacatggaaactatcctttgatgaagaaatgcaaccttttaaaatacaacattgaaacttttgtgctttaattttgcttttcaacattttttctttttaaaagaagaaatttatttgtttttttaaattttaatggccacggcatatggaagttctcaggccagggatagaattcaagccacaggtgcgacccatgccacaactgctgcaacaccagatcctttaacccactgcaccaggccagggattgaagccttgccttactgacaatctgagccacttcagtcagataaagaaatttcttcattaagcagagtattcacatggtttaaacttcaaaatattaaagtgtaaactctttccccaccactgtccccagctcaccaactctacttaccacagacaactgatgtggttagggtatttaaatagtaaatccaagaaaatataaacaaatccgtatatataggtttcaccccattttattatcctaatgttgcatatcatataaactatactgtcccttgggtattcacttagtaaaatattttgatcataatttcctatcagtatttaaagagctttctgaaattatttctgtataacatttcttttctcatcggtaggggggggggggggggggggggggggggggggggggggggggggggggggggggggggggggggggggggggggggggggggggggggggggggggggggaatgggaagaaaaaaccaccatggttaattttttttatccctctacacccgggaaaattacccttggggccacacttttctatagaaaggggattatttaaaagggtctgaaaaagaatttttttttcgaaaggggaaatatttggcctaacttagtcacataagccatgttctctggcaagttaggtaacatacatttttgtcattgggggcaacaaaaacaattttccttttggaccttttgggactccgcattggttagggaaggggaagtatattggaattcggaaaattccttccaaattaaaaaaggtttgttattttcatattaacctatttcatattaattagcatgaattccagcgccattaaaagggaaaacacctggagtggtaagaaaaaagtttttttttctctttttttttttttttttttaatggccacatctgtggcatgtgaagttcccaggctaggggtcgaataggagctacagctgccagcttgcaccacagccacaacaatgccagagccaagcctcatctgcgacctataccacaactcatggcaatgctggttccttaaccccctgagtgaggcctggggtcaaacccacatcctcatggatactaaccggctttgttaccgctgagccatgagggaaactccctttttctcattgaaaataagtcaaatagataagcagcttaaggctgtttgggtgattctgtggtccagtaattatcaaatcctactggacaagaatagagaatgtgcaaatgagggaacgtgttggtgagatcaggctctgcccactgagctatcctctgtcatgggccctgtgctgttctcagagctgtacttcctagggcattgttctcatttcaattctgagttcagtgtggagagtatacgtgtgtgggggctgcacgcttttcacaacccactttctgctgatactgatttagggatccttggattgctttacagttgagtcatcattaactagtgtcacttgccttcaaagtcagcaaaataattgtctccaaactagtaggcttctagtgtatttgctttaatccaatgccatgtgaaagtaacatggtcaaagaataagttatataccttgacctaccctgtgaccaggctcttcctcttaatttattgaccactgccttaaggtcatttgaaaccatgggtttgggaggaaggcaaggcctaaatcccgtctttgttggaaggctcactgtccttgtctttagagcatcatttttttttaaactggggtacagtttatttacagtgttgtgtcaatttctgctgtacagcacagtgacccagtcatacacatacatacattctttttctcatactatcttcaattttattttctgctaagtctgccattttatcatcacctcagtttgaaggacaggatatttagagtttgttttttttttccccccaatcctgcaatttctaaattataagactctcaattagccgtatataacagctgcaggcacaggatgtctccctcacaaaattggtatttttccttccatttcttcttgcagtttggctatttcttgtctgagttcatctctctttttaagtgttaaaaagggcaaggaggattcatgctatgtcaacattatgattttttcttttctatacttgataagagtatacttttcccaaatgtcatccaacttttcagcatcagtttggacatggttttcttttcaaggtggtatttctctaatgtcacttgaataacaagactcgttagttctccaggctacaatatcctagtctgagtatattctgcatgttaattctattcagccacatccataatttaggttttattcctggaacacctcacttttttttttttttttggtctttttatagccataaccatggcatatggaggttcccaggctaggggtctaatctgagctttagccactggcccatgccacagccacagccatgccacatctgagccacatctgtgaccttttccacagctcacagaaacaccagatccctaacccactgagtgaggccaggggtcaaacctgtaacccttccatggttcctagtcagattcgttcctctgtaccacgatgggaattcctaatacctcacttatgataacacattctgaattatttaggattctattatactgcatgtaatagaaatcccaaatagcaaaatttgcaacttaaggcaggttcctgtctttacaaaatcatgttttcctttgctatatgtgcactttgctttcctctgtgaattcccttttttgttatatttctatagcttttggaaacacttttacttatttgggggggcctagatttttaaccctctccttgtttttctagaaatagagtttataattttatttcttcatttacttgatactttcaagagattcccaggaaaaaaattatggaaatactgtctctgtgcctgccaagttcaaactaagaattgtataatctgttttaattcttaagcatttatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttggaggggacattccctgctctggtcgtgttgaagtacaacatggagacacgtggggcaccgtctgtgattctgacttctctctggaggcggccagcgtgctgtgcagggaactacagtgcggcactgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgggtaagttctgcacatcacttcgggttacagtgatttaagaaacaactaaggtggggcaaagggtagtgaggcatatccatcagagcaaattccttgaaatacggactcagagggaaccattgtgagattgaggttcccagaggtgtggatttaatgaattagtgttacctcatgtacaaggtagtatactaccagaaagataaaaattcagaagcgagtttgcagcaaaactcatagggagaacttcttttataaataatatgaagctggatatttagtgcaccacctgatgaccactttattaataaataaagagttcctgttgtggcgcagcggaaatgaatccgacaaataatcatgagtttgcgggtttgatccctgacctcgctcagtgggttggggatctggtgttgccatgagctgtggtgtaggtcgcagatgctgcttggatcctgctttgctgtggctgtggtataggcttgtggctacagctccgatttgaccgctagcctgggaacctccatatgctgcgggggtggccctcaaaagaaaaataaataaataagtaaataaataagtagtttaaaaaggacaagaagaaatatatttggtgttatattctacagagacaaagataatcaccatgcccgattgatttttcaaggcatataaatgagacgtcatgggagcaaaaatggtcataatacaatgcccttgttttgtgtacatggtaagattttagaaagcattgtgaggtaaaaaagtgtactcagttataatatattggggaaaacagtactatgagaagtaaaaaaatctacatgccggaagttatttttttaatgtctcttttagagtcgcacatgcggcatatggaggttcccaggctaggggtcgaatcagagctatagccactggcttatggcacagccacaacaacactagatctgagccacatcagcgacctatactatagctcatggcaatgccagatccttaacctactgagccaagccatgggtcaaatccaggtcctcacggatcctaggcaaattcatttctgctgagccacgaagggaactcctcagaagtgattttgatgttactttcttttcatgacaaatctggtaaagtacatacacatagaaactgaagtgtcagaaagggaaatatttcattttaaggtaatgtatacaaaacagtggttttaccatctgagtatctcgctaaattttaactatcaaggacaattgccaaaaaaaaaaaaaaaagagagagagagagaacagaatagggttatgaagctaaaatcacagggttatgaagctaaaatcacagtaatttagggagaaaaaaatccaaagcatgtaattgataaaaggttctgagcctttgtttgagatttagaattcaacttagaaataccggtggtattttaaagcagtccataagtataaaatccaaggctaaaaaaccagaaggtatttgtagaacaaatatattttaataagctctaccaagtcatccagaagctattaaagaattactggtcactgacatagtgtacctgttttcaaggccattcttacatcagaataaagggagagcaccctctgaatcttcagaaaagatgtgaaagtgctaattctctatttcatcccagagttcatgtctctgagactgatcagtgaaaacagcagagagacctgtgcagggcgcctggaagttttttacaacggagcttggggcagcgttggcaagaatagcatgtctccagccacagtgggggtggtatgcaggcagctaggctgtgcagacagaggggacatcagccctgcatcttcagacaagacagtgtccaggcacatgtgggtggacaatgttcagtgtcctaaaggacctgacacactatggcagtgcccatcatctccatggaagaagagactggccagcccctcagaggagacatggatcacatgtgccagtgagtatccattctttagcgccactgttatcttctgatctacctaagcagaagttttataatctgtagttaatccctattctacctggatgatgggattcattctgtttaatttggtgtgcaggtattcagcatcagtgatcattttcccaaagaccatcatgctctgatggtcttctcaaaagttctaatcagttgcttcctccgtgaacagttgaggagcagagaatatgtaattcagaatttgactattgaatcatcccatttttctttcacatagtcttttgttgcactgaatataaggagagaagcagtcagaaagatcaatcctgaattatttctccattctacatctgttttaaatttcaaaaaaaaaaattgttataggtgatttacaatgtctgtcaatttctgctctacagcaaagtgacccagttatttacatatacattctgtttctcatatttttaaaccaggagatttctatctgcctggcggtttgagggaatttaacattatgcatttatgttaactttattcacctgatgttttctaagtcatactgagattcttatgcccaggatggaatacacctggtttgctggaaagacatgtgctttcataaagacgaattttggaaaaaatataaaatttaaaaggcccattaaataagcaaagttttaagagatttcaaaaaaaatttcatctctctcttttcctctttgacctcttgggcacgttcatcttctcaaatatgatcttggtgtttctgacttttcagacaaaataagacttcaagaaggaaacactaattgttctggacgtgtggagatctggtacggaggttcctggggcactgtgtgtgacgactcctgggaccttgaagatgctcaggtggtgtgccgacagctgggctgtggctcagctttggaggcaggaaaagaggccgcatttggccaggggactgggcccatatggctcaatgaagtgaagtgcaaggggaatgaaacctccttgtgggattgtcctgccagatcctggggccacagtgactgtggacacaaggaggatgctgctgtgacgtgttcaggtgagggcagagagtctggattgagcttggaagctctggcagcaaagagagggtgggcggtgacctgcattgggtaaagattggaaggtccagcctaaggatctggtggtggggggagacatgatgtttcagtctgaagaatgatgaaaacctgtgtggttacgcatgggccttcgccgaggaaagggacataactaccatgtatcctcctgcagagggaggaagaactaggggattctagttttgtgtgggaaggagcagtttacttggctcaggaggcactaaaggctcagataggaaacagagatctgttccattcttactcccagaactgattctcttctcttttctcctacagaaattgcaaagagccgagaatccctacatgccacaggtatatcaaaaagtttaagaacatgggacccattgtctgcattttgtggaatccctcttattaagacattctgggtcagaagttctgaggatttgacatttacttcagctatctgttatcttacccaagagagggatggtaactaggaacccaggtcttttagctaagacattatcacctcttgtgatgtttacttgttctcaggtcgctcatcttttgttgcacttgcaatctttggggtcattctgttggcctgtctcatcgcattcctcatttggactcagaagcgaagacagaggcagcggctctcaggtctgaacaaaattacggtctctctaatgtttctatgggagaagaagcctctctggataataaaacaaaaaaattacattcaagtatcagttggccagaaagagggaacctagaagaggtttaagcagtttctccgaaacagggaacaagaattcagagaagaaaaggcacattggctgtactgatgatacctgcactcgctatgtatgtttaatgggggacagtagagaattgatagtttagaaggagtatgcttatatggttctggatgaatcctgtatccccccaaacatttattttctcttactatatacttattactaatttaactcttctgtcaagccatgtgctaggttctgaagatggttcagacttggataaccaagtgcttttgttttcatggaatttccagtttagtggaagagataaatatgtaaacaaataaatggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggggttggcgggcccccctcgaggtcgacggtatcgataagcttgatatcgaattcgtgagccagaggacgagactagagatggatgatgactacgttatgcttgcactgctggggaaaagcacacatagggagggaacgttttattatgacccagtccctaacctatgacctctgttatcagttttctcaggaggagagaattctgtccatcaaattcaataccgggagatgaattcttgcctgaaagcagatgaaacggatatgctaaatccctcaggtccgtgggttctttgaggggctgtagccctggggttcagatcagcagctgcagttgaggttgaggcatgctactttgcatagcagtagaaagaaatctcaactgtaataggaagcttgggatgcatatgaggaagaaaggcaagaatgaactacaaattattcttagggaagataaaaattgcagtcatggggagacctctggctgagagggccgtgattatttctgacagagggattatggagtagaatatgatggcttggaccttttttcactaaaacaagtcagtcttctcaaaggtagtttagcttttcatatatctttcacagtttcttccattcccatttcctgccattttcctttctctaacttttatttattatattttttcctaaaagtttaaattttctatatctttatcccttcagaagccatccctagtcacaggactagtctcatttcccattatgtaatgcttctttctctgtctgttgacttctatttagaaccagtgcactaaatctgcctttaggaacatacctctgctaggttgcaagaaatatcccattccccactcactctgtgaagactcaatgcttctcaatattccttacctcctgagagggacttgcctcacttctttaatccaagggactcgatttttgccaaaactaagtcaggaaaacctacataagacataggaaagacttgctgtgcttcttaaaccccactgtttgttttcctaattgtgaacagtatttttaaagttaacaagagagcttctaaggcacttgaggggagatctgatttatttcccagtaattattttcttcctttcagaaaattccactgaataagatggttttaacggatgtgggactaatttttgtgtctaaatctcttcctatttctggatgaaaaaaaggagaccactctgaagtacaatgaaaaggaaaatgggaattataacctggtgaggtgagtaggaagaatttattcatcattgctgaaaacaggtacattccttttgaaagttgagaactcctctggtattagaaaaaaaaaaagaacgtatatacacatatatttccatgtctatgtttatgtttgtaaatccatattcagaatatgcaacaactttttataactatgacttcagtccatcttttagttacatatatattctaaacaacaactattgctaagagaagctgggtaagtaaatgtgaataaatcttctaaagatattacaggaagttcctgctgcggctcagtgggttaaagacttgatgtctttgtgaagatgagggctcgagccctggcctcactcagtgagttaaggatctagcattgctgtaagctgcagcgtaggttgcagatagggctcagatccagtgttgctgtggctgtggcctcagttgcagctctgattcaacccttaggcgaggaacttccatatgcagcaaatgtggccattaaaaaaaaaaaaaacattataggagtcatttcataaaagagataagacgtttctatagttatatagtgcatactctggtaaagatagtataggatactataggaatatagaaagcttgcctatgaaaatttgggaagattgtggaaaagacatctcaaaatatggcatagaaaagaatcatatctttgaggaacagtaagtttttcattcaaaaccgtgtattgaacatacttgtggtgacaagtggtgtcctgagtactaaaaattcagtgataaaagatgctcttgacaaagacatggctgttgaatagaaggtctcactgtcaatgtgtgggaattatggacagcctatgtggacacagggaatagatgagactctaggctggaaggctgcattgagcccaataatgaatggtcctgtctgatatatttcatgctcatattttattttagggactattggggaggtggtgggttttggaagattaagctgaggcaagacacaatcagattgccttttataatttactttcaggaggaaagtctaactaaaaaaagaattcgatatcaagcttatcgataccgtcgacctcgaggaggcccgcctgcccttttggggggggggggggggggggggggggggggggggggggggggggggggggggggggggggggggggggggggggggggggggggggggggggggggggggcagcaccaattttattattggcgggaataaagagaaaaatgtaatttcaaagattgctgttggaaatgaggggtgtggtagcttttggagaaagcattctggagacttctattaattttttttttttaagtgcttcaaagatcctttgatccaacaattctactcctaaaaatttcttccatacagataaagccatttgtctgtatataacaaatagaagagaattcctttttgcagccttgttagtagtgcccccaaactggaaacaaagtgaatatcagtcagtggggtagcggctggaaaaattttagtgcacccaaccaacaaagaaaaaccatgcacaaaaattcaataaatatcatctcacttttgtgttcatgttattgaatataattaaacataatgtttacatctataaaattatcatatgtatacatgtaaagaaacattaaaacatttttaacagactgtaaacttgaggactgtgaatgacttttgattgataatctcaaacatatggatactattctgatgtaataaataatgattaaattttttccctaaagagtaatcactactgaatcgttgcctcagaatcatatggaggtgcttttaaaaaaggcatttctgcactgttgttctctggaatagaagtaattcttatgtacactgaagtttgaaaatcattgcatttaagtgttctgttcaggaaagtagtgtgctttttaatatttgtgagtgaatgagtaacacaatacattatatcacattttaatgtaattctacacatgtgcatatgaagagaaaagtaacatttttttctatttatgtctttagttcagcctttaagataccttgatgaagacctggactattgaatgagcaagaatctgcctcttacactgaagattacaatacagtcctctgtctcctggtattccaaagactgctgttgaatttctaaaaaatagattggtgaatgtgactactcaaagttgtatgtaagactttcaagggcattaaataaaaaagaatattgctgattcttgttcttgattttctgaatttctgaatctcttattgggcttctaatttaaaaaaaaatatctgggcgcccgcagatatcgaactcttgggcagtgtgaccaaacgaagacatatccaatcaagcatgcaaatggaccagcccactgtactagcacgctgtggcagccaatctgaccgagaaagcagacaaccgcagggagcaacg

SEQ ID NO: 16 - DNA, 55 bp, Sus scrofa
ggtcgccaccatggtgagcaagggcgaggagctgttcaccggggtggtgcccatc

SEQ ID NO: 17 - DNA, 54 bp, Sus scrofa
ggtcgccaccatggccatgagcaagggcgaggagctgttcaccggggtggtgcc

SEQ ID NO: 18 - DNA, 48 bp, Sus scrofa
ggtcgccaccatggtgagcaagggcgaggagctgttcaccggggtggt

SEQ ID NO: 19 - DNA, 55 bp, Sus scrofa
ggtcgccaccatggttgagcaagggcgaggagctgttcaccggggtggtgcccat

SEQ ID NO: 20 - DNA, 43 bp, Sus scrofa
tgcagggaactacagtgcggcactgtggtttccctcctggggg

SEQ ID NO: 21 - DNA, 60 bp, Sus scrofa
tgcagggaactacagtgcggcactgtaaaccactactactgtggtttccctcctgggggg

SEQ ID NO: 22 - DNA, 41 bp, Sus scrofa
tgcagggaactacagtgcggctgtggtttccctcctggggg

SEQ ID NO: 23 - DNA, 46 bp, Sus scrofa
tgcagggaactacagtgcggaactactgtggtttccctcctggggg

SEQ ID NO: 24 - DNA, 31 bp, Sus scrofa
gaaacccaggctggttggaggggacattccc

SEQ ID NO: 25 - DNA, 24 bp, Sus scrofa
gaaacccaggctggggacattccc

SEQ ID NO: 26 - DNA, 13 bp, Sus scrofa
aggggacattccc

SEQ ID NO: 27 - DNA, 13 bp, Sus scrofa
gaaacccattccc

SEQ ID NO: 28 - DNA, 31 bp, Sus scrofa
ggtcgccaccatggtgagcaagggcgaggag

SEQ ID NO: 29 - DNA, 32 bp, Sus scrofa
ggtcgccaccatggctgagcaagggcgaggag

SEQ ID NO: 30 - DNA, 29 bp, Sus scrofa
ggtcgccaccatggtgagagggcgaggag

SEQ ID NO: 31 - DNA, 32 bp, Sus scrofa
ggtcgccaccatggttgagcaagggcgaggag

SEQ ID NO: 32 - DNA, 48 bp, Sus scrofa
ggtcgccaccatggtgagcaagggcgaggagaacccaggctggttgga

SEQ ID NO: 33 - DNA, 49 bp, Sus scrofa
tgctgtgcagggaactacagtgcggcactgtggtttccctcctgggggg

SEQ ID NO: 34 - DNA, 38 bp, Sus scrofa
tgctgtgcagggaactctgtggtttccctcctgggggg

SEQ ID NO: 35 - DNA, 22 bp, Sus scrofa
ctgtggtttccctcctgggggg

SEQ ID NO: 36 - DNA, 23 bp, Sus scrofa
actgtggtttccctcctgggggg

SEQ ID NO: 37 - DNA, 50 bp, Sus scrofa
tgctgtgcagggaactacagtgcggcaactgtggtttccctcctgggggg

SEQ ID NO: 38 - DNA, 10 bp, Sus scrofa
tcctgggggg

SEQ ID NO: 39 - DNA, 8 bp, Sus scrofa
ctgggggg

SEQ ID NO: 40 - DNA, 52 bp, Sus scrofa
agagagcagagccagcgactcgcccagcgacatggggtacctgccgtttgtg

SEQ ID NO: 41 - DNA, 33 bp, Sus scrofa
agagagcagagccagcgactcgcccagcgagat

SEQ ID NO: 42 - DNA, 30 bp, Sus scrofa
agagagcagagccagcgactcgcccagcga

SEQ ID NO: 43 - DNA, 50 bp, Sus scrofa
agagccagcctcgcccagcaggggtaccatggggtacctgccgtttgtgt

SEQ ID NO: 44 - DNA, 53 bp, Sus scrofa
agagagcagagccagcgactcgcccagcgagcagtgggtacctgccgtttgtg

SEQ ID NO: 45 - DNA, 53 bp, Sus scrofa
agagagcagagccagcgactcgcccagcgatcagtgggtacctgccgtttgtg

SEQ ID NO: 46 - DNA, 53 bp, Sus scrofa
agagagcagagccagcgactcgcccagcgaacatggggtacctgccgtttgtg

SEQ ID NO: 47 - DNA, 4990 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttggaggggacattccctgctctggtcgtgttgaagtacaacatggagacacgtggggcaccgtctgtgattctgacttctctctggaggcggccagcgtgctgtgcagggaactacagtgcggcactgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 48 - DNA, 24 bp, Artificial sequence
  Synthetic oligonucleotide
caccggaaacccaggctggttgga

SEQ ID NO: 49 - DNA, 24 bp, Artificial sequence
  Synthetic oligonucleotide
aaactccaaccagcctgggtttcc

SEQ ID NO: 50 - DNA, 24 bp, Artificial sequence
  Synthetic oligonucleotide
caccggaactacagtgcggcactg

SEQ ID NO: 51 - DNA, 24 bp, Artificial sequence
  Synthetic oligonucleotide
aaaccagtgccgcactgtagttcc

SEQ ID NO: 52 - DNA, 25 bp, Artificial sequence
  Synthetic oligonucleotide
caccgcagtagcaccccgccctgac

SEQ ID NO: 53 - DNA, 25 bp, Artificial sequence
  Synthetic oligonucleotide
aaacgtcagggcggggtgctactgc

SEQ ID NO: 54 - DNA, 25 bp, Artificial sequence
  Synthetic oligonucleotide
caccgtgtagccacagcagggacgt

SEQ ID NO: 55 - DNA, 25 bp, Artificial sequence
  Synthetic oligonucleotide
aaacacgtccctgctgtggctacac

SEQ ID NO: 56 - DNA, 25 bp, Artificial sequence
  Synthetic oligonucleotide
caccgccagcctcgcccagcgacat

SEQ ID NO: 57 - DNA, 25 bp, Artificial sequence
  Synthetic oligonucleotide
aaacatgtcgctgggcgaggctggc

SEQ ID NO: 58 - DNA, 25 bp, Artificial sequence
  Synthetic oligonucleotide
caccgcagctgcagcatatatttaa

SEQ ID NO: 59 - DNA, 25 bp, Artificial sequence
  Synthetic oligonucleotide
aaacttaaatatatgctgcagctgc

SEQ ID NO: 60 - DNA, 25 bp, Artificial sequence
  Synthetic oligonucleotide
caccgctttcatttatctgaactca

SEQ ID NO: 61 - DNA, 25 bp, Artificial sequence
  Synthetic oligonucleotide
aaactgagttcagataaatgaaagc

SEQ ID NO: 62 - DNA, 25 bp, Artificial sequence
  Synthetic oligonucleotide
caccgttatctgaactcagggtccc

SEQ ID NO: 63 - DNA, 25 bp, Artificial sequence
  Synthetic oligonucleotide
aaacgggaccctgagttcagataac

SEQ ID NO: 64 - DNA, 25 bp, Artificial sequence
  Synthetic oligonucleotide
caccgctcctcgcccttgctcacca

SEQ ID NO: 65 - DNA, 25 bp, Artificial sequence
  Synthetic oligonucleotide
aaactggtgagcaagggcgaggagc

SEQ ID NO: 66 - DNA, 25 bp, Artificial sequence
  Synthetic oligonucleotide
caccggaccaggatgggcaccaccc

SEQ ID NO: 67 - DNA, 25 bp, Artificial sequence
  Synthetic oligonucleotide
aaacgggtggtgcccatcctggtcc

SEQ ID NO: 68 - DNA, 24 bp, Artificial sequence
  Synthetic oligonucleotide
ttgttggaaggctcactgtccttg

SEQ ID NO: 69 - DNA, 20 bp, Artificial sequence
  Synthetic oligonucleotide
acaactaaggtggggcaaag

SEQ ID NO: 70 - DNA, 24 bp, Artificial sequence
  Synthetic oligonucleotide
ttgttggaaggctcactgtccttg

SEQ ID NO: 71 - DNA, 23 bp, Artificial sequence
  Synthetic oligonucleotide
ggagctcaacattcttgggtcct

SEQ ID NO: 72 - DNA, 23 bp, Artificial sequence
  Synthetic oligonucleotide
ggcaaaattttcatgctgaggtg

SEQ ID NO: 73 - DNA, 23 bp, Artificial sequence
  Synthetic oligonucleotide
gcacatcacttcgggttacagtg

SEQ ID NO: 74 - DNA, 23 bp, Artificial sequence
  Synthetic oligonucleotide
cccaagtatcttcagttctgcag

SEQ ID NO: 75 - DNA, 23 bp, Artificial sequence
  Synthetic oligonucleotide
tacaggtaggagagcctgttttg

SEQ ID NO: 76 - DNA, 23 bp, Artificial sequence
  Synthetic oligonucleotide
cccaagtatcttcagttctgcag

SEQ ID NO: 77 - DNA, 23 bp, Artificial sequence
  Synthetic oligonucleotide
ctcaaaaggatgtaaaccctgga

SEQ ID NO: 78 - DNA, 22 bp, Artificial sequence
  Synthetic oligonucleotide
tgttgatgtggtttgtttgccc

SEQ ID NO: 79 - DNA, 23 bp, Artificial sequence
  Synthetic oligonucleotide
tacaggtaggagagcctgttttg

SEQ ID NO: 80 - DNA, 23 bp, Artificial sequence
  Synthetic oligonucleotide
ggaggtctagaatcggctaagcc

SEQ ID NO: 81 - DNA, 20 bp, Artificial sequence
  Synthetic oligonucleotide
ggctacatgtcccgtcaggg

SEQ ID NO: 82 - DNA, 21 bp, Artificial sequence
  Synthetic oligonucleotide
gcaggccactaggcagatgaa

SEQ ID NO: 83 - DNA, 23 bp, Artificial sequence
  Synthetic oligonucleotide
gagctgacacccaagaagttcct

SEQ ID NO: 84 - DNA, 22 bp, Artificial sequence
  Synthetic oligonucleotide
ggctctagagcctctgctaacc

SEQ ID NO: 85 - DNA, 22 bp, Artificial sequence
  Synthetic oligonucleotide
ggacttgaagaagtcgtgctgc

SEQ ID NO: 86 - DNA, 44 bp, Artificial sequence
  Synthetic oligonucleotide
taatacgactcactatagggagaatggactataaggaccacgac

SEQ ID NO: 87 - DNA, 21 bp, Artificial sequence
  Synthetic oligonucleotide
gcgagctctaggaattcttac

SEQ ID NO: 88 - DNA, 40 bp, Artificial sequence
  Synthetic oligonucleotide
ttaatacgactcactataggctcctcgcccttgctcacca

SEQ ID NO: 89 - DNA, 20 bp, Artificial sequence
  Synthetic oligonucleotide
aaaagcaccgactcggtgcc

SEQ ID NO: 90 - DNA, 38 bp, Artificial sequence
  Synthetic oligonucleotide
ttaatacgactcactataggaaacccaggctggttgga

SEQ ID NO: 91 - DNA, 20 bp, Artificial sequence
  Synthetic oligonucleotide
aaaagcaccgactcggtgcc

SEQ ID NO: 92 - DNA, 38 bp, Artificial sequence
  Synthetic oligonucleotide
ttaatacgactcactataggaactacagtgcggcactg

SEQ ID NO: 93 - DNA, 20 bp, Artificial sequence
  Synthetic oligonucleotide
aaaagcaccgactcggtgcc

SEQ ID NO: 94 - DNA, 40 bp, Artificial sequence
  Synthetic oligonucleotide
ttaatacgactcactataggccagcctcgcccagcgacat

SEQ ID NO: 95 - DNA, 20 bp, Artificial sequence
  Synthetic oligonucleotide
aaaagcaccgactcggtgcc

SEQ ID NO: 96 - DNA, 40 bp, Artificial sequence
  Synthetic oligonucleotide
ttaatacgactcactataggcagctgcagcatatatttaa

SEQ ID NO: 97 - DNA, 20 bp, Artificial sequence
  Synthetic oligonucleotide
aaaagcaccgactcggtgcc

SEQ ID NO: 98 - DNA, 3484 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggcacattccctgctctggtcgtgttgaagtacaacatggagacacgtggggcaccgtctgtgattctgacttctctctggaggcggccagcgtgctgtgcagggaactacagtgcggcactgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 99 - DNA, 4997 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttggaggggacattccctgctctggtcgtgttgaagtacaacatggagacacgtggggcaccgtctgtgattctgacttctctctggaggcggccagcgtgctgtgcagggaactacagtgcggctactactactgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 100 - DNA, 3710 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaagggaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 101 - DNA, 3617 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggattgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 102 - DNA, 4979 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttggaggggacattccctgctctggtcgtgttgaagtacaacatggagacacgtggggcaccgtctgtgattctgacttctctctggaggcggccagcgtgctgtgcagggaactctgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 103 - DNA, 4615 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaagaaggaaaatattggaatcatattctccctcaccgaaatgctatttttcagcccacaggaaacccaggctggttggaggggacattccctgctctggtcgtgttgaagtacaacatggagacacgtggggcaccgtctgtgattctgacttctctctggaggcggccagcgtgctgtgcagggaactacagtgcggcacagtgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 104 - DNA, 4866 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttctgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 105 - DNA, 4867 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttactgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 106 - DNA, 4991 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttggaggggacattccctgctctggtcgtgttgaagtacaacatggagacacgtggggcaccgtctgtgattctgacttctctctggaggcggccagcgtgctgtgcagggaactacagtgcggcaactgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 107 - DNA, 4860 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttggagggtcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 108 - DNA, 4858 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttggagggctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 109 - DNA, 3523 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 110 - DNA, 3603 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgttgtggagaattccacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttatatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgttgtggagaattccacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttggaggggacattccctgctctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 111 - DNA, 4962 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttggaggggacattccctgctctggtcgtgttgaagtacaacatggagacacgtggggcaccgtctgtgattctgacttctctctggaggcggccagcgtgctgtgcagggaactacagtgcgtcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 112 - DNA, 3603 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttggaggggacattccctgctctggtcgtgttgaagtacaacatggagacacgtggggcaccgtctgtgattctgacttctctctggaggcggccagcgtgctgtgcagggaactacagtgcgattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 113 - DNA, 3619 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttggaggggacattccctgctctggtcgtgttgaagtacaacatggagacacgtggggcaccgtctgtgattctgacttctctctggaggcagccagcgtgctttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 114 - DNA, 3270 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 115 - DNA, 7 bp, Sus scrofa
tactact

SEQ ID NO: 116 - DNA, 12 bp, Sus scrofa
tgtggagaattc

SEQ ID NO: 117 - DNA, 11 bp, Sus scrofa
agccagcgtgc

SEQ ID NO: 118 - DNA, 8532 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttggagctgacatgccctgctctggtcgtgttgaagtaaaacatgcagacacgtggggctccgtctgtgattctgacttctctctgcatgcggccaacgtgctgtgcagggaactaaattgcggcgatgcgatttccctctcggtgggagatcactttggaaaaggaaatggactgacctgggctgaaaaattccagtgtgaggggagcgagacccaccttgcactctgcccaatagtacaacaccctgaagacacatgtatccacagcagggaagtcggcgtagtctgctcaagtaagagtttactgaaaataacactcttaaaatcttgttatgtttttattcataatgtgaatgagtagtagtggaaaataactaccagtttcctaagcttataacttcgtatagcatacattatacgaagttataagcctgcaggaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaccatggccatgattgaacaggatggcctgcatgcaggttctccagctgcctgggtggagagactgtttggctatgactgggcacagcagaccattggttgctctgatgcagcagtgttcagactgtcagcccagggcaggccagtcctgtttgtgaagacagacctcagtggggctctcaatgagctgcaggatgaggctgccagactctcctggctggcaacaactggggtcccctgtgcagctgtcctggatgtggtcacagaagctggaagggactggctcctgctgggtgaggtgcctgggcaggacctcctgtcctctcacctggctccagctgagaaagtgtcaatcatggctgatgccatgagaagactccacaccctggacccagccacctgcccctttgaccaccaggccaagcacaggattgagagggccagaaccaggatggaggctggcctggtggaccaggatgacctggatgaagaacaccagggcctggcccctgctgaactgtttgccaggctcaaggcatccatgccagatggtgaggacctggtggtgactcatggggatgcctgcctgcccaacatcatggtggaaaatggaaggttctctggcttcattgactgtggcaggctgggagtggctgacaggtaccaggacattgccctggcaaccagggacattgcagaagagctggggggagaatgggcagacaggttcctggtgctctatggcattgcagcccctgactcccagagaattgccttctacagactgctggatgagttcttctaaatcgattataatcagccataccacatttgtagaggttttacttgctttaaaaaacctcccacacctccccctgaacctgaaacataaaatgaatgcaatgttgttgttaaacttgtttattgcagcttataatggttacaaataaagcaatagcatcacaaatttcacaaataaagcatttttttcactgcattctagttgtggtttgtccaaactcatcaatgtggaaaataactaccagtttcctaagcttataacttcgtatagcatacattatacgaagttataagctagacaaaagtatcttaccccaatggtagccctgtacccaataaaagtaggtgttcagtttcatatcctatgaaataccctcttgatacttttactttgcatgaggatttagaagaaaaaagttttactataatccttaacttaggaaattcttttgaattggaaatgaaacacaaattgcttttcattgatatgccatatgattatatgaataaaacatgaaatcttcatattggattctagtatatacccaagtaaatattttttccctagaagagtgccaagtgtgttaaaaccttttggtttaataaagcagaaaaaaataaactctaaaaatcataattaaaaatgaaatgcttttatttatagcaattaactacaacatgtttagacttacatactattaaatataatatatttaagatcccctcatgataaatatgttcattattttgtaggctgttgatgcactaatatgtatgtagattactttgtgaattgcccttaataaaatttaaaactttaggctagtaaacctgtaacactcaacttagttctgaactatctcactattcttttgcaagaatttacttaggtaatgccaactaatttattccaaggccaaaaagatgacaatgtcttatatattataaaaactaataaaaaccattttaaaacctagtataaatttaaaggtacttgctcttctggttcatctcttcttttgtttacttctgctttcaaaaacttatttattgtgaccatattctttacttccatttattgttataatttataagatactatacttgcaagcaataaatgttatctttttagcttttaaatggtctcatttgaaaagaatatataattagtaagtcatagctactttaaataaaaacttattctttaagagattaaacacttctccaagtgatctgtttttctttaattaaaacgttattaactcccaaaatgatgttattgtttttttataatcttaaataccaataattaccaggtctattttgattttgatacaggataaaaactactattaattacttaagaatgtgttcttttttatatgtaccattttcatgatcaaagttggtgatatgactgaggttttgattattattaaacagatagttaatatgatatattcctcatttttccaaatgaaaggaaaaatgtcttatatggaggaaaagattggggcagggggattagtaaattattacttaaatatctgaataggaggatttttcaatgaaaggataaaggaagaatgattgtatcatctgaatctttccctccctttcctggagtttgtcctttcaacccagtatacctaccactcccttcatcacctactttcccattacagtccctatgtgttgggtggtaactattttgttttggtgttaatatccaagtttcccttaataacacctagtgaatggaggaaggatgagcatacctacccatcagacatatttagccaccatatttaatcaacaagcatgaagaaaggaagctagcctctccccttcctttcctcctgcctctctctctcttctctgtcctcgctccctttcttcccatcaatattttcagagcacctcttatgcgccaggcattgggatactcaaactggaggaaacaagaaaaaaaaaaaaaaaaggcgaagacctcagggaaatttatattgctgctatatttttttgagcctagtgtaaattaaaattccttaatgctgtgccttttaaaaacacaaataagcaaaatagtttatttcttcaacagttaaatccttagggtaggaaagtgattcaggatctattgctactattaactcttctttcattttcacacaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 119 - DNA, 4538 bp, Sus scrofa
tatagatgacaaggctttgtgtctgataggggccagcgaactcagtaaagagggaagatgagaaagataatggcaagaatttatccctgaagtgtagttttgacaaaccagtcacaaagaggtctaagaaattttggtcacaaagttgttttgaatcccaggcattttatttgcaatgattgcatatgttctggaaaggacatctgaacctaagaaatagttcatttgcattgtgttatattttactaaggtctgagaaataatcttgagatgagaatgaactctacttcttcagagtctggaaggaataaattatgaaaatgtattaatgcttctttaaaccatattgtatatttatctattactaaacaaaaagaagtagctctatttatttatttatttatttatttatttatgtcttttgtctctttagggccacacctgtggcatatggaggttcccaggctagaggtccaattggagatgtagcagccagcctatgccagagccaccgcaacacgggatctgagccacgtctgtgacttacaccacagctcacagcaacgcctgatcctcaacccactgagcgaggccagggatcgaacccatgtcctcatggatgctagttgggttcgttaactgctgagccatgatgggaactccaaattaattatttcttatatttgttcttcatatattcatttctatagaaagaaataaatacagattcagttaatgatggcaggtaaaagcttaacttattaatcaaaggagttaatccaggcacaaaaattcaattcatggctctctgttaaaatttaggtataggtttagcaggaagaaaaggttagtagatgcagactattacatttagaatggatggacaatgaagtcctactatacagcacagggaactatatccaatctcttgggatagaatatgatggaagacaaaatcagaacaagagagtatatatatatgtgtgtgtgtgtgtgtgtgtgtgtgtgtgtgtgtgtgtgtgtgtgactgggtcaccctgcggcacagcagaaattggcagaacattgtaaatcaactatactttaataggaaaaatacttttaagggctaaatttccaatattctaaccatgtacacagagtaaatgtcataaggatgccagtctgtgtagagattgatgtgttactagcagattcatgaaataaaggctgaggatgtagtccccaagtcacttctgagtggaagaatttctcctttgtcctggactcaaatattttaggataaaggaaaaaagaagatatttatagaagggacttgttttcaagtacttgacaaaatttcaccattaaagagaaatttgtgggagttcccatcgtggctcagtggaaacaaatccaactaggaaccatgaggttgtgggtttgatccctggcctcactcagtgggttaaggatccggtgttgccgtgagctgtggtgtaggttgcagacacggttctgatcctgcgttgctgtggctgtggctgtggtgtaggccagcagcaaacagctctgattagacccctagcctggaaacctccatatgccacaggtgcagccctaaaaagacaaaaaaagagaaaagacaaaaaaaaaaaaaaaaaaaaaaaaaaaaaaaaaaaaaaaaaaaagaacccccagaggtatttatttgtttttgccttttttcactgactgttctttgtttgtttgtttgagactgatctagaagactagagattacaagaaatatggatttggctcactctaagaaactgctttcattccaaggtttgggtctatccaaaagtggaatagaatcatatgaatactagtttatgagtatttagtgagaggaatttcaagctcaaataatgattcagcaagattaaattaaggagggaattttccttgtggctgagtgggttaaggacccaatgttgtctctgtgaggatgtaggttccatcctgggctttgctcattaggttaaggatctggcattgctgcagctcagacccagtgctgccctggttgtggcttaggccaaagctgcagctccaattcaatctctggcctgggaacctccatgtgctacaaggtgcggccttaaaaggaaaaaaaaaaaattaaatcaaggactcaagagtctttcattatttgtgttgtggaagctatatttgttttaaagtcttagttgtgtttagaaagcaagatgttcttcaactcaaatttgggagggaacttgtttcatacatttttaatggataagtggcaaaattttcatgctgaggtgatctatagtgttgtaatgcagaatatagtcagatcttgaacattttaggaagttggtgagggccaattgtgtatctgtgccatgctgataagaatgtcaagggatcacaagaattcgtgttatttgacagcagtcatctttaaaaggcatttgagaaagtccaatttcaaatgcatttcctttctttaaaagataaattgaagaaaataagtctttatttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtacctaggagttcccattgtggctcagtggtaacaaactcgactggtatccatgagggtatgggtttgatccctggccttgctcaatgggttaaggatccagcattgctgtgagctgtggtataggttgcagactctgctcaggtcccatgttgctgtgattgtggtgtaggctgactgctgcagcttcaatttgacccctagcccgggaatttccataggccacacgtgcagcactaaggaaggaaaaaaagaaaaaaaaaaaaaaagagtgggtgtgcctatagtgaagaacagatgtaaaagggaagtgaaagggattcccccattctgagggattgtgagaagtgtgccagaatattaacttcatttgacttgttacagggaaagtaaacttgactttcacggacctcctagttacctggtgcttactatatgtcttctcagagtacctgattcattcccagcctggttgacccatccccctatctctatggctatgtttatccagagcacatctatctaacactccagctgatcttcctgacacagctgtggcaaccctggatcctttaaccaactgtgccaggctggagatcaaacctaagcctctgcagcaacccaagctgctgcagtcagatttttaaccccctgtgccactgtgggtatctccgatattttgtatcttctgtgactgagtggtttgctgtttgcagggaaccagagtcagacactatccccgtgcaattcatcatcctcggacccatcaagctctattatttcagaagaaaatggtgttgcctgcataggtgagaatcagtgaccaacctatgaaaatgatctcaatcctctgaaatgcattttattcatgttttatttcctctttgcagggagtggtcaacttcgcctggtcgatggaggtggtcgttgtgctgggagagtagaggtctatcatgagggctcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcgg

SEQ ID NO: 120 - protein, 101 aa, Sus scrofa
PRLVGGDIPCSGRVEVQHGDTWGTVCDSDFSLEAASVLCRELQCGTVVSLLGGAHFGEGSGQIWAEEFQCEGHESHLSLCPVAPRPDGTCSHSRDVGVVCS

SEQ ID NO: 121 - protein, 100 aa, Homo sapiens
LRLVDGDSRCAGRVEIYHDGFWGTICDDGWDLSDAHVVCQKLGCGVAFNATVSAHFGEGSGPIWLDDLNCTGMESHLWQCPSRGWGQHDCRHKEDAGVIC

SEQ ID NO: 122 - DNA, 7278 bp, Sus scrofa
tcagagtaggaaagggggacctttcagggatgcactgggaaccccatctcatgggaaatggggagccacagaaggttttcaagcaggggagggactcaaaggcagcactttggtatgtgctcagctctatagctggtaaggctgctgatagggagcaagtctcaaacaagaaagcatcatgtgtgctggatgccttggagtaagtcgctgggcctctctgggctcccatttctgttaacttgggtgcagagacagggcagggaagtggggaaagagaaacagctatttatccttgaggggctgggaaagcagaacttagggtgtggccaagtgacagaggactttctggggcatgccatcaagtgaaggcattcctaggcacagcctgagctggggaagtgggctcctggctccctgctggcagctgccctgccctctcactgggttggccagcgtagcacgtggggaaacaggactgggttcccagcaggctctgagacccaggtaccctgaggttccccaggaggctcccgtgtgccctgccagcccactgtgtgccaccctcttaggctccagaagcagcgccagaacctgctatggacttcctgctcctgctcctcctcctggcttcatctgctctagcaggtaagttgttctggcttttttcttctcacagggcctggctcagcaaggccagcccttggctggcctgggcacactggactgtatctggtcacaggtttagctgctgaggttgaagaagggcatggggaagcagtgacagggagccatgaccctccctggggtgccccgtggggctgcattgctgaggacacggcagggttcgggggcccatctggagcacctcctatgtgacagatcaggaggtgaccctggcctgcatcacactccaccctgtcctgtccctccctcaggcctggcctcgtggacggtttccagccccgagaccgtgcagggcatcaagggctcctgcctcatcatcccctgcaccttcggcttcccggccaacgtggaggtgccccatggcatcacagccatctggtactatgactactcaggcaagcgcctggtagtgagccactccaggaacccaaaggtggtggagaaccacttccaaggccgggccctgctgttggggcaggttgagcagaggacgtgcagcctgctgctgaaggacctgcagccccaggactcgggctcctataacttccgctttgagatcagcgagggcaaccgctggtcagatgtcaaaggcacagttgtcaccgtgacaggtgaggagcagggtggcccagccactgcctgggctcccaggccacccctggccccactggctcacagttcccatcctggtctgaaagccaaccccagccccaatcttggccgcagttccagtcctagccctgtgccccctggcattcgcccctgtgaactacttgcattctccttctccttgaagctccagcctggagccagggtttccaaggcaggagggctcaggtgtggtgaacgggagtccccagcctcctctctcatccttagaggtgcccagcgtgcccaccattgccttgccagccaagctgcatgagggcatggaggtggacttcaactgctccactccctatgtgtgcccgacggagccggtcaacctacagtggcaaggccaggatcccacccgctccgtcacctcccacctccagaagcttgagccctcgggcaccagccacatggagaccctgcacatggccctgtcctggcaggaccatggccggatcctgagctgccaggtctcagcagccgaacgcaggatgcagaaggagattcacctccaagtgcagtgtgagtgcacagggatggggggacatgcccttcccactttggtggggcaggtggagagtgttaagccctaagcaccacggtgaaagtctccctttcatctcctgaacctgcagccaggctgggctgagaacgggagacagaaaaaccagagaatttggagtctggcaacaggggccctgggatttagcccagctctggaggaggccagggaggaaggggatcacccctttcctgcctcaggccacttgcacacatgaccggggtaacatctggacgtggcaaggaggcaatgggagaaagccggagttttcacttgattatgcatcttcccatcacagtaaaagagggcagcctgttctagaaccaccactatacacagacgtcaggctgctggctctgccatccactagatgagcaaacttcaggcaagttccttgcctcttggagcctctgttttcatatctgtatactggcagtaacagtacatatgtttccatctcacaggattgttatgaaaattaagagtgcttggtacctattaagcactcaaaaataccaacttcttggacttccctggtggtacagtgggttaaggacctagtgttgtcactgctgtgacatgggttcgatccctggcccaggaacttctgcatgtcatgggggcagccaaaaatattagcttctatcattttcatatttatatagattaagcacttgaaaacatgatcttttattagcatagttacatagagagaggctttggaacaagatttaaagacaggtcatttcttcgtattgtggacaccctcccagcaaaggagttaaccattcttagagctactttgacactgcttttggccccctgcattaggtggcggttccttgtcaaccgctaccacaagccaggcgaaaaagcagtgcggtgcccatgacccttaaatagaagggagggctatgggaggcatcatgaggcagaaaaccaaggacggtagtgcaggctggctgtaggccaaccctttgcaatatcaggctctcggtggagaaggaaggggtcagtattcctcacaaggtggggagcagggcctgccctggacaggggaactagtgggacttaagatttatctgggtggaattcccctggtggcgcagcagtaacggaccccactagtacccatgaggacaagaattcaatccctggccctgctcagtgggttaaggatctggtgctcccgcgagctgcggtataggtcgcagatgcggcttggatctggtgttgctgtggctgtggcataggccggctgctgtagctctgattggaccttagcctgggaacttccattcgctgcgtgtgcggccctaaaagaccccgatccggcaaaaaaaaagatttatctggatgggctttctagggcagagggttgaaagaggagagggacatgccccctgccctccctgggcccttgctcagcatccccccatcatgccttgcagatgcccccaagggtgtggagatccttttcagccactccggacggaacgtccttccaggtgatctggtcaccctcagctgccaggtgaatagcagcaaccctcaggtcagttccgtgcagtgggtcaaggatgggacgaagctcaaagaccagaaacgtgtacttcagttgcgccgggcagcctgggctgatgctggcgtctacacctgccaagccgggaatgccgtgggctcttcagtctcacccccggtcagcctccacgtcttcagtgagtcctgaaggagcctgggtcttgatcagagggcagcgttggggttaaggaaaccaggccaaggcacctctggggatgcgggtggggagtcccaacccggcctcagacaggtgatcgcctgggatgtatggaggatcccagtgaacagggccaagagatgcggagccaatccgagaggcgcctccgcagaaggggcggggcgtagactgtgcagagcttaaggctgattggctgcaaccatgaggggttgggcctggaagtcctctgggtatcccccagcgtgtcctatgctgtccccacagtggctgaggtccaggtaagccctgtgggctccatcctggagaaccagacggtgacgctggcctgcaatacacctaaggaagcgcccagcgagctgcgctacagctggtacaagaaccacgccctgctggagggctctcacagccgcaccctccggctgcactcagtcaccagggcggattcgggcttctacttctgcgaggtgcagaacgcccggggcagagagcgctctccccctgtcagcgtggtggtcagccgtaagtgagggtggagggaccggtggggagtctggacctgggaactgagccaaaaagtaggaggccctgcaggacgtttggggtcctgggcccaggtgcccttgagctcacatcttggagaggccttgaccttcctgggcctcagtttcatcctatttttattactattttttaaaatttattttgtctttgtttgttttgtcttttctagggcatatggaggttcccaggctaggggtctaactggcactgtagccacccgcctacgccacagccacagcaatgcaggatcctagctacatctgtgacctacaccacagctcatggcaacgccggatccttaacccactgagtgaggccagggatcaaacccgcaacctcatggttcctagttggtttcgttcaccactgagccacgacaggaactgctatttattttgtcttttgtcttccctagattctccatttgttaacattttgatacctttgcttttctctctctgtgtattatgtatgtgtgtgtataggtgctcacacatgcacacacatgcatgtatgtgcatatgtacatatacacatgaaagtgtgcatacaggcatacattttctccacataaacacatagacatttatactctctctagccacatgcatatacacacatgcactctatatgtatacacacacacacatgctatctattatcaatctatctatttatctacctatctatctatctatcaatcctcctgaacgtgcaggcataatgcctctttactccttaatttctcagcatgtatttcctaaggtcattcttgtgcataactgcagtacaataactatagtcaggaaacttaacattgataccattgatataagactcttatccaatccacaacccacatttaaattttaccaggtggcccaacaatgtcctttatggcaattatttttctgctccaggatttggtctgggatcctgccttgcattcagttgtcgagtgtctttatcctccctcagtctgggacagctcctaagtctctccttgttttccttgacattgaatcccaaagatcacaggcctgttgctctgtgctgtcctcagtgtgcgttgatctgagtggggcagactgaggctatgcacgtgagcagggacatcgtggctgtgatgctgtgtctgtctccccctgcatcacaacaggaggcacgtggggtctgtttgtcccattgctgggagcattcactttgatcatttgctgcagtgctggccgctggctggtttctttgttagatttcccgtctttatttttatttcattttatgtttttagggccgcacctgcggcatatggaggttcccaggctaggggtcgaattagagctatagccactggcctatgccacagccacagcaatgccagatccgagttggatctgcagcctacaccacagctcacagcaacaccagatccttaacccactgagcaaggccaggaactgaacctgtgtcctcatgggtactagtcagattcgtttctgctgagccacgacaggaactctagttttcccgtctttaaaatgggtccctggagccccattctcagggttgccacaaggattgagtaggatggcagggcaatttacccagttcagggacaggcactcaggagccattggggtgccccctttcccagggcattatgggacaaaaatgggtgcaggggtcataggctataaagggagatccaggagtgcccctgttgagagtgacaggccttccttcatccccacgcagacccacccctcaccccggacctaactgccttcctggagacacaggcggggctggtgggcatcctccaatgctctgtggtcagcgagcccccagctactctggtgttgtcacacgggggcctcatcttggcctctacctccggggagggtgaccacagcccacgcttcagtgtcgcctctgcccccaactccctgcgcctggagattcaagacctggggccaacagacagtggggaatacatgtgctcagccagcagttctcttgggaatgcgtcctccaccctggacttccatgccaatggtaagacggccacagcagggctagtggagggagcctcaggtttgttctgccccctcctcctgggagtccagagacctcaaccaagggcctgaactagctgtctagaaaaaaggaatatcaaaaatctgagctgtaccgtcatcccaggctcagaggttcctagaagtcaaggcaaaaaggggaacatggtcgattgcattattccttttttacccattctgactctgaacttggagggttcctcgatatgcatttatttgtctttaaattattgaacatttgggggcagactctgtgctggggtcttctagttcttggtgcatatgctgctcttccctccttctgagacaggcagagctaatcaatcaagccaggtttttaacataagcctgggattgggctcagaatccttctcaatcatgctgcagccagctacgaccaattagatccaaatgggcattcaaagccaaactctcctgatcattgagagattcgcagagctgatgtaatgaagctcagacctccctacattgtatggggagaggggaaccctggcgggcttcctgcagtggggggcatggaaccgcggatgtgtgggggagctgagcctccctctctccctctctgccagcagcccgcctcctcatcagcccagcagcagaggtggtggaagggcaggcggtgacactgagctgcaggagcagcctgagcctgatgcctgacacccgtttttcctggtaccggaacggggccctgcttctcgaggggcccagcagcagcctcctgctcccagcagcctccagcacagatgccggctcataccactgccgggcccagagcagccacagcgccagtgggccctcctcacctgctgttctcaccgtgctctgtgagtagccggcctgccagccgtgcctgctagggggagggtcagctcccacctcctccaggccctcctgactctgccctgtctgggagtcttgataaggaggcagcccaggcaccc

SEQ ID NO: 123 - DNA, 11527 bp, Sus scrofa
  misc_feature (43)..(43): n is a, c, g, or t
  misc_feature (46)..(46): n is a, c, g, or t
gcatatgaggttccagggctaggggtctaatcggagctgtagncancagtctacaccagagccacagcaacgcgggatctgagctgcggctgcaacctacactaaccactgcgccatgacaggaactccttgccttctcttttttccaggaaagttctctgggttgaggctggtcttggggtggaacaggctgagccaataagctggactctggtgggacaaccccctgggtactggggctgggctaagtataggtgacacccatgtaatggaatttggggcggttctagaaagagtcctctcgaaaatccctgctcctttctggggtcagtgtgtcaaaactcccaccattcctcctgtaggttcagcactggcctttccagggccctgaatgggaacagtgggtgagttggggacatgggcactggtgatgtctcagctatagagggagctgatcctgatgcccagtgtagctctgggccctggggtcacaccagcctccaaacgtgtccagcaggctgccactcagtctagcatcaggccacagcatccgggtggtggtgcccagcctggccagcatgatcctctcctgtggccaaggaccaggtgtggagggtctggatacctcttcctgggagacgagcaggactagaactgctccaagaactgaggatgaggagttcccactgtgggcagcgggttaagtatccagcattgccacagctatggtataggttgcagctgcggctcagattcagtccccagcccaggaacttctataggtcacagatgtagcaaaaaacgaaaacaaacaaaaaaacccaaaccaaacaaccaatcaaaaaaaaaaactcccccaaaaccaacagaggacaagccaccgggcctcagcaaatgaccagatctgtctgcaggagtagcccagccaggggcccagattccctgagaacaaggctctgcctggctttcccacagctcgcctttgctctgctccctcctccccttgtacttggctgagtagggcatcatcatccctgcaggcgattcaggccccatcccgacctgggtgtctgttcagcctctccccaggtgtgaaccctggtagccaccagaatcagcccccgcccccaggctgggagctgagagatgtcatgtgtttcctgtccccacgtccagcctccccaggcccctcctttcactgcagagtcagcacttaggagcagggaggattgcaaagttggcaacatgtgttttgtacatgtatgtggtaaaatatacacaagataacttttcttgttttcaccacttttaagtgtgcaattcagtggcattaaatacagtcacgatatggtgtaaccatcattaccgtttattcccagaactttttcatcatccaaaatagagactctatacctattaaacactaacttcctagtccctcctctcttcagcccctggtaacctctattctactttctctttctatgaatttgcctattcgaggcatctcatgtacatggactcataaaacatttactcttttgtgcctggcttattttacttagtacaatgttttcattacttaggatcatgacaatgttgtacatgtgtcagaatttctttctttttgttcttttgtttgtttgcttttgctttttagggccgcactcgtggtgtatggaggttcccaggctaggggtcaaatgggagctacagctgccagcctacaccgcagccacagcaactagggatccaagctgcatctgtgacctacacagctctcggctggcaacaccagatccttaacccaatgagcgaggccagggatcaaacccacatcctcatggatcctagtcaggtttgttaaccactgagccacgaaaggaagtccagaatttattccttcttatggctgaataatattctattgcatggacataccacatttaaaaaaatccattcgtctgtggatggacactcggaaacactaaatggttccacaggagctgcatcattttacattcccaccaacagtacacgaatcttccatttttccacagcctcgctgacatgtgttattttctggttttgtttgtttgttttttacagtcattctaatgggtatgaagtggtagcttattgtggttttgatttgctggtgactaatgatgttgagcatcttttcatgggtttatccgccatttgtacgtatcttggagaaatgtctatacaaatctgtagcctattttttgaatttgggttgtttgggttttttagttgttgaattgtagcagttctttacatattccaaatggtaatcccttatcagatatttggatttttttttttttttttttttttagggctgcaggtgcagcatatggaagttcccaggctagtggttgaatcagagctacagatgccggcctatgctacagccacagcaccaccagatctgaaccatatctgtgacctgcaccacagctcatggcaatgccagatccttaacccaccgagtgaggccagggattgaacctgcattctcatggataccaattgggtttgtttccactgagccacaatggaaactccctttatcagatgtatgatgtgcacgtatttttgcccattctgtgggttggcttttcactctcctgatattgtctttttttttttttttttttttttttttggccttttgccttttctagggccactcctgtggcatatgaaggttcccaggctaggggtctaatcaaagctgtagccaccggcctacgccagagccacagccatgtgggatccgagccacatcttcgacctacaccacagctcatggcaacgccagatcctcaacccactgagtgaggccagggatcaaacccgcaacctcatggttcctattcggattcgttaaccactgagccacgacaggagctccctgatagtgtcctttgatgcaaaaagattttcattttgattcagttcagtttatctgttttttcttttgttgcttgtgttcttagtgtcatacttaagaaatcattgcctgaagttaggtcatgaagattttcacccattttcttctaagagttttatagtttctgttatttgtgtttctggcatctctcccctttggattcacatctgtgtccccatccccatcaggggcactccatggtggagggcctgccctcttgtgcatagtatacagtaggctttcaactcatgtttgtgggatatgtgttgggcagggtccttgtgctccctgccacacctagacttgccccgtcatcaactttaaatccccaagaaccacacccctctcttgcctgccactgaaaggcctgggggtgctctgccctgctgcaggacaatgtcctcagttccaggtcttgagaagtgggtgctcaccagggacaaggcaaccacagagatggttctggattgactagagaccccaaaagggacgtttcacctggaagccaccttgaagactgaatgagatttcagcaagaaaagatcatttttggtgatgccagacctggccagctggagagcccatgggcagacaggactggccccagagtgagactggtcctagggcgaacccagggggagcagggaatggggcagagctcagagtaggaaagggggacctttcagggatgcactgggaaccccatctcatgggaaatggggagccacagaaggttttcaagcaggggagggactcaaaggcagcactttggtatgtgctcagctctatagctggtaaggctgctgatagggagcaagtctcaaacaagaaagcatcatgtgtgctggatgccttggagtaagtcgctgggcctctctgggctcccatttctgttaacttgggtgcagagacagggcagggaagtggggaaagagaaacagctatttatccttgaggggctgggaaagcagaacttagggtgtggccaagtgacagaggactttctggggcatgccatcaagtgaaggcattcctaggcacagcctgagctggggaagtgggctcctggctccctgctggcagctgccctgccctctcactgggttggccagcgtagcacgtggggaaacaggactgggttcccagcaggctctgagacccaggtaccctgaggttccccaggaggctcccgtgtgccctgccagcccactgtgtgccaccctcttaggctccagaagcagcgccagaacctgctatggacttcctgctcctgctcctcctcctggcttcatctgcttaatgaagcttctgatggaattagaacttggcaaaacaatactgagaatgaagtgtatgtggaacagaggctgctgatctcgttcttcaggctatgaaactgacacatttggaaaccacagtacttagaaccacaaagtgggaatcaagagaaaaacaatgatcccacgagagatctatagatctatagatcatgagtgggaggaatgagctggcccttaatttggttttgcttgtttaaattatgatatccaactatgaaacattatcataaagcaatagtaaagagccttcagtaaagagcaggcatttatctaatcccaccccacccccacccccgtagctccaatccttccattcaaaatgtaggtactctgttctccccttcttaacaaagtatgacaggaaaaacttccattttagtggacatctttattggttaatagatcatcaatttctgcagacttacagcggatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggccgatcccatattggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaattcctgcagcccgggggatccactagttctagagcggccgctgactaactagtgcccttcccactttggtggggcaggtggagagtgttaagccctaagcaccacggtgaaagtctccctttcatctcctgaacctgcagccaggctgggctgagaacgggagacagaaaaaccagagaatttggagtctggcaacaggggccctgggatttagcccagctctggaggaggccagggaggaaggggatcacccctttcctgcctcaggccacttgcacacatgaccggggtaacatctggacgtggcaaggaggcaatgggagaaagccggagttttcacttgattatgcatcttcccatcacagtaaaagagggcagcctgttctagaaccaccactatacacagacgtcaggctgctggctctgccatccactagatgagcaaacttcaggcaagttccttgcctcttggagcctctgttttcatatctgtatactggcagtaacagtacatatgtttccatctcacaggattgttatgaaaattaagagtgcttggtacctattaagcactcaaaaataccaacttcttggacttccctggtggtacagtgggttaaggacctagtgttgtcactgctgtgacatgggttcgatccctggcccaggaacttctgcatgtcatgggggcagccaaaaatattagcttctatcattttcatatttatatagattaagcacttgaaaacatgatcttttattagcatagttacatagagagaggctttggaacaagatttaaagacaggtcatttcttcgtattgtggacaccctcccagcaaaggagttaaccattcttagagctactttgacactgcttttggccccctgcattaggtggcggttccttgtcaaccgctaccacaagccaggcgaaaaagcagtgcggtgcccatgacccttaaatagaagggagggctatgggaggcatcatgaggcagaaaaccaaggacggtagtgcaggctggctgtaggccaaccctttgcaatatcaggctctcggtggagaaggaaggggtcagtattcctcacaaggtggggagcagggcctgccctggacaggggaactagtgggacttaagatttatctgggtggaattcccctggtggcgcagcagtaacggaccccactagtacccatgaggacaagaattcaatccctggccctgctcagtgggttaaggatctggtgctcccgcgagctgcggtataggtcgcagatgcggcttggatctggtgttgctgtggctgtggcataggccggctgctgtagctctgattggaccttagcctgggaacttccattcgctgcgtgtgcggccctaaaagaccccgatccggcaaaaaaaaagatttatctggatgggctttctagggcagagggttgaaagaggagagggacatgccccctgccctccctgggcccttgctcagcatccccccatcatgccttgcagatgcccccaagggtgtggagatccttttcagccactccggacggaacgtccttccaggtgatctggtcaccctcagctgccaggtgaatagcagcaaccctcaggtcagttccgtgcagtgggtcaaggatgggacgaagctcaaagaccagaaacgtgtacttcagttgcgccgggcagcctgggctgatgctggcgtctacacctgccaagccgggaatgccgtgggctcttcagtctcacccccggtcagcctccacgtcttcagtgagtcctgaaggagcctgggtcttgatcagagggcagcgttggggttaaggaaaccaggccaaggcacctctggggatgcgggtggggagtcccaacccggcctcagacaggtgatcgcctgggatgtatggaggatcccagtgaacagggccaagagatgcggagccaatccgagaggcgcctccgcagaaggggcggggcgtagactgtgcagagcttaaggctgattggctgcaaccatgaggggttgggcctggaagtcctctgggtatcccccagcgtgtcctatgctgtccccacagtggctgaggtccaggtaagccctgtgggctccatcctggagaaccagacggtgacgctggcctgcaatacacctaaggaagcgcccagcgagctgcgctacagctggtacaagaaccacgccctgctggagggctctcacagccgcaccctccggctgcactcagtcaccagggcggattcgggcttctacttctgcgaggtgcagaacgcccggggcagagagcgctctccccctgtcagcgtggtggtcagccgtaagtgagggtggagggaccggtggggagtctggacctgggaactgagccaaaaagtaggaggccctgcaggacgtttggggtcctgggcccaggtgcccttgagctcacatcttggagaggccttgaccttcctgggcctcagtttcatcctatttttattactattttttaaaatttattttgtctttgtttgttttgtcttttctagggcatatggaggttcccaggctaggggtctaactggcactgtagccacccgcctacgccacagccacagcaatgcaggatcctagctacatctgtgacctacaccacagctcatggcaacgccggatccttaacccactgagtgaggccagggatcaaacccgcaacctcatggttcctagttggtttcgttcaccactgagccacgacaggaactgctatttattttgtcttttgtcttccctagattctccatttgttaacattttgatacctttgcttttctctctctgtgtattatgtatgtgtgtgtataggtgctcacacatgcacacacatgcatgtatgtgcatatgtacatatacacatgaaagtgtgcatacaggcatacattttctccacataaacacatagacatttatactctctctagccacatgcatatacacacatgcactctatatgtatacacacacacacatgctatctattatcaatctatctatttatctacctatctatctatctatcaatcctcctgaacgtgcaggcataatgcctctttactccttaatttctcagcatgtatttcctaaggtcattcttgtgcataactgcagtacaataactatagtcaggaaacttaacattgataccattgatataagactcttatccaatccacaacccacatttaaattttaccaggtggcccaacaatgtcctttatggcaattatttttctgctccaggatttggtctgggatcctgccttgcattcagttgtcgagtgtctttatcctccctcagtctgggacagctcctaagtctctccttgttttccttgacattgaatcccaaagatcacaggcctgttgctctgtgctgtcctcagtgtgcgttgatctgagtggggcagactgaggctatgcacgtgagcagggacatcgtggctgtgatgctgtgtctgtctccccctgcatcacaacaggaggcacgtggggtctgtttgtcccattgctgggagcattcactttgatcatttgctgcagtgctggccgctggctggtttctttgttagatttcccgtctttatttttatttcattttatgtttttagggccgcacctgcggcatatggaggttcccaggctaggggtcgaattagagctatagccactggcctatgccacagccacagcaatgccagatccgagttggatctgcagcctacaccacagctcacagcaacaccagatccttaacccactgagcaaggccaggaactgaacctgtgtcctcatgggtactagtcagattcgtttctgctgagccacgacaggaactctagttttcccgtctttaaaatgggtccctggagccccattctcagggttgccacaaggattgagtaggatggcagggcaatttacccagttcagggacaggcactcaggagccattggggtgccccctttcccagggcattatgggacaaaaatgggtgcaggggtcataggctataaagggagatccaggagtgcccctgttgagagtgacaggccttccttcatccccacgcagacccacccctcaccccggacctaactgccttcctggagacacaggcggggctggtgggcatcctccaatgctctgtggtcagcgagcccccagctactctggtgttgtcacacgggggcctcatcttggcctctacctccggggagggtgaccacagcccacgcttcagtgtcgcctctgcccccaactccctgcgcctggagattcaagacctggggccaacagacagtggggaatacatgtgctcagccagcagttctcttgggaatgcgtcctccaccctggacttccatgccaatggtaagacggccacagcagggctagtggagggagcctcaggtttgttctgccccctcctcctgggagtccagagacctcaaccaagggcctgaactagctgtctagaaaaaaggaatatcaaaaatctgagctgtaccgtcatcccaggctcagaggttcctagaagtcaaggcaaaaaggggaacatggtcgattgcattattccttttttacccattctgactctgaacttggagggttcctcgatatgcatttatttgtctttaaattattgaacatttgggggcagactctgtgctggggtcttctagttcttggtgcatatgctgctcttccctccttctgagacaggcagagctaatcaatcaagccaggtttttaacataagcctgggattgggctcagaatccttctcaatcatgctgcagccagctacgaccaattagatccaaatgggcattcaaagccaaactctcctgatcattgagagattcgcagagctgatgtaatgaagctcagacctccctacaattgtatggggagaggggaaccctggcgggcttcctgcagtggggggcatggaaccgcggatgtgtgggggagctgagcctccctctctccctctcctgccagcagcccgcctcctcatcagcccagcagcagaggtggtggaagggcaggcggtgacactgagctgcaggagcagcctgagcctgatgcctgacacccgtttttcctggtaccggaacggggccctgcttctcgaggggcccagcagcagcctcctgctcccagcagcctccagcacagatgccggctcataccactgccgggcccagagcagccacagcgccagtgggccctcctcacctgctgttctcaccgtgctctgtgagtagccggcctgccagccgtgcctgctagggggagggtcagctcccacctcctccaggccctcctgactctgccctgtctgggagtcttgataaggaggcagcccaggcaccc

SEQ ID NO: 124 - DNA, 20 bp, Artificial sequence
  Synthetic oligonucleotide
agaggccacttgtgtagcgc

SEQ ID NO: 125 - DNA, 21 bp, Artificial sequence
  Synthetic oligonucleotide
caggtaccaggaaaaacgggt

SEQ ID NO: 126 - DNA, 20 bp, Artificial sequence
  Synthetic oligonucleotide
ggaacaggctgagccaataa

SEQ ID NO: 127 - DNA, 22 bp, Artificial sequence
  Synthetic oligonucleotide
ggttctaagtactgtggtttcc

SEQ ID NO: 128 - DNA, 17 bp, Artificial sequence
  Synthetic oligonucleotide
gcattcctaggcacagc

SEQ ID NO: 129 - DNA, 17 bp, Artificial sequence
  Synthetic oligonucleotide
ctccttgccatgtccag

SEQ ID NO: 130 - DNA, 19 bp, Artificial sequence
  Synthetic oligonucleotide
gatctggtcaccctcagct

SEQ ID NO: 131 - DNA, 20 bp, Artificial sequence
  Synthetic oligonucleotide
gcgcttccttaggtgtattg

SEQ ID NO: 132 - DNA, 29999 bp, Sus scrofa
cagtagtcataaaacaggatttgtttttcacagtaggatgggatttcatatttcatttcaaatatggccagaccataatttactcaaccaatcacctacagctgaatatttgggttatttctgatttttcctgaattataaatatcactgcaggagttcccgtcatcgtggtgcctcgaaatcgaatccgactaggaaccatgaggttgcaggtttgatccctggcctcgagcagtaggttaaggatcctcgtttccatgagctgtggtgtaggtcacagacgcggcttggatctggtgttgctgtggctgtggtgtatgccggcagctgtagctccgattcgacgcctagtctgggaacctccatatgccacaggtgcggccctaaaaagcaaaacaatcaaacgaaaaaaactcactgcagtaaaagctttatccataaatgttctacttcattaggctaaattctgaaagttaccttcacttctatccccaaactgccctccagaagatccagctacccttcggccacagggatatgtgtactactttccaagtgccctcagagcctaaggtggacaggcaggaacccaggattcagattgtctcctggccttgctgagcctttgtatggcttgtccgccatagagagaattatggcaaataggctggaggaggggtctaggcatcttgagatcggagggagagaaggaagccccaaagaagctgctgggatctcgctcagagccaacttattcaccttacacacaattctatattccagtaaacttgtgacctggcttttaagagcacagcaacgaattaggaagaataggaaggagaagcaaggctgcttcttagaggcaccctggcccatggtgctgtttccagaagagcaagcacaagtcagacaacaagcaaccacccacagaccagtgctaaactgtcccacagaccccaggaatggatgaggaagagtcaattagattgggtgatctcaatttgcttctaaacctgagattctggaaggtatcttgaacaatgtggggggcagaataatgccccctcctcaaagaagttcttgtcctaatccctagattctgtaaatacgctttgctatgtggcaaagaggtattaaagttgcagatggaaataattgctaatcagctggccttaaaataaggagattggtgttcccattgtggctcagaggtaacaaacctgactagtatccatgaggatgtgggttcaatccatggcctcgctctgtgggttaaggatcctgcgttgctgtggcagtggtggatgggcctttgcctgctttgaaaatggtagggtgccacaagccaagggatgtgggcaggctctaaaagctggaaaaggcaaagaaatggatgtttcttatgtagactgtccagaaggaagtcctgtggacacccatgaatataacctagtgacacccaatttggacttgacttacagaactgcaagataataatgtgcattaaatgtgtatttatttgttacagcagccataggtaaggaatacaaataatctggcccagtcaaaagatgctcagagaagaggagtgcccttcccctggccacacagcttctgaaagcagcatacctctggctccagtttttgttgttgttgtccttgtttttgctaggtcctgctgcttctctgccttcttaataatgagccccatgtctcacataagttaaattatgtccttctgagattcagttatctggaacagccatctccagggaacgtcggatttctttttctgaatgagtggttggttccccttgccccagatcgaaaggcgaggctctgtgcccctccgccaagggcagatgcccgccagatgtgtaggggcggggccctggggaagggggtgcggtagcaggaggtctggaacggaagagccactgcggtggctctacgtctgggggcctccagggagtggaggtggggtcagtcggggcggggctgcgggcgggcaggggttaactcgggaggaatagtggtggggtcaggttacttccctttaaaaagacgcggaaaggaggagtcaggttctggtgggcagtgtcggtggctggcctggctcttcgtcccagggaggaggctctgagccggctggggttgagcgcccctgcggccgcaggcaagtggagtgcgggttcggaggggctgcggggctgctgcgggcggcagctggagtgaggcagggacctggacggaagcgcgcgcgcggaactccccgcgacggccgccgcggacgctctcttctcagactggtacactgaggtcagccgagagtgaagaattcttttggtcaatgtcccgctttgatctctcggccaaattggtccccgcagcgcgcctatcagcgggccaggggaccacccacctggagaggtggccccccagggtggggaggggggcttctggcgcctctgggggaggacagatggagagacccgggtggtgccgcagactctgccctgctgggatctgaggcagggtggtctgggggactcaaccccagccgagtgccagctgggtgactgtgaggcacagcagctgagtcctccagtgggtggggcacagccaaagatgagcgggaattccagcggccccagggatccgcacagcccccaggagaggagagggcccccttcgccaaccttggacatttgcctctctccaaaggcttccacctaacccctattttaaagagaattcctgtttttaagcatccattttaaaatcgtagtctcccttcttacaccgaccccattgggatgcatgtgggttttccagatgcgggaacagaggctctgggattatggagtctgctccagccctccttggggagcagccaagacagatcaatgttccaaccaacattgaaaggggaggggtccccaaatggggccaacggggtccgtaccctgcacctccaaaaccctcagtagcgcagtgggccagggtgcccagctgggtcatgccttgcacttttgtgacttgaatccgcctcctctctctagcccagcccaggccaagctgttcaattgtcccagcattgttctcagtgagacctgaggtctcattgtagctttgtccctgagtctttatatgacctggacctctctgagcctggctttattcacctgtgaaaggggagtgttggaccagatatgggcccttgagtccgtccatctgtgtttgaggagttggctgtagaggaatgaggtggaggaaggggaaatgatgatgtggctgggaggaagtggaaatgggcctccaggccaaggaagcctgagggtaagggagctgggaaaggcagggcaccctccactaccgtggactggcatcctccactagcccacctcccgaaggagccctagcatcgaaggctggatgagtttggataatagcctacctgagagcactgccccatttcccagatggagaaacgccccctgccctgtctcagagccttgggagagggggcagctccttcccacttccacaagtcctccagcccctttgcttcaagagttttattgctaagtattctgaggagataagtgggggcccccgcatgcgtaggcactagggcactggaactaggcctagttcctgggcctgcggctctgctggaagatattagacaggaccccatgctgggggctattcgagaatcttggagagaaacatggacggtgggccagctggtcacgctgaggccgctgggtctttcattcacacgggcagacagccattgcctccagcccaggcccagccctgggtgtctgtcctcagagatgctgcatgatttggctgcacccttcaggacaggccagagtccctccggaggcctgtgcctggtagtgcacccctgctgagagaaagtgcccctccctaggaccacccctagcttctggtcctggtctgggggtagggagttcatgtcccctgggtaaagtccccaaactgacttccaccaggttcccagtctctcagtcattcctttgcagcagccactcccatccctgccctttagtatgcctataaaattcagtaaaagggacacacagttcccttaaaaggtgatcagacatttcatcctgcccagatcagccttgctttagttgactaaaaggcgtatgtctcctcctgtggtgttttagccctcctgggcactgctcaggaaatgtggggagatgtatgggaggatgctttaggaggtgtatatagttaagcgagagaatctgccactaactagctgtgtgaccttgggcaagtcacttaacttctctgtgccttggtttcctaaatagccaaacagtgatgattatcctggtcgtgattatctgctcagttagatagatgggatttaacgggcaggatccgaagcccagaatggcccttccaaagctagtgtgcatcccccctattccacgagggcctcttctgctcgttttgttctgagtgccctgccactgggctccagaggccacgacaggaaccgactctccactggggccgtctgtggttctcacaggatggaccatgttcacggctcccagaggaagcaccctgcggctggccccaggcccagcctcttggagctgtgggtggagacctcagaagcctgggagaaggccaggggcctgaagggccagtgtgtgccctgtgttggagccccagccccagcctcatccccctccccactctctgctccttcccagcagacagatgggctttgggttgttccctggctcccagcctgcccagccctgcagctgagggggctggggggtagcactgggggcggggccggcaggaggcaccgagtgagcagcagttatcccaagacccagggatctgcgaggaagcctgggctgcacagttgtttgcctgcctgctgatcctaggctccccctcccagcccctcgcaggctcctggcgagttcatgtttgaaacgaaggcaacttgtaccctcacatctgagattgagctgcctttggctcttcatttgatgtcttctatgtgagtggctatgccttaaagattctctaatacaattgttatcaaccttaaattttttttttttaatttagaacttttgttctcaaacacaaacataggcagggcaggcataatacataatttctgtatgtatacataaacccatagatagactgctttggttgagactggggcttggtactaggcttctccctcatcaagagatgggcagtgaaaggaatgtgcacattctgctcacctgtccacccccaagacagccggcaggtgatgccacccccagaatcatgtaggatggagtagtgattcccaaagtaagtgcagctatacaagatcattttagggaatacctagatgtatagttttttttttttttttttttttttttttttggtctttttagggccacaaccgtggcacatggaagttcccaggctagggttgaattagagctgtggctgccagcctataccacagccagagcaacgccagatccgaaccacatttgcaacctacaccacagctcatggcaacaccggatccttagcccactgagccgggccagggatcaaacctgcaccctcctggatactatgttaccactgagccacaatgggaactcctgaatcatttttaaatcagtagtcataaatggaaagtgtatcaggaaaaatataatgtaatacatccaaccatgactttatggtcattactattcagaaggagagcagtaaattgatttgaagaaaagtataagtaaataatgtacaggaggatcacggatatggcaaaactcatgcaagtggtttgtatgcacagccttggagcccgagaaggagtgtgagccacagagggaatctgtgctgagagtcaagagtttgagttacaccgagttccatctttaaccgctgccactcattttcttactgctgcttccattccactagcgtgtgtgacttgcctgctgcatactagctgcttgcctggtgtggaggatactgtcctcacagcgctgggtcttggggaagcaggagggctgtgctgggacagcatgactgaagtgagcatccagcatgagggggtgataggtgagggaaagacagaagctttccaggcccagagggtgggtggggggagggtgggtgctggaagagggaagagatgatgggcttgagaaaccgaaagaggccaaagcgttgagacaagactggagactcatggggctgtgtcacaccggacctctaagccatggcaaacatcgtggacattgtcattcgtttagccaacatagggcttctgccctgtgctaggcatggaggggtgttcgcagagccttcccttgaggtgccttggtctagcgagaagctttagaacacatggatggccttttcttaagtgctgcgatagatgaatgaaccagaggccagggaagcactggggtaaggggctgcccactcatgagaaggtagtgacatctgagctgtgttttcaagaatgagtaggaaggagcttgacaggagggagaggaaaggcattcctcagcagtggaaataggatgggtcaagaggtatgaaaaagtggagagtatggagaaatgctaacagctggagccatgttgggcattggaagcagaggccagagggttaacttcacagaaggttttcagactcaagccagaaatgaggtttcattctgccagcagtgaggaactggagaccaggttcaagcaggagaggaagatccctctgcggagctgtgtgggcaggctgggggacagggaggacagctcagttattgctgttccctgggggtggagatctgtgcctgggtcaggggctgaaagaagggtcccttttcattatcaaagaaaatggggactgtggagcaggcccctctgtgcccccagcctgggctggcatggtgcccagaacagggacctctcattacagggcaatgggtggcccgctgcgccaacttggaggaccagaggccctttccaaaggagtaaagactccagccaaccaggcccgtggtctgtgtccaaagcaaacaggaagaggccatggtaggccaggccgggcctcttcctttgtttggagtagggattaggccaggcaggcttgtgtgtctttctgcagcaaagggagggggctgaaggccctgcagagtgaaactcagtgcaggggatacatggcccctgggtgccagaccatcggcctataaaggtaaataaaggggacctgccttggcaaagtctagctctgatggagggaggccagaagtatatacttctcaccctcggtcaacttcaggggctgctgttccttctgtcactgtgcccaagctggagtcctcccccgggtacttggcttctcactcatgccccctggccatcagcccaggcctgatggaggtggctgtaggtcaccgaggtggtaagtccacactgcccagcctagggccctgagggcaagtctccagagagaaggagggaagtgctaagggctcagagcccccttaagctgaacgataggtacctgggatccctctgaagaccctgcactcaagtccctggcctccactggccataggacaggtagcagtcattcactcttacaagcatgcctgaaggaattaatctatgcatacatttaatccatgcatgcattcattcaccaggctttcaggcaggctcggggggtggccagcccaagccagagcagggagtggcagatgaacaaggtcctcaaggcgttgacaagataggcataagccctgagatgcgtttggccccaggagagtcttgcccatctgccatggtcggaggagggctcaaaagaaaggggctgacaattctcccagggcaagggtgggaatggggacaggggacagatgggctagaaagacttcagcaaggagtgactcttgaattgactcatttgtctatgcacccattcatgcatgtgtgtattcactcaacaaatatgtattgagcaactactgtgtgctgggcaccatgcctagtgccagtgagcagaagcagccaaggatctgttctccaggggcctgtcctgcaggagttcccatcgtggctcagcagaaatgaatctgactagtaaccatgaggatgcagattcgatccctggcttcacttggtgggttaagcatccggtgttgctgtgagctgtggtgtagatcgcagatgtggctcaaatcctgtgtggctgtggctgtggtgtaggctggcagctacagctctgattggacccctagcctgggaacttccatatggcgcaggtgcagccctaagaagacaaaaggggggggtcctgcagttagtgggggagccaggcagtccttgagtcatcaccacacagataaatggtgctcagttatagggctttaaggaagagatttgtggtgctgaaggggcatgactggtcagggaggtcagggtgacttctctgaggtggaatggggggataaagaggatattaaccagggcaagaagaggaaaaggaggatccaagctgagggaactgcacgtgcaaggtcctgtggtggaggggctggatgaaggccagagtggttacagttctgcacagagctgggtatggtgagaatgaggttgggaaattagccagccgatagcacttgaacttgtgggtgtcttttaaaagactgggtctgaattccaagagcaaaaggaaggtcttaagcagtgggtaaaatgatccaatttgtgtggcaagcttgtttgagggggccctagtgcaagtgattatccactaggataacgtgaggatccagccttgcccacataaatgatgagaatggcttttgccaaggacacggtgagaatgggggagagggtgagtcagatgttggggtgagggtcaggcgttgaggtccaagaaactggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacatggagggcaacgtcaaaaagtaagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccctgtgcttgagtcgtgaggcagtgtttgaattccagctctgagtcatcttgggcaaatgtcccaagttctctgaccttcagtctctgcatctgaaaaatgggaccctcctcatgaagggagttcctggcccctgaatgccagacagatagcagctgagtctgtggttattccccaaaggctcaaagctccgcagggacaccccctttaccgccccaccgcccccgccaccctcttctctgctgaccaaacctccactttaacctggtttgtccccctgactctgggacttggcccaccagcaccaggacccaaggggggccctgacccacctctatctttgcagatccggatctgggctcggcctaatgcaattgcagagggccatggcatgtatgccctgaatgtgacaggtcccatcctaaacttctttgccaatcattataatacaccctacccactccccaaatccggtgagtgaggggccttgcatggggggaggcggactggtctccccccacctccccatacccagagaccatcctgccaacaccctggcctccccagaccagattgccttgcccgacttcaatgccggtgccatggagaactgggggctggtgacctaccgggagaacgcgctgctgtttgacccacagtcctcctccatcagcaacaaagagcgagttgtcactgtgattgctcacgagctggcccaccaggtagcccccatagcagggcgtgcagacagagaagggaggggggctctgggaggagatggcacaggtcctggctctgtccttggcagccaaggagggaggaggggtccacctggcaggtggcagggaggagtcacaattaggtcagtaaagcctcacactcaagcctggcatgcgaagaacacttggtcaagagtagctctgggtacttagcgacctccctcccccacagtggtttggcaacctggtgaccctggcctggtggaatgacctgtggctgaatgagggctttgcctcctatgtggagtacctgggtgctgaccacgcagagcccacctggaatctggtaagcccactgcccgggggccctgtggtctgggatgggggagggcctgcgtcacccttcccaggccagtctcctgatggctctcagggctttgcagaaagacctcatcgtgccaggcgacgtgtaccgagtgatggctgtggatgctctggcttcctcccacccgctgaccacccctgctgaggaggtcaacacacctgcccagatcagcgagatgtttgactccatctcctacagcaaggtgctgcccagcctccgtgctggtgctgagacagtggggggcgcagggaaggaggtggggaccctgcccatgcggacccactgaagctggggctcccagagcgcctgtcccagcccagtggggacacagggtcctgctggctctgcgcagccttctgagccccctcccctctaccagggagcctcggttatcaggatgctctccaacttcctgactgaggacctgttcaaggagggcctggcggtgagtacccttggccagctgttgggtggggggcgtttcctccagccctggccaattgcagaggtccattgctagagcctggcagcaccggctttcccctcactgtgccctgtctttccacagtcctacttgcatgcctttgcctatcagaacaccacctacctggacctgtgggagcacctgcagaaggtcagtgatagccagccaccaccccactccccgccgccaccccgcccctgccccccccccgccctcccaacccaagctgttctatgggggctctgagatgctggaagtccctaggggcagcacgcatataagagaacaggcccagcccccctgcttaccctcccacacaggtcacgtggagcctcagttgctcatctctaaaatgggctaggtgatggctcctcgaggaggcatggtgagagcctgtgttgggagctctggcagagggctggccctaccactgtagtgagctctgaggggtcccctcatctaggtggggaggacatgggctctcaggtttcccagggccaagccttcctctgccaggaactttggagcctgtggttcacctgctcagcacttctggctgccctgtgcccggccagcccctcccaggcatgacttcgcacagaccagcaccctcagcacagctttgaccctcccacccagctccactccaccgtctgtgagggcaaacctttgggccaagtcccccctccctccctccagcctgcccctccctgccctcctgaggctggagttgcaccctctccaccatcccccaccacagtctctccttgacagctgccgccacccaccccttctctatggccctcatcagtcccactgacccctccacgtgcccatgctccatgaccagcctgaccagggaggggagccctggggccttgttccagagccccggccaggctgcagggccctcccagcgccagctcaacaccctctaccacgagtctggtcccctcccccacctcccatagcctcagtccctcccctctgcttccacatcttccttgccctgcctggatttctcactccatccctcctccacgccagagccccttcagccccatctctgcctccccttccctctttccctgagcgccccctcccccagctcctctcctgctcagcttgccccttttaacgtggtcttctgccctctttttatgctgttgttagtttttattttctcagttattaacagaggtggttgcccttttgttattacaaaagcaatgagggagttcccgtcgtggcgcagtggttaacgaatccgactaggaaccatgaggttgcaggttcggtccctgcccttgctcagtgggttaacgatccggcgttgccgtgagctgtggtgtaggccagtggctacagctctgattcgacccctagcctgggaacctccatatgccgcgggagcagcccaagaaatagctaaaaaaaaagcagtgggactctcttgtgggttaaggatctggcatagccactatagtggccaaggtcgctgctgtggcacagattcaatccctggcctgggaacttccacgtgctgtgggggcagccaaaaaagacaagacaaaacaaagctttaagacaataatagccacccaaagcccatccccacattctcagcctggtgcacttcctttgcatcgaccacagcccgctccttggccctgcctgtgacatcttccttcatccctgcccatcctcccagatccgttcaggctgacccatggcatcctggatgcccctccccgacacacacccacctcaggttggaccacagaaaaggagggccagggatgtgttgaatcagtgcagaataaaccggaggctggggcactcttggctcatccagtgccaggcagctgaggggagagatggccaggccgggaccccagctcctgcctcccagcccagtgctcctccactgccttccagcaaggcaccactagggctagtccaggggtaccagggctgccgagatgggcaggtgggagaagagcagtctgctgacggctgcgctatctcactctacccaaggctgtggatgctcagacgtccatcaggctgccagacactgtgagagccatcatggatcgatggaccctgcagatgggcttccccgtcatcaccgtggacaccaagacaggaaacatctcacagaagcacttcctcctcgactccgaatccaacgtcacccgctcctcagcgttcgagtgagcagatggagtcattgagccaggcgttctcccgagggttctgaggactatttgctgtgggacactctgaccatagcagggtctggcctctgtggcctcggccacaatagttgttgacagtcttcctcttggggccagagtcgagcagaaaccgagaggccgggcagggctgccttcgctggcggtggtcacaagctgccgtctctcacacctggcctctggatctcttgttgcagctacctctggattgttcccatctcatctattaaaaatggtgtgatgcaggatcactactggctgcgggatgtttcccaaggtaagcccctctccttggcattgccccagtggcctgagggccagctgctgccaggctgcaggagggactgctctgctgaagcctggggagaggagggccgggggtcaggtcagtccatggccttctcagcagcccctccccggctctgctccctccccacagcccagaatgatttgttcaaaaccgcatcggacgattgggtcttgctgaacatcaacgtgacaggctatttccaggtgaactacgacgaggacaactggaggatgattcagcatcagctgcagacaaacctgtcggtgggtacctgtcgccccaccccattcccgggatgttgccccagactgcaggccctgcctgcatcgccagcctgaggtcacacgctctgttgcccctcctaggtcatccctgtcatcaatcgggctcaggtcatctacgacagcttcaacctggccacgtgagtgtcccttcctcctccagccctgccagctcagccacagaagcctagtgacttggcaagtgtgactccatagtgagagccagtgctcctccccctgtcctcaccgcgagcggcacagcacagcgggctccctgggcttccggacacccctcccctggcctcacatccccagagattgttagtgttacagcccacatttcccagagaaggaatccgagactcagagtttgagttccttgtctgaggtcacatagttgcccaggacagccagacacaggcctgtccttctctaaaactacactgaattccatggcaccaaggttctcctgagggctaactatgggccaggccactgtgctagaggctaaaaaacatctgggaaggtcggtacaggagagacgcagtatggatgaacaaatatttatcaagcagctgctgtgtgtgtgaacactggcaggtgcgaggttctttgccatagatgtcacccatagacacggacattgctcataaatgtcaaaatggtcaccgatggaggaggaggagggaggaaagaccagagagccaagcagatcagagaagagagggcacaggaagaagcacagtcttcttgtctttttgaacctcagttctgtctcttatgggctgtgtggctttaggcagggcatgtcacctctctgagcctcagtttcctcacctgtaaaacggggaccatgagcaaggacatgtgactggcagtggatgagggccaggcctgggctgcccttgtttgtggcaccacccagccgccatgtctcccactcctggtgaatgttggagctggctcaactgcagctctgggacctcaggctctaggaggaggctgaggttcctacagggaggatgggctggctgggggtgcttatttgagcccctctatctccccccagtgcccacatggtccctgtcaccctggctctggacaacaccctcttcctgaacggagagaaagagtacatgccctggcaggccgccctgagcagcctgagctacttcagcctcatgttcgaccgctccgaggtctatggccccatgaaggtacagaggagcatggcagggttcagggaccagttcctccgtgtttgaaattacgcctcaaggaggtgaagagaaatattgccgggagtggtgaaactgccaggctgacacgacaccaggtccttggctgagagtagccttggcagtggtgagagagcgggagggatggaggggtgaaaggagcaggagctgggaaggtgagcaggttgggcaacacaaagaaggggcacagacttctagcaaagtttgctagatttggaaattagcactttcagaaggcaacagaaaaagccaccaaacgggggtggggccgggtagggaggccagtctttcacccgaggtgtggccttggacaatcctcttcaggcccagagaactcctccgtgaaaggggtggacctgtgcctgtctagcctgccctaagggccaggagggagttgcagatgtcgcagggaggcgggaggatcagagggaaactgcttgctctggatgaagctgaatctgtaaaggatgttcactgcagtgtggtttataaaagacaagaaaaccaaagccggagttcccgttgtggctcagcgctaatgaacccgactagtgttcatgaggatgtgggttcgatccctcacctcaatcagtgggtcaggaatctggcgttgccgtgagctgtggtgtagcttgcagacgtgccttggatctggcgttgctgtgactgtggcgcaggccagcagccgtagctcccattcgacctctaaactagaataagcatttggtggaatattatgcactcttagatcacacttagagaggtcatttaaaaatgttttggtttttttttcacattattaacttcctggtatggtaaatgaggttttaacctatttttaatttataagccttggagctctgattcgacccctagcctgggaacttccgtatgtcaagggtgcggccctagaaaaggcaaaaagaccaaaaaaaaaaaaaaaaaaatgtattagcttacctacaccttggatcaaccgcatcctcgtaatgtagttaaatcacagtgtttggtcaactcagtatcaatatttactttatgtgatttctaaactgtcattctctgctaagccacatagagtgctgtttacatcccctttttatatatcttatccaatgtagcaggtaatacatcagttttgctttctcctggaggcatcctcctggagccctctgctagccggcccctctggggctgctcctcagctcccacgccagaggcgctcccttgttggtcttgtttcctgaacccacggttccttccttgagtttgttctctattggggcacatcttccagtttggcaggaagggttcgtaagaggtggattgttttcaggtagattgtctgacatgtctttattctaccttcacactggattgcgagtttacttgggtttagaattttctaggttagaaattattctccagaaaatttcaggttgaaattgacctcaagtctctcgtgctactgtcaaaactcccagagccgttctcatttccgcttctttcagtgtggccaggttcccccttctgggagcttatgggcttttctctccatcctggtgggcttttctcatccttctgtgctttcaatctgggcaactttttggagttcccattgtggcttatcagtaatgaatctgactaatatccatgaggatgaaggtttgatccctggccctgctcagtgggttaaggatctagcattgccgtgggctgtggtataggttgcagatgcggctcagatcccacgtggctgtggctgtggtgtaggccagcagctgcagctccgattcgacccctagcctgggaacctccatgcaccacaggtttggccctaaaaaagcaaaaaaaaaaaaaaaaaaacacccaatctggacaacgttgtctcgctcctttaactgttcttcccccacccccagcccagttttctccctctggggcccttgttaattcaatttactgttcttacactggtcctctagttttcttctctcctatcttccagcttgttgactttttgttctgtcctctgggatatgctctcagcttagcacaccacctctctacagactgttttcatttttcctatcgtatttttaagcttccaagtgctattcctggctccagcatcctatgcaatatcccctctgcactctcagaggatatttatcataggcttttatgaagttttattttcctttcactgacttatttcctctgagttttttgctttggtctcctctttcaagttggaggctgagctgcccctttgaatttgagaatgaggcaatgaaaagtgggaattcccactgtggctcagtgggttaagaacccgattagtatccatgaggacacgggttcgatccctgccttccctcactgggttgaggatctggcgttgccgtggctgtggtgtaggctggcagctgtagctctgattcaacccctggcctgggaacttcctcatgccacatgtgtggccctaaaaagaaaaaagaaagaaagaaagctccatggggaggtggagtttgtcacctgtagtctccttgtggggtgactaggtggccaagtaattcctgatggggaacctcaaaaattagtatccatgagtgtgttccctggggactcttcagtgtccagagagtgaccctctctcttatgcaccgaggctcactttctgggatcagggtgaggaagaagactgggggtacctttgttcagaatgcaaatattcacctctggccccctctgcagtccagcaaccctcccactgtgtctggggaccagaggtctgagtccctctgatttagccttgcccatggaaggagggaggggtgctgctcgatgcagggagggaagtggaggcttgaaagtttaattcatccttctacagactttgactccatcgacctgttcacggccctgcctcactcctgtcccctgcatgcccttctctctaatcctgagccttgtcacagcaagtctccggcttctgtctccatctcccccatggcaggtgctctgctgcagcctctccccctctgccaggcctgttccacctccctcttacaccttccacctcctaaatgtgcgaactctctcatcctctgtggcttcctttcctattctctttgttctgggctggggggtgagggtgttccttttacattcggatgtggatgctagttgacgtacatgacaatctactctgctgaaccaaggtcggcaaagaattttaataacgtggaatagggcttacacggggatgccagctaaaaagatcagcatactacattgcagacctgctgggcagaaaagaggaaagagggggcaaatgcaccaatttggaaatcttgggtttggtacattctagagtcgttagtgttttctgcctatgctttaaggcgttttctgaattcctcacaataaaggggcctaacacttattaggaaatggtgacacttagacacaagagtatcagaagagatcaaggggagttccctggtagcctaatgcttaaggatccggcattgtcactgctgtgatgcaggttcagtccctggcccgggaacttctgcctgccacaggtatgacccaaaacagcaaaaaaaagtaaaaaataaaatgaagatatcctttaaaaaaaagaagaagaagaactggtcaaggatttgtggaggagaacctcagatcagtcatctgaaacctggacgagtgccagcccctttgagcacagtctggccttgtgcgaggcctttagcctctggcctcttgctcctgtagccattagctcttgctacatctgcccacccacatcagaggctccatgggtctccagatgactcaggcatgagtctcttctttgaagctatttttagggctgcatcctcggcatgtggaggttcccaagctaggggttgaatcggagctgtagccgccagcctacaccacagccacagcaacacgggatccgagccacatctgcgacctacaccacagctcacagcaatgccagatccttaacccactgagtggggccagggttgaacccatgtcctcatgtttcccagtcagattcgtttctgctgtgccatgacgggaactctggaacttcctctttgaagctctttatgttttgttcttgttttttgtttttgtttttctagaaatacctcaggaagcaggtcgaacccctcttccaacatttcgaaactctcactaaaaactggaccgagcgcccagaaaatctgatggaccagtgagtatgagctcgcttggtctggagatcatgggtggtgcaggtagcctgacctgggggcccatagcaagtccagcagcatcctctctggagctcccaactcctggccggaccagggccacagtcagggagagcgacccctcccaaccccactcccggccccaggagtagggactctgctctgaggctctgtgtggcctatgaaccatctggcctctttgggcaaaggaccaaactgaacctctgagggtccctcacccgcatggtgaggttctaggtgttaaagctggggctggagcctgtgccagccctccccaggctgcccaagggcaagaagcaaagaagggaacccaaaggtggctggtgggctatacctgcagagtgcgggtctgcctccctgttgggagttgtgtgtcagcaggggagtcttggtcagcgtcaggtccaggcgtgctgacagagtgtcacccccggggtaggtacagtgagattaatgccatcagcactgcctgctccaatggattgcctcaatgtgagaatctggccaagacccttttcgaccagtggatgagcgacccagaaaataacccgtgagtgtgtcctttgttcctcccttgattttcatctgcccctcaagcccacaaccctgtccgcctcaggccctcatcccaccctccctgctgctgctccatccctgacacccctaccccacgccaggatccaccccaacctgcggtccaccatctactgcaatgccatagcccagggcggccaggaccagtgggactttgcctgggggcagttacaacaagcccagctggtaaatgaggccgacaaactccgctcagcgctggcctgcagcaacgaggtctggctcctgaacaggtgaggactgcagccagacagggctgggtaagctcactgccccccgaccccagggaccaggctcccagcgtgggaggagcaagaaccagccaggcctccacattccctcgtgggatccccacaagtctgtgcagtggggcagtacctaccccatttgagagatgaagctgctgaggctgcgagaagcaagacaacttgctcgaggcccagaagcttaaccaagtaaaaaagccaagacttgaatctgggtccaggtccatgtcctgtgctcaaaactgctgcagcctcctagcctttacgtctctgtcaagggctctggtgcagctagacctgagtccttctgttcatccagctgggcaccccacccccacccacaacctttgaaggagggccctactgtgtgccatctctgggacctcagtaaggaagagccaccctcagtgccaaaggcaggagtggccaccctatctgactcagggctcaccccttccttggctcattcattgacaggggtttatcaggcacctggtctaagtcaggcctgacacctggcgaagccagggaccctgcttagtggactcagtgtctcatccccacaaaggaggtaggaccaaggggtctttaaccctcagccggctgaagggatgctgatgggatgcaggacaggtgtgtggggttggggagagggcagggtccttctatgaacacagattctgctctcaaaggtacctgggttacaccctgaacccggacctcattcggaagcaagacgccacctccactattaacagcattgccagcaatgtcatcgggcagcctctggcctgggattttgtccagagcaactggaagaagctctttcaggagtgagtctcccgagaatgtgttaggggaagcacctgccaggcctgggtgtccctggggatggcccgtgtgcacaggaccccacggggtcaggactggaaagtagaccctgcctaggcgtggtgaaaaagaacccaactttaggccaggtaggggcaggaattgaaagataggacagtggtgagagttcccactgtagctcgggggtaacaaacccaagtaggatccatgagaacatgggttcgatccctggccctgctcagtgggttaaggatctggcattgccttgagctatggtgtagattccagatgcagcttggatcccacgttgctgtggctgtggtgtaggccggtagctgcagctccaattcgacccctagcctgggaacctccatatgtcatggatgcggccataaaaagccaaaaagaaaaaggaaggaaggaagggaaggagggagggagggaaggaaaggaaggaagaaagaaagaggaaaaaaggaagaaagagagggaaaaaggaagaaagctagacagggcagcgggaacggctatccctttttccacttgggggacatctggctggtgggagtactctctgaagaggtgggaggaaggacattgcagtcaaacctgtgttcgcgtctccagtggtgtctcctcaggtgtgtcgttgttttgcatctacaagacagggatggtcacagggtcctgtcacctgccagtcactaaccattccctgtcccctgatggtgcccctctctccctgccctgcagctatggcggtggttccttctccttctccaacctcatccagggtgtgacccgaagattctcctctgagtttgagctgcagcaggtaggaagccctgggccccggggctctcgcctggagagggaggacagaggccctggcagccctctcctcagggctgtattagcgacccagcctgccgctcagaatcacccttgtcctggcagctggagcagttcaagaagaacaacatggatgtgggcttcggctccggcacccgggctctggagcaagccctggagaagaccaaggccaacatcaagtgggtgaaggagaacaaggaggtggtgttgaattggttcatagagcacagctaatagtgcctggtccttcccgccacctggccccccgcacaagatgcccgcatgtgtccatcccagggcccacggcagggcccatgttcctgaagcccgaggcacctgcgtcctccctttagggacaaagcctgtggcccatgttatctccattctgccctggggcccaatccagtttctggtgaccagactgtccaggtgtctcccagccactgccccttgtgccaaccccaccctgggcctggcccagggcccttctcagggaagtccagctccaaggccagatgagcagaagcccttgatggatgatggatggccttgaagaactgccctctaccctctctcccctttttccataaagaccctgaacctgagaatcaacagggcatcagatctgtatatttttttcctaggagtaaatgtaaataaaggatttctagatgagcttccaggctctttactaaactcaagaaggaatgagtctgcataatagctggagagacagggatggagggaacctggggtgcatgggggtctggggacccccaggaagcctaggagggaggtacccagcctgctagatcttcctacaggacacccgtgtggctctagcttggagatggaggacagtgtctgcctgcagtgttcattgccttggcaggattgaaactctgggtgctggggagccaggggaggtagagggaatgagacacagcggtccccatggtggagagagccagggcctgacggtggaggcagtgcttttctagaaagcaagaagtgccatggttcgagaagctaggattggcacctgaactaacatgaggcaagatagtagcgtgccggctggcccttttcctgctccgccgctgggacgtctgggacagtgagcgaaaggggcttgcagggaggacagcagccctgggtcggggcctggtgcgaccatgcactggccaaggtaatggccaggatcgctggtggagggcggcatattgatttgtaaaccggtgttttcaagagtagcaggcagaaaaaggcaggtgtcctccagatcatctggggcagccagtggtgaaggccacctctggggagaaagtttgggacccagaagactccaagccccacatccattttggtggaactttcttggccaataaggccctgccgagcatccaaggctggttggagggaaagaaggagggaatagacagacaggccagggctcctctggggtcacctccagtcgacaccactgtgagctctgctccagctccagccctggaatccagccactgttccttgaacaagtgacctaaacccttaggcgatggttttctcatttggacgctagagctggtctatcctagggttggggtgtggatctaaggagatgcacggacttagctcacagtctgacgcttaaatggagctgggtgttactaacatccttagcccggtgtctgctcaaagcccctccaccttttccctcccagcctccttcccctcctgagaaggtgcaaacaggcctggggccaggaaggctccccccagctctgggggctcctgcacatcaaagggtggggcgggccaaggggcggtcccagccctggttatggatccctccaccaatcaggaagctcccccccagcccagtccctctagaccagctcttcctgtcgccagggcccagcagccttggctggcacttgaccttctgcggaccagagggcaggagggccccctccggtggctcctctgggggagggactagccttgccatcaagggtcccagagggagagctggcctccaggtaggagagtcagcagggaccctagtctctgtgcctggcactgggctaaggctttatgcgcattatctcatttggttctcaacagcgccagaagataaggactgtctttatctcagtgttgcagataagtaaactgaggttcaggaaggttaaggccctggctctgatgccctgtgcatgagtgggagccaggtctccatctggggccatggcagagtcatccacatcttctcgtctgacagcttcaatgcaggcattcagaacggcctgtgcggctggaggaaatcattctggggaggcagggaggacacgtagagccagagacctgcttgcggaacctcgttcaaggttgtatgcaggatgggatctgactagaactcaggctgctcatttcctgtccagaggtgggttgacctctcatggctacagggggatagcaggagctgaggatcagtccagagcagtgggaggtggcaccctctcccacctccccaccctctgggaggctcctcaggaggcaggtcctctgcactgcaggattattgaaaggcctggatgcagtactcagtgaatgtcccgtcatttagggatggtgactgggtccctaggaaggtatgggtagaccagttatcccagacaggacactagcatcagccatgctccctcctctgccaggcacttagcagagaggagcagccagcagcagtctgcctgggatggagccacagatggggacaggggctgctgctcctgagaggcagggctgagcctccctcctgagggtgaggacaggggtgggggattagcccaaaccccccaggccctgctccctccctgtagcagcatctaggaagggtccaggctgctccatctgcagtgatggcagccttttgccctggcctttgtatctcccagaggagcacctcttgacgagagctgggagaacaaggctgctaagttcctgaagatgctcacctctggctgctgctggtggaggctgcaacgcagtccttattgtgactccagccgtcatcagcagcttgaccagggatccccctctctgtctctgtaaggactaagcccagataggtgggagccaaaggaaggaggcaggcaactgttcagatctgtctttggtgcaagtgccgtgacactgtggagccaagaatttgggtcttctctggaaggagagcgctctggacagggagtcagggggcctgatgtccaaggagggctggagaaaactgttcccctccttgggcctgctttctcaactgtcaaatggaggagcagggagttcccgttgtggcaaaggggaaatgaatacgactagtatctgtgagggtgcaggttcggtccctggccttgctcggttggttaaggatccagcgtttctgtgacctgtggtgtagctcgcagatgcgactcggatctggtgttgctgtggttgtggtggaggcgggcagctacagctccgattcgaccccctagcctggaaacctccatatgccacagctgcggccctaaaaagcaaaaaaaattaaaaaaaaaaatagaatgaaggagcaaaaaatatccattgctcctaaacttggctgggcctcagccccaggtcccaggtcccaacccatgcactgagtcatgccctcagggtaggaccagatggctcaccttgcagccagtttagggaagcactagattggatcatcagctatcatgtactgagccattactatgtgccaagcctgagagccctgagaggtcagtattttactgtctccatttcacagctgagaagaccaaggctcagggaggtttgcagcttgcgggcgcttgtgcagaaaataggtgcatgaaggagcacctggggcagctgtgccctgggtctgtctccctgccatgtggaatttcatgtgaggtttcctcgccctctgctgccgcgggcagcccctgcagaaggccctggggtaggaagcagaacctccttttagggagttaggagaaggaggcacgtggtcacttaggccagttcctctttaagagatggggacactgagacatggagggggagtggcttgtctgatgtccaagggcacaagggtcagatatgaggctcgggtggggtagaccaggctcttgtctccctccctcccttctctcgccctgcactgttacaggacctcctgcctggctccatctctgtgaagcagccagagaccacgagtggctctaatgagcacacctatccccaaccttcactgcctcctccacccaggacccagaggccagatgcttccctgtgctgccgcatgcagttccccacagccctgcctccaaactccctttccaactcatcactttccgcatcgactctgaacttcagccacctctgttcccacttggctcctgctgcttctcctgcttactcctttttggcaaactccggcacatccttcgatattcattgcaactcttcccctcctggaatccggagaacgaagtcactatcactgctggctcccaagcccacgacagcctttgctaatggatcgtggcaccctttctccaagatcctggaggtgcctcttagtcttgctcaaggctgcataaatcaatgcagcaggactgatggatcaggtaactcaagaatgaaactgtgtcatcaatcccatcatcaggggttggcacatcccctctggtcccaaagtaccttgtatgaacccccacctcggcctctgatcatttctttcctttaattcacccactagtcaggaaacatgggaaagtttgcgggtttttttgtctttttagggctgcacccacagcatatggaagttcccaggcaagaggtcaaatcagagggcagccaatggcctacaccacagccacagcaactcgggatccgagccacatctgcaacttataccacagctcagagcaacgctggatcactgagcaaggccagggagcaaacccgtgtcctcatggatatgagtcaggcttgttaccactgagccacgacggaacttccatagactgttttctctggcacctagtactgggtaccatgggctgaagggtatgccccaaaactcgtggtgaagttctaaaccccagtgcctgagaatgtgactatatttggaaacagagcctggaagaggtaattaaggtaaaatgaggtcatatgggtggacccaaatccaattggactggtgcccctgtaagaagattaagacacagacacactggaccacaggataaccatgtgaggacacaacaagaaggcggccatctgcaagccacagagagaggcctcagaactcaccaaagctgctgaccccttggtcttggactcccagtctccagaactgtgagaaaattaatttttgttaagccacccaatttggcatattgtctcatggcagtcctagtacactagtacactgggcccagcaggcgggagccactcacaagcgccctctggaaacagtttcacataatcgtgaaagatcccttcaggttggcaggaatgcatccaggagaaggaaaaagggcagtggtgagggggcggaggccaggggggagaggcccgagcatgatcccacatgtgccgccttgcacagcaggccaggtggagcccctgcacagtcattgccaaatcaacaagtgagggcccacatcaaccttggtaggcttagattccaggaggggaaaagcaaacgcgacttgttccctgcgggatgggaacattttttccttttccagagcaataacaac

SEQ ID NO: 133 - protein, 1017 aa, Sus scrofa
MAKGFYISKALGILGILLGVAAVATIIALSVVYAQEKNKNAEHVPQAPTSPTITTTAAITLDQSKPWNRYRLPTTLLPDSYNVTLRPYLTPNADGLYIFKGKSIVRFICQEPTDVIIIHSKKLNYTTQGHMVVLRGVGDSQVPEIDRTELVELTEYLVVHLKGSLQPGHMYEMESEFQGELADDLAGFYRSEYMEGNVKKVLATTQMQSTDARKSFPCFDEPAMKATFNITLIHPNNLTALSNMPPKGSSTPLAEDPNWSVTEFETTPVMSTYLLAYIVSEFQSVNETAQNGVLIRIWARPNAIAEGHGMYALNVTGPILNFFANHYNTPYPLPKSDQIALPDFNAGAMENWGLVTYRENALLFDPQSSSISNKERVVTVIAHELAHQWFGNLVTLAWWNDLWLNEGFASYVEYLGADHAEPTWNLKDLIVPGDVYRVMAVDALASSHPLTTPAEEVNTPAQISEMFDSISYSKGASVIRMLSNFLTEDLFKEGLASYLHAFAYQNTTYLDLWEHLQKAVDAQTSIRLPDTVRAIMDRWTLQMGFPVITVDTKTGNISQKHFLLDSESNVTRSSAFDYLWIVPISSIKNGVMQDHYWLRDVSQGKPLSLALPQWPEGQLLPGCRRDCSAEAWGEEGRGSGQSMAFSAAPPRLCSLPTAQNDLFKTASDDWVLLNINVTGYFQVNYDEDNWRMIQHQLQTNLSVIPVINRAQVIYDSFNLATAHMVPVTLALDNTLFLNGEKEYMPWQAALSSLSYFSLMFDRSEVYGPMKKYLRKQVEPLFQHFETLTKNWTERPENLMDQYSEINAISTACSNGLPQCENLAKTLFDQWMSDPENNPIHPNLRSTIYCNAIAQGGQDQWDFAWGQLQQAQLVNEADKLRSALACSNEVWLLNRYLGYTLNPDLIRKQDATSTINSIASNVIGQPLAWDFVQSNWKKLFQDYGGGSFSFSNLIQGVTRRFSSEFELQQLEQFKKNNMDVGFGSGTRALEQALEKTKANIKWVKENKEVVLNWFIEHS

SEQ ID NO: 134 - protein, 963 aa, Sus scrofa
MAKGFYISKALGILGILLGVAAVATIIALSVVYAQEKNKNAEHVPQAPTSPTITTTAAITLDQSKPWNRYRLPTTLLPDSYNVTLRPYLTPNADGLYIFKGKSIVRFICQEPTDVIIIHSKKLNYTTQGHMVVLRGVGDSQVPEIDRTELVELTEYLVVHLKGSLQPGHMYEMESEFQGELADDLAGFYRSEYMEGNVKKVLATTQMQSTDARKSFPCFDEPAMKATFNITLIHPNNLTALSNMPPKGSSTPLAEDPNWSVTEFETTPVMSTYLLAYIVSEFQSVNETAQNGVLIRIWARPNAIAEGHGMYALNVTGPILNFFANHYNTPYPLPKSDQIALPDFNAGAMENWGLVTYRENALLFDPQSSSISNKERVVTVIAHELAHQWFGNLVTLAWWNDLWLNEGFASYVEYLGADHAEPTWNLKDLIVPGDVYRVMAVDALASSHPLTTPAEEVNTPAQISEMFDSISYSKGASVIRMLSNFLTEDLFKEGLASYLHAFAYQNTTYLDLWEHLQKAVDAQTSIRLPDTVRAIMDRWTLQMGFPVITVDTKTGNISQKHFLLDSESNVTRSSAFDYLWIVPISSIKNGVMQDHYWLRDVSQAQNDLFKTASDDWVLLNINVTGYFQVNYDEDNWRMIQHQLQTNLSVIPVINRAQVIYDSFNLATAHMVPVTLALDNTLFLNGEKEYMPWQAALSSLSYFSLMFDRSEVYGPMKKYLRKQVEPLFQHFETLTKNWTERPENLMDQYSEINAISTACSNGLPQCENLAKTLFDQWMSDPENNPIHPNLRSTIYCNAIAQGGQDQWDFAWGQLQQAQLVNEADKLRSALACSNEVWLLNRYLGYTLNPDLIRKQDATSTINSIASNVIGQPLAWDFVQSNWKKLFQDYGGGSFSFSNLIQGVTRRFSSEFELQQLEQFKKNNMDVGFGSGTRALEQALEKTKANIKWVKENKEVVLNWFIEHS

SEQ ID NO: 135 - DNA, 2599 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacatggagggcaacgtcaaaaagtaagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 136 - DNA, 23 bp, Artificial sequence
  Synthetic oligonucleotide
cttctaccgcagcgagtacatgg

SEQ ID NO: 137 - DNA, 23 bp, Artificial sequence
  Synthetic oligonucleotide
taccgcagcgagtacatggaggg

SEQ ID NO: 138 - DNA, 23 bp, Artificial sequence
  Synthetic oligonucleotide
cctcctcggcgtggcggccgtgg

SEQ ID NO: 139 - DNA, 23 bp, Artificial sequence
  Synthetic oligonucleotide
caccatcatcgctctgtctgtgg

SEQ ID NO: 140 - DNA, 23 bp, Artificial sequence
  Synthetic oligonucleotide
tacctcactcccaacgcggatgg

SEQ ID NO: 141 - DNA, 23 bp, Artificial sequence
  Synthetic oligonucleotide
agctcaactacaccacccagggg

SEQ ID NO: 142 - DNA, 25 bp, Artificial sequence
  Synthetic oligonucleotide
caccgcttctaccgcagcgagtaca

SEQ ID NO: 143 - DNA, 25 bp, Artificial sequence
  Synthetic oligonucleotide
aaactgtactcgctgcggtagaagc

SEQ ID NO: 144 - DNA, 25 bp, Artificial sequence
  Synthetic oligonucleotide
caccgtaccgcagcgagtacatgga

SEQ ID NO: 145 - DNA, 25 bp, Artificial sequence
  Synthetic oligonucleotide
aaactccatgtactcgctgcggtac

SEQ ID NO: 146 - DNA, 25 bp, Artificial sequence
  Synthetic oligonucleotide
caccgcctcctcggcgtggcggccg

SEQ ID NO: 147 - DNA, 25 bp, Artificial sequence
  Synthetic oligonucleotide
aaaccggccgccacgccgaggaggc

SEQ ID NO: 148 - DNA, 25 bp, Artificial sequence
  Synthetic oligonucleotide
caccgcaccatcatcgctctgtctg

SEQ ID NO: 149 - DNA, 25 bp, Artificial sequence
  Synthetic oligonucleotide
aaaccagacagagcgatgatggtgc

SEQ ID NO: 150 - DNA, 25 bp, Artificial sequence
  Synthetic oligonucleotide
caccgtacctcactcccaacgcgga

SEQ ID NO: 151 - DNA, 25 bp, Artificial sequence
  Synthetic oligonucleotide
aaactccgcgttgggagtgaggtac

SEQ ID NO: 152 - DNA, 25 bp, Artificial sequence
  Synthetic oligonucleotide
caccgagctcaactacaccacccag

SEQ ID NO: 153 - DNA, 25 bp, Artificial sequence
  Synthetic oligonucleotide
aaacctgggtggtgtagttgagctc

SEQ ID NO: 154 - DNA, 40 bp, Artificial sequence
  Synthetic oligonucleotide
ttaatacgactcactataggcttctaccgcagcgagtaca

SEQ ID NO: 155 - DNA, 40 bp, Artificial sequence
  Synthetic oligonucleotide
ttaatacgactcactataggtaccgcagcgagtacatgga

SEQ ID NO: 156 - DNA, 40 bp, Artificial sequence
  Synthetic oligonucleotide
ttaatacgactcactataggcctcctcggcgtggcggccg

SEQ ID NO: 157 - DNA, 40 bp, Artificial sequence
  Synthetic oligonucleotide
ttaatacgactcactataggcaccatcatcgctctgtctg

SEQ ID NO: 158 - DNA, 40 bp, Artificial sequence; identical to SEQ ID NO: 157
  Synthetic oligonucleotide

SEQ ID NO: 159 - DNA, 40 bp, Artificial sequence
  Synthetic oligonucleotide
ttaatacgactcactataggagctcaactacaccacccag

SEQ ID NO: 160 - DNA, 20 bp, Artificial sequence
  Synthetic oligonucleotide
aaaagcaccgactcggtgcc

SEQ ID NO: 161 - DNA, 17 bp, Artificial sequence
  Synthetic oligonucleotide
acgctgttcctgaatct

SEQ ID NO: 162 - DNA, 20 bp, Artificial sequence
  Synthetic oligonucleotide
gggaaagggctgattgtcta

SEQ ID NO: 163 - DNA, 2422 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtgccctccatggagggcaacgtcaaaaagtaagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 164 - DNA, 2590 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagggcaacgtcaaaaagtaagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 165 - DNA, 1732 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctccccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 166 - DNA, 2600 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacattggagggcaacgtcaaaaagtaagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 167 - DNA, 2601 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacatatggagggcaacgtcaaaaagtaagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 168 - DNA, 2332 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcaacgtcaaaaagtaagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 169 - DNA, 5 bp, Sus scrofa
ccctc

SEQ ID NO: 170 - DNA, 2590 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacaacgtcaaaaagtaagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 171 - DNA, 2598 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacaggagggcaacgtcaaaaagtaagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 172 - DNA, 2587 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacatcaaaaagtaagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 173 - DNA, 2574 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggggcaacgtcaaaaagtaagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 174 - DNA, 2591 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcggagggcaacgtcaaaaagtaagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 175 - DNA, 2599 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacaacgagggcaacgtcaaaaagtaagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 176 - DNA, 2600 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtacaatggagggcaacgtcaaaaagtaagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 177 - DNA, 1946 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtggggcttataagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 178 - DNA, 2595 bp, Sus scrofa
actggtggatgggaagggtgacagtgaacattgttttcctgtaaggacatgtgctgttgagtataaggagtaccttcatttctaccacggatagaatgggtgaccctctggatgagaaagaagggaaggattttgaggttctactatatggtgtttaatatgttttctaacattaaatccgctcaccaaatctgagacgtaaattctagtatttatttatgtgaacagggttctcagaaaggagaacttacctgccagaggtcatggctgggaagaggttaagccgccgctagcctcccttctttaaaaaaaaaaaaaaaaaaaaaaaaaggcaaaacaacttatttcattctactcagtgagctgataattgaggggaaagtttttggcaagaagggaaagtggcggggggaggacctggaagaactccctgctctggaagaatgcgggaggctgggaccatgtccctgaggagcgccgggcatccctccaactgcagggctgacccggtgtggtcttgacccgagccagaggccggctctccccgtcttttcacctcccacctcttgctcctgggacgtccttcgaccctcctggatctaacctcagtcttcctgctcctgtgcctgttgtcatagctcacagctcacagggagatccaagccacctggccgctccctctccccgctgggccagctgcctgccacctgcccttcagcccttggtgggctcccaggctcctgcagcctgtaaccagaccctgtttgctcccagcaggcacccctgagccgcactccgcacgctgttcctgaatctcccctccagaaccggagcagtgtctctacccagttcagtgaccttcgtctgtctgagccctggttaatttttgcccagtctgcaggctgtggggctcctccccttcagggatataagcctggtccgaagctgccctgtcccctgcccgtcctgagcctccccgagctcccttctcaccctcaccatggccaagggattctacatttccaaggccctgggcatcctgggcatcctcctcggcgtggcggccgtggccaccatcatcgctctgtctgtggtgtacgcccaggagaagaacaagaatgccgagcatgtcccccaggcccccacgtcgcccaccatcaccaccacagccgccatcaccttggaccagagcaagccgtggaaccggtaccgcctacccacaacgctgttgcctgattcctacaacgtgacgctgagaccctacctcactcccaacgcggatggcctgtacatcttcaagggcaaaagcatcgtccgcttcatctgccaggagcccaccgatgtcatcatcatccatagcaagaagctcaactacaccacccaggggcacatggtggtcctgcggggcgtgggggactcccaggtcccagagatcgacaggactgagctggtagagctcactgagtacctggtggtccacctcaagggctcgctgcagcccggccacatgtacgagatggagagtgaattccagggggaacttgccgacgacctggcaggcttctaccgcagcgagtactcgtcaacgtcaaaaagtaagtcaggtgggggcacaccctagatgctgaggcagagctggatcctgggggccaaggaagggcttggattcgggaccttggaaccttctggagactttggctggcccgtcgctccatccgcagctctggtagagaagctatctagacaatcagccctttcccggagagcccccctaaccttagggagtcaggggtgagtgatccaagtgcccccttgggtagaaaggaaaacaggctctgaggacagaaatttgcccaaggtctcccagctaattcaggggtggagcctgcccggactttgaccccaagtccagaaggagctctgctctcccaagtcagctggcctgtcagcctggaggcggcctgggggaggcggggagggcagggatggggctgtgcacccctttccatgcccagccagccatggcctacaccccccacccccggccacccccatgggcacaggcattttgctggcataccttctaaccccctgcttcgggcagggtgctggccacgacacagatgcagtctacagatgcccggaaatccttcccatgctttgacgagccagccatgaaggccacgttcaacatcactctcatccaccctaacaacctcacggccctgtccaatatgccgcccaaaggtgagcgggcctggcggggaccacacggcctgggaaagcaggtccctggggctggggtgcaggtccctgttgctggggtgcaggcccaggaagagggcacccctccacgcctgcgtgtcgcacccaggttccagcaccccacttgcagaagaccccaactggtctgtcactgagttcgaaaccacacctgtgatgtccacgtaccttctggcctacatcgtgagcgagttccagagcgtgaatgaaacggcccaaaatggcgtcctggtaaggggctgagcccacctgcccttccccacattggccctggcctgggaagtattcccatttatcctcatccttgtccct

SEQ ID NO: 179 - DNA, 8 bp, Sus scrofa
ggggctta

SEQ ID NO: 180 - DNA, 4 bp, Sus scrofa
tcgt

SEQ ID NO: 181 - DNA, 21 bp, Artificial sequence
  Synthetic oligonucleotide
atggcttctgtcagttttcag

SEQ ID NO: 182 - DNA, 21 bp, Artificial sequence
  Synthetic oligonucleotide
ttaatttcctgtgtcgaagat

SEQ ID NO: 183 - DNA, 24 bp, Artificial sequence
  Synthetic oligonucleotide
tctgctgaaggtgctattatatgc

SEQ ID NO: 184 - DNA, 24 bp, Artificial sequence
  Synthetic oligonucleotide
cacaatttgcctctgaattagaag

SEQ ID NO: 185 - DNA, 26 bp, Artificial sequence
  Synthetic oligonucleotide
yaagggctcaccacctactaccacca

SEQ ID NO: 186 - DNA, 8 bp, Artificial sequence
  Synthetic construct
  misc_feature (1)..(2): n = any purine
  misc_feature (5)..(7): n = any purine
  misc_feature (8)..(8): n = any pyrimidine
nngtnnnn

(19) United States
(12) Patent Application Publication
(10) Pub. No.: US 2020/0236914 A1
(43) Pub. Date: Jul. 30, 2020

(54) PATHOGEN-RESISTANT ANIMALS HAVING MODIFIED AMINOPEPTIDASE N (ANPEP) GENES

(71) Applicant: The Curators of the University of Missouri, Columbia, MO (US)

(72) Inventors: Randall S. Prather, Rocheport, MO (US); Kevin D. Wells, Columbia, MO (US); Kristin M. Whitworth, Columbia, MO (US)

(21) Appl. No.: 16/614,116

(22) PCT Filed: Apr. 26, 2019
(86) PCT No.: PCT/US19/29356

§ 371 Date: Nov. 15, 2019

Related U.S. Application Data
(60) Provisional application No. 62/663,495, filed on Apr. 27, 2018.

(51) Int. Cl. A01K 67/027; C07K 14/705; C12N 15/90; C12N 9/48

(52) U.S. Cl. CPC A01K 67/0275; C07K 14/70596; C12N 15/90; C12N 2310/20; A01K 2217/072; A01K 2227/108; A01K 2267/02; C12N 9/485

(57) ABSTRACT

Livestock animals and offspring thereof comprising at least one modified chromosomal sequence in a gene encoding an aminopeptidase N (ANPEP) protein are provided. Animal cells that contain such modified chromosomal sequences are also provided. The animals, offspring, and cells have increased resistance to pathogens, including transmissible gastroenteritis virus (TGEV) and porcine respiratory coronavirus (PRCV). The animals, offspring, and cells can optionally further comprise at least one modified chromosomal sequence in a gene encoding a CD163 protein and/or a SIGLEC1 protein. Methods for producing pathogen-resistant non-human animals or lineages of non-human animals are also provided.

### REFERENCE TO SEQUENCE LISTING SUBMITTED ELECTRONICALLY

The official copy of the sequence listing is submitted electronically via EFS-Web as an ASCII-formatted sequence listing with a file named “(16UMC002-WO) Sequence Listing filed 4.26.19”, created on Apr. 26, 2019 and having a size of 318.7 kilobytes, and is filed concurrently with the specification. The sequence listing contained in this ASCII-formatted document is part of the specification and is herein incorporated by reference in its entirety.

### FIELD OF THE INVENTION

The present invention relates to livestock animals and offspring thereof comprising at least one modified chromosomal sequence in a gene encoding an aminopeptidase N (ANPEP) protein. The invention further relates to animal cells comprising at least one modified chromosomal sequence in a gene encoding an ANPEP protein. The animals and cells have increased resistance to pathogens, including transmissible gastroenteritis virus (TGEV) and porcine respiratory coronavirus (PRCV). The invention further relates to livestock animals, offspring, and animal cells that comprise at least one modified chromosomal sequence in a gene encoding an aminopeptidase N (ANPEP) protein and also comprise at least one modified chromosomal sequence in a gene encoding a CD163 protein and/or at least one modified chromosomal sequence in a gene encoding a SIGLEC1 protein. The invention further relates to methods for producing pathogen-resistant non-human animals or lineages of non-human animals.

### BACKGROUND OF THE INVENTION

Respiratory and enteric infections caused by coronaviruses have important impacts to both human and animal health. Infection of immunologically naïve newborn pigs with transmissible gastroenteritis virus (TGEV) or porcine epidemic diarrhea virus (PEDV) can incur losses approaching 100% mortality; the result of dehydration caused by the virus-mediated destruction of enterocytes resulting in a malabsorptive diarrhea and dehydration (Madson et al., 2016; Saif et al., 2012). TGEV first appeared in the US in the 1940s (Doyle and Hutchings., 1946). The more recent emergence of porcine epidemic diarrhea virus (PEDV) in 2013 was responsible for the death of nearly seven million pigs in the US, an estimated 10% loss in pig production (Stevenson et al., 2013). TGEV can also cause 100% neonatal mortality. In older pigs, infection with TGEV or PEDV results in only mild clinical signs followed by complete recovery.

Along with the human, canine and feline coronaviruses, PEDV and TGEV belong to the genus Alphacoronavirus in the family Coronaviridae (Lin et al., 2015). Porcine respiratory coronavirus (PRCV) is also an Alphacoronavirus and is closely related to TGEV. PRCV generally causes subclinical infection or mild respiratory disease, but severe cases have been described and there is evidence that it may worsen the severity of disease when pigs are dually infected with both PRCV and another virus such as porcine respiratory and reproductive syndrome virus (PRRSV) (Killoran et al., 2016; Van Reeth et al., 1996). Moreover, PRCV-positive status of a herd may have economic implications, because some countries will not import animals that are PRCV-positive.

Coronaviruses are enveloped, single stranded, positive sense RNA viruses, placed in the order, Nidovirales. The characteristic hallmark of nidoviruses is the synthesis of a nested set of subgenomic mRNAs. The unique structural feature of coronaviruses is the “corona” formed by the spike proteins protruding from the surface of the virion. Even though the viral spike protein is the primary receptor protein for all coronaviruses, the corresponding cell surface receptors vary (Li, 2015). Delmas et al. was the first to characterize porcine aminopeptidase N (ANPEP, APN or CD13) as a candidate receptor for TGEV (Delmas et al., 1992). Porcine ANPEP is a type II membrane metallopeptidase responsible for removing N-terminal amino acids from protein substrates during digestion in the gut.

ANPEP is expressed in a variety of cell types and tissues, including small intestinal and renal tubular epithelial cells, granulocytes, macrophages, and on synaptic membranes. ANPEP is abundantly expressed in the epithelial cells of the small intestine (enterocytes). ANPEP is highly expressed during tissue vascularization, such as with endothelium maintenance, tumor formation (Bhagwat et al., 2001; Guzman-Rojas et al., 2012) and mammogenesis.

While the epithelial cells of the small intestine appears to be the main site of PED virus clinical infection, other sites such as alveolar macrophages can also become infected (Park and Shin, 2014). Indeed, deep sequencing data from alveolar macrophages has identified message for ANPEP (unpublished). It was been proposed that other sites of infection may serve as a reservoir for persistent infection (Park and Shin, 2014).

ANPEP is a membrane-bound zinc-dependent metalloprotease that hydrolyzes unsubstituted N-terminal residues with neutral side chains. Its only known substrate in the renal proximal tubule is angiotensin III; which it cleaves to angiotensin IV. It also metabolizes enkephalins and endorphins. Finally, it functions in signal transduction, cell cycle control and differentiation.

In addition to its role as a receptor for certain coronaviruses, ANPEP also plays important roles in many physiological processes, including peptide metabolism, cell motility and adhesion, pain sensation, blood pressure regulation, tumor angiogenesis and metastasis, immune cell chemotaxis, sperm motility, cell-cell adhesion, and mood regulation (Chen et al., 2012).

Porcine and human ANPEP share high sequence identity, and indistinguishable biochemical and kinetic properties (Chen et al., 2012). The ANPEP gene is located on chromosome 7 in the pig, and has at least three splice variants. Two promoters of ANPEP have been identified in myeloid/fibroblast cells and in intestinal epithelial cells (Shapiro et al., 1991). They are about 8 kb apart and yield transcripts with varying 5′ non-coding regions. The epithelial promoter is located closer to the coding region, while the myeloid promoter is distal (Shapiro et al., 1991). There are three publically accepted transcripts/splice variants associated with the ANPEP gene: X1, X2 and X3. Variant X1 has 20 exons and encodes a 1017 amino acid protein. Variant X2 and X3 both have 21 exons and each encode a 963 amino acid protein. The mature ANPEP protein has a 24 amino acid hydrophobic segment near its N terminus and serves as a signal for membrane insertion. The large extracellular C-terminal domain contains a zinc-binding metalloproteinase superfamily domain like region, a cytosolic Ser/Thr-rich junction, and a transition state stabilizer.

As can be appreciated from the foregoing, a need exists in the art for development of strategies to induce resistance to TGEV and related viruses such as PRCV in animals.

Another economically important disease of swine in North America, Europe and Asia is porcine reproductive and respiratory syndrome (PRRS), which costs North American producers approximately $600 million annually (Holtkamp et al., 2013). Clinical disease syndromes caused by infection with porcine reproductive and respiratory syndrome virus (PRRSV) were first reported in the United States in 1987 (Keffaber, 1989) and later in Europe in 1990 (Wensvoort et al., 1991). Infection with PRRSV results in respiratory disease including cough and fever, reproductive failure during late gestation, and reduced growth performance. The virus also participates in a variety of polymicrobial disease syndrome interactions while maintaining a life-long subclinical infection (Rowland et al., 2012). Losses are the result of respiratory disease in young pigs, poor growth performance, reproductive failure, and in utero infection (Keffaber, 1989).

Porcine reproductive and respiratory syndrome virus (PRRSV) belongs to the family Arterividae along with murine lactate dehydrogenase-elevating virus, simian hemorrhagic fever virus, and equine arteritis virus. Structurally, the arteriviruses resemble togaviruses, but similar to coronaviruses, replicate via a nested 3′-co-terminal set of subgenomic mRNAs, which possess a common leader and a poly-A tail. The arteriviruses share important properties related to viral pathogenesis, including a tropism for macrophages and the capacity to cause severe disease and persistent infection (Plagemann, 1996). Molecular comparisons between North American and European viruses place all PRRSV isolates into one of two genotypes, Type 2 or Type 1, respectively. Even though the two genotypes possess only about 70% identity at the nucleotide level (Nelsen et al., 1999), both share a tropism for CD163-positive cells, establish long-term infections, and produce similar clinical signs.

CD163 is a 130 kDa type 1 membrane protein composed of nine scavenger receptor cysteine-rich (SRCR) domains and two spacer domains along with a transmembrane domain and a short cytoplasmic tail (Fabriek et al., 2005). Porcine CD163 contains 17 exons that code for a peptide signal sequence followed by nine SRCR domains, two linker domains (also referred to as proline serine threonine (PST) domains, located after SRCR 6 and SRCR 9), and a cytoplasmic domain followed by a short cytoplasmic tail. Surface expression of CD163 is restricted to cells of the monocyte-macrophage lineage. In addition to functioning as a virus receptor, CD163 exhibits several important functions related to maintaining normal homeostasis. For instance, following infection or tissue damage, CD163 functions as a scavenger molecule, removing haptoglobin-hemoglobin complexes from the blood (Kristiansen et al., 2001). The resulting heme degradation products regulate the associated inflammatory response (Fabriek et al., 2005). HbHp scavenging is a major function of CD163 and locates to SRCR 3 (Madsen et al., 2004). Metabolites released by macrophages following HbHp degradation include bilirubin, CO, and free iron. One important function of CD163 the prevention of oxidative toxicity that results from free hemoglobin (Kristiansen et al., 2001; Soares et al., 2009).

Other important functions of C163 include erythroblast adhesion (SRCR2), being a TWEAK (tumor necrosis factor-like weak inducer of apoptosis) receptor (SRCR1-4 & 6-9), being a bacterial receptor (SRCR5), and being an African Swine Virus receptor (Sanchez-Torres et al. 2003). CD163 also has a potential role as an immune-modulator (discussed in Van Gorp et al. 2010).

CD163 was first described as a receptor for PRRSV by Calvert et. al. (2007). Transfection of non-permissive cell lines with CD163 cDNAs from a variety of species, including simian, human, canine, and mouse, can make cells permissive for PRRSV infection (Calvert et al., 2007). In addition to CD163, a second receptor protein, CD169 (also known as sialoadhesin or SIGLEC1), was identified as being a primary PRRSV receptor involved in forming the initial interaction with the GP5-matrix (M) heterodimer, the major protein on the surface of the virion (Delputte et al., 2002). In this model, the subsequent interaction between CD163 and the GP2, 3, 4 heterotrimer in an endosomal compartment mediates uncoating and the release of the viral genome into the cytoplasm (Van Breedam et al., 2010, Allende et al., 1999). These results supported previous in vitro studies showing that PRRSV-resistant cell lines lacking surface CD169 and CD163 supported virus replication after transfection with a CD163 plasmid (Welch et al., 2010).

Another receptor for PRRSV has been identified, purified, sequenced, and named SIGLEC1, CD169, or sialoadhesin (Vanderheijden et al., 2003; Wissink et al., 2003). SIGLEC1 is a transmembrane protein belonging to a family of sialic acid binding immunoglobulin-like lectins. It was first described as a sheep erythrocyte binding receptor on macrophages of hematopoietic and lymphoid tissues (Delputte et al., 2004). SIGLEC proteins contain an N-terminal V-set domain containing the sialic acid binding site, followed by a variable number of C2-set domains, a transmembrane domain, and a cytoplasmic tail. In contrast to other SIGLEC proteins, SIGLEC1 does not have a tyrosine-based motif in the cytoplasmic tail (Oetke et al., 2006). SIGLEC1, which is expressed on macrophages, functions in cell-to-cell interactions through the binding of sialic acid ligands on erythrocytes, neutrophils, monocytes, NK cells, B cells, and some cytotoxic T cells. The SIGLEC1-sialic acid interaction participates in several aspects of adaptive immunity, such as antigen processing and presentation to T cells and activation of B cells and CD8 T cells (reviewed in Martinez-Pomares et al., 2012 and O'Neill et al., 2013).

An intact N-terminal domain on SIGLEC1 has been suggested to be both necessary and sufficient for PRRSV binding and internalization by cultured macrophages (An et al., 2010; Delputte et al., 2007). Transfection of SIGLEC1-negative cells, such as PK-15, with SIGLEC1 is sufficient to mediate virus internalization. Incubation of PRRSV-permissive cells with anti-SIGLEC1 monoclonal antibody (MAb) blocks PRRSV binding and internalization (Vanderheijden N et al., 2003). On the virus side, removal of the sialic acid from the surface of the virion or preincubation of the virus with sialic acid-specific lectins blocks infection (Delputte et al., 2004; Delputte et al., 2007; Van Breedam et al., 2010).

Many characteristics of both PRRSV pathogenesis (especially at the molecular level) and epizootiology are poorly understood, thus making control efforts difficult. Currently, producers often vaccinate swine against PRRSV with modified-live attenuated strains or killed virus vaccines, however, current vaccines often do not provide satisfactory protection. This is due to both the strain variation and inadequate stimulation of the immune system. In addition to concerns about the efficacy of the available PRRSV vaccines, there is strong evidence that the modified-live vaccine currently in use can persist in individual pigs and swine herds and accumulate mutations (Mengeling et al. 1999), as has been demonstrated with virulent field isolates following experimental infection of pigs (Rowland et al., 1999). Furthermore, it has been shown that vaccine virus is shed in the semen of vaccinated boars (Christopher-Hennings et al., 1997). As an alternative to vaccination, some experts are advocating a “test and removal” strategy in breeding herds (Dee et al., 1998). Successful use of this strategy depends on removal of all pigs that are either acutely or persistently infected with PRRSV, followed by strict controls to prevent reintroduction of the virus. The difficulty, and much of the expense, associated with this strategy is that there is little known about the pathogenesis of persistent PRRSV infection and thus there are no reliable techniques to identify persistently infected pigs.

Thus, a need also exists in the art to induce resistance to PRRSV in animals. It would also be beneficial to induce PRRSV and TGEV and/or PRCV resistance in the same animal.

### BRIEF SUMMARY OF THE INVENTION

Livestock animals and offspring thereof are provided. The animals and offspring comprise at least one modified chromosomal sequence in a gene encoding an aminopeptidase N (ANPEP) protein.

Animal cells are also provided. The animal cells comprise at least one modified chromosomal sequence in a gene encoding an ANPEP protein.

Further livestock animals and offspring thereof are provided. The animals and offspring comprise at least one modified chromosomal sequence in a gene encoding an ANPEP protein and at least one modified chromosomal sequence in a gene encoding a CD163 protein.

Further animal cells are provided. The animal cells comprise at least one modified chromosomal sequence in a gene encoding an ANPEP protein and at least one modified chromosomal sequence in a gene encoding a CD163 protein.

Additional livestock animals and offspring thereof are provided. The animals and offspring comprise at least one modified chromosomal sequence in a gene encoding an ANPEP protein and at least one modified chromosomal sequence in a gene encoding a SIGLEC1 protein.

Additional animal cells are provided. The animal cells comprise at least one modified chromosomal sequence in a gene encoding an ANPEP protein and at least one modified chromosomal sequence in a gene encoding a SIGLEC1 protein.

Further livestock animals and offspring thereof are provided. The animals and offspring comprise at least one modified chromosomal sequence in a gene encoding an ANPEP protein, at least one modified chromosomal sequence in a gene encoding a CD163 protein, and at least one modified chromosomal sequence in a gene encoding a SIGLEC1 protein.

Further animal cells are provided. The animal cells comprise at least one modified chromosomal sequence in a gene encoding an ANPEP protein, at least one modified chromosomal sequence in a gene encoding a CD163 protein, and at least one modified chromosomal sequence in a gene encoding a SIGLEC1 protein.

A method for producing a non-human animal or a lineage of non-human animals is provided. The animal or lineage has reduced susceptibility to a pathogen. The method comprises modifying an oocyte or a sperm cell to introduce a modified chromosomal sequence in a gene encoding an aminopeptidase N (ANPEP) protein into at least one of the oocyte and the sperm cell, and fertilizing the oocyte with the sperm cell to create a fertilized egg containing the modified chromosomal sequence in the gene encoding a ANPEP protein. The method further comprises transferring the fertilized egg into a surrogate female animal, wherein gestation and term delivery produces a progeny animal. The method additionally comprises screening the progeny animal for susceptibility to the pathogen, and selecting progeny animals that have reduced susceptibility to the pathogen as compared to animals that do not comprise a modified chromosomal sequence in a gene encoding an ANPEP protein.

Another method for producing a non-human animal or a lineage of non-human animals is provided. The animal or lineage has reduced susceptibility to a pathogen. The method comprises modifying a fertilized egg to introduce a modified chromosomal sequence in a gene encoding an ANPEP protein into the fertilized egg. The method further comprises transferring the fertilized egg into a surrogate female animal, wherein gestation and term delivery produces a progeny animal. The method additionally comprises screening the progeny animal for susceptibility to the pathogen, and selecting progeny animals that have reduced susceptibility to the pathogen as compared to animals that do not comprise a modified chromosomal sequence in a gene encoding an ANPEP protein.

A method of increasing a livestock animal's resistance to infection with a pathogen is provided. The method comprises modifying at least one chromosomal sequence in a gene encoding an aminopeptidase N (ANPEP) protein so that ANPEP protein production or activity is reduced, as compared to ANPEP protein production or activity in a livestock animal that does not comprise a modified chromosomal sequence in a gene encoding an ANPEP protein.

A population of livestock animals is provided. The population comprises two or more of any of the livestock animals and/or offspring thereof described herein.

Another population of animals is provided. The population comprises two or more animals made by any of the methods described herein and/or offspring thereof.

A nucleic acid molecule is provided. The nucleic acid molecule comprises a nucleotide sequence selected from the group consisting of:

(a) a nucleotide sequence having at least 80% sequence identity to the sequence of SEQ ID NO: 135, wherein the nucleotide sequence comprises at least one substitution, insertion, or deletion relative to SEQ ID NO: 135;

(b) a nucleotide sequence having at least 80% sequence identity to the sequence of SEQ ID NO: 132, wherein the nucleotide sequence comprises at least one substitution, insertion, or deletion relative to SEQ ID NO: 132; and

(c) a cDNA of (a) or (b).

Other objects and features will be in part apparent and in part pointed out hereinafter.

### BRIEF DESCRIPTION OF THE DRAWINGS

FIG. 1. Targeting vectors and CRISPRs used to modify CD163. Panel A depicts wild type exons 7, 8 and 9 of the CD163 gene that was targeted for modification using CRISPRs. Panel B shows the targeting vector designed to replace pig exon 7 (pig domain SRCR5 of CD163) with DNA that encodes human SRCR8 of CD163L. This targeting vector was used in transfections with drug selection by G418. PCR primers for the long range, left arm and right arm assay are labelled with arrows for 1230, 3752, 8791, 7765 and 7775. Panel C depicts a targeting vector identical to the one shown in panel B, but wherein the Neo cassette was removed. This targeting vector was used to target CD163 in cells that were already neomycin resistant. Primers used in small deletions assays are illustrated with arrows and labeled GCD163F and GCD163R. Panel D emphasizes the exons targeted by CRISPRs. Location of CRISPRs 10, 131, 256 and 282 are represented by the downward facing arrows on exon 7. The CRISPR numbers represent the number of base pairs from the intron-exon junction of intron 6 and exon 7.

FIG. 2. Targeting vector and CRISPRs used to modify CD1D. Panel A depicts wild type exons 3, 4, 5, 6 and 7 of the CD1D gene that was targeted for modification by CRISPRs. Panel B shows the targeting vector designed to replace exon 3 with the selectable marker Neo. This targeting vector was used in combination with CRISPRs to modify CD1D. PCR primers for the long range, left arm and right arm assay are labeled with arrows for 3991, 4363, 7373 and 12806. Panel C depicts the exons targeted by CRISPRs. Locations of CRISPRs 4800, 5350, 5620 and 5626 are represented by the downward facing arrows on exon 3. Primers used in small deletions assays are illustrated with arrows and labelled GCD1DF and GCD1DR.

FIG. 3. Generation of CD163 and CD1D knockout pigs by CRISPR/Cas9 and SCNT. A) Targeted deletion of CD163 in somatic cells after transfection with CRISPR/Cas9 and donor DNA. A wild-type (WT) genotype results in a 6545 base pair (bp) band. Lanes 1-6 represent six different colonies from a single transfection with CRISPR 10 with Cas9 and donor DNA containing Neo. Lanes 1, 4, and 5 show a large homozygous deletion of 1500-2000 bp. Lane 2 represents a smaller homozygous deletion. Lanes 3 and 6 represent either a WT allele and a small deletion or a biallelic modification of both alleles. The exact modifications of each colony were only determined by sequencing for colonies used for SCNT. The faint WT band in some of the lanes may represent cross-contamination of fetal fibroblasts from a neighboring WT colony. NTC=no template control. B) Targeted deletion of CD1D in somatic cells after transfection with CRISPR/Cas9 and donor DNA. A WT genotype results in an 8729 bp band. Lanes 1-4 represent colonies with a 500-2000 bp deletion of CD1D. Lane 4 appears to be a WT colony. NTC=no template control. C) Image of CD163 knockout pig produced by SCNT during the study. This male piglet contains a homozygous 1506 bp deletion of CD163. D) Image of CD1D pigs produced during the study. These piglets contain a 1653 bp deletion of CD1D. E) Genotype of two SCNT litters containing the 1506 bp deletion of CD163. Lanes 1-3 (litter 63) and lanes 1-4 (litter 64) represent the genotype for each piglet from each litter. Sow indicates the recipient female of the SCNT embryos, and WT represents a WT control. NTC=no template control. F) Genotype of two SCNT litters containing the 1653 bp deletion of CD1D. Lanes 1-7 (litter 158) and lanes 1-4 (litter 159) represent the genotype for each piglet.

FIG. 4. Effect of CRISPR/Cas9 system in porcine embryos. A) Frequency of blastocyst formation after injection of different concentrations of CRISPR/Cas9 system into zygotes. Toxicity of the CRISPR/Cas9 system was lowest at 10 ng/μl. B) The CRISPR/Cas9 system can successfully disrupt expression of eGFP in blastocysts when introduced into zygotes. Original magnification X4. C) Types of mutations on eGFP generated using the CRISPR/Cas9 system: WT genotype (SEQ ID NO:16), #1 (SEQ ID NO:17), #2 (SEQ ID NO:18), and #3 (SEQ ID NO:19).

FIG. 5. Effect of CRISPR/Cas9 system in targeting CD163 in porcine embryos. A) Examples of mutations generated on CD163 by the CRISPR/Cas9 system: WT genotype (SEQ ID NO:20), #1-1 (SEQ ID NO:21), #1-4 (SEQ ID NO:22), and #2-2 (SEQ ID NO:23). All the embryos examined by DNA sequencing showed mutation on the CD163 (18/18). CRISPR 131 is highlighted in bold. B) Sequencing read of a homozygous deletion caused by the CRISPR/Cas9 system. The image represents #1-4 from panel A carrying a 2 bp deletion of CD163.

FIG. 6. Effect of CRISPR/Cas9 system when introduced with two types of CRISPRs. A) PCR amplification of CD163 in blastocysts injected with CRISPR/Cas9 as zygotes. Lanes 1,3,6, and 12 show the designed deletion between two different CRISPRs. B) PCR amplification of CD1D in blastocysts injected with CRISPR/Cas9 as zygotes. CD1D had a lower frequency of deletion as determined by gel electrophoresis when compared to CD163 (3/23); lanes 1,8, and 15 show obvious deletions in CD1D. C) CRISPR/Cas9 system successfully targeted two genes when the system was provided with two CRISPRs targeting CD163 and eGFP. The modifications of CD163 and eGFP are shown: CD163 WT (SEQ ID NO:24), CD163 #1 (SEQ ID NO:25), CD163 #2 (SEQ ID NO:26), CD163 #3 (SEQ ID NO:27), eGFP WT (SEQ ID NO:28), eGFP #1-1 (SEQ ID NO:29), eGFP #1-2 (SEQ ID NO: 30), eGFP #2 (SEQ ID NO:31), and eGFP #3 (SEQ ID NO:32).

FIG. 7. CD163 knockout pigs generated by CRISPR/Cas9 system injected into zygotes. A) PCR amplification of CD163 from the knockout pigs; a clear sign of deletion was detected in litters 67-2 and 67-4. B) Image of CD163 knockout pigs with a surrogate. All the animals are healthy and show no signs of abnormalities. C) Genotype of CD163 knockout pigs. Wild-type (WT) sequence is shown as SEQ ID NO: 33. Two animals (from litters 67-1 (SEQ ID NO:34) and 67-3 (SEQ ID NO:37)) are carrying a homozygous deletion or insertion in CD163. The other two animals (from litters 67-2 and 67-4) are carrying a biallelic modification of CD163: #67-2 A1 (SEQ ID NO:35), #67-2 A2 (SEQ ID NO:36), #67-4 A1 (SEQ ID NO:38), and #67-4 a2 (SEQ ID NO:39). The deletion was caused by introducing two different CRISPRs with Cas9 system. No animals from the zygote injection for CD163 showed a mosaic genotype.

FIG. 8. CD1D knockout pigs generated by CRISPR/Cas9 system injected into zygotes. A) PCR amplification of CD1D from knockout pigs; 166-1 shows a mosaic genotype for CD1D. 166-2, 166-3, and 166-4 do not show a change in size for the amplicon, but sequencing of the amplicon revealed modifications. WT FF=wild-type fetal fibroblasts. B) PCR amplification of the long-range assay showed a clear deletion of one allele in piglets 166-1 and 166-2. C) Image of CD1D knockout pigs with surrogate. D) Sequence data of CD1D knock out pigs; WT (SEQ ID NO:40), #166-1.1 (SEQ ID NO: 41), #166-1.2 (SEQ ID NO:42), #166-2 (SEQ ID NO:43), #166-3.1 (SEQ ID NO:44), #166-3.2 (SEQID NO:45), and #166-4 (SEQ ID NO:46). The atg start codon in exon 3 is shown in bold and also lower case.

FIG. 9. Clinical signs during acute PRRSV infection. Results for daily assessment for the presence of respiratory signs and fever for CD163+/+(n=6) and CD163−/− (n=3).

FIG. 10. Lung histopathology during acute PRRSV infection. Representative photomicrographs of H and E stained tissues from wild-type and knockout pigs. The left panel shows edema and infiltration of mononuclear cells. The right panel from a knockout pig shows lung architecture of a normal lung.

FIG. 11. Viremia in the various genotypes. Note that the CD163−/− piglet data lies along the X axis.

FIG. 12. Antibody production in null, wild type and uncharacterized allele pigs.

FIG. 13. Cell surface expression of CD163 in individual pigs. Lines appearing towards the right in the uncharacterized A, uncharacterized B, and CD163+/+ panels represent the CD163 antibody while the lines appearing towards the left-hand sides of these panels are the no antibody controls (background). Note that in the CD163−/− animals, the CD163 staining overlaps with the background control, and that the CD163 staining in the uncharacterized alleles is roughly half way between the WT level and the background (also note that this is a log scale, thus less than ˜10%).

FIG. 14. Level of CD169 on alveolar macrophages from three representative pigs and the no antibody control (FITC labelled anti-CD169).

FIG. 15. Viremia in the various genotypes. Note that the 443 amino acid piglet data lies along the X-axis.

FIG. 16. Genomic Sequence of wild type CD163 exons 7-10 used as a reference sequence (SEQ ID NO: 47). The sequence includes 3000 bp upstream of exon 7 to the last base of exon 10. The underlined regions show the locations of exons 7, 8, 9, and 10, respectively.

FIG. 17. Diagram of CD163 modifications illustrating several CD163 chromosomal modifications, the predicted protein product for each modification, and relative macrophage expression for each modification, as measured by the level of surface CD163 on porcine alveolar macrophages (PAMs). Black regions indicate introns and white regions indicate exons. The hatched region indicates the hCD163L1 exon 11 mimic, the homolog of porcine exon 7. The grey region indicates the synthesized intron with PGK Neo construct.

FIG. 18. Diagram of the porcine CD163 protein and gene sequence. A) CD163 protein SRCR (ovals) and PST (squares) domains along with the corresponding gene exons. B) Comparison of the porcine CD163 SRCR 5 (SEQ ID NO: 120) with the human CD163L1 SRCR 8 (SEQ ID NO: 121) homolog.

FIG. 19. Representative results for surface expression of CD163 and CD169 on PAMs from wild-type and CD163-modified pigs. Panels A-E show results for the CD163 modifications as illustrated in FIG. 17. Pooled data for d7(1467) and d7(1280) are shown in panel D.

FIG. 20. Serum haptoglobin levels in wild-type and CD163-modified pigs.

FIG. 21. Relative permissiveness of wild-type and HL11m PAMs to infection with Type 2 PRRSV isolates.

FIG. 22. Infection of CD163 modified pigs with Type 1 and Type 2 PRRSV isolates.

FIG. 23. Virus load for WT and CD163-modified pigs infected with Type 2 viruses.

FIG. 24. SIGLEC1 knockout strategy. Panel A shows the organization of porcine SIGLEC1, which contains 21 exons and spans approximately 20 kb (GenBank accession no. CU467609). Panel B illustrates the targeting construct used for homologous recombination. The primer sequences for PCR amplification and cloning are labeled (F) and (R). The ‘upper arm’ DNA fragment is ˜3.5 kbp upstream of exon 1 and includes part of exon 1 (after the start codon). The sialic binding domain is located in exon 2. The ‘lower arm’ DNA fragment includes exons 4, 5, 6 and part of exon 7. Most of exon 1 and all of exons 2 and 3 were substituted with a neomycin (neo) cassette under the control of a PGK promoter. A thymidine kinase (TK) cassette was available immediately downstream of the lower arm but was not used for selection. Three in frame stop codons (sss) were introduced into the end of the upper and lower arms by including them in the antisense and sense PCR primers used to amplify the region. Panel C shows the mutated SIGLEC1 gene after homologous recombination. The horizontal arrows show the location of PCR primers used for screening (see Table 17 for primer sequences).

FIG. 25. PCR screening of wild-type and targeted SIGLEC1^{+/-} alleles in transgenic founder pigs. PCR primers, “c” and “d” (see labeled arrows in FIG. 24) were used to amplify genomic DNA from the eight founder pigs, derived from the male 4-18 clone. Panel A shows DNA from KW2 cells (the initial cells used for transfection), the targeting plasmid, the targeted cells 4-18 (note the two bands, ˜2,400 and ˜2,900 bp), a non-targeted fibroblast and water blank as a negative PCR control. Arrow shows the location of a faint 2,900 bp band for the 4-18 clone. Panel B shows the results for eight F0 transgenic pigs. Note the presence of two bands (˜2,400 and 2,900 bp) for each piglet. A wild-type 4-18 clone, 11-1 and targeting plasmid show only a single band. Some fragment sizes from the molecular size markers are indicated.

FIG. 26. Southern blot identification of knockout pigs in F2 litter #52. The upper arrow points to the location of the wild-type band (7,892 bp), while the lower arrow identifies the predicted location of the gene knockout (7,204 bp). Molecular size standards are shown (STD). In addition to the SIGLEC1 (−/−) pigs, examples of wild-type (+/+), and heterozygous (+/−) pigs are also depicted.

FIG. 27. Expression of SIGLEC1 (CD169) and CD163 on the surface of PAM cells. Fresh PAM cells were stained for CD169 (mAb 3B11/11) or CD163 (mAb 2A10/11). PAM cells stained with only FITC-conjugated goat-anti mouse IgG were included as a background control.

FIG. 28. Genomic sequence of wildtype ANPEP exons 2-4 used as a reference sequence (SEQ ID NO: 135). The sequence includes the last 773 base pairs in intron 2, exon 2, intron 3, exon 3, intron 4, exon 4, and 81 base pairs of intron 5. The underlined regions show the locations of exons 2, 3, and 4, respectively. CRISPR Guides 2 and 3 (Table 20) targeting exon 2 are each bolded and double underlined.

FIG. 29. Illustrative PCR results for SCNT-derived fetuses detecting modified ANPEP alleles.

FIG. 30. Illustrative PCR results for zygote-injected fetuses detecting modified ANPEP alleles.

FIGS. 31 and 32. Illustrative PCR results for live pigs born from zygote injections detecting modified ANPEP alleles.

FIG. 33. Schematic diagram of the wild-type and modified ANPEP alleles present in animals used in TGEV and PEDV challenge studies.

FIG. 34. Illustrative immunohistochemistry results for ANPEP staining of ileum from wild-type pigs (+/+), pigs having two null ANPEP alleles (−/−), or a null ANPEP allele in combination with an allele having a 9 base pair (3 amino acid deletion, −/d9) or a 12 base pair (4 amino acid, −/d12) in-frame deletion.

FIG. 35. Photograph of pig 158-1, having a modified chromosomal sequence for ANPEP, at sexual maturity.

FIG. 36. Illustrative PCR results measuring levels of PEDV virus in serum and feces of wild-type pigs and pigs having a knockout or in-frame deletion in ANPEP, measured 0, 7, and 9 days after exposure to PEDV.

FIG. 37. Illustrative immunohistochemistry results for PEDV antigen staining of ileum from wild-type pigs and pigs having a knockout (KO) or in-frame deletion in ANPEP, 9 days after initial exposure to PEDV.

FIG. 38. Illustrative PCR results measuring the levels of TGEV virus in feces of wild-type pigs and pigs having a knockout or in-frame deletion in ANPEP, measured 0, 3, 6, and 7 days after exposure to TGEV.

FIG. 39. Illustrative immunohistochemistry results for TGEV antigen staining of ileum from wild-type pigs and pigs having a knockout (KO) or in-frame deletion in ANPEP, 9 days after initial exposure to the virus.

FIG. 40. Illustrative ELISA assay data showing the presence or absence of TGEV-specific antibody in wild-type pigs and pigs having a knockout (KO) or in frame deletion in ANPEP.

FIG. 41. Illustrative PCR results showing modified CD163 alleles (Panel A), ANPEP alleles (Panel B) and SIGLEC1 alleles (Panel C) in a litter of animals generated by crossing pigs having modified chromosomal sequences for ANPEP, CD163 and/or SIGLEC1. ANPEP modifications were confirmed from Panel B by Sanger sequencing (Panel D).

FIG. 42. Illustrative fluorescent microscopy images of porcine lung alveolar cells obtained from ANPEP^{-/-} (KO, Panel A) and wild-type (WT, Panel B) animals. Cells were infected with TGEV, PRCV, and PEDV, as indicated. Nuclei were stained with propidium iodide (left columns in Panels A and B). Virus-infected cells were detected using FITC-labeled coronavirus anti-N protein antibodies (middle columns in Panels A and B). Merged images are shown in right columns in Panels A and B.

### DETAILED DESCRIPTION OF THE INVENTION

The present invention is directed to livestock animals and offspring thereof comprising at least one modified chromosomal sequence in a gene encoding an aminopeptidase N (ANPEP) protein. The invention further relates to animal cells comprising at least one modified chromosomal sequence in a gene encoding an ANPEP protein. The animals and cells have increased resistance to pathogens, including transmissible gastroenteritis virus (TGEV) and porcine respiratory coronavirus (PRCV).

The animals and cells have chromosomal modifications (e.g., insertions, deletions, or substitutions) that inactivate or otherwise modulate ANPEP gene activity. ANPEP is involved in entry of TGEV into cells. Thus, animals or cells having inactivated ANPEP genes display resistance to TGEV when challenged. The animals and cells can be created using any number of protocols, including those that make use of gene editing.

In addition to the at least one modified chromosomal sequence in a gene encoding an aminopeptidase N (ANPEP) protein, the animals, offspring, and animals can further comprise at least one modified chromosomal sequence in a gene encoding a CD163 protein and/or at least one modified chromosomal sequence in a gene encoding a SIGLEC1 protein. Such animals suitably have increased resistance to additional pathogens, e.g., porcine reproductive and respiratory syndrome virus (PRRSV).

Populations of any of the animals described herein are also provided.

The present invention is further directed to methods for producing pathogen-resistant non-human animals or lineages of non-human animals comprising introducing a modified chromosomal sequence in a gene encoding an ANPEP protein.

The methods can comprise introducing into an animal cell or an oocyte or embryo an agent that specifically binds to a chromosomal target site of the cell and causes a double-stranded DNA break or otherwise inactivates or reduces activity of an ANPEP gene or protein therein using gene editing methods such as the Clustered Regularly Interspaced Short Palindromic Repeats (CRISPR)/Cas system, Transcription Activator-Like Effector Nucleases (TALENs), Zinc Finger Nucleases (ZFN), recombinase fusion proteins, or meganucleases.

Also described herein is the use of one or more particular ANPEP loci in tandem with a polypeptide capable of effecting cleavage and/or integration of specific nucleic acid sequences within the ANPEP loci. Examples of the use of ANPEP loci in tandem with a polypeptide or RNA capable of effecting cleavage and/or integration of the ANPEP loci include a polypeptide selected from the group consisting of zinc finger proteins, meganucleases, TAL domains, TALENs, RNA-guided CRISPR/Cas recombinases, leucine zippers, and others known to those in the art. Particular examples include a chimeric (“fusion”) protein comprising a site-specific DNA binding domain polypeptide and cleavage domain polypeptide (e.g., a nuclease), such as a ZFN protein comprising a zinc-finger polypeptide and a FokI nuclease polypeptide. Described herein are polypeptides comprising a DNA-binding domain that specifically binds to an ANPEP gene. Such a polypeptide can also comprise a nuclease (cleavage) domain or half-domain (e.g., a homing endonuclease, including a homing endonuclease with a modified DNA-binding domain), and/or a ligase domain, such that the polypeptide may induce a targeted double-stranded break, and/or facilitate recombination of a nucleic acid of interest at the site of the break. A DNA-binding domain that targets an ANPEP locus can be a DNA-cleaving functional domain. The foregoing polypeptides can be used to introduce an exogenous nucleic acid into the genome of a host organism (e.g., an animal species) at one or more ANPEP loci. The DNA-binding domains can comprise a zinc finger protein with one or more zinc fingers (e.g., 2, 3, 4, 5, 6, 7, 8, 9 or more zinc fingers), which is engineered (non-naturally occurring) to bind to any sequence within an ANPEP gene. Any of the zinc finger proteins described herein may bind to a target site within the coding sequence of the target gene or within adjacent sequences (e.g., promoter or other expression elements). The zinc finger protein can bind to a target site in an ANPEP gene.

### Definitions

When introducing elements of the present invention or the preferred embodiments(s) thereof, the articles “a”, “an”, “the”, and “said” are intended to mean that there are one or more of the elements.

The term “and/or” means any one of the items, any combination of the items, or all of the items with which this term is associated.

A “binding protein” is a protein that is able to bind to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

The terms “comprising”, “including”, and “having” are intended to be inclusive and mean that there may be additional elements other than the listed elements.

The term “CRISPR” stands for “clustered regularly interspaced short palindromic repeats.” CRISPR systems include Type I, Type II, and Type III CRISPR systems.

The term “Cas” refers to “CRISPR associated protein.” Cas proteins include but are not limited to Cas9 family member proteins, Cas6 family member proteins (e.g., Csy4 and Cas6), and Cas5 family member proteins.

The term “Cas9” can generally refer to a polypeptide with at least about 5%, 10%, 20%, 30%, 40%, 50%, 60%, 70%, 80%, 90%, or 100% sequence identity and/or sequence similarity to a wild-type Cas9 polypeptide (e.g., Cas9 from S. pyogenes). Illustrative Cas9 sequences are provided by SEQ ID NOs. 1-256 and 795-1346 of U.S. Patent Publication No. 2016/0046963. SEQ ID NOs. 1-256 and 795-1346 of U.S. Patent Publication No. 2016/0046963 are hereby incorporated herein by reference. “Cas9” can refer to can refer to a polypeptide with at most about 5%, 10%, 20%, 30%, 40%, 50%, 60%, 70%, 80%, 90%, or 100% sequence identity and/or sequence similarity to a wild type Cas9 polypeptide (e.g., from S. pyogenes). “Cas9” can refer to the wild-type or a modified form of the Cas9 protein that can comprise an amino acid change such as a deletion, insertion, substitution, variant, mutation, fusion, chimera, or any combination thereof.

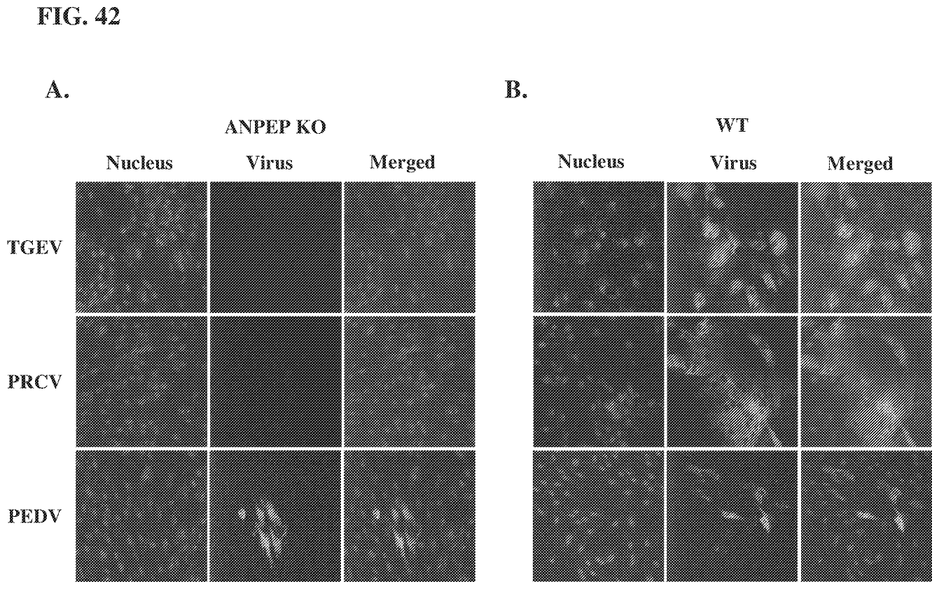

The term “Cas5” can generally refer to can refer to a polypeptide with at least about 5%, 10%, 20%, 30%, 40%, 50%, 60%, 70%, 80%, 90%, or 100% sequence identity and/or sequence similarity to a wild type illustrative Cas5 polypeptide (e.g., Cas5 from D. vulgaris). Illustrative Cas5 sequences are provided in FIG. 42 of U.S. Patent Publication No. 2016/0046963. FIG. 42 of U.S. Patent Publication No. 2016/0046963 is hereby incorporated herein by reference. “Cas5” can generally refer to can refer to a polypeptide with at most about 5%, 10%, 20%, 30%, 40%, 50%, 60%, 70%, 80%, 90%, or 100% sequence identity and/or sequence similarity to a wild-type Cas5 polypeptide (e.g., a Cas5 from D. vulgaris). “Cas5” can refer to the wild-type or a modified form of the Cas5 protein that can comprise an amino acid change such as a deletion, insertion, substitution, variant, mutation, fusion, chimera, or any combination thereof.

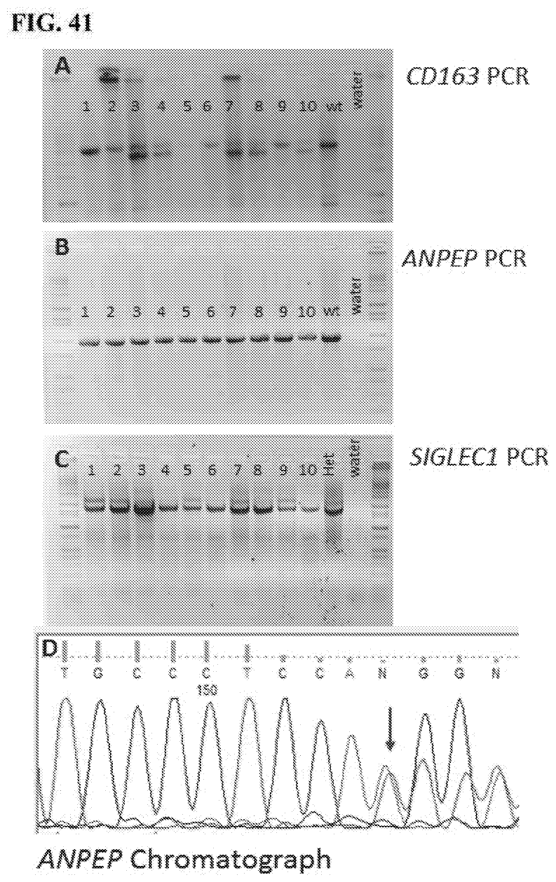

The term “Cas6” can generally refer to can refer to a polypeptide with at least about 5%, 10%, 20%, 30%, 40%, 50%, 60%, 70%, 80%, 90%, or 100% sequence identity and/or sequence similarity to a wild type illustrative Cas6 polypeptide (e.g., a Cas6 from T. thermophilus). Illustrative Cas6 sequences are provided in FIG. 41 of U.S. Patent Publication No. 2016/0046963. FIG. 41 of U.S. Patent Publication No. 2016/0046963 is hereby incorporated herein by reference. “Cas6” can generally refer to can refer to a polypeptide with at most about 5%, 10%, 20%, 30%, 40%, 50%, 60%, 70%, 80%, 90%, or 100% sequence identity and/or sequence similarity to a wild-type Cas6 polypeptide (e.g., from T. thermophilus). “Cas6” can refer to the wildtype or a modified form of the Cas6 protein that can comprise an amino acid change such as a deletion, insertion, substitution, variant, mutation, fusion, chimera, or any combination thereof.

The terms “CRISPR/Cas9” or “CRISPR/Cas9 system” refer to a programmable nuclease system for genetic engineering that includes a Cas9 protein, or derivative thereof, and one or more non-coding RNAs that can provide the function of a CRISPR RNA (crRNA) and trans-activating RNA (tracrRNA) for the Cas9. The crRNA and tracrRNA can be used individually or can be combined to produce a “guide RNA” (gRNA). The crRNA or gRNA provide sequence that is complementary to the genomic target.

“Disease resistance” is a characteristic of an animal, wherein the animal avoids the disease symptoms that are the outcome of animal-pathogen interactions, such as interactions between a porcine animal and TGEV, PRCV, or PRRSV. That is, pathogens are prevented from causing animal diseases and the associated disease symptoms, or alternatively, a reduction of the incidence and/or severity of clinical signs or reduction of clinical symptoms. One of skill in the art will appreciate that the compositions and methods disclosed herein can be used with other compositions and methods available in the art for protecting animals from pathogen attack.

By “encoding” or “encoded”, with respect to a specified nucleic acid, is meant comprising the information for translation into the specified protein. A nucleic acid encoding a protein may comprise intervening sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. When the nucleic acid is prepared or altered synthetically, advantage can be taken of known codon preferences of the intended host where the nucleic acid is to be expressed.

As used herein, “gene editing,” “gene edited”, “genetically edited” and “gene editing effectors” refer to the use of homing technology with naturally occurring or artificially engineered nucleases, also referred to as “molecular scissors,” “homing endonucleases,” or “targeting endonucleases.” The nucleases create specific double-stranded chromosomal breaks (DSBs) at desired locations in the genome, which in some cases harnesses the cell's endogenous mechanisms to repair the induced break by natural processes of homologous recombination (HR) and/or nonhomologous end-joining (NHEJ). Gene editing effectors include Zinc Finger Nucleases (ZFNs), Transcription Activator-Like Effector Nucleases (TALENs), Clustered Regularly Interspaced Short Palindromic Repeats (CRISPR) systems (e.g., the CRISPR/Cas9 system), and meganucleases (e.g., meganucleases re-engineered as homing endonucleases). The terms also include the use of transgenic procedures and techniques, including, for example, where the change is a deletion or relatively small insertion (typically less than 20 nt) and/or does not introduce DNA from a foreign species. The term also encompasses progeny animals such as those created by sexual crosses or asexual propagation from the initial gene edited animal.

The terms “genome engineering,” “genetic engineering,” “genetically engineered,” “genetically altered,” “genetic alteration,” “genome modification,” “genome modification,” and “genomically modified” can refer to altering the genome by deleting, inserting, mutating, or substituting specific nucleic acid sequences. The altering can be gene or location specific. Genome engineering can use nucleases to cut a nucleic acid thereby generating a site for the alteration. Engineering of non-genomic nucleic acid is also contemplated. A protein containing a nuclease domain can bind and cleave a target nucleic acid by forming a complex with a nucleic acid-targeting nucleic acid. In one example, the cleavage can introduce double stranded breaks in the target nucleic acid. A nucleic acid can be repaired e.g. by endogenous non-homologous end joining (NHEJ) machinery. In a further example, a piece of nucleic acid can be inserted. Modifications of nucleic acid-targeting nucleic acids and site-directed polypeptides can introduce new functions to be used for genome engineering.

As used herein “homing DNA technology,” “homing technology” and “homing endonuclease” include any mechanisms that allow a specified molecule to be targeted to a specified DNA sequence including Zinc Finger (ZF) proteins, Transcription Activator-Like Effectors (TALEs) meganucleases, and CRISPR systems (e.g., the CRISPR/Cas9 system).

The terms “increased resistance” and “reduced susceptibility” herein mean, but are not limited to, a statistically significant reduction of the incidence and/or severity of clinical signs or clinical symptoms which are associated with infection by pathogen. For example, “increased resistance” or “reduced susceptibility” can refer to a statistically significant reduction of the incidence and/or severity of clinical signs or clinical symptoms which are associated with infection by TGEV, PRCV, or PRRSV in an animal comprising a modified chromosomal sequence in a CD163 gene protein as compared to a control animal having an unmodified chromosomal sequence. The term “statistically significant reduction of clinical symptoms” means, but is not limited to, the frequency in the incidence of at least one clinical symptom in the modified group of subjects is at least 10%, preferably at least 20%, more preferably at least 30%, even more preferably at least 50%, and even more preferably at least 70% lower than in the non-modified control group after the challenge with the infectious agent.

“Knock-out” means disruption of the structure or regulatory mechanism of a gene. Knock-outs may be generated through homologous recombination of targeting vectors, replacement vectors, or hit-and-run vectors or random insertion of a gene trap vector resulting in complete, partial or conditional loss of gene function.

The term “livestock animal” includes any animals traditionally raised in livestock farming, for example an ungulate (e.g., an artiodactyl), an avian animal (e.g., chickens, turkeys, ducks, geese, guinea fowl, or squabs), an equine animal (e.g., horses or donkeys). Artiodactyls include, but are not limited to porcine animals (e.g., pigs), bovine animals (e.g., beef of dairy cattle), ovine animals, caprine animals, buffalo, camels, llamas, alpacas, and deer. The term “livestock animal” does not include rats, mice, or other rodents.

As used herein, the term “mutation” includes alterations in the nucleotide sequence of a polynucleotide, such as for example a gene or coding DNA sequence (CDS), compared to the wild-type sequence. The term includes, without limitation, substitutions, insertions, frameshifts, deletions, inversions, translocations, duplications, splice-donor site mutations, point-mutations and the like.

Herein, “reduction of the incidence and/or severity of clinical signs” or “reduction of clinical symptoms” means, but is not limited to, reducing the number of infected subjects in a group, reducing or eliminating the number of subjects exhibiting clinical signs of infection, or reducing the severity of any clinical signs that are present in one or more subjects, in comparison to wild-type infection. For example, these terms encompass any clinical signs of infection, lung pathology, viremia, antibody production, reduction of pathogen load, pathogen shedding, reduction in pathogen transmission, or reduction of any clinical sign symptomatic of TGEV, PRCV, or PRRSV. Preferably these clinical signs are reduced in one or more animals of the invention by at least 10% in comparison to subjects not having a modification in the CD163 gene and that become infected. More preferably clinical signs are reduced in subjects of the invention by at least 20%, preferably by at least 30%, more preferably by at least 40%, and even more preferably by at least 50%.

References herein to a deletion in a nucleotide sequence from nucleotide x to nucleotide y mean that all of the nucleotides in the range have been deleted, including x and y. Thus, for example, the phrase “a 182 base pair deletion from nucleotide 1,397 to nucleotide 1,578 as compared to SEQ ID NO: 135” means that each of nucleotides 1,397 through 1,578 have been deleted, including nucleotides 1,397 and 1,578.

“Resistance” of an animal to a disease is a characteristic of an animal, wherein the animal avoids the disease symptoms that are the outcome of animal-pathogen interactions, such as interactions between a porcine animal and TGEV, PRCV, or PRRSV. That is, pathogens are prevented from causing animal diseases and the associated disease symptoms, or alternatively, a reduction of the incidence and/or severity of clinical signs or reduction of clinical symptoms. One of skill in the art will appreciate that the methods disclosed herein can be used with other compositions and methods available in the art for protecting animals from pathogen attack.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Zinc finger and TALE binding domains can be “engineered” to bind to a predetermined nucleotide sequence, for example via engineering (altering one or more amino acids) of the recognition helix region of naturally occurring zinc finger or TALE proteins. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “selected” zinc finger protein or TALE is a protein not found in nature whose production results primarily from an empirical process such as phage display, interaction trap or hybrid selection. See e.g., U.S. Pat. Nos. 5,789,538; 5,925,523; 6,007,988; 6,013,453; 6,200,759; WO 95/19431; WO 96/06166; WO 98/53057; WO 98/54311; WO 00/27878; WO 01/60970 WO 01/88197, WO 02/099084 and U.S. Publication No. 20110301073.

Various other terms are defined hereinbelow.

### Animals and Cells Having a Modified Chromosomal Sequence in a Gene Encoding an ANPEP Protein

Described herein are livestock animals and offspring thereof and animal cells comprising at least one modified chromosomal sequence in a gene encoding an ANPEP protein, e.g., an insertion or a deletion (“INDEL”), which confers improved or complete resistance to infection by a pathogen (e.g., transmissible gastroenteritis virus (TGEV) or porcine respiratory coronavirus (PRCV)).

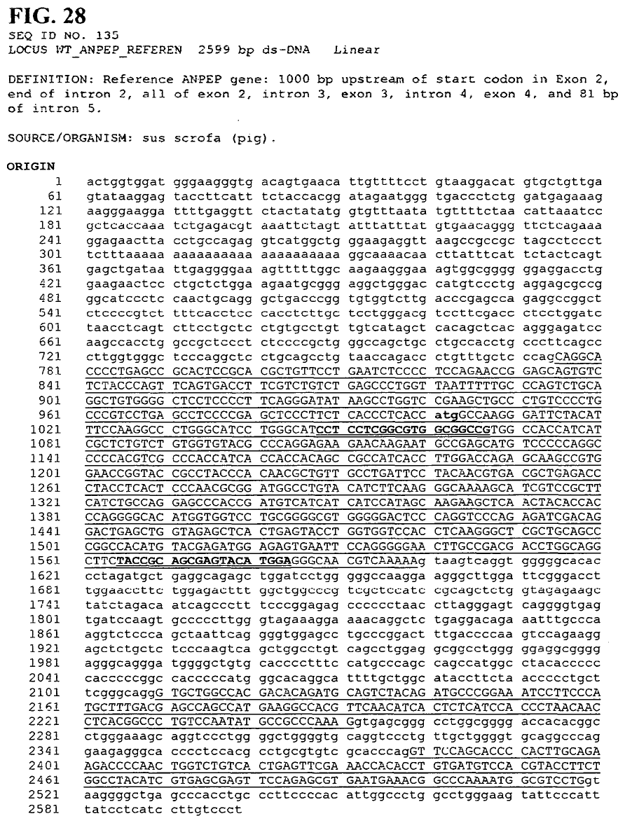

The full-length porcine ANPEP gene (SEQ ID NO: 132) is almost 30,000 base pairs long and has at least three splice variants. Depending on the splice variant, the porcine ANPEP gene contains 20 or 21 exons. However, the three splice variants are virtually identical across exon 2, the region that was targeted to make most of the genetically edited animals described herein. For ease of reference, a reference sequence is provided (SEQ ID NO: 135) that includes the coding region of exon 2, 1000 nucleotides preceding the start codon, and 1000 nucleotides following the end of exon 2. Since the start codon occurs within exon 2, reference sequence SEQ ID NO: 135 contains the last 773 base pairs in intron 2, exon 2, intron 3, exon 3, intron 4, exon 4, and 81 base pairs of intron 5. An annotated version of reference sequence SEQ ID NO: 135 is provided in FIG. 28. In FIG. 28, the locations of exons 2, 3, and 4 are marked with underlined text and the start codon is shown in bold lowercase text (“atg”).

A nucleotide sequence for full-length wild-type porcine ANPEP (SEQ ID NO: 132) is also provided, as are amino acid sequences for the full-length wild-type porcine ANPEP protein encoded by splice variants X2 and X3 (963 amino acids; SEQ ID NO:134) and the full-length wild-type porcine ANPEP protein encoded by splice variant X1 (1017 amino acids; SEQ ID NO:133). Splice variants X2 and X3 produce identical amino acid sequences.

Table 1 provides the locations of the exons in SEQ ID NO: 132 for each of the three splice variants.

TABLE 1
ANPEP exons

| | Variant X1 | Variant X2 | Variant X3 |
|---|---|---|---|
| Exon | Nucleotides in SEQ | Nucleotides in SEQ | Nucleotides in SEQ |
| Number | ID NO: 132 | ID NO: 132 | ID NO: 132 |
| 1 | 2092-2176 | 2083 . . . 2176 | 2082 . . . 2176 |
| 2* | 9760 . . . 10584 | 9760 . . . 10584 | 9763 . . . 10584 |
| 3 | 11094 . . . 11236 | 11094 . . . 11236 | 11094 . . . 11236 |
| 4 | 11364 . . . 11503 | 11364 . . . 11503 | 11364 . . . 11503 |
| 5 | 11927 . . . 12053 | 11927 . . . 12053 | 11927 . . . 12053 |
| 6 | 12148-12302 | 12148 . . . 12302 | 12148 . . . 12302 |
| 7 | 12532-12645 | 12532 . . . 12645 | 12532 . . . 12645 |
| 8 | 12743-12886 | 12743 . . . 12886 | 12743 . . . 12886 |
| 9 | 13064-13129 | 13064 . . . 13129 | 13064 . . . 13129 |
| 10 | 13253 . . . 13318 | 13253 . . . 13318 | 13253 . . . 13318 |
| 11 | 15209 . . . 15384 | 15209 . . . 15384 | 15209 . . . 15384 |
| 12 | 15624 . . . 15999 | 15624 . . . 15703 | 15624 . . . 15703 |
| 13 | 16102 . . . 16157 | 15866 . . . 15999 | 15866 . . . 15999 |
| 14 | 17087 . . . 17234 | 16102 . . . 16157 | 16102 . . . 16157 |
| 15 | 21446 . . . 21537 | 17087 . . . 17234 | 17087 . . . 17234 |
| 16 | 22017 . . . 22127 | 21446 . . . 21537 | 21446 . . . 21537 |
| 17 | 22255 . . . 22422 | 22017 . . . 22127 | 22017 . . . 22127 |
| 18 | 23148 . . . 23288 | 22255 . . . 22422 | 22255 . . . 22422 |
| 19 | 24061 . . . 24142 | 23148 . . . 23288 | 23148 . . . 23288 |
| 20 | 24265 . . . 24857 | 24061 . . . 24142 | 24061 . . . 24142 |
| 21 | none | 24265 . . . 24857 | 24265 . . . 24857 |

*The start codon occurs at nucleotide 9986 in all three variants.

Livestock animals and offspring thereof comprising at least one modified chromosomal sequence in a gene encoding an aminopeptidase N (ANPEP) protein are provided.

Animal cells comprising at least one modified chromosomal sequence in a gene encoding an ANPEP protein are also provided.

The modified chromosomal sequences can be sequences that are altered such that an ANPEP protein function as it relates to TGEV and/or PRCV infection is impaired, reduced, or eliminated. Thus, animals and cells described herein can be referred to as “knock-out” animals or cells.

The modified chromosomal sequence in the gene encoding the ANPEP protein reduces the susceptibility of the animal, offspring, or cell to infection by a pathogen, as compared to the susceptibility of a livestock animal, offspring, or cell that does not comprise a modified chromosomal sequence in a gene encoding an ANPEP protein to infection by the pathogen.

The modification preferably substantially eliminates susceptibility of the animal, offspring, or cell to the pathogen. The modification more preferably completely eliminates susceptibility of the animal, offspring, or cell to the pathogen, such that animals do not show any clinical signs of disease following exposure to the pathogen.

For example, where the animal is a porcine animal and the pathogen is TGEV, porcine animals having the modification do not show any clinical signs of TGEV infection (e.g., vomiting, diarrhea, dehydration, excessive thirst) following exposure to TGEV. In addition, in porcine animals having the modification, TGEV nucleic acid cannot be detected in the feces or serum, TGEV antigen cannot be detected in the ileum, and serum is negative for TGEV-specific antibody.

Similarly, cells having the modification that are exposed to the pathogen do not become infected with the pathogen.

The pathogen can comprise a virus. For example, the pathogen can comprise a Coronaviridae family virus, e.g., a Coronavirinae subfamily virus.

The virus preferably comprises a coronavirus (e.g., an Alphacoronavirus genus virus).

Where the virus comprises an Alphacoronavirus genus virus, the Alphacoronavirus genus virus preferably comprises a transmissible gastroenteritis virus (TGEV).

For example, the transmissible gastroenteritis virus can comprise TGEV Purdue strain.

Alternatively or in addition, the virus can comprise a porcine respiratory coronavirus (PRCV).

The livestock animal or offspring can comprise an ungulate, an avian animal, or an equine animal. The cell can be derived from an ungulate, an avian animal, or an equine animal.

Where the animal or offspring is an avian animal or where the cell is a cell derived from an avian animal, the avian animal can comprise a chicken, a turkey, a duck, a goose, a guinea fowl, or a squab.

Where the animal or offspring is an equine animal or where the cell is a cell derived from an equine animal, the equine animal can comprise a horse or a donkey.

Where the animal or offspring is an ungulate or where the cell is a cell derived from an ungulate, the ungulate can comprise an artiodactyl. For example, the artiodactyl can comprise a porcine animal (e.g., a pig), a bovine animal (e.g., beef cattle or dairy cattle), an ovine animal, a caprine animal, a buffalo, a camel, a llama, an alpaca, or a deer.

The animal or offspring preferably comprises a porcine animal. The cell preferably comprises a cell derived from a porcine animal.

The animal or offspring can be an embryo, a juvenile, or an adult.

Similarly, the cell can comprises an embryonic cell, a cell derived from a juvenile animal, or a cell derived from an adult animal.

For example, the cell can comprise an embryonic cell.

The cell can comprise a cell derived from a juvenile animal.

The animal, offspring, or cell can be heterozygous for the modified chromosomal sequence in the gene encoding the ANPEP protein.

The animal, offspring, or cell can be homozygous for the modified chromosomal sequence in the gene encoding the ANPEP protein.

The modified chromosomal sequence in the gene encoding the ANPEP protein can comprise an insertion in an allele of the gene encoding the ANPEP protein, a deletion in an allele of the gene encoding the ANPEP protein, a substitution in an allele of the gene encoding the ANPEP protein, or a combination of any thereof.

For example, the modified chromosomal sequence can comprise a deletion in an allele of the gene encoding the ANPEP protein.

The deletion can comprise an in-frame deletion.

The modified chromosomal sequence can comprise an insertion in an allele of the gene encoding the ANPEP protein.

The insertion, the deletion, the substitution, or the combination of any thereof can result in a miscoding in the allele of the gene encoding the ANPEP protein.

Where the insertion, the deletion, the substitution, or the combination of any thereof results in a miscoding in the allele of the gene encoding the ANPEP protein, the miscoding can result in a premature stop codon in the allele of the gene encoding the ANPEP protein.

Where the modified chromosomal sequence comprises a deletion, the deletion can comprise a deletion of the start codon of the allele of the gene encoding the ANPEP protein. When the start codon is deleted, no ANPEP protein is produced.

Where the modified chromosomal sequence comprises a deletion, the deletion can comprise a deletion of the entire coding sequence of the allele of the gene encoding the ANPEP protein.

The modified chromosomal sequence can comprise a substitution in an allele of the gene encoding the ANPEP protein.

In any of the animals, offspring, or cells described herein, the modified chromosomal sequence in the gene encoding the ANPEP protein preferably causes ANPEP protein production or activity to be reduced, as compared to ANPEP protein production or activity in an animal, offspring, or cell that lacks the modified chromosomal sequence in the gene encoding the ANPEP protein.

Preferably, the modified chromosomal sequence in the gene encoding the ANPEP protein results in production of substantially no functional ANPEP protein by the animal, offspring or cell. By “substantially no functional ANPEP protein,” it is meant that the level of ANPEP protein in the animal, offspring, or cell is undetectable, or if detectable, is at least about 90% lower, preferably at least about 95% lower, more preferably at least about 98%, lower, and even more preferably at least about 99% lower than the level observed in an animal, offspring, or cell that does not comprise the modified chromosomal sequences.

For any of the animals, offspring, or cells described herein, the animal, offspring, or cell preferably does not produce ANPEP protein.

In any of the animals, offspring, or cells, the modified chromosomal sequence comprises a modification in: exon 2 of an allele of the gene encoding the ANPEP protein; exon 4 of an allele of the gene encoding the ANPEP protein; an intron that is contiguous with exon 2 or exon 4 of the allele of the gene encoding the ANPEP protein; or a combination of any thereof.

The modified chromosomal sequence suitably comprises a modification in exon 2 of the allele of the gene encoding the ANPEP protein, a modification in intron 1 of the allele of the gene encoding the ANPEP protein, or a combination thereof.

As one example, the modified chromosomal sequence can comprise a deletion that begins in intron 1 of the allele of the gene encoding the ANPEP protein and ends in exon 2 of the allele of the gene encoding the ANPEP protein.

The modified chromosomal sequence can comprise an insertion or a deletion in exon 2 of the allele of the gene encoding the ANPEP protein. For example, the insertion or deletion in exon 2 of the allele of the gene encoding the ANPEP protein can be downstream of the start codon.

The modified chromosomal sequence can comprise a deletion in exon 2 of the allele of the gene encoding the ANPEP protein.

Where the modified chromosomal sequence comprises a deletion in exon 2 of the allele of the gene encoding the ANPEP protein, the deletion can comprise an in-frame deletion in exon 2.

For example, the in-frame deletion in exon 2 of the allele of the gene encoding the ANPEP protein can result in deletion of amino acids 194 through 196 of the ANPEP protein.

Alternatively, the in-frame deletion in exon 2 of the allele of the gene encoding the ANPEP protein can result in deletion of amino acids 194 through 197 of the ANPEP protein. The in-frame deletion can further result in substitution of the valine residue at position 198 of the ANPEP protein with another amino acid, e.g., an isoleucine residue.

The modified chromosomal sequence can comprise an insertion in exon 2 of the allele of the gene encoding the ANPEP protein.

In any of the animals, offspring, or cells described herein, the modified chromosomal sequence can comprise a modification selected from the group consisting of: a 182 base pair deletion from nucleotide 1,397 to nucleotide 1,578, as compared to reference sequence SEQ ID NO: 135, wherein the deleted sequence is replaced with a 5 base pair insertion beginning at nucleotide 1,397; a 9 base pair deletion from nucleotide 1,574 to nucleotide 1,582, as compared to reference sequence SEQ ID NO: 135; a 9 base pair deletion from nucleotide 1,577 to nucleotide 1,585, as compared to reference sequence SEQ ID NO: 135; a 9 base pair deletion from nucleotide 1,581 to nucleotide 1,589, as compared to reference sequence SEQ ID NO: 135; an 867 base pair deletion from nucleotide 819 to nucleotide 1,685, as compared to reference sequence SEQ ID NO: 135; an 867 base pair deletion from nucleotide 882 to nucleotide 1,688, as compared to reference sequence SEQ ID NO: 135; a 1 base pair insertion between nucleotides 1,581 and 1,582, as compared to reference sequence SEQ ID NO: 135; a 1 base pair insertion between nucleotides 1,580 and 1,581, as compared to reference sequence SEQ ID NO: 135; a 1 base pair insertion between nucleotides 1,579 and 1,580, as compared to reference sequence SEQ ID NO: 135; a 2 base pair insertion between nucleotides 1,581 and 1,582, as compared to reference sequence SEQ ID NO: 135; a 267 base pair deletion from nucleotide 1,321 to nucleotide 1,587, as compared to reference sequence SEQ ID NO: 135; a 267 base pair deletion from nucleotide 1,323 to nucleotide 1,589, as compared to reference sequence SEQ ID NO: 135; a 1 base pair deletion of nucleotide 1,581, as compared to reference sequence SEQ ID NO: 135; a 12 base pair deletion from nucleotide 1,582 to nucleotide 1,593, as compared to reference sequence SEQ ID NO: 135; a 25 base pair deletion from nucleotide 1,561 to nucleotide 1,585, as compared to reference sequence SEQ ID NO: 135; a 25 base pair deletion from nucleotide 1,560 to nucleotide 1,584, as compared to reference sequence SEQ ID NO: 135; an 8 base pair deletion from nucleotide 1,575 to nucleotide 1,582, as compared to reference sequence SEQ ID NO: 135; an 8 base pair deletion from nucleotide 1,574 to nucleotide 1,581, as compared to reference sequence SEQ ID NO: 135; a 661 base pair deletion from nucleotide 940 to nucleotide 1,600, as compared to reference sequence SEQ ID NO: 135, wherein the deleted sequence is replaced with an 8 base pair insertion beginning at nucleotide 940; an 8 base pair deletion from nucleotide 1,580 to nucleotide 1,587, as compared to reference sequence SEQ ID NO: 135, wherein the deleted sequence is replaced with a 4 base pair insertion beginning at nucleotide 1,580; and combinations of any thereof.

For example, in any of the animals, offspring, or cells, the modified chromosomal sequence can comprise a modification selected from the group consisting of: the 661 base pair deletion from nucleotide 940 to nucleotide 1,600, as compared to reference sequence SEQ ID NO: 135, wherein the deleted sequence is replaced with the 8 base pair insertion beginning at nucleotide 940; the 8 base pair deletion from nucleotide 1,580 to nucleotide 1,587, as compared to reference sequence SEQ ID NO: 135, wherein the deleted sequence is replaced with the 4 base pair insertion beginning at nucleotide 1,580; the 1 base pair insertion between nucleotides 1,581 and 1,582, as compared to reference sequence SEQ ID NO: 135; the 2 base pair insertion between nucleotides 1,581 and 1,582, as compared to reference sequence SEQ ID NO: 135; the 9 base pair deletion from nucleotide 1,581 to nucleotide 1,589, as compared to reference sequence SEQ ID NO: 135; the 12 base pair deletion from nucleotide 1,582 to nucleotide 1,593, as compared to reference sequence SEQ ID NO: 135; the 1 base pair deletion of nucleotide 1,581, as compared to reference sequence SEQ ID NO: 135; and combinations of any thereof.

In any of the animals, offspring, or cells, the modified chromosomal sequence can comprise a modification selected from the group consisting of: the 661 base pair deletion from nucleotide 940 to nucleotide 1,600, as compared to reference sequence SEQ ID NO: 135, wherein the deleted sequence is replaced with the 8 base pair insertion beginning at nucleotide 940; the 8 base pair deletion from nucleotide 1,580 to nucleotide 1,587, as compared to reference sequence SEQ ID NO: 135, wherein the deleted sequence is replaced with the 4 base pair insertion beginning at nucleotide 1,580; the 1 base pair insertion between nucleotides 1,581 and 1,582, as compared to reference sequence SEQ ID NO: 135; the 2 base pair insertion between nucleotides 1,581 and 1,582, as compared to reference sequence SEQ ID NO: 135; the 1 base pair deletion of nucleotide 1,581, as compared to reference sequence SEQ ID NO: 135; and combinations of any thereof.

The modified chromosomal sequence can comprise a 182 base pair deletion from nucleotide 1,397 to nucleotide 1,578, as compared to reference sequence SEQ ID NO: 135, wherein the deleted sequence is replaced with a 5 base pair insertion beginning at nucleotide 1,397.

Where the modified chromosomal sequence comprises the 182 base pair deletion from nucleotide 1,397 to nucleotide 1,578, as compared to reference sequence SEQ ID NO: 135, wherein the deleted sequence is replaced with a 5 base pair insertion beginning at nucleotide 1,397, the 5 base pair insertion can comprise the sequence CCCTC (SEQ ID NO: 169).

The modified chromosomal sequence can comprise a 9 base pair deletion from nucleotide 1,574 to nucleotide 1,582, as compared to reference sequence SEQ ID NO: 135.

The modified chromosomal sequence can comprise a 9 base pair deletion from nucleotide 1,577 to nucleotide 1,585, as compared to reference sequence SEQ ID NO: 135.

The modified chromosomal sequence can comprise a 9 base pair deletion from nucleotide 1,581 to nucleotide 1,589, as compared to reference sequence SEQ ID NO: 135.

The modified chromosomal sequence can comprise an 867 base pair deletion from nucleotide 819 to nucleotide 1,685, as compared to reference sequence SEQ ID NO: 135.

The modified chromosomal sequence can comprise an 867 base pair deletion from nucleotide 882 to nucleotide 1,688, as compared to reference sequence SEQ ID NO: 135.

The modified chromosomal sequence can comprise a 1 base pair insertion between nucleotides 1,581 and 1,582, as compared to reference sequence SEQ ID NO: 135.

Where the modified chromosomal sequence comprises the 1 base pair insertion between nucleotides 1,581 and 1,582, as compared to reference sequence SEQ ID NO: 135, the insertion can comprise a single thymine (T) residue.

The modified chromosomal sequence can comprise a 1 base pair insertion between nucleotides 1,580 and 1,581, as compared to reference sequence SEQ ID NO: 135.

Where the modified chromosomal sequence comprises the 1 base pair insertion between nucleotides 1,580 and 1,581, as compared to reference sequence SEQ ID NO: 135, the insertion can comprise a single thymine (T) residue or a single adenine (A) residue.

The modified chromosomal sequence can comprise a 1 base pair insertion between nucleotides 1,579 and 1,580, as compared to reference sequence SEQ ID NO: 135.

Where the modified chromosomal sequence comprises the 1 base pair insertion between nucleotides 1,579 and 1,580, as compared to reference sequence SEQ ID NO: 135, the insertion can comprise a single adenine (A) residue.

The modified chromosomal sequence can comprise a 2 base pair insertion between nucleotides 1,581 and 1,582, as compared to reference sequence SEQ ID NO: 135.

Where the modified chromosomal sequence comprises the 2 base pair insertion between nucleotides 1,581 and 1,582, as compared to reference sequence SEQ ID NO: 135, the 2 base pair insertion can comprise an AT dinucleotide.

The modified chromosomal sequence can comprise a 267 base pair deletion from nucleotide 1,321 to nucleotide 1,587, as compared to reference sequence SEQ ID NO: 135.

The modified chromosomal sequence can comprise a 267 base pair deletion from nucleotide 1,323 to nucleotide 1,589, as compared to reference sequence SEQ ID NO: 135.

The modified chromosomal sequence can comprise a 1 base pair deletion of nucleotide 1,581, as compared to reference sequence SEQ ID NO: 135.

The modified chromosomal sequence can comprise a 12 base pair deletion from nucleotide 1,582 to nucleotide 1,593, as compared to reference sequence SEQ ID NO: 135.

The modified chromosomal sequence can comprise a 25 base pair deletion from nucleotide 1,561 to nucleotide 1,585, as compared to reference sequence SEQ ID NO: 135.

The modified chromosomal sequence can comprise a 25 base pair deletion from nucleotide 1,560 to nucleotide 1,584, as compared to reference sequence SEQ ID NO: 135.

The modified chromosomal sequence can comprise an 8 base pair deletion from nucleotide 1,575 to nucleotide 1,582, as compared to reference sequence SEQ ID NO: 135.

The modified chromosomal sequence can comprise an 8 base pair deletion from nucleotide 1,574 to nucleotide 1,581, as compared to reference sequence SEQ ID NO: 135.

The modified chromosomal sequence can comprise a 661 base pair deletion from nucleotide 940 to nucleotide 1,600, as compared to reference sequence SEQ ID NO: 135, wherein the deleted sequence is replaced with an 8 base pair insertion beginning at nucleotide 940.

When the modified chromosomal sequence comprises the 661 base pair deletion from nucleotide 940 to nucleotide 1,600, as compared to reference sequence SEQ ID NO: 135, wherein the deleted sequence is replaced with an 8 base pair insertion beginning at nucleotide 940, the 8 base pair insertion can comprise the sequence GGGGCTTA (SEQ ID NO: 179).

The modified chromosomal sequence can comprise an 8 base pair deletion from nucleotide 1,580 to nucleotide 1,587, as compared to reference sequence SEQ ID NO: 135, wherein the deleted sequence is replaced with a 4 base pair insertion beginning at nucleotide 1,580.

When the modified chromosomal sequence comprises the 8 base pair deletion from nucleotide 1,580 to nucleotide 1,587, as compared to reference sequence SEQ ID NO: 135, wherein the deleted sequence is replaced with a 4 base pair insertion beginning at nucleotide 1,580, the 4 base pair insertion can comprise the sequence TCGT (SEQ ID NO: 180).

The ANPEP gene in the animal, offspring, or cell can comprise any combination of any of the modified chromosomal sequences described herein.

For example, the animal, offspring, or cell can comprise the 661 base pair deletion from nucleotide 940 to nucleotide 1,600, as compared to reference sequence SEQ ID NO: 135 in one allele of the gene encoding the ANPEP protein, wherein the deleted sequence is replaced with the 8 base pair insertion beginning at nucleotide 940; and the 2 base pair insertion between nucleotides 1,581 and 1,582, as compared to reference sequence SEQ ID NO: 135 in the other allele of the gene encoding the ANPEP protein.

The animal, offspring, or cell can comprise the 8 base pair deletion from nucleotide 1,580 to nucleotide 1,587, as compared to reference sequence SEQ ID NO: 135 in one allele of the gene encoding the ANPEP protein, wherein the deleted sequence is replaced with the 4 base pair insertion beginning at nucleotide 1,580; and the 1 base pair insertion between nucleotides 1,581 and 1,582, as compared to reference sequence SEQ ID NO: 135 in the other allele of the gene encoding the ANPEP protein.

The animal, offspring, or cell can comprise the 8 base pair deletion from nucleotide 1,580 to nucleotide 1,587, as compared to reference sequence SEQ ID NO: 135 in one allele of the gene encoding the ANPEP protein, wherein the deleted sequence is replaced with the 4 base pair insertion beginning at nucleotide 1,580; and the 1 base pair deletion of nucleotide 1,581, as compared to reference sequence SEQ ID NO: 135 in the other allele of the gene encoding the ANPEP protein.

The animal, offspring, or cell can comprise the 8 base pair deletion from nucleotide 1,580 to nucleotide 1,587, as compared to reference sequence SEQ ID NO: 135 in one allele of the gene encoding the ANPEP protein, wherein the deleted sequence is replaced with the 4 base pair insertion beginning at nucleotide 1,580; and the 2 base pair insertion between nucleotides 1,581 and 1,582, as compared to reference sequence SEQ ID NO: 135 in the other allele of the gene encoding the ANPEP protein.

The animal, offspring, or cell can comprise the 661 base pair deletion from nucleotide 940 to nucleotide 1,600, as compared to reference sequence SEQ ID NO: 135 in one allele of the gene encoding the ANPEP protein, wherein the deleted sequence is replaced with the 8 base pair insertion beginning at nucleotide 940; and the 9 base pair deletion from nucleotide 1,581 to nucleotide 1,589, as compared to reference sequence SEQ ID NO: 135 in the other allele of the gene encoding the ANPEP protein.

In any of the animals, offspring, or cells described herein, the modified chromosomal sequence comprises a modification within the region comprising nucleotides 17,235 through 22,422 of reference sequence SEQ ID NO: 132.

For example, the modified chromosomal sequence can comprise a modification within the region comprising nucleotides 17,235 through 22,016 of reference sequence SEQ ID NO: 132.

The modified chromosomal sequence can comprise a modification within the region comprising nucleotides 21,446 through 21,537 of reference sequence SEQ ID NO: 132.

The modified chromosomal sequence can comprise a modification within the region comprising nucleotides 21,479 through 21,529 of reference sequence SEQ ID NO: 132.

For example, the modified chromosomal sequence can comprise a 51 base pair deletion from nucleotide 21,479 to nucleotide 21,529 of reference sequence SEQ ID NO: 132.

The modified chromosomal sequence can comprise a modification within the region comprising nucleotides 21,479 through 21,523 of reference sequence SEQ ID NO: 132.

For example, the modified chromosomal sequence can comprise a 45 base pair deletion from nucleotide 21,479 to nucleotide 21,523 of reference sequence SEQ ID NO: 132.

As a further example, the modified chromosomal sequence can comprise a 3 base pair deletion from nucleotide 21,509 to nucleotide 21,511 of reference sequence SEQ ID NO: 132.

The modified chromosomal sequence can comprise a modification within the region comprising nucleotides 21,538 through 22,422 of reference sequence SEQ ID NO: 132.

The modified chromosomal sequence can comprise a modification within the region comprising nucleotides 22,017 through 22,422 of reference sequence SEQ ID NO: 132.

The modified chromosomal sequence can comprise a modification within the region comprising nucleotides 22,054 through 22,256 of reference sequence SEQ ID NO: 132.

The modified chromosomal sequence can comprise a modification within the region comprising nucleotides 22,054 through 22,126 of reference sequence SEQ ID NO: 132.

Where the modified chromosomal sequence comprises a modification anywhere within the region comprising nucleotides 17,235 through 22,422 of reference sequence SEQ ID NO: 132, the modified chromosomal sequence can comprise an insertion or a deletion.

For example, the modified chromosomal sequence can comprise a deletion. The deletion can optionally comprise an in-frame deletion.

Where the modified chromosomal sequence comprises a modification anywhere within the region comprising nucleotides 17,235 through 22,422 of reference sequence SEQ ID NO: 132, the modified chromosomal sequence can comprise a substitution.

For example, the substitution can comprise a substitution of one or more of the nucleotides in the ACC codon at nucleotides 21,509 through 21,511 of SEQ ID NO: 132 with a different nucleotide, to produce a codon that encodes a different amino acid.

Where the substitution comprises a substitution of one or more of the nucleotides in the ACC codon at nucleotides 21,509 through 21,511 of SEQ ID NO: 132 with a different nucleotide, to produce a codon that encodes a different amino acid, the substitution of the one or more nucleotides can result in replacement of the threonine (T) at amino acid 738 of SEQ ID NO: 134 or the threonine (T) at amino acid 792 of SEQ ID NO: 133 with a glycine (G), alanine (A), cysteine (C), valine (V), leucine (L), isoleucine (I), methionine (M), proline phenylalanine (F), tyrosine (Y), tryptophan (W), aspartic acid (D), glutamic acid (E), asparagine (N), glutamine (Q), histidine (H), lysine (K), or arginine (R) residue.

For example, the substitution results in replacement of the threonine (T) at amino acid 738 of SEQ ID NO: 134 or the threonine (T) at amino acid 792 of SEQ ID NO: 133 with a glycine (G), alanine (A), cysteine (C), valine (V), leucine (L), isoleucine (I), methionine (M), proline phenylalanine (F), tryptophan (W), asparagine (N), glutamine (Q), histidine (H), lysine (K), or arginine (R) residue.

The substitution suitably results in replacement of the threonine (T) at amino acid 738 of SEQ ID NO: 134 or the threonine (T) at amino acid 792 of SEQ ID NO: 133 with a valine (V) or arginine (R) residue.

In any of the animals, offspring, or cells described herein, the modified chromosomal sequence can disrupt an intron-exon splice region. Disruption of an intron-exon splice region can result in exon skipping or intron inclusion due to lack of splicing downstream of the intron-exon splice region, as well as additional downstream exons in the resulting mRNA.

In order to disrupt an intron-exon splice region, any nucleotide that is required for splicing can be altered. For example, most introns end in the sequence “AG.” If the guanine (G) residue in this sequence is replaced with a different base, the splice will not occur at this site and will instead occur at the next downstream AG dinucleotide.

Intron-exon splice regions can also be disrupted by modifying the sequence at the beginning of the intron. Most introns begin with the consensus sequence RRGTRRRY (SEQ ID NO: 186), where “R” is any purine and “Y” is any pyrimidine. If the guanine (G) residue in this sequence is modified and/or if two or more of the other bases are modified, the intron can be rendered non-functional and will not splice.

Intron-exon splice regions can also be disrupted by any other methods known in the art.

Any of the modified chromosomal sequences in the gene encoding the ANPEP protein described herein can consist of the deletion, insertion or substitution.

In any of the animals, offspring, or cells described herein, the animal, offspring, or cell can comprise a chromosomal sequence in the gene encoding the ANPEP protein having at least 80% sequence identity to SEQ ID NO: 135 in the regions of the chromosomal sequence outside of the insertion, the deletion, or the substitution.

The animal, offspring, or cell can comprise a chromosomal sequence in the gene encoding the ANPEP protein having at least 85% sequence identity to SEQ ID NO: 135 in the regions of the chromosomal sequence outside of the insertion, the deletion, or the substitution.

The animal, offspring, or cell can comprise a chromosomal sequence in the gene encoding the ANPEP protein having at least 90% sequence identity to SEQ ID NO: 135 in the regions of the chromosomal sequence outside of the insertion, the deletion, or the substitution.

The animal, offspring, or cell can comprise a chromosomal sequence in the gene encoding the ANPEP protein having at least 95% sequence identity to SEQ ID NO: 135 in the regions of the chromosomal sequence outside of the insertion, the deletion, or the substitution.

The animal, offspring, or cell can comprise a chromosomal sequence in the gene encoding the ANPEP protein having at least 98% sequence identity to SEQ ID NO: 135 in the regions of the chromosomal sequence outside of the insertion, the deletion, or the substitution.

The animal, offspring, or cell can comprise a chromosomal sequence in the gene encoding the ANPEP protein having at least 99% sequence identity to SEQ ID NO: 135 in the regions of the chromosomal sequence outside of the insertion, the deletion, or the substitution.

The animal, offspring, or cell can comprise a chromosomal sequence in the gene encoding the ANPEP protein having at least 99.9% sequence identity to SEQ ID NO: 135 in the regions of the chromosomal sequence outside of the insertion, the deletion, or the substitution.

The animal, offspring, or cell can comprise a chromosomal sequence in the gene encoding the ANPEP protein having 100% sequence identity to SEQ ID NO: 135 in the regions of the chromosomal sequence outside of the insertion, the deletion, or the substitution.

In any of the animals, offspring, or cells described herein, the animal, offspring, or cell can comprise a chromosomal sequence in the gene encoding the ANPEP protein having at least 80% sequence identity to SEQ ID NO: 132 in the regions of the chromosomal sequence outside of the insertion, the deletion, or the substitution.

The animal, offspring, or cell can comprise a chromosomal sequence in the gene encoding the ANPEP protein having at least 85% sequence identity to SEQ ID NO: 132 in the regions of the chromosomal sequence outside of the insertion, the deletion, or the substitution.

The animal, offspring, or cell can comprise a chromosomal sequence in the gene encoding the ANPEP protein having at least 90% sequence identity to SEQ ID NO: 132 in the regions of the chromosomal sequence outside of the insertion, the deletion, or the substitution.

The animal, offspring, or cell can comprise a chromosomal sequence in the gene encoding the ANPEP protein having at least 95% sequence identity to SEQ ID NO: 132 in the regions of the chromosomal sequence outside of the insertion, the deletion, or the substitution.

The animal, offspring, or cell can comprise a chromosomal sequence in the gene encoding the ANPEP protein having at least 98% sequence identity to SEQ ID NO: 132 in the regions of the chromosomal sequence outside of the insertion, the deletion, or the substitution.

The animal, offspring, or cell can comprise a chromosomal sequence in the gene encoding the ANPEP protein having at least 99% sequence identity to SEQ ID NO: 132 in the regions of the chromosomal sequence outside of the insertion, the deletion, or the substitution.

The animal, offspring, or cell can comprise a chromosomal sequence in the gene encoding the ANPEP protein having at least 99.9% sequence identity to SEQ ID NO: 132 in the regions of the chromosomal sequence outside of the insertion, the deletion, or the substitution.

The animal, offspring, or cell can comprise a chromosomal sequence in the gene encoding the ANPEP protein having 100% sequence identity to SEQ ID NO: 132 in the regions of the chromosomal sequence outside of the insertion, the deletion, or the substitution.

Any of the animals, offspring, or cells can comprise a chromosomal sequence comprising SEQ ID NO: 163, 164, 165, 166, 167, 168, 170, 171, 172, 173, 174, 176, 177, or 178.

For example, any of the animals, offspring, or cells can comprise a chromosomal sequence comprising SEQ ID NO: 177, 178, 166, 167, 170, 172, or 171.

Any of the animals, offspring, or cells can comprise a chromosomal sequence comprising SEQ ID NO: 177, 178, 166, 167, or 171.

### Animals and Cells Having a Modified Chromosomal Sequence in a Gene Encoding an ANPEP and Further Comprising a Modified Chromosomal Sequence in a Gene Encoding a CD163 Protein

Any of the livestock animals, offspring, or cells that comprise at least one modified chromosomal sequence in a gene encoding an ANPEP protein can further comprise at least one modified chromosomal sequence in a gene encoding a CD163 protein.

CD163 has 17 exons and the protein is composed of an extracellular region with 9 scavenger receptor cysteine-rich (SRCR) domains, a transmembrane segment, and a short cytoplasmic tail. Several different variants result from differential splicing of a single gene (Ritter et al. 1999a; Ritter et al. 1999b). Much of this variation is accounted for by the length of the cytoplasmic tail.

CD163 has a number of important functions, including acting as a haptoglobin-hemoglobin scavenger receptor. Elimination of free hemoglobin in the blood is an important function of CD163 as the heme group can be very toxic (Kristiansen et al. 2001). CD163 has a cytoplasmic tail that facilitates endocytosis. Mutation of this tail results in decreased haptoglobin-hemoglobin complex uptake (Nielsen et al. 2006). Other functions of C163 include erythroblast adhesion (SRCR2), being a TWEAK receptor (SRCR1-4 & 6-9), a bacterial receptor (SRCR5), an African Swine Virus receptor (Sanchez-Torres et al. 2003), and a potential role as an immune-modulator (discussed in Van Gorp et al. 2010).

CD163 is a member of the scavenger receptor cysteine-rich (SRCR) superfamily and has an intracellular domain and 9 extracellular SRCR domains. In humans, endocytosis of CD163 mediated hemoglobin-heme uptake via SRCR3 protects cells from oxidative stress (Schaer et al., 2006a; Schaer et al., 2006b; Schaer et al., 2006c). CD163 also serves as a receptor for tumor necrosis factor-like weak inducer of apoptosis (TWEAK: SRCR1-4 & 6-9), a pathogen receptor (African Swine Fever Virus; bacteria: SRCR2), and erythroblast binding (SRCR2).

CD163 plays a role in infection by porcine reproductive and respiratory syndrome virus (PRRSV) as well as many other pathogens. Therefore, animals, offspring, and cells having a modified chromosomal sequence in a gene encoding a CD163 protein can have reduced susceptibility to PRRSV infection, as well as reduced susceptibility to infection by other pathogens that rely on CD163 for entry into a cell or for later replication and/or persistence in the cell. The infection process of the PRRSV begins with initial binding to heparan sulfate on the surface of the alveolar macrophage. The virus is then internalized via clatherin-mediated endocytosis. Another molecule, CD163, then facilitates the uncoating of the virus in the endosome (Van Breedam et al. 2010). The viral genome is released and the cell infected.

Described herein are animals and offspring thereof and cells comprising at least one modified chromosomal sequence in a gene encoding a CD163 protein, e.g., an insertion or a deletion (“INDEL”), which confers improved or complete resistance to infection by a pathogen (e.g., PRRSV) upon the animal. Applicant has demonstrated that that CD163 is the critical gene in PRRSV infection and have created founder resistant animals and lines (see, e.g., PCT Publication No. WO 2017/023570 and U.S. Patent Application Publication No. 2017/0035035, the contents of which are incorporated herein by reference in their entirety).

Thus, where the animal, offspring, or cell comprises both a modified chromosomal sequence in a gene encoding an ANPEP protein and a modified chromosomal sequence in a gene encoding a CD163 protein, the animal, offspring, or cell will be resistant infection to multiple pathogens. For example, where the animal or offspring is a porcine animal or where the cell is a porcine cell, the animal, offspring, or cell will be resistant to infection by TGEV due to the modified chromosomal sequence in the gene encoding the ANPEP protein and will also be resistant to infection by PRRSV due to the modified chromosomal sequence in the gene encoding the CD163 protein.

The modified chromosomal sequence in the gene encoding the CD163 protein reduces the susceptibility of the animal, offspring, or cell to infection by a pathogen (e.g., a virus such as a porcine reproductive and respiratory syndrome virus (PRRSV)), as compared to the susceptibility of an animal, offspring, or cell that does not comprise a modified chromosomal sequence in a gene encoding a CD163 protein to infection by the pathogen.

The modified chromosomal sequence in the gene encoding the CD163 protein preferably substantially eliminates susceptibility of the animal, offspring, or cell to the pathogen. The modification more preferably completely eliminates susceptibility of the animal, offspring, or cell to the pathogen, such that animals do not show any clinical signs of disease following exposure to the pathogen.

For example, where the animal is a porcine animal and the pathogen is PRRSV, porcine animals having the modified chromosomal sequence in the gene encoding the CD163 protein do not show any clinical signs of PRRSV infection (e.g., respiratory distress, inappetence, lethargy, fever, reproductive failure during late gestation) following exposure to PRRSV. In addition, in porcine animals having the modification, PRRSV nucleic acid cannot be detected in serum and do not produce PRRSV-specific antibody.

The pathogen can comprise a virus.

The virus can comprise a porcine reproductive and respiratory syndrome virus (PRRSV).

The modified chromosomal sequence in the gene encoding the CD163 protein can reduce the susceptibility of the animal, offspring, or cell to a Type 1 PRRSV virus, a Type 2 PRRSV, or to both Type 1 and Type 2 PRRSV viruses.

The modified chromosomal sequence in the gene encoding the CD163 protein can reduce the susceptibility of the animal, offspring, or cell to a PRRSV isolate selected from the group consisting of NVSL 97-7895, KS06-72109, P129, VR2332, CO90, AZ25, MLV-ResPRRS, KS62-06274, KS483 (SD23983), C084, SD13-15, Lelystad, 03-1059, 03-1060, SD01-08, 4353PZ, and combinations of any thereof.

The animal, offspring, or cell can be heterozygous for the modified chromosomal sequence in the gene encoding the CD163 protein.

The animal, offspring, or cell can be homozygous for the modified chromosomal sequence in the gene encoding the CD163 protein.

In any of the animals, offspring, or cells comprising a modified chromosomal sequence in the gene encoding the CD163 protein, the modified chromosomal sequence can comprise an insertion in an allele of the gene encoding the CD163 protein, a deletion in an allele of the gene encoding the CD163 protein, a substitution in an allele of the gene encoding the CD163 protein, or a combination of any thereof.

For example, the modified chromosomal sequence in the gene encoding the CD163 protein can comprise a deletion in an allele of the gene encoding the CD163 protein.

Alternatively or in addition, the modified chromosomal sequence in the gene encoding the CD163 protein can comprise an insertion in an allele of the gene encoding the CD163 protein.

The deletion, the substitution, or the combination of any thereof can result in a miscoding in the allele of the gene encoding the CD163 protein.

The insertion, the deletion, the substitution, or the miscoding can result in a premature stop codon in the allele of the gene encoding the CD163 protein.

In any of the animals, offspring, or cells described herein, the modified chromosomal sequence in the gene encoding the CD163 protein preferably causes CD163 protein production or activity to be reduced, as compared to CD163 protein production or activity in an animal, offspring, or cell that lacks the modified chromosomal sequence in the gene encoding the CD163 protein.

Preferably, the modified chromosomal sequence in the gene encoding the CD163 protein results in production of substantially no functional CD163 protein by the animal, offspring or cell. By “substantially no functional CD163 protein,” it is meant that the level of CD163 protein in the animal, offspring, or cell is undetectable, or if detectable, is at least about 90% lower, preferably at least about 95% lower, more preferably at least about 98%, lower, and even more preferably at least about 99% lower than the level observed in an animal, offspring, or cell that does not comprise the modified chromosomal sequences.

Where the animal, offspring, or cell comprises a modified chromosomal sequence in a gene encoding a CD163 protein, the animal, offspring, or cell preferably does not produce CD163 protein.

The animal or offspring comprising a modified chromosomal sequence in a gene encoding a CD163 protein can comprise a porcine animal.

Similarly, the cell comprising a modified chromosomal sequence in a gene encoding a CD163 protein can comprise a porcine cell.

Where the animal or offspring comprises a porcine animal or where the cell comprises a porcine cell, the modified chromosomal sequence in the gene encoding the CD163 protein can comprise a modification in: exon 7 of an allele of the gene encoding the CD163 protein; exon 8 of an allele of the gene encoding the CD163 protein; an intron that is contiguous with exon 7 or exon 8 of the allele of the gene encoding the CD163 protein; or a combination of any thereof.

For example, the modified chromosomal sequence in the gene encoding the CD163 protein can comprise a modification in exon 7 of the allele of the gene encoding the CD163 protein

The modification in exon 7 of the allele of the gene encoding the CD163 protein can comprise an insertion.

The modification in exon 7 of the allele of the gene encoding the CD163 protein can comprise a deletion.

Where the animal, offspring, or cell comprises a deletion in an allele of the gene encoding the CD163 protein, the deletion can optionally comprise an in-frame deletion.

Where the animal or offspring comprises a porcine animal or where the cell comprises a porcine cell, the modified chromosomal sequence in the gene encoding the CD163 protein can comprise a modification selected from the group consisting of: an 11 base pair deletion from nucleotide 3,137 to nucleotide 3,147 as compared to reference sequence SEQ ID NO: 47; a 2 base pair insertion between nucleotides 3,149 and 3,150 as compared to reference sequence SEQ ID NO: 47, with a 377 base pair deletion from nucleotide 2,573 to nucleotide 2,949 as compared to reference sequence SEQ ID NO: 47 on the same allele; a 124 base pair deletion from nucleotide 3,024 to nucleotide 3,147 as compared to reference sequence SEQ ID NO: 47; a 123 base pair deletion from nucleotide 3,024 to nucleotide 3,146 as compared to reference sequence SEQ ID NO: 47; a 1 base pair insertion between nucleotides 3,147 and 3,148 as compared to reference sequence SEQ ID NO: 47; a 130 base pair deletion from nucleotide 3,030 to nucleotide 3,159 as compared to reference sequence SEQ ID NO: 47; a 132 base pair deletion from nucleotide 3,030 to nucleotide 3,161 as compared to reference sequence SEQ ID NO: 47; a 1506 base pair deletion from nucleotide 1,525 to nucleotide 3,030 as compared to reference sequence SEQ ID NO: 47; a 7 base pair insertion between nucleotide 3,148 and nucleotide 3,149 as compared to reference sequence SEQ ID NO: 47; a 1280 base pair deletion from nucleotide 2,818 to nucleotide 4,097 as compared to reference sequence SEQ ID NO: 47; a 1373 base pair deletion from nucleotide 2,724 to nucleotide 4,096 as compared to reference sequence SEQ ID NO: 47; a 1467 base pair deletion from nucleotide 2,431 to nucleotide 3,897 as compared to reference sequence SEQ ID NO: 47; a 1930 base pair deletion from nucleotide 488 to nucleotide 2,417 as compared to reference sequence SEQ ID NO: 47, wherein the deleted sequence is replaced with a 12 base pair insertion beginning at nucleotide 488, and wherein there is a further 129 base pair deletion in exon 7 from nucleotide 3,044 to nucleotide 3,172 as compared to reference sequence SEQ ID NO: 47; a 28 base pair deletion from nucleotide 3,145 to nucleotide 3,172 as compared to reference sequence SEQ ID NO: 47; a 1387 base pair deletion from nucleotide 3,145 to nucleotide 4,531 as compared to reference sequence SEQ ID NO: 47; a 1382 base pair deletion from nucleotide 3,113 to nucleotide 4,494 as compared to reference sequence SEQ ID NO: 47, wherein the deleted sequence is replaced with an 11 base pair insertion beginning at nucleotide 3,113; a 1720 base pair deletion from nucleotide 2,440 to nucleotide 4,160 as compared to reference sequence SEQ ID NO: 47; a 452 base pair deletion from nucleotide 3,015 to nucleotide 3,466 as compared to reference sequence SEQ ID NO: 47; and combinations of any thereof.

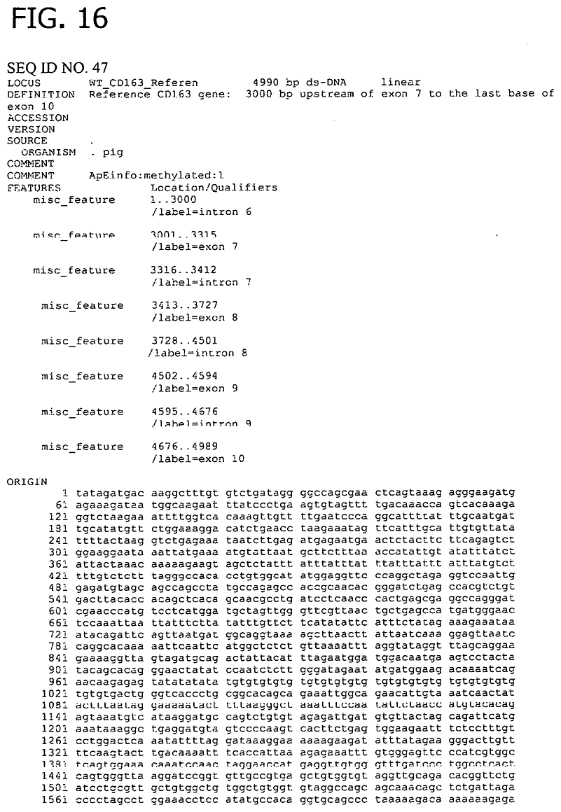

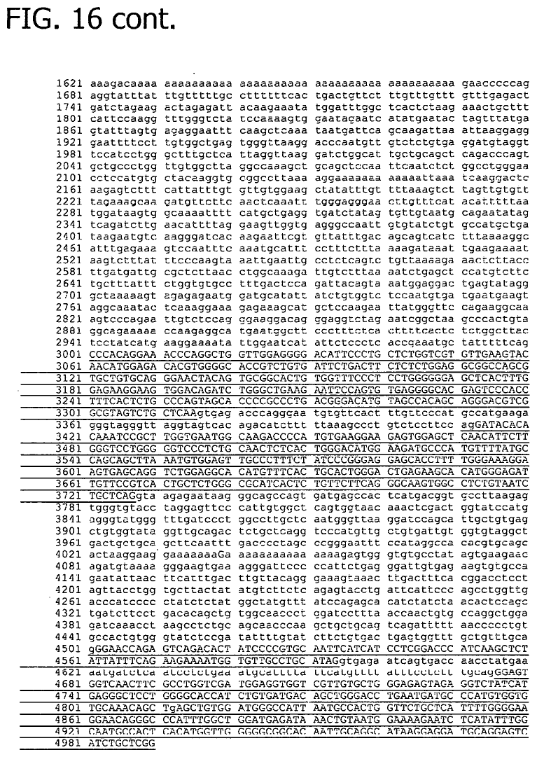

SEQ ID NO: 47 provides a partial nucleotide sequence for wild-type porcine CD163. SEQ ID NO: 47 includes a region beginning 3000 base pairs (bp) upstream of exon 7 of the wild-type porcine CD163 gene through the last base of exon 10 of this gene. SEQ ID NO: 47 is used as a reference sequence herein and is shown in FIG. 16.

For example, the modified chromosomal sequence in the gene encoding the CD163 protein can comprise a modification selected from the group consisting of: the 7 base pair insertion between nucleotide 3,148 and nucleotide 3,149 as compared to reference sequence SEQ ID NO: 47; the 2 base pair insertion between nucleotides 3,149 and 3,150 as compared to reference sequence SEQ ID NO: 47, with the 377 base pair deletion from nucleotide 2,573 to nucleotide 2,949 as compared to reference sequence SEQ ID NO: 47 on the same allele; the 11 base pair deletion from nucleotide 3,137 to nucleotide 3,147 as compared to reference sequence SEQ ID NO: 47; the 1382 base pair deletion from nucleotide 3,113 to nucleotide 4,494 as compared to reference sequence SEQ ID NO: 47, wherein the deleted sequence is replaced with the 11 base pair insertion beginning at nucleotide 3,113; the 1387 base pair deletion from nucleotide 3,145 to nucleotide 4,531 as compared to reference sequence SEQ ID NO: 47; and combinations of any thereof.

The modified chromosomal sequence can comprise an 11 base pair deletion from nucleotide 3,137 to nucleotide 3,147 as compared to reference sequence SEQ ID NO: 47.

The modified chromosomal sequence can comprise a 2 base pair insertion between nucleotides 3,149 and 3,150 as compared to reference sequence SEQ ID NO: 47, with a 377 base pair deletion from nucleotide 2,573 to nucleotide 2,949 as compared to reference sequence SEQ ID NO: 47 on the same allele.

Where the modified chromosomal sequence comprises the 2 base pair insertion between nucleotides 3,149 and 3,150 as compared to reference sequence SEQ ID NO: 47, with a 377 base pair deletion from nucleotide 2,573 to nucleotide 2,949 as compared to reference sequence SEQ ID NO: 47 on the same allele, the 2 base pair insertion can comprise the dinucleotide AG.

The modified chromosomal sequence can comprise a 124 base pair deletion from nucleotide 3,024 to nucleotide 3,147 as compared to reference sequence SEQ ID NO: 47.

The modified chromosomal sequence can comprise a 123 base pair deletion from nucleotide 3,024 to nucleotide 3,146 as compared to reference sequence SEQ ID NO: 47.

The modified chromosomal sequence can comprise a 1 base pair insertion between nucleotides 3,147 and 3,148 as compared to reference sequence SEQ ID NO: 47.

Where the modified chromosomal sequence comprises the 1 base pair insertion between nucleotides 3,147 and 3,148 as compared to reference sequence SEQ ID NO: 47, the 1 base pair insertion can comprise a single adenine residue.

The modified chromosomal sequence can comprise a 130 base pair deletion from nucleotide 3,030 to nucleotide 3,159 as compared to reference sequence SEQ ID NO: 47.

The modified chromosomal sequence can comprise a 132 base pair deletion from nucleotide 3,030 to nucleotide 3,161 as compared to reference sequence SEQ ID NO: 47.

The modified chromosomal sequence can comprise a 1506 base pair deletion from nucleotide 1,525 to nucleotide 3,030 as compared to reference sequence SEQ ID NO: 47.

The modified chromosomal sequence can comprise a 7 base pair insertion between nucleotide 3,148 and nucleotide 3,149 as compared to reference sequence SEQ ID NO:

47.

Where the modified chromosomal sequence comprises the 7 base pair insertion between nucleotide 3,148 and nucleotide 3,149 as compared to reference sequence SEQ ID NO: 47, the 7 base pair insertion can comprise the sequence TACTACT (SEQ ID NO: 115).

The modified chromosomal sequence can comprise a 1280 base pair deletion from nucleotide 2,818 to nucleotide 4,097 as compared to reference sequence SEQ ID NO: 47.

The modified chromosomal sequence can comprise a 1373 base pair deletion from nucleotide 2,724 to nucleotide 4,096 as compared to reference sequence SEQ ID NO: 47.

The modified chromosomal sequence can comprise a 1467 base pair deletion from nucleotide 2,431 to nucleotide 3,897 as compared to reference sequence SEQ ID NO: 47.

The modified chromosomal sequence can comprise a 1930 base pair deletion from nucleotide 488 to nucleotide 2,417 as compared to reference sequence SEQ ID NO: 47, wherein the deleted sequence is replaced with a 12 base pair insertion beginning at nucleotide 488, and wherein there is a further 129 base pair deletion in exon 7 from nucleotide 3,044 to nucleotide 3,172 as compared to reference sequence SEQ ID NO: 47.

Where the modified chromosomal sequence comprises the 1930 base pair deletion from nucleotide 488 to nucleotide 2,417 as compared to reference sequence SEQ ID NO: 47, wherein the deleted sequence is replaced with a 12 base pair insertion beginning at nucleotide 488, and wherein there is a further 129 base pair deletion in exon 7 from nucleotide 3,044 to nucleotide 3,172 as compared to reference sequence SEQ ID NO: 47, the 12 base pair insertion can comprise the sequence TGTGGAGAATTC (SEQ ID NO: 116).

The modified chromosomal sequence can comprise a 28 base pair deletion from nucleotide 3,145 to nucleotide 3,172 as compared to reference sequence SEQ ID NO: 47.

The modified chromosomal sequence can comprise a 1387 base pair deletion from nucleotide 3,145 to nucleotide 4,531 as compared to reference sequence SEQ ID NO: 47.

The modified chromosomal sequence can comprise a 1382 base pair deletion from nucleotide 3,113 to nucleotide 4,494 as compared to reference sequence SEQ ID NO: 47, wherein the deleted sequence is replaced with an 11 base pair insertion beginning at nucleotide 3,113.

Where the modified chromosomal sequence comprises the 1382 base pair deletion from nucleotide 3,113 to nucleotide 4,494 as compared to reference sequence SEQ ID NO: 47, wherein the deleted sequence is replaced with an 11 base pair insertion beginning at nucleotide 3,113, the 11 base pair insertion can comprise the sequence AGCCAGCGTGC (SEQ ID NO: 117).

The modified chromosomal sequence can comprise a 1720 base pair deletion from nucleotide 2,440 to nucleotide 4,160 as compared to reference sequence SEQ ID NO: 47.

The modified chromosomal sequence can comprise a 452 base pair deletion from nucleotide 3,015 to nucleotide 3,466 as compared to reference sequence SEQ ID NO: 47.

The CD163 gene in the animal, offspring, or cell can comprise any combination of any of the modified chromosomal sequences described herein.

For example, the animal, offspring or cell can comprise the 7 base pair insertion between nucleotide 3,148 and nucleotide 3,149 as compared to reference sequence SEQ ID NO: 47 in one allele of the gene encoding the CD163 protein; and the 11 base pair deletion from nucleotide 3,137 to nucleotide 3,147 as compared to reference sequence SEQ ID NO: 47 in the other allele of the gene encoding the CD163 protein.

The animal, offspring, or cell can comprise the 7 base pair insertion between nucleotide 3,148 and nucleotide 3,149 as compared to reference sequence SEQ ID NO: 47 in one allele of the gene encoding the CD163 protein; and the 1382 base pair deletion from nucleotide 3,113 to nucleotide 4,494 as compared to reference sequence SEQ ID NO: 47, wherein the deleted sequence is replaced with an 11 base pair insertion beginning at nucleotide 3,113 in the other allele of the gene encoding the CD163 protein.

The animal, offspring, or cell can comprise the 1280 base pair deletion from nucleotide 2,818 to nucleotide 4,097 as compared to reference sequence SEQ ID NO: 47 in one allele of the gene encoding the CD163 protein; and the 11 base pair deletion from nucleotide 3,137 to nucleotide 3,147 as compared to reference sequence SEQ ID NO: 47 in the other allele of the gene encoding the CD163 protein.

The animal, offspring, or cell can comprise the 1280 base pair deletion from nucleotide 2,818 to nucleotide 4,097 as compared to reference sequence SEQ ID NO: 47 in one allele of the gene encoding the CD163 protein; and the 2 base pair insertion between nucleotides 3,149 and 3,150 as compared to reference sequence SEQ ID NO: 47, with the 377 base pair deletion from nucleotide 2,573 to nucleotide 2,949 as compared to reference sequence SEQ ID NO: 47 in the other allele of the gene encoding the CD163 protein.

The animal, offspring, or cell can comprise the 1930 base pair deletion from nucleotide 488 to nucleotide 2,417 as compared to reference sequence SEQ ID NO: 47, wherein the deleted sequence is replaced with a 12 base pair insertion beginning at nucleotide 488, and wherein there is a further 129 base pair deletion in exon 7 from nucleotide 3,044 to nucleotide 3,172 as compared to reference sequence SEQ ID NO: 47 in one allele of the gene encoding the CD163 protein; and the 2 base pair insertion between nucleotides 3,149 and 3,150 as compared to reference sequence SEQ ID NO: 47, with the 377 base pair deletion from nucleotide 2,573 to nucleotide 2,949 as compared to reference sequence SEQ ID NO: 47 in the other allele of the gene encoding the CD163 protein.

The animal, offspring, or cell can comprise the 1930 base pair deletion from nucleotide 488 to nucleotide 2,417 as compared to reference sequence SEQ ID NO: 47, wherein the deleted sequence is replaced with a 12 base pair insertion beginning at nucleotide 488, and wherein there is a further 129 base pair deletion in exon 7 from nucleotide 3,044 to nucleotide 3,172 as compared to reference sequence SEQ ID NO: 47 in one allele of the gene encoding the CD163 protein; and the 11 base pair deletion from nucleotide 3,137 to nucleotide 3,147 as compared to reference sequence SEQ ID NO: 47 in the other allele of the gene encoding the CD163 protein.

The animal, offspring, or cell can comprise the 1467 base pair deletion from nucleotide 2,431 to nucleotide 3,897 as compared to reference sequence SEQ ID NO: 47 in one allele of the gene encoding the CD163 protein; and the 2 base pair insertion between nucleotides 3,149 and 3,150 as compared to reference sequence SEQ ID NO: 47, with the 377 base pair deletion from nucleotide 2,573 to nucleotide 2,949 as compared to reference sequence SEQ ID NO: 47 in the other allele of the gene encoding the CD163 protein.

The animal, offspring, or cell can comprise the 1467 base pair deletion from nucleotide 2,431 to nucleotide 3,897 as compared to reference sequence SEQ ID NO: 47 in one allele of the gene encoding the CD163 protein; and the 11 base pair deletion from nucleotide 3,137 to nucleotide 3,147 as compared to reference sequence SEQ ID NO: 47 in the other allele of the gene encoding the CD163 protein.

The animal, offspring, or cell can comprise the 11 base pair deletion from nucleotide 2,431 to nucleotide 3,897 as compared to reference sequence SEQ ID NO: 47 in one allele of the gene encoding the CD163 protein; and the 2 base pair insertion between nucleotides 3,149 and 3,150 as compared to reference sequence SEQ ID NO: 47, with the 377 base pair deletion from nucleotide 2,573 to nucleotide 2,949 as compared to reference sequence SEQ ID NO: 47 in the other allele of the gene encoding the CD163 protein.

The animal, offspring, or cell can comprise the 124 base pair deletion from nucleotide 3,024 to nucleotide 3,147 as compared to reference sequence SEQ ID NO: 47 in one allele of the gene encoding the CD163 protein; and the 123 base pair deletion from nucleotide 3,024 to nucleotide 3,146 as compared to reference sequence SEQ ID NO: 47 in the other allele of the gene encoding the CD163 protein.

The animal, offspring, or cell can comprise the 130 base pair deletion from nucleotide 3,030 to nucleotide 3,159 as compared to reference sequence SEQ ID NO: 47 in one allele of the gene encoding the CD163 protein; and the 132 base pair deletion from nucleotide 3,030 to nucleotide 3,161 as compared to reference sequence SEQ ID NO: 47 in the other allele of the gene encoding the CD163 protein.

The animal, offspring, or cell can comprise the 1280 base pair deletion from nucleotide 2,818 to nucleotide 4,097 as compared to reference sequence SEQ ID NO: 47 in one allele of the gene encoding the CD163 protein; and the 1373 base pair deletion from nucleotide 2,724 to nucleotide 4,096 as compared to reference sequence SEQ ID NO: 47 in the other allele of the gene encoding the CD163 protein.

The animal, offspring, or cell can comprise the 28 base pair deletion from nucleotide 3,145 to nucleotide 3,172 as compared to reference sequence SEQ ID NO: 47 in one allele of the gene encoding the CD163 protein; and the 1387 base pair deletion from nucleotide 3,145 to nucleotide 4,531 as compared to reference sequence SEQ ID NO: 47 in the other allele of the gene encoding the CD163 protein.

The animal, offspring, or cell can comprise the 1382 base pair deletion from nucleotide 3,113 to nucleotide 4,494 as compared to reference sequence SEQ ID NO: 47, wherein the deleted sequence is replaced with an 11 base pair insertion beginning at nucleotide 3,113, in one allele of the gene encoding the CD163 protein; and the 1720 base pair deletion from nucleotide 2,440 to nucleotide 4,160 as compared to reference sequence SEQ ID NO: 47 in the other allele of the gene encoding the CD163 protein.

The animal, offspring, or cell can comprise the 7 base pair insertion between nucleotide 3,148 and nucleotide 3,149 as compared to reference sequence SEQ ID NO: 47 in one allele of the CD163 gene; and the 2 base pair insertion between nucleotides 3,149 and 3,150 as compared to reference sequence SEQ ID NO: 47, with the 377 base pair deletion from nucleotide 2,573 to nucleotide 2,949 as compared to reference sequence SEQ ID NO: 47, in the other allele of the CD163 gene.

The animal, offspring, or cell can comprise the 1382 base pair deletion from nucleotide 3,113 to nucleotide 4,494 as compared to reference sequence SEQ ID NO: 47, wherein the deleted sequence is replaced with the 11 base pair insertion beginning at nucleotide 3,113, in one allele of the CD163 gene; and the 2 base pair insertion between nucleotides 3,149 and 3,150 as compared to reference sequence SEQ ID NO: 47, with the 377 base pair deletion from nucleotide 2,573 to nucleotide 2,949 as compared to reference sequence SEQ ID NO: 47, in the other allele of the CD163 gene.

The animal, offspring, or cell can comprise the 1382 base pair deletion from nucleotide 3,113 to nucleotide 4,494 as compared to reference sequence SEQ ID NO: 47, wherein the deleted sequence is replaced with the 11 base pair insertion beginning at nucleotide 3,113, in one allele of the CD163 gene; and the 11 base pair deletion from nucleotide 3,137 to nucleotide 3,147 as compared to reference sequence SEQ ID NO: 47 in the other allele of the CD163 gene.

Any of the modified chromosomal sequences in the gene encoding the CD163 protein described herein can consist of the deletion, insertion or substitution.

In any of the animals, offspring, or cells described herein, the animal, offspring, or cell can comprise a chromosomal sequence in the gene encoding the CD163 protein having at least 80% sequence identity to SEQ ID NO: 47 in the regions of the chromosomal sequence outside of the insertion, the deletion, or the substitution.

The animal, offspring, or cell can comprise a chromosomal sequence in the gene encoding the CD163 protein having at least 85% sequence identity to SEQ ID NO: 47 in the regions of the chromosomal sequence outside of the insertion, the deletion, or the substitution.

The animal, offspring, or cell can comprise a chromosomal sequence in the gene encoding the CD163 protein having at least 90% sequence identity to SEQ ID NO: 47 in the regions of the chromosomal sequence outside of the insertion, the deletion, or the substitution.

The animal, offspring, or cell can comprise a chromosomal sequence in the gene encoding the CD163 protein having at least 95% sequence identity to SEQ ID NO: 47 in the regions of the chromosomal sequence outside of the insertion, the deletion, or the substitution.

The animal, offspring, or cell can comprise a chromosomal sequence in the gene encoding the CD163 protein having at least 98% sequence identity to SEQ ID NO: 47 in the regions of the chromosomal sequence outside of the insertion, the deletion, or the substitution.

The animal, offspring, or cell can comprise a chromosomal sequence in the gene encoding the CD163 protein having at least 99% sequence identity to SEQ ID NO: 47 in the regions of the chromosomal sequence outside of the insertion, the deletion, or the substitution.

The animal, offspring, or cell can comprise a chromosomal sequence in the gene encoding the CD163 protein having at least 99.9% sequence identity to SEQ ID NO: 47 in the regions of the chromosomal sequence outside of the insertion, the deletion, or the substitution.

The animal, offspring, or cell can comprise a chromosomal sequence in the gene encoding the CD163 protein having 100% sequence identity to SEQ ID NO: 47 in the regions of the chromosomal sequence outside of the insertion, the deletion, or the substitution.

Any of the animals, offspring, or cells can comprise a chromosomal sequence comprising SEQ ID NO: 98, 99, 100, 101, 102, 103, 104, 105, 106, 107, 108, 109, 110, 111, 112, 113, 114, or 119.

In any of the animals, offspring, or cells comprising modified chromosomal sequences in both a gene encoding an ANPEP protein and a gene encoding a CD163 protein, the animal, offspring, or cell can comprise any combination of any of the modified chromosomal sequences in a gene encoding an ANPEP protein described herein and any of the modified chromosomal sequences in a gene encoding a CD163 protein described herein.

For example, the modified chromosomal sequence in the gene encoding the ANPEP protein can comprises the 1 base pair insertion between nucleotides 1,581 and 1,582, as compared to reference sequence SEQ ID NO: 135, and the modified chromosomal sequence in the gene encoding the CD163 protein can comprise the 1387 base pair deletion from nucleotide 3,145 to nucleotide 4,531 as compared to reference sequence SEQ ID NO: 47.

### Animals and Cells Having a Modified Chromosomal Sequence in a Gene Encoding an ANPEP and Further Comprising a Modified Chromosomal Sequence in a Gene Encoding a SIGLEC1 Protein

Any of the animals, offspring, or cells that comprise at least one modified chromosomal sequence in a gene encoding an ANPEP protein can further comprise at least one modified chromosomal sequence in a gene encoding a SIGLEC1 protein.

The animal, offspring, or cell can be heterozygous for the modified chromosomal sequence in the gene encoding the SIGLEC1 protein.

The animal, offspring, or cell can be homozygous for the modified chromosomal sequence in the gene encoding the SIGLEC1 protein.

The modified chromosomal sequence in the gene encoding the SIGLEC1 protein can comprise an insertion in an allele of the gene encoding the SIGLEC1 protein, a deletion in an allele of the gene encoding the SIGLEC1 protein, a substitution in an allele of the gene encoding the SIGLEC1 protein, or a combination of any thereof.

For example, the modified chromosomal sequence in the gene encoding the SIGLEC1 protein can comprise a deletion in an allele of the gene encoding the SIGLEC1 protein.

Where the modified chromosomal sequence in the gene encoding the SIGLEC protein comprises a deletion in an allele of the gene encoding the SIGLEC1 protein, the deletion can comprise an in-frame deletion.

The modified chromosomal sequence in the gene encoding the SIGLEC1 protein can comprise an insertion in an allele of the gene encoding the SIGLEC1 protein.

The modified chromosomal sequence in the gene encoding the SIGLEC1 protein can comprise a substitution in an allele of the gene encoding the SIGLEC1 protein.

The deletion, the substitution, or the combination of any thereof can result in a miscoding in the allele of the gene encoding the SIGLEC1 protein.

The insertion, the deletion, the substitution, or the miscoding can result in a premature stop codon in the allele of the gene encoding the SIGLEC1 protein.

In any of the animals, offspring, or cells described herein, the modified chromosomal sequence in the gene encoding the SIGLEC1 protein preferably causes SIGLEC1 protein production or activity to be reduced, as compared to SIGLEC1 protein production or activity in an animal, offspring, or cell that lacks the modified chromosomal sequence in the gene encoding the SIGLEC1 protein.

Preferably, the modified chromosomal sequence in the gene encoding the SIGLEC1 protein results in production of substantially no functional SIGLEC1 protein by the animal, offspring or cell. By “substantially no functional SIGLEC1 protein,” it is meant that the level of SIGLEC1 protein in the animal, offspring, or cell is undetectable, or if detectable, is at least about 90% lower, preferably at least about 95% lower, more preferably at least about 98%, lower, and even more preferably at least about 99% lower than the level observed in an animal, offspring, or cell that does not comprise the modified chromosomal sequences.

Where the animal, offspring, or cell comprises a modified chromosomal sequence in a gene encoding a SIGLEC1 protein, the animal, offspring, or cell preferably does not produce SIGLEC1 protein.

The animal or offspring comprising a modified chromosomal sequence in a gene encoding a SIGLEC1 protein can comprise a porcine animal.

Similarly, the cell comprising a modified chromosomal sequence in a gene encoding a SIGLEC1 protein can comprise a porcine cell.

Where the animal or offspring comprises a porcine animal or where the cell comprises a porcine cell, the modified chromosomal sequence in the gene encoding the SIGLEC1 protein can comprise a modification in: exon 1 of an allele of the gene encoding the SIGLEC1 protein; exon 2 of an allele of the gene encoding the SIGLEC1 protein; exon 3 of an allele of the gene encoding the SIGLEC1 protein; an intron that is contiguous with exon 1, exon 2, or exon 3 of an allele of the gene encoding the SIGLEC1 protein; or a combination of any thereof.

For example, the modified chromosomal sequence in the gene encoding the SIGLEC1 protein can comprises a deletion in exon 1, exon 2, and/or exon 3 of an allele of the gene encoding the SIGLEC1 protein.

The modified chromosomal sequence in the gene encoding the SIGLEC1 protein can comprise a deletion of part of exon 1 and all of exons 2 and 3 of an allele of the gene encoding the SIGLEC1 protein.

For example, the modified chromosomal sequence comprises a 1,247 base pair deletion from nucleotide 4,279 to nucleotide 5,525 as compared to reference sequence SEQ ID NO: 122.

SEQ ID NO: 122 provides a partial nucleotide sequence for wild-type porcine SIGLEC1. SEQ ID NO: 122 begins 4,236 nucleotides upstream of exon 1, includes all introns and exons through exon 7, and 1,008 nucleotides following the end of exon 7. SEQ ID NO: 122 is used as a reference sequence herein.

Where the modified chromosomal sequence in the gene encoding the SIGLEC1 protein comprises a deletion, the deleted sequence can optionally be replaced with a neomycin cassette. For example, the animal, offspring, or cell can comprise a chromosomal sequence comprising SEQ ID NO: 123. SEQ ID NO: 123 provides a partial nucleotide sequence wherein, as compared to reference sequence SEQ ID NO: 122, there is a 1,247 base pair deletion from nucleotide 4,279 to 5,525 and the deleted sequence is replaced with a 1,855 base pair neomycin selectable cassette oriented in the opposite direction as compared to SEQ ID NO: 122. This insertion/deletion results in the loss of part of exon 1 and all of exon 2 and 3 of the SIGLEC1 gene.

Any of the modified chromosomal sequences in the gene encoding the SIGLEC1 protein described herein can consist of the deletion, insertion or substitution.

In any of the animals, offspring, or cells described herein, the animal, offspring, or cell can comprise a chromosomal sequence in the gene encoding the SIGLEC1 protein having at least 80% sequence identity to SEQ ID NO: 122 in the regions of the chromosomal sequence outside of the insertion, the deletion, or the substitution.

The animal, offspring, or cell can comprise a chromosomal sequence in the gene encoding the SIGLEC1protein having at least 85% sequence identity to SEQ ID NO: 122 in the regions of the chromosomal sequence outside of the insertion, the deletion, or the substitution.

The animal, offspring, or cell can comprise a chromosomal sequence in the gene encoding the SIGLEC1protein having at least 90% sequence identity to SEQ ID NO: 122 in the regions of the chromosomal sequence outside of the insertion, the deletion, or the substitution.

The animal, offspring, or cell can comprise a chromosomal sequence in the gene encoding the SIGLEC1protein having at least 95% sequence identity to SEQ ID NO: 122 in the regions of the chromosomal sequence outside of the insertion, the deletion, or the substitution.

The animal, offspring, or cell can comprise a chromosomal sequence in the gene encoding the SIGLEC1protein having at least 98% sequence identity to SEQ ID NO: 122 in the regions of the chromosomal sequence outside of the insertion, the deletion, or the substitution.

The animal, offspring, or cell can comprise a chromosomal sequence in the gene encoding the SIGLEC1protein having at least 99% sequence identity to SEQ ID NO: 122 in the regions of the chromosomal sequence outside of the insertion, the deletion, or the substitution.

The animal, offspring, or cell can comprise a chromosomal sequence in the gene encoding the SIGLEC1protein having at least 99.9% sequence identity to SEQ ID NO: 122 in the regions of the chromosomal sequence outside of the insertion, the deletion, or the substitution.

The animal, offspring, or cell can comprise a chromosomal sequence in the gene encoding the SIGLEC1protein having 100% sequence identity to SEQ ID NO: 122 in the regions of the chromosomal sequence outside of the insertion, the deletion, or the substitution.

In any of the animals, offspring, or cells comprising modified chromosomal sequences in both a gene encoding an ANPEP protein and a gene encoding a SIGLEC1 protein, the animal, offspring, or cell can comprise any combination of any of the modified chromosomal sequences in a gene encoding an ANPEP protein described herein and any of the modified chromosomal sequences in a gene encoding a SIGLEC1 protein described herein.

For example, the modified chromosomal sequence in the gene encoding the ANPEP protein can comprise the 1 base pair insertion between nucleotides 1,581 and 1,582, as compared to reference sequence SEQ ID NO: 135, and the modified chromosomal sequence in the gene encoding the SIGLEC1 protein can comprise the 1,247 base pair deletion from nucleotide 4,279 to nucleotide 5,525 as compared to reference sequence SEQ ID NO: 122.

### Animals and Cells Having a Modified Chromosomal Sequence in a Gene Encoding an ANPEP and Further Comprising a Modified Chromosomal Sequence in a Gene Encoding a CD163 Protein and a Modified Chromosomal Sequence in a Gene Encoding a SIGLEC1 Protein

Any of the animals, offspring, or cells that comprise at least one modified chromosomal sequence in a gene encoding an ANPEP protein can further comprise at least one modified chromosomal sequence in a gene encoding a CD163 protein and at least one modified chromosomal sequence in a gene encoding a SIGLEC1 protein.

Where the animal, offspring, or cell comprises a modified chromosomal sequence in a gene encoding an ANPEP protein, a modified chromosomal sequence in a gene encoding a CD163 protein, and a modified chromosomal sequence in a gene encoding a SIGLEC1 protein, the animal, offspring, or cell can comprise any combination of any of the modified chromosomal sequences in a gene encoding an ANPEP protein described herein, any of the modified chromosomal sequences in a gene encoding a CD163 protein described herein, and any of the modified chromosomal sequences in a gene encoding a SIGLEC1 protein described herein.

For example, the modified chromosomal sequence in the gene encoding the ANPEP protein can comprise the 1 base pair insertion between nucleotides 1,581 and 1,582, as compared to reference sequence SEQ ID NO: 135, the modified chromosomal sequence in the gene encoding the SIGLEC1 protein can comprise the 1,247 base pair deletion from nucleotide 4,279 to nucleotide 5,525 as compared to reference sequence SEQ ID NO: 122, and the modified chromosomal sequence in the gene encoding the CD163 protein can comprise the 1387 base pair deletion from nucleotide 3,145 to nucleotide 4,531 as compared to reference sequence SEQ ID NO: 47.

### Genetically Edited Animals and Cells

Any of the animals or offspring described herein can be a genetically edited animal.

Likewise, any of the cells described herein can be a genetically edited cell.

The animal, offspring, or cell can be an animal, offspring, or cell that has been edited using a homing endonuclease. The homing endonuclease can be a naturally occurring endonuclease but is preferably a rationally designed, non-naturally occurring homing endonuclease that has a DNA recognition sequence that has been designed so that the endonuclease targets a chromosomal sequence in a gene encoding an ANPEP, CD163, or SIGLEC1 protein.

Thus, the homing endonuclease can be a designed homing endonuclease. The homing endonuclease can comprise, for example, a Clustered Regularly Interspaced Short Palindromic Repeats (CRISPR) system, a Transcription Activator-Like Effector Nuclease (TALEN), a Zinc Finger Nuclease (ZFN), a recombinase fusion protein, a meganuclease, or a combination of any thereof.

The homing nuclease preferably comprises a CRISPR system. Examples of CRISPR systems that can be used to create the female porcine animals for use in the methods described herein include, but are not limited to CRISPR/Cas9, CRISPR/Cas5, and CRISPR/Cas6.

The use of various homing endonucleases, including CRISPR systems and TALENs, to generate genetically edited animals is discussed further hereinbelow.

The edited chromosomal sequence may be (1) inactivated, (2) modified, or (3) comprise an integrated sequence resulting in a null mutation. Where the edited chromosomal sequence is in an ANPEP gene, an inactivated chromosomal sequence is altered such that an ANPEP protein function as it relates to TGEV and/or PRCV infection is impaired, reduced, or eliminated. Where the edited chromosomal sequence is in a CD163 gene, an inactivated chromosomal sequence is altered such that a CD163 protein function as it relates to PRRSV infection is impaired, reduced or eliminated. Thus, a genetically edited animal comprising an inactivated chromosomal sequence may be termed a “knock out” or a “conditional knock out.” Similarly, a genetically edited animal comprising an integrated sequence may be termed a “knock in” or a “conditional knock in.” Furthermore, a genetically edited animal comprising a modified chromosomal sequence may comprise a targeted point mutation(s) or other modification such that an altered protein product is produced. Briefly, the process can comprise introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with germline development using targeted zinc finger nuclease technology is rapid, precise, and highly efficient.

Alternatively, the process can comprise using a CRISPR system (e.g., a CRISPR/Cas9 system) to modify the genomic sequence. To use Cas9 to modify genomic sequences, the protein can be delivered directly to a cell. Alternatively, an mRNA that encodes Cas9 can be delivered to a cell, or a gene that provides for expression of an mRNA that encodes Cas9 can be delivered to a cell. In addition, either target specific crRNA and a tracrRNA can be delivered directly to a cell or target specific gRNA(s) can be to a cell (these RNAs can alternatively be produced by a gene constructed to express these RNAs). Selection of target sites and designed of crRNA/gRNA are well known in the art. A discussion of construction and cloning of gRNAs can be found at http://www.genome-engineering.org/crispr/wp-content/uploads/2014/05/CRISPR-Reagent-Description-Rev20140509.pdf.

At least one ANPEP, CD163, or SIGLEC1 locus can be used as a target site for the site-specific editing. The site-specific editing can include insertion of an exogenous nucleic acid (e.g., a nucleic acid comprising a nucleotide sequence encoding a polypeptide of interest) or deletions of nucleic acids from the locus. For example, integration of the exogenous nucleic acid and/or deletion of part of the genomic nucleic acid can modify the locus so as to produce a disrupted (i.e., reduced activity of ANPEP, CD163, or SIGLEC1 protein) ANPEP, CD163, or SIGLEC I gene.

### Cell Types

Any of the cells described herein can comprise a germ cell or a gamete.

For example, any of the cells described herein can comprise a sperm cell.

Alternatively, any of the cells described herein can comprise an egg cell (e.g., a fertilized egg).

Any of the cells described herein can comprise a somatic cell.

For example, any of the cells described herein can comprise a fibroblast (e.g., a fetal fibroblast).

Any of the cells described herein can comprise an embryonic cell.

Any of the cells described herein can comprise a cell derived from a juvenile animal.

Any of the cells described herein can comprise a cell derived from an adult animal.

### Methods for Producing Animals and Lineages Having Reduced Susceptibility to a Pathogen

A method for producing a non-human animal or a lineage of non-human animals having reduced susceptibility to a pathogen is provided. The method comprises modifying an oocyte or a sperm cell to introduce a modified chromosomal sequence in a gene encoding an aminopeptidase N (ANPEP) protein into at least one of the oocyte and the sperm cell, and fertilizing the oocyte with the sperm cell to create a fertilized egg containing the modified chromosomal sequence in the gene encoding a ANPEP protein. The method further comprises transferring the fertilized egg into a surrogate female animal, wherein gestation and term delivery produces a progeny animal. The method additionally comprises screening the progeny animal for susceptibility to the pathogen, and selecting progeny animals that have reduced susceptibility to the pathogen as compared to animals that do not comprise a modified chromosomal sequence in a gene encoding an ANPEP protein.

Another method for producing a non-human animal or a lineage of non-human animals having reduced susceptibility to a pathogen is provided. The method comprises modifying a fertilized egg to introduce a modified chromosomal sequence in a gene encoding an ANPEP protein into the fertilized egg. The method further comprises transferring the fertilized egg into a surrogate female animal, wherein gestation and term delivery produces a progeny animal. The method additionally comprises screening the progeny animal for susceptibility to the pathogen, and selecting progeny animals that have reduced susceptibility to the pathogen as compared to animals that do not comprise a modified chromosomal sequence in a gene encoding an ANPEP protein.

In either of these methods, the animal can comprise a livestock animal.

The step of modifying the oocyte, sperm cell, or fertilized egg can comprise genetic editing of the oocyte, sperm cell, or fertilized egg.

The oocyte, sperm cell, or fertilized egg can be heterozygous for the modified chromosomal sequence.

The oocyte, sperm cell, or fertilized egg can be homozygous for the modified chromosomal sequence.

The fertilizing can comprise artificial insemination.

In any of the methods for producing a non-human animal or a lineage of non-human animals having reduced susceptibility to a pathogen, the method can further comprise modifying the oocyte, sperm cell, or fertilized egg to introduce a modified chromosomal sequence in a gene encoding a CD163 protein into the oocyte, the sperm cell, or the fertilized egg.

Alternatively or in addition, in any of the methods for producing a non-human animal or a lineage of non-human animals having reduced susceptibility to a pathogen, the method can further comprise modifying the oocyte, sperm cell, or fertilized egg to introduce a modified chromosomal sequence in a gene encoding a SIGLEC1 protein into the oocyte, the sperm cell, or the fertilized egg.

A method of increasing a livestock animal's resistance to infection with a pathogen is provided. The method comprises modifying at least one chromosomal sequence in a gene encoding an aminopeptidase N (ANPEP) protein so that ANPEP protein production or activity is reduced, as compared to ANPEP protein production or activity in a livestock animal that does not comprise a modified chromosomal sequence in a gene encoding an ANPEP protein.

The method can further optionally comprise modifying at least one chromosomal sequence in a gene encoding a CD163 protein, so that CD163 protein production or activity is reduced, as compared to CD163 protein production or activity in a livestock animal that does not comprise a modified chromosomal sequence in a gene encoding a CD163 protein.

Alternatively or in addition, the method can further optionally comprise modifying at least one chromosomal sequence in a gene encoding a SIGLEC1 protein, so that SIGLEC1 protein production or activity is reduced, as compared to SIGELC1 protein production or activity in a livestock animal that does not comprise a modified chromosomal sequence in a gene encoding a SIGLEC1 protein.

The step of modifying the at least one chromosomal sequence in the gene encoding the ANPEP protein can comprise genetic editing of the chromosomal sequence.

In any of the methods described herein comprising genetic editing, the genetic editing can comprise use of a homing endonuclease. The homing endonuclease can be a naturally occurring endonuclease but is preferably a rationally designed, non-naturally occurring homing endonuclease that has a DNA recognition sequence that has been designed so that the endonuclease targets a chromosomal sequence in a gene encoding an ANPEP protein.

Thus, the homing endonuclease can be a designed homing endonuclease. The homing endonuclease can comprise, for example, a Clustered Regularly Interspaced Short Palindromic Repeats (CRISPR) system, a Transcription Activator-Like Effector Nuclease (TALEN), a Zinc Finger Nuclease (ZFN), a recombinase fusion protein, a meganuclease, or a combination of any thereof.

The homing nuclease preferably comprises a CRISPR system. Examples of CRISPR systems that include, but are not limited to CRISPR/Cas9, CRISPR/Cas5, and CRISPR/Cas6.

Any of the methods described herein can produce any of the animals described herein.

Any of the methods described herein can further comprise using the animal as a founder animal.

### Populations of Animals

Populations of animals are also provided herein.

A population of livestock animals is provided. The population comprises two or more of any of the livestock animals and/or offspring thereof described herein.

Another population of animals is provided. The population comprises two or more animals made by any of the methods described herein and/or offspring thereof.

Thus, the animals in the population will all comprise a modified chromosomal sequence in a gene encoding an ANPEP protein. The animals in the population can also optionally comprise modified chromosomal sequences in an gene encoding a CD163 protein and/or a gene encoding a SIGELC1 protein.

The populations are resistant to infection by a pathogen.

The pathogen can comprise a virus. For example, the pathogen can comprise a Coronaviridae family virus, e.g., a Coronavirinae subfamily virus.

The virus preferably comprises a coronavirus (e.g., an Alphacoronavirus genus virus).

Where the virus comprises an Alphacoronavirus genus virus, the Alphacoronavirus genus virus preferably comprises a transmissible gastroenteritis virus (TGEV).

For example, the transmissible gastroenteritis virus can comprise TGEV Purdue strain.

Alternatively or in addition, the virus can comprise a porcine respiratory coronavirus (PRCV).

Where the animals in the population also comprise a modified chromosomal sequence in a gene encoding a CD163 protein, the population will also be resistant to infection by a porcine reproductive and respiratory syndrome virus (PRRSV) (e.g., Type 1 PRRSV viruses, Type 2 PRRSV viruses, or both Type 1 and Type 2 PRRSV viruses, and/or a PRRSV isolate selected from the group consisting of NVSL 97-7895, KS06-72109, P129, VR2332, C090, AZ25, MLV-ResPRRS, KS62-06274, KS483 (SD23983), C084, SD13-15, Lelystad, 03-1059, 03-1060, SD01-08, 4353PZ, and combinations of any thereof).

### Nucleic Acids

Nucleic acid molecules are also provided herein.

A nucleic acid molecule is provided. The nucleic acid molecule comprises a nucleotide sequence selected from the group consisting of:

(a) a nucleotide sequence having at least 80% sequence identity to the sequence of SEQ ID NO: 135, wherein the nucleotide sequence comprises at least one substitution, insertion, or deletion relative to SEQ ID NO: 135;

(b) a nucleotide sequence having at least 80% sequence identity to the sequence of SEQ ID NO: 132, wherein the nucleotide sequence comprises at least one substitution, insertion, or deletion relative to SEQ ID NO: 132; and

(c) a cDNA of (a) or (b).

Any of the nucleic acid molecules can be an isolated nucleic acid molecule.

The nucleic acid molecule can comprise a nucleotide sequence having at least 80% identity to SEQ ID NO: 132, wherein the nucleotide sequence comprises at least one substitution, insertion, or deletion relative to SEQ ID NO: 132.

The nucleic acid molecule can comprise a nucleotide sequence having at least 85% identity to SEQ ID NO: 132, wherein the nucleotide sequence comprises at least one substitution, insertion, or deletion relative to SEQ ID NO: 132.

The nucleic acid molecule can comprise a nucleotide sequence having at least 87.5% identity to SEQ ID NO: 132, wherein the nucleotide sequence comprises at least one substitution, insertion, or deletion relative to SEQ ID NO: 132.

The nucleic acid molecule can comprise a nucleotide sequence having at least 90% identity to SEQ ID NO: 132, wherein the nucleotide sequence comprises at least one substitution, insertion, or deletion relative to SEQ ID NO: 132.

The nucleic acid molecule can comprise a nucleotide sequence having at least 95% identity to SEQ ID NO: 132, wherein the nucleotide sequence comprises at least one substitution, insertion, or deletion relative to SEQ ID NO: 132.

The nucleic acid molecule can comprise a nucleotide sequence having at least 98% identity to SEQ ID NO: 132, wherein the nucleotide sequence comprises at least one substitution, insertion, or deletion relative to SEQ ID NO: 132.

The nucleic acid molecule can comprise a nucleotide sequence having at least 99% identity to SEQ ID NO: 132, wherein the nucleotide sequence comprises at least one substitution, insertion, or deletion relative to SEQ ID NO: 132.

The nucleic acid molecule can comprise a nucleotide sequence having at least 99.9% identity to SEQ ID NO: 132, wherein the nucleotide sequence comprises at least one substitution, insertion, or deletion relative to SEQ ID NO: 132.

The nucleic acid molecule can comprise a nucleotide sequence having at least 80% identity to SEQ ID NO: 135, wherein the nucleotide sequence comprises at least one substitution, insertion, or deletion relative to SEQ ID NO: 135.

The nucleic acid molecule can comprise a nucleotide sequence having at least 85% identity to SEQ ID NO: 135, wherein the nucleotide sequence comprises at least one substitution, insertion, or deletion relative to SEQ ID NO: 135.

The nucleic acid molecule can comprise a nucleotide sequence having at least 87.5% identity to SEQ ID NO: 135, wherein the nucleotide sequence comprises at least one substitution, insertion, or deletion relative to SEQ ID NO: 135.

The nucleic acid molecule can comprise a nucleotide sequence having at least 90% identity to SEQ ID NO: 135, wherein the nucleotide sequence comprises at least one substitution, insertion, or deletion relative to SEQ ID NO: 135.

The nucleic acid molecule can comprise a nucleotide sequence having at least 95% identity to SEQ ID NO: 135, wherein the nucleotide sequence comprises at least one substitution, insertion, or deletion relative to SEQ ID NO: 135.

The nucleic acid molecule can comprise a nucleotide sequence having at least 98% identity to SEQ ID NO: 135, wherein the nucleotide sequence comprises at least one substitution, insertion, or deletion relative to SEQ ID NO: 135.

The nucleic acid molecule can comprise a nucleotide sequence having at least 99% identity to SEQ ID NO: 135, wherein the nucleotide sequence comprises at least one substitution, insertion, or deletion relative to SEQ ID NO: 135.

The nucleic acid molecule can comprise a nucleotide sequence having at least 99.9% identity to SEQ ID NO: 135, wherein the nucleotide sequence comprises at least one substitution, insertion, or deletion relative to SEQ ID NO: 135.

The substitution, insertion, or deletion reduces or eliminates ANPEP protein production or activity, as compared to a nucleic acid that does not comprise the substitution, insertion, or deletion.

The nucleic acid molecule can comprise SEQ ID NO. 163, 164, 165, 166, 167, 168, 170, 171, 172, 173, 174, 176, 177, or 178.

For example, the nucleic acid molecule can comprise SEQ ID NO: 177, 178, 166, 167, or 171.

Affinity Tags

An “affinity tag” can be either a peptide affinity tag or a nucleic acid affinity tag. The term “affinity tag” generally refers to a protein or nucleic acid sequence that can be bound to a molecule (e.g., bound by a small molecule, protein, or covalent bond). An affinity tag can be a non-native sequence. A peptide affinity tag can comprise a peptide. A peptide affinity tag can be one that is able to be part of a split system (e.g., two inactive peptide fragments can combine together in trans to form an active affinity tag). A nucleic acid affinity tag can comprise a nucleic acid. A nucleic acid affinity tag can be a sequence that can selectively bind to a known nucleic acid sequence (e.g. through hybridization). A nucleic acid affinity tag can be a sequence that can selectively bind to a protein. An affinity tag can be fused to a native protein. An affinity tag can be fused to a nucleotide sequence.

Sometimes, one, two, or a plurality of affinity tags can be fused to a native protein or nucleotide sequence. An affinity tag can be introduced into a nucleic acid-targeting nucleic acid using methods of in vitro or in vivo transcription. Nucleic acid affinity tags can include, for example, a chemical tag, an RNA-binding protein binding sequence, a DNA-binding protein binding sequence, a sequence hybridizable to an affinity-tagged polynucleotide, a synthetic RNA aptamer, or a synthetic DNA aptamer. Examples of chemical nucleic acid affinity tags can include, but are not limited to, ribo-nucleotriphosphates containing biotin, fluorescent dyes, and digoxeginin. Examples of protein-binding nucleic acid affinity tags can include, but are not limited to, the MS2 binding sequence, the U1A binding sequence, stem-loop binding protein sequences, the boxB sequence, the eIF4A sequence, or any sequence recognized by an RNA binding protein. Examples of nucleic acid affinity-tagged oligonucleotides can include, but are not limited to, biotinylated oligonucleotides, 2, 4-dinitrophenyl oligonucleotides, fluorescein oligonucleotides, and primary amine-conjugated oligonucleotides.

A nucleic acid affinity tag can be an RNA aptamer. Aptamers can include, aptamers that bind to theophylline, streptavidin, dextran B512, adenosine, guanosine, guanine/xanthine, 7-methyl-GTP, amino acid aptamers such as aptamers that bind to arginine, citrulline, valine, tryptophan, cyanocobalamine, N-methylmesoporphyrin IX, flavin, NAD, and antibiotic aptamers such as aptamers that bind to tobramycin, neomycin, lividomycin, kanamycin, streptomycin, viomycin, and chloramphenicol.

A nucleic acid affinity tag can comprise an RNA sequence that can be bound by a site-directed polypeptide. The site-directed polypeptide can be conditionally enzymatically inactive. The RNA sequence can comprise a sequence that can be bound by a member of Type I, Type II, and/or Type III CRISPR systems. The RNA sequence can be bound by a RAMP family member protein. The RNA sequence can be bound by a Cas9 family member protein, a Cas6 family member protein (e.g., Csy4, Cas6). The RNA sequence can be bound by a Cas5 family member protein (e.g., Cas5). For example, Csy4 can bind to a specific RNA hairpin sequence with high affinity (Kd ˜50 pM) and can cleave RNA at a site 3′ to the hairpin.

A nucleic acid affinity tag can comprise a DNA sequence that can be bound by a site-directed polypeptide. The site-directed polypeptide can be conditionally enzymatically inactive. The DNA sequence can comprise a sequence that can be bound by a member of the Type I, Type II, and/or Type III CRISPR systems. The DNA sequence can be bound by an Argonaut protein. The DNA sequence can be bound by a protein containing a zinc finger domain, a TALE domain, or any other DNA-binding domain.

A nucleic acid affinity tag can comprise a ribozyme sequence. Suitable ribozymes can include peptidyl transferase 23 SrRNA, RnaseP, Group I introns, Group II introns, GIR1 branching ribozyme, Leadzyme, hairpin ribozymes, hammerhead ribozymes, HDV ribozymes, CPEB3 ribozymes, VS ribozymes, glmS ribozyme, CoTC ribozyme, and synthetic ribozymes.

Peptide affinity tags can comprise tags that can be used for tracking or purification (e.g., a fluorescent protein such as green fluorescent protein (GFP), YFP, RFP, CFP, mCherry, tdTomato; a His tag, (e.g., a 6XHis tag); a hemagglutinin (HA) tag; a FLAG tag; a Myc tag; a GST tag; a MBP tag; a chitin binding protein tag; a calmodulin tag; a V5 tag; a streptavidin binding tag; and the like).

Both nucleic acid and peptide affinity tags can comprise small molecule tags such as biotin, or digitoxin, and fluorescent label tags, such as for example, fluoroscein, rhodamin, Alexa fluor dyes, Cyanine3 dye, Cyanine5 dye.

Nucleic acid affinity tags can be located 5′ to a nucleic acid (e.g., a nucleic acid-targeting nucleic acid). Nucleic acid affinity tags can be located 3′ to a nucleic acid. Nucleic acid affinity tags can be located 5′ and 3′ to a nucleic acid. Nucleic acid affinity tags can be located within a nucleic acid. Peptide affinity tags can be located N-terminal to a polypeptide sequence. Peptide affinity tags can be located C-terminal to a polypeptide sequence. Peptide affinity tags can be located N-terminal and C-terminal to a polypeptide sequence. A plurality of affinity tags can be fused to a nucleic acid and/or a polypeptide sequence.

### Capture Agents

As used herein, “capture agent” can generally refer to an agent that can purify a polypeptide and/or a nucleic acid. A capture agent can be a biologically active molecule or material (e.g. any biological substance found in nature or synthetic, and includes but is not limited to cells, viruses, subcellular particles, proteins, including more specifically antibodies, immunoglobulins, antigens, lipoproteins, glycoproteins, peptides, polypeptides, protein complexes, (strept)avidin-biotin complexes, ligands, receptors, or small molecules, aptamers, nucleic acids, DNA, RNA, peptidic nucleic acids, oligosaccharides, polysaccharides, lipopolysccharides, cellular metabolites, haptens, pharmacologically active substances, alkaloids, steroids, vitamins, amino acids, and sugars). In some embodiments, the capture agent can comprise an affinity tag. In some embodiments, a capture agent can preferentially bind to a target polypeptide or nucleic acid of interest. Capture agents can be free floating in a mixture. Capture agents can be bound to a particle (e.g. a bead, a microbead, a nanoparticle). Capture agents can be bound to a solid or semisolid surface. In some instances, capture agents are irreversibly bound to a target. In other instances, capture agents are reversibly bound to a target (e.g., if a target can be eluted, or by use of a chemical such as imidazole).

### Targeted Integration of a Nucleic Acid at a CD163 Locus

Site-specific integration of an exogenous nucleic acid at an ANPEP, CD163, or SIGLEC1 locus may be accomplished by any technique known to those of skill in the art. For example, integration of an exogenous nucleic acid at an ANPEP, CD163, or SIGLEC1 locus can comprise contacting a cell (e.g., an isolated cell or a cell in a tissue or organism) with a nucleic acid molecule comprising the exogenous nucleic acid. Such a nucleic acid molecule can comprise nucleotide sequences flanking the exogenous nucleic acid that facilitate homologous recombination between the nucleic acid molecule and at least one ANPEP, CD163, or SIGLEC1 locus. The nucleotide sequences flanking the exogenous nucleic acid that facilitate homologous recombination can be complementary to endogenous nucleotides of the ANPEP, CD163, or SIGLEC1 locus. Alternatively, the nucleotide sequences flanking the exogenous nucleic acid that facilitate homologous recombination can be complementary to previously integrated exogenous nucleotides. A plurality of exogenous nucleic acids can be integrated at one ANPEP, CD163, or SIGLEC1 locus, such as in gene stacking.

Integration of a nucleic acid at an ANPEP, CD163, or SIGLEC1 locus can be facilitated (e.g., catalyzed) by endogenous cellular machinery of a host cell, such as, for example and without limitation, endogenous DNA and endogenous recombinase enzymes. Alternatively, integration of a nucleic acid at a ANPEP, CD163, or SIGLEC1 locus can be facilitated by one or more factors (e.g., polypeptides) that are provided to a host cell. For example, nuclease(s), recombinase(s), and/or ligase polypeptides may be provided (either independently or as part of a chimeric polypeptide) by contacting the polypeptides with the host cell, or by expressing the polypeptides within the host cell. Accordingly, a nucleic acid comprising a nucleotide sequence encoding at least one nuclease, recombinase, and/or ligase polypeptide may be introduced into the host cell, either concurrently or sequentially with a nucleic acid to be integrated site-specifically at an ANPEP, CD163, or SIGLEC1 locus, wherein the at least one nuclease, recombinase, and/or ligase polypeptide is expressed from the nucleotide sequence in the host cell.

DNA-Binding Polypeptides

Site-specific integration can be accomplished by using factors that are capable of recognizing and binding to particular nucleotide sequences, for example, in the genome of a host organism. For instance, many proteins comprise polypeptide domains that are capable of recognizing and binding to DNA in a site-specific manner. A DNA sequence that is recognized by a DNA-binding polypeptide may be referred to as a “target” sequence. Polypeptide domains that are capable of recognizing and binding to DNA in a site-specific manner generally fold correctly and function independently to bind DNA in a site-specific manner, even when expressed in a polypeptide other than the protein from which the domain was originally isolated. Similarly, target sequences for recognition and binding by DNA-binding polypeptides are generally able to be recognized and bound by such polypeptides, even when present in large DNA structures (e.g., a chromosome), particularly when the site where the target sequence is located is one known to be accessible to soluble cellular proteins (e.g., a gene).

While DNA-binding polypeptides identified from proteins that exist in nature typically bind to a discrete nucleotide sequence or motif (e.g., a consensus recognition sequence), methods exist and are known in the art for modifying many such DNA-binding polypeptides to recognize a different nucleotide sequence or motif. DNA-binding polypeptides include, for example and without limitation: zinc finger DNA-binding domains; leucine zippers; UPA DNA-binding domains; GAL4; TAL; LexA; Tet repressors; Lad; and steroid hormone receptors.

For example, the DNA-binding polypeptide can be a zinc finger. Individual zinc finger motifs can be designed to target and bind specifically to any of a large range of DNA sites. Canonical Cys2His2 (as well as non-canonical Cys3His) zinc finger polypeptides bind DNA by inserting an α-helix into the major groove of the target DNA double helix. Recognition of DNA by a zinc finger is modular; each finger contacts primarily three consecutive base pairs in the target, and a few key residues in the polypeptide mediate recognition. By including multiple zinc finger DNA-binding domains in a targeting endonuclease, the DNA-binding specificity of the targeting endonuclease may be further increased (and hence the specificity of any gene regulatory effects conferred thereby may also be increased). See, e.g., Urnov et al. (2005) Nature 435:646-51. Thus, one or more zinc finger DNA-binding polypeptides may be engineered and utilized such that a targeting endonuclease introduced into a host cell interacts with a DNA sequence that is unique within the genome of the host cell.

Preferably, the zinc finger protein is non-naturally occurring in that it is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

Selection of target sites: ZFPs and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; 6,200,759; WO 95/19431; WO 96/06166; WO 98/53057; WO 98/54311; WO 00/27878; WO 01/60970 WO 01/88197; WO 02/099084; WO 98/53058; WO 98/53059; WO 98/53060; WO 02/016536 and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

Where an animal or cell as described herein has been genetically edited using a zinc-finger nuclease, the animal or cell can be created using a process comprising introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with germline development using targeted zinc finger nuclease technology is rapid, precise, and highly efficient.

Alternatively, the DNA-binding polypeptide is a DNA-binding domain from GAL4. GAL4 is a modular transactivator in Saccharomyces cerevisiae, but it also operates as a transactivator in many other organisms. See, e.g., Sadowski et al. (1988) Nature 335:563-4. In this regulatory system, the expression of genes encoding enzymes of the galactose metabolic pathway in S. cerevisiae is stringently regulated by the available carbon source. Johnston (1987) Microbiol. Rev. 51:458-76. Transcriptional control of these metabolic enzymes is mediated by the interaction between the positive regulatory protein, GAL4, and a 17 bp symmetrical DNA sequence to which GAL4 specifically binds (the upstream activation sequence (UAS)).

Native GAL4 consists of 881 amino acid residues, with a molecular weight of 99 kDa. GAL4 comprises functionally autonomous domains, the combined activities of which account for activity of GAL4 in vivo. Ma and Ptashne (1987) Cell 48:847-53); Brent and Ptashne (1985) Cell 43(3 Pt 2):729-36. The N-terminal 65 amino acids of GAL4 comprise the GAL4 DNA-binding domain. Keegan et al. (1986) Science 231:699-704; Johnston (1987) Nature 328:353-5. Sequence-specific binding requires the presence of a divalent cation coordinated by six Cys residues present in the DNA binding domain. The coordinated cation-containing domain interacts with and recognizes a conserved CCG triplet at each end of the 17 bp UAS via direct contacts with the major groove of the DNA helix. Marmorstein et al. (1992) Nature 356:408-14. The DNA-binding function of the protein positions C-terminal transcriptional activating domains in the vicinity of the promoter, such that the activating domains can direct transcription.

Additional DNA-binding polypeptides that can be used include, for example and without limitation, a binding sequence from a AVRBS3-inducible gene; a consensus binding sequence from a AVRBS3-inducible gene or synthetic binding sequence engineered therefrom (e.g., UPA DNA-binding domain); TAL; LexA (see, e.g., Brent & Ptashne (1985), supra); LacR (see, e.g., Labow et al. (1990) Mol. Cell. Biol. 10:3343-56; Baim et al. (1991) Proc. Natl. Acad. Sci. USA 88(12):5072-6); a steroid hormone receptor (Elliston et al. (1990) J. Biol. Chem. 265:11517-121); the Tet repressor (U.S. Pat. No. 6,271,341) and a mutated Tet repressor that binds to a tet operator sequence in the presence, but not the absence, of tetracycline (Tc); the DNA-binding domain of NF-kappaB; and components of the regulatory system described in Wang et al. (1994) Proc. Natl. Acad. Sci. USA 91(17):8180-4, which utilizes a fusion of GAL4, a hormone receptor, and VP16.

The DNA-binding domain of one or more of the nucleases used in the methods and compositions described herein can comprise a naturally occurring or engineered (non-naturally occurring) TAL effector DNA binding domain. See, e.g., U.S. Patent Publication No. 2011/0301073.

Alternatively, the nuclease can comprise a CRISPR system. For example, the nuclease can comprise a CRISPR/Cas system.

The (CRISPR-associated) system evolved in bacteria and archaea as an adaptive immune system to defend against viral attack. Upon exposure to a virus, short segments of viral DNA are integrated into the CRISPR locus. RNA is transcribed from a portion of the CRISPR locus that includes the viral sequence. That RNA, which contains sequence complementary to the viral genome, mediates targeting of a Cas protein (e.g., Cas9 protein) to the sequence in the viral genome. The Cas protein cleaves and thereby silences the viral target. Recently, the CRISPR/Cas system has been adapted for genome editing in eukaryotic cells. The introduction of site-specific double strand breaks (DSBs) enables target sequence alteration through one of two endogenous DNA repair mechanisms—either non-homologous end-joining (NHEJ) or homology-directed repair (HDR). The CRISPR/Cas system has also been used for gene regulation including transcription repression and activation without altering the target sequence. Targeted gene regulation based on the CRISPR/Cas system can, for example, use an enzymatically inactive Cas9 (also known as a catalytically dead Cas9).

CRISPR/Cas systems include a CRISPR (clustered regularly interspaced short palindromic repeats) locus, which encodes RNA components of the system, and a Cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60). CRISPR loci in microbial hosts contain a combination of Cas genes as well as non-coding RNA elements capable of programming the specificity of the CRISPR-mediated nucleic acid cleavage.

The Type II CRISPR is one of the most well characterized systems and carries out targeted DNA double-strand break in nature in four sequential steps. First, two non-coding RNAs, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

For use of the CRISPR/Cas system to create targeted insertions and deletions, the two non-coding RNAs (crRNA and the TracrRNA) can be replaced by a single RNA referred to as a guide RNA (gRNA). Activity of the CRISPR/Cas system comprises of three steps: (i) insertion of exogenous DNA sequences into the CRISPR array to prevent future attacks, in a process called “adaptation,” (ii) expression of the relevant proteins, as well as expression and processing of the array, followed by (iii) RNA-mediated interference with the foreign nucleic acid. In the bacterial cell, several Cas proteins are involved with the natural function of the CRISPR/Cas system and serve roles in functions such as insertion of the foreign DNA etc.

The Cas protein can be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

Where an animal or cell as described herein has been genetically edited using a CRISPR system, a CRISPR/Cas9 system can be used to generate the animal or cell. To use Cas9 to edit genomic sequences, the protein can be delivered directly to a cell. Alternatively, an mRNA that encodes Cas9 can be delivered to a cell, or a gene that provides for expression of an mRNA that encodes Cas9 can be delivered to a cell. In addition, either target specific crRNA and a tracrRNA can be delivered directly to a cell or target specific gRNA(s) can be to a cell (these RNAs can alternatively be produced by a gene constructed to express these RNAs). Selection of target sites and designed of crRNA/gRNA are well known in the art. A discussion of construction and cloning of gRNAs can be found at http://www.genome-engineering.org/crispr/wp-content/uploads/2014/05/CRISPR-Reagent-Description-Rev20140509.pdf.

A DNA-binding polypeptide can specifically recognize and bind to a target nucleotide sequence comprised within a genomic nucleic acid of a host organism. Any number of discrete instances of the target nucleotide sequence may be found in the host genome in some examples. The target nucleotide sequence may be rare within the genome of the organism (e.g., fewer than about 10, about 9, about 8, about 7, about 6, about 5, about 4, about 3, about 2, or about 1 copy(ies) of the target sequence may exist in the genome). For example, the target nucleotide sequence may be located at a unique site within the genome of the organism. Target nucleotide sequences may be, for example and without limitation, randomly dispersed throughout the genome with respect to one another; located in different linkage groups in the genome; located in the same linkage group; located on different chromosomes; located on the same chromosome; located in the genome at sites that are expressed under similar conditions in the organism (e.g., under the control of the same, or substantially functionally identical, regulatory factors); and located closely to one another in the genome (e.g., target sequences may be comprised within nucleic acids integrated as concatemers at genomic loci).

### Targeting Endonucleases

A DNA-binding polypeptide that specifically recognizes and binds to a target nucleotide sequence can be comprised within a chimeric polypeptide, so as to confer specific binding to the target sequence upon the chimeric polypeptide. In examples, such a chimeric polypeptide may comprise, for example and without limitation, nuclease, recombinase, and/or ligase polypeptides, as these polypeptides are described above. Chimeric polypeptides comprising a DNA-binding polypeptide and a nuclease, recombinase, and/or ligase polypeptide may also comprise other functional polypeptide motifs and/or domains, such as for example and without limitation: a spacer sequence positioned between the functional polypeptides in the chimeric protein; a leader peptide; a peptide that targets the fusion protein to an organelle (e.g., the nucleus); polypeptides that are cleaved by a cellular enzyme; peptide tags (e.g., Myc, His, etc.); and other amino acid sequences that do not interfere with the function of the chimeric polypeptide.

Functional polypeptides (e.g., DNA-binding polypeptides and nuclease polypeptides) in a chimeric polypeptide may be operatively linked. Functional polypeptides of a chimeric polypeptide can be operatively linked by their expression from a single polynucleotide encoding at least the functional polypeptides ligated to each other in-frame, so as to create a chimeric gene encoding a chimeric protein. Alternatively, the functional polypeptides of a chimeric polypeptide can be operatively linked by other means, such as by cross-linkage of independently expressed polypeptides.

A DNA-binding polypeptide, or guide RNA that specifically recognizes and binds to a target nucleotide sequence can be comprised within a natural isolated protein (or mutant thereof), wherein the natural isolated protein or mutant thereof also comprises a nuclease polypeptide (and may also comprise a recombinase and/or ligase polypeptide). Examples of such isolated proteins include TALENs, recombinases (e.g., Cre, Hin, Tre, and FLP recombinase), RNA-guided CRISPR/Cas9, and meganucleases.

As used herein, the term “targeting endonuclease” refers to natural or engineered isolated proteins and mutants thereof that comprise a DNA-binding polypeptide or guide RNA and a nuclease polypeptide, as well as to chimeric polypeptides comprising a DNA-binding polypeptide or guide RNA and a nuclease. Any targeting endonuclease comprising a DNA-binding polypeptide or guide RNA that specifically recognizes and binds to a target nucleotide sequence comprised within an ANPEP, CD163, or SIGLEC1 locus (e.g., either because the target sequence is comprised within the native sequence at the locus, or because the target sequence has been introduced into the locus, for example, by recombination) can be used.

Some examples of suitable chimeric polypeptides include, without limitation, combinations of the following polypeptides: zinc finger DNA-binding polypeptides; a FokI nuclease polypeptide; TALE domains; leucine zippers; transcription factor DNA-binding motifs; and DNA recognition and/or cleavage domains isolated from, for example and without limitation, a TALEN, a recombinase (e.g., Cre, Hin, RecA, Tre, and FLP recombinases), RNA-guided CRISPR/Cas9, a meganuclease; and others known to those in the art. Particular examples include a chimeric protein comprising a site-specific DNA binding polypeptide and a nuclease polypeptide. Chimeric polypeptides may be engineered by methods known to those of skill in the art to alter the recognition sequence of a DNA-binding polypeptide comprised within the chimeric polypeptide, so as to target the chimeric polypeptide to a particular nucleotide sequence of interest.

The chimeric polypeptide can comprise a DNA-binding domain (e.g., zinc finger, TAL-effector domain, etc.) and a nuclease (cleavage) domain. The cleavage domain may be heterologous to the DNA-binding domain, for example a zinc finger DNA-binding domain and a cleavage domain from a nuclease or a TALEN DNA-binding domain and a cleavage domain, or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., 51 Nuclease; mung bean nuclease; pancreatic DNAse I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, the near edges of the target sites can be separated by 5-8 nucleotides or by 15-18 nucleotides. However any integral number of nucleotides, or nucleotide pairs, can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding, for example, such that one or more exogenous sequences (donors/transgenes) are integrated at or near the binding (target) sites. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, fusion proteins can comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a DNA binding domain and two Fok I cleavage half-domains can also be used.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in U.S. Patent Publication No. 2007/0134796. Additional restriction enzymes also contain separable binding and cleavage domains, and these are contemplated by the present disclosure. See, for example, Roberts et al. (2003) Nucleic Acids Res. 31:418-420.

The cleavage domain can comprise one or more engineered cleavage half-domain (also referred to as dimerization domain mutants) that minimize or prevent homodimerization, as described, for example, in U.S. Patent Publication Nos. 2005/0064474; 2006/0188987 and 2008/0131962.

Alternatively, nucleases may be assembled in vivo at the nucleic acid target site using so-called “split-enzyme” technology (see e.g. U.S. Patent Publication No. 20090068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

### Zinc Finger Nucleases

A chimeric polypeptide can comprise a custom-designed zinc finger nuclease (ZFN) that may be designed to deliver a targeted site-specific double-strand DNA break into which an exogenous nucleic acid, or donor DNA, may be integrated (see US Patent publication 2010/0257638). ZFNs are chimeric polypeptides containing a non-specific cleavage domain from a restriction endonuclease (for example, FokI) and a zinc finger DNA-binding domain polypeptide. See, e.g., Huang et al. (1996) J. Protein Chem. 15:481-9; Kim et al. (1997a) Proc. Natl. Acad. Sci. USA 94:3616-20; Kim et al. (1996) Proc. Natl. Acad. Sci. USA 93:1156-60; Kim et al. (1994) Proc Natl. Acad. Sci. USA 91:883-7; Kim et al. (1997b) Proc. Natl. Acad. Sci. USA 94:12875-9; Kim et al. (1997c) Gene 203:43-9; Kim et al. (1998) Biol. Chem. 379:489-95; Nahon and Raveh (1998) Nucleic Acids Res. 26:1233-9; Smith et al. (1999) Nucleic Acids Res. 27:674-81. The ZFNs can comprise non-canonical zinc finger DNA binding domains (see US Patent publication 2008/0182332). The FokI restriction endonuclease must dimerize via the nuclease domain in order to cleave DNA and introduce a double-strand break. Consequently, ZFNs containing a nuclease domain from such an endonuclease also require dimerization of the nuclease domain in order to cleave target DNA. Mani et al. (2005) Biochem. Biophys. Res. Commun. 334:1191-7; Smith et al. (2000) Nucleic Acids Res. 28:3361-9. Dimerization of the ZFN can be facilitated by two adjacent, oppositely oriented DNA-binding sites. Id.

A method for the site-specific integration of an exogenous nucleic acid into at least one ANPEP, CD163, or SIGLEC1 locus of a host can comprise introducing into a cell of the host a ZFN, wherein the ZFN recognizes and binds to a target nucleotide sequence, wherein the target nucleotide sequence is comprised within at least one ANPEP, CD163, or SIGLEC1 locus of the host. In certain examples, the target nucleotide sequence is not comprised within the genome of the host at any other position than the at least one ANPEP, CD163, or SIGLEC1 locus. For example, a DNA-binding polypeptide of the ZFN may be engineered to recognize and bind to a target nucleotide sequence identified within the at least one ANPEP, CD163, or SIGLEC1 locus (e.g., by sequencing the ANPEP, CD163, or SIGLEC1 locus). A method for the site-specific integration of an exogenous nucleic acid into at least one ANPEP, CD163, or SIGLEC1 performance locus of a host that comprises introducing into a cell of the host a ZFN may also comprise introducing into the cell an exogenous nucleic acid, wherein recombination of the exogenous nucleic acid into a nucleic acid of the host comprising the at least one ANPEP, CD163, or SIGLEC1 locus is facilitated by site-specific recognition and binding of the ZFN to the target sequence (and subsequent cleavage of the nucleic acid comprising the ANPEP, CD163, or SIGLEC1 locus).

### Optional Exogenous Nucleic Acids for Integration at an ANPEP, CD163, or SIGLEC1 Locus

Exogenous nucleic acids for integration at an ANPEP, CD163, or SIGLEC1 locus include: an exogenous nucleic acid for site-specific integration in at least one ANPEP, CD163, or SIGLEC1 locus, for example and without limitation, an ORF; a nucleic acid comprising a nucleotide sequence encoding a targeting endonuclease; and a vector comprising at least one of either or both of the foregoing. Thus, particular nucleic acids include nucleotide sequences encoding a polypeptide, structural nucleotide sequences, and/or DNA-binding polypeptide recognition and binding sites.

### Optional Exogenous Nucleic Acid Molecules for Site-Specific Integration

As noted above, insertion of an exogenous sequence (also called a “donor sequence” or “donor” or “transgene”) is provided, for example for expression of a polypeptide, correction of a mutant gene or for increased expression of a wild-type gene. It will be readily apparent that the donor sequence is typically not identical to the genomic sequence where it is placed. A donor sequence can contain a non-homologous sequence flanked by two regions of homology to allow for efficient homology-directed repair (HDR) at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded or double-stranded and can be introduced into a cell in linear or circular form. See e.g., U.S. Patent Publication Nos. 2010/0047805, 2011/0281361, 2011/0207221, and 2013/0326645. If introduced in linear form, the ends of the donor sequence can be protected (e.g. from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally integrated so that its expression is driven by the endogenous promoter at the integration site, namely the promoter that drives expression of the endogenous gene into which the donor is integrated (e.g., ANPEP, CD163, or SIGLEC1). However, it will be apparent that the donor may comprise a promoter and/or enhancer, for example a constitutive promoter or an inducible or tissue specific promoter.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

Exogenous nucleic acids that may be integrated in a site-specific manner into at least one ANPEP, CD163, or SIGLEC1 locus, so as to modify the ANPEP, CD163, or SIGLEC1 locus include, for example and without limitation, nucleic acids comprising a nucleotide sequence encoding a polypeptide of interest; nucleic acids comprising an agronomic gene; nucleic acids comprising a nucleotide sequence encoding an RNAi molecule; or nucleic acids that disrupt the ANPEP, CD163, or SIGLEC1 gene.

An exogenous nucleic acid can be integrated at a ANPEP, CD163, or SIGLEC1 locus, so as to modify the ANPEP, CD163, or SIGLEC1 locus, wherein the nucleic acid comprises a nucleotide sequence encoding a polypeptide of interest, such that the nucleotide sequence is expressed in the host from the ANPEP, CD163, or SIGLEC1 locus. In some examples, the polypeptide of interest (e.g., a foreign protein) is expressed from a nucleotide sequence encoding the polypeptide of interest in commercial quantities. In such examples, the polypeptide of interest may be extracted from the host cell, tissue, or biomass.

Nucleic Acid Molecules Comprising a Nucleotide Sequence Encoding a Targeting Endonuclease

A nucleotide sequence encoding a targeting endonuclease can be engineered by manipulation (e.g., ligation) of native nucleotide sequences encoding polypeptides comprised within the targeting endonuclease. For example, the nucleotide sequence of a gene encoding a protein comprising a DNA-binding polypeptide may be inspected to identify the nucleotide sequence of the gene that corresponds to the DNA-binding polypeptide, and that nucleotide sequence may be used as an element of a nucleotide sequence encoding a targeting endonuclease comprising the DNA-binding polypeptide. Alternatively, the amino acid sequence of a targeting endonuclease may be used to deduce a nucleotide sequence encoding the targeting endonuclease, for example, according to the degeneracy of the genetic code.

In exemplary nucleic acid molecules comprising a nucleotide sequence encoding a targeting endonuclease, the last codon of a first polynucleotide sequence encoding a nuclease polypeptide, and the first codon of a second polynucleotide sequence encoding a DNA-binding polypeptide, may be separated by any number of nucleotide triplets, e.g., without coding for an intron or a “STOP.” Likewise, the last codon of a nucleotide sequence encoding a first polynucleotide sequence encoding a DNA-binding polypeptide, and the first codon of a second polynucleotide sequence encoding a nuclease polypeptide, may be separated by any number of nucleotide triplets. The last codon (i.e., most 3′ in the nucleic acid sequence) of a first polynucleotide sequence encoding a nuclease polypeptide, and a second polynucleotide sequence encoding a DNA-binding polypeptide, can be fused in phase-register with the first codon of a further polynucleotide coding sequence directly contiguous thereto, or separated therefrom by no more than a short peptide sequence, such as that encoded by a synthetic nucleotide linker (e.g., a nucleotide linker that may have been used to achieve the fusion). Examples of such further polynucleotide sequences include, for example and without limitation, tags, targeting peptides, and enzymatic cleavage sites. Likewise, the first codon of the most 5′ (in the nucleic acid sequence) of the first and second polynucleotide sequences may be fused in phase-register with the last codon of a further polynucleotide coding sequence directly contiguous thereto, or separated therefrom by no more than a short peptide sequence.

A sequence separating polynucleotide sequences encoding functional polypeptides in a targeting endonuclease (e.g., a DNA-binding polypeptide and a nuclease polypeptide) may, for example, consist of any sequence, such that the amino acid sequence encoded is not likely to significantly alter the translation of the targeting endonuclease. Due to the autonomous nature of known nuclease polypeptides and known DNA-binding polypeptides, intervening sequences will not interfere with the respective functions of these structures.

### Other Knockout Methods

Various other techniques known in the art can be used to inactivate genes to make knock-out animals and/or to introduce nucleic acid constructs into animals to produce founder animals and to make animal lines, in which the knockout or nucleic acid construct is integrated into the genome. Such techniques include, without limitation, pronuclear microinjection (U.S. Pat. No. 4,873,191), retrovirus mediated gene transfer into germ lines (Van der Putten et al. (1985) Proc. Natl. Acad. Sci. USA 82, 6148-1652), gene targeting into embryonic stem cells (Thompson et al. (1989) Cell 56, 313-321), electroporation of embryos (Lo (1983) Mol. Cell. Biol. 3, 1803-1814), sperm-mediated gene transfer (Lavitrano et al. (2002) Proc. Natl. Acad. Sci. USA 99, 14230-14235; Lavitrano et al. (2006) Reprod. Fert. Develop. 18, 19-23), and in vitro transformation of somatic cells, such as cumulus or mammary cells, or adult, fetal, or embryonic stem cells, followed by nuclear transplantation (Wilmut et al. (1997) Nature 385, 810-813; and Wakayama et al. (1998) Nature 394, 369-374). Pronuclear microinjection, sperm mediated gene transfer, and somatic cell nuclear transfer are particularly useful techniques. An animal that is genomically modified is an animal wherein all of its cells have the modification, including its germ line cells. When methods are used that produce an animal that is mosaic in its modification, the animals may be inbred and progeny that are genomically modified may be selected. Cloning, for instance, may be used to make a mosaic animal if its cells are modified at the blastocyst state, or genomic modification can take place when a single-cell is modified. Animals that are modified so they do not sexually mature can be homozygous or heterozygous for the modification, depending on the specific approach that is used. If a particular gene is inactivated by a knock out modification, homozygosity would normally be required. If a particular gene is inactivated by an RNA interference or dominant negative strategy, then heterozygosity is often adequate.

Typically, in embryo/zygote microinjection, a nucleic acid construct or mRNA is introduced into a fertilized egg; one or two cell fertilized eggs are used as the nuclear structure containing the genetic material from the sperm head and the egg are visible within the protoplasm. Pronuclear staged fertilized eggs can be obtained in vitro or in vivo (i.e., surgically recovered from the oviduct of donor animals). In vitro fertilized eggs can be produced as follows. For example, swine ovaries can be collected at an abattoir, and maintained at 22-28° C. during transport. Ovaries can be washed and isolated for follicular aspiration, and follicles ranging from 4-8 mm can be aspirated into 50 mL conical centrifuge tubes using 18 gauge needles and under vacuum. Follicular fluid and aspirated oocytes can be rinsed through pre-filters with commercial TL-HEPES (Minitube, Verona, Wis.). Oocytes surrounded by a compact cumulus mass can be selected and placed into TCM-199 OOCYTE MATURATION MEDIUM (Minitube, Verona, Wis.) supplemented with 0.1 mg/mL cysteine, 10 ng/mL epidermal growth factor, 10% porcine follicular fluid, 50 μM 2-mercaptoethanol, 0.5 mg/ml cAMP, 10 IU/mL each of pregnant mare serum gonadotropin (PMSG) and human chorionic gonadotropin (hCG) for approximately 22 hours in humidified air at 38.7° C. and 5% CO2. Subsequently, the oocytes can be moved to fresh TCM-199 maturation medium, which will not contain cAMP, PMSG or hCG and incubated for an additional 22 hours. Matured oocytes can be stripped of their cumulus cells by vortexing in 0.1% hyaluronidase for 1 minute.

For swine, mature oocytes can be fertilized in 500 μl Minitube PORCPRO IVF MEDIUM SYSTEM (Minitube, Verona, Wis.) in Minitube 5-well fertilization dishes. In preparation for in vitro fertilization (IVF), freshly-collected or frozen boar semen can be washed and resuspended in PORCPRO IVF Medium to 400,000 sperm. Sperm concentrations can be analyzed by computer assisted semen analysis (SPERMVISION, Minitube, Verona, Wis.). Final in vitro insemination can be performed in a 10 μl volume at a final concentration of approximately 40 motile sperm/oocyte, depending on boar. All fertilizing oocytes can be incubated at 38.7° C. in 5.0% CO2 atmosphere for six hours. Six hours post-insemination, presumptive zygotes can be washed twice in NCSU-23 and moved to 0.5 mL of the same medium. This system can produce 20-30% blastocysts routinely across most boars with a 10-30% polyspermic insemination rate.

Linearized nucleic acid constructs or mRNA can be injected into one of the pronuclei or into the cytoplasm. Then the injected eggs can be transferred to a recipient female (e.g., into the oviducts of a recipient female) and allowed to develop in the recipient female to produce the transgenic or gene edited animals. In particular, in vitro fertilized embryos can be centrifuged at 15,000×g for 5 minutes to sediment lipids allowing visualization of the pronucleus. The embryos can be injected with using an Eppendorf FEMTOJET injector and can be cultured until blastocyst formation. Rates of embryo cleavage and blastocyst formation and quality can be recorded.

Embryos can be surgically transferred into uteri of asynchronous recipients. Typically, 100-200 (e.g., 150-200) embryos can be deposited into the ampulla-isthmus junction of the oviduct using a 5.5-inch TOMCAT® catheter. After surgery, real-time ultrasound examination of pregnancy can be performed.

In somatic cell nuclear transfer, a transgenic or gene edited cell such as an embryonic blastomere, fetal fibroblast, adult ear fibroblast, or granulosa cell that includes a nucleic acid construct described above, can be introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the transgenic or gene edited cell into an enucleated oocyte arrested at meiosis 2. In some conventions, oocytes arrested at meiosis-2 are termed eggs. After producing a porcine or bovine embryo (e.g., by fusing and activating the oocyte), the embryo is transferred to the oviducts of a recipient female, about 20 to 24 hours after activation. See, for example, Cibelli et al. (1998) Science 280, 1256-1258 and U.S. Pat. Nos. 6,548,741, 7,547,816, 7,989,657, or 6,211,429. For pigs, recipient females can be checked for pregnancy approximately 20-21 days after transfer of the embryos.

Standard breeding techniques can be used to create animals that are homozygous for the inactivated gene from the initial heterozygous founder animals. Homozygosity may not be required, however. Gene edited pigs described herein can be bred with other pigs of interest.

Once gene edited animals have been generated, inactivation of an endogenous nucleic acid can be assessed using standard techniques. Initial screening can be accomplished by Southern blot analysis to determine whether or not inactivation has taken place. For a description of Southern analysis, see sections 9.37-9.52 of Sambrook et al., 1989, Molecular Cloning, A Laboratory Manual, second edition, Cold Spring Harbor Press, Plainview; N.Y. Polymerase chain reaction (PCR) techniques also can be used in the initial screening PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. PCR is described in, for example PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis (1992) Genetic Engineering News 12,1; Guatelli et al. (1990) Proc. Natl. Acad. Sci. USA 87:1874; and Weiss (1991) Science 254:1292. At the blastocyst stage, embryos can be individually processed for analysis by PCR, Southern hybridization and splinkerette PCR (see, e.g., Dupuy et al. Proc Natl Acad Sci USA (2002) 99:4495).

### Interfering RNAs

A variety of interfering RNA (RNAi) systems are known. Double-stranded RNA (dsRNA) induces sequence-specific degradation of homologous gene transcripts. RNA-induced silencing complex (RISC) metabolizes dsRNA to small 21-23-nucleotide small interfering RNAs (siRNAs). RISC contains a double stranded RNAse (dsRNAse, e.g., Dicer) and ssRNAse (e.g., Argonaut 2 or Ago2). RISC utilizes antisense strand as a guide to find a cleavable target. Both siRNAs and microRNAs (miRNAs) are known. A method of inactivating a gene in a genetically edited animal comprises inducing RNA interference against a target gene and/or nucleic acid such that expression of the target gene and/or nucleic acid is reduced.

For example the exogenous nucleic acid sequence can induce RNA interference against a nucleic acid encoding a polypeptide. For example, double-stranded small interfering RNA (siRNA) or small hairpin RNA (shRNA) homologous to a target DNA can be used to reduce expression of that DNA. Constructs for siRNA can be produced as described, for example, in Fire et al. (1998) Nature 391:806; Romano and Masino (1992) Mol. Microbiol. 6:3343; Cogoni et al. (1996) EMBO J. 15:3153; Cogoni and Masino (1999) Nature 399:166; Misquitta and Paterson (1999) Proc. Natl. Acad. Sci. USA 96:1451; and Kennerdell and Carthew (1998) Cell 95:1017. Constructs for shRNA can be produced as described by McIntyre and Fanning (2006) BMC Biotechnology 6:1. In general, shRNAs are transcribed as a single-stranded RNA molecule containing complementary regions, which can anneal and form short hairpins.

The probability of finding a single, individual functional siRNA or miRNA directed to a specific gene is high. The predictability of a specific sequence of siRNA, for instance, is about 50% but a number of interfering RNAs may be made with good confidence that at least one of them will be effective.

In vitro cells, in vivo cells, or a genetically edited animal such as a livestock animal that express an RNAi directed against a gene encoding ANPEP, CD163, or SIGLEC1 can be used. The RNAi may be, for instance, selected from the group consisting of siRNA, shRNA, dsRNA, RISC and miRNA.

### Inducible Systems

An inducible system may be used to inactivate a ANPEP, CD163, or SIGLEC1 gene. Various inducible systems are known that allow spatial and temporal control of inactivation of a gene. Several have been proven to be functional in vivo in porcine animals.

An example of an inducible system is the tetracycline (tet)-on promoter system, which can be used to regulate transcription of the nucleic acid. In this system, a mutated Tet repressor (TetR) is fused to the activation domain of herpes simplex virus VP 16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

The tetracycline-inducible system and the Cre/loxP recombinase system (either constitutive or inducible) are among the more commonly used inducible systems. The tetracycline-inducible system involves a tetracycline-controlled transactivator (tTA)/reverse tTA (rtTA). A method to use these systems in vivo involves generating two lines of genetically edited animals. One animal line expresses the activator (tTA, rtTA, or Cre recombinase) under the control of a selected promoter. Another line of animals expresses the acceptor, in which the expression of the gene of interest (or the gene to be altered) is under the control of the target sequence for the tTA/rtTA transactivators (or is flanked by loxP sequences). Mating the two of animals provides control of gene expression.

The tetracycline-dependent regulatory systems (tet systems) rely on two components, i.e., a tetracycline-controlled transactivator (tTA or rtTA) and a tTA/rtTA-dependent promoter that controls expression of a downstream cDNA, in a tetracycline-dependent manner. In the absence of tetracycline or its derivatives (such as doxycycline), tTA binds to tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. Administration of tetracycline or its derivatives allows temporal control of transgene expression in vivo. rtTA is a variant of tTA that is not functional in the absence of doxycycline but requires the presence of the ligand for transactivation. This tet system is therefore termed tet-ON. The tet systems have been used in vivo for the inducible expression of several transgenes, encoding, e.g., reporter genes, oncogenes, or proteins involved in a signaling cascade.

The Cre/lox system uses the Cre recombinase, which catalyzes site-specific recombination by crossover between two distant Cre recognition sequences, i.e., loxP sites. A DNA sequence introduced between the two loxP sequences (termed floxed DNA) is excised by Cre-mediated recombination. Control of Cre expression in a transgenic and/or gene edited animal, using either spatial control (with a tissue- or cell-specific promoter), or temporal control (with an inducible system), results in control of DNA excision between the two loxP sites. One application is for conditional gene inactivation (conditional knockout). Another approach is for protein over-expression, wherein a floxed stop codon is inserted between the promoter sequence and the DNA of interest. Genetically edited animals do not express the transgene until Cre is expressed, leading to excision of the floxed stop codon. This system has been applied to tissue-specific oncogenesis and controlled antigene receptor expression in B lymphocytes. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein.

In vitro cells, in vivo cells, or a genetically edited animal such as a livestock animal that comprises a ANPEP, CD163, or SIGLEC1 gene under control of an inducible system can be used. The chromosomal modification of an animal may be genomic or mosaic. The inducible system may be, for instance, selected from the group consisting of Tet-On, Tet-Off, Cre-lox, and Hif1 alpha.

### Vectors and Nucleic Acids

A variety of nucleic acids may be introduced into cells for knockout purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, Summerton and Weller (1997) Antisense Nucleic Acid Drug Dev. 7(3):187; and Hyrup et al. (1996) Bioorgan. Med. Chem. 4:5. In addition, the deoxyphosphate backbone can be replaced with, for example, a phosphorothioate or phosphorodithioate backbone, a phosphoroamidite, or an alkyl phosphotriester backbone.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

Any type of promoter can be operably linked to a target nucleic acid sequence. Examples of promoters include, without limitation, tissue-specific promoters, constitutive promoters, inducible promoters, and promoters responsive or unresponsive to a particular stimulus. Suitable tissue specific promoters can result in preferential expression of a nucleic acid transcript in beta cells and include, for example, the human insulin promoter. Other tissue specific promoters can result in preferential expression in, for example, hepatocytes or heart tissue and can include the albumin or alpha-myosin heavy chain promoters, respectively. A promoter that facilitates the expression of a nucleic acid molecule without significant tissue or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. For example, a fusion of the chicken beta actin gene promoter and the CMV enhancer can be used as a promoter. See, for example, Xu et al. (2001) Hum. Gene Ther. 12:563; and Kiwaki et al. (1996) Hum. Gene Ther. 7:821.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

A sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban, et al., Proc. Natl. Acad. Sci. (1992) 89:6861, for a review of Cre/lox technology, and Brand and Dymecki, Dev. Cell (2004) 6:7. A transposon containing a Cre- or Flp-activatable transgene interrupted by a selectable marker gene also can be used to obtain animals with conditional expression of a transgene. For example, a promoter driving expression of the marker/transgene can be either ubiquitous or tissue-specific, which would result in the ubiquitous or tissue-specific expression of the marker in F0 animals (e.g., pigs). Tissue specific activation of the transgene can be accomplished, for example, by crossing a pig that ubiquitously expresses a marker-interrupted transgene to a pig expressing Cre or Flp in a tissue-specific manner, or by crossing a pig that expresses a marker-interrupted transgene in a tissue-specific manner to a pig that ubiquitously expresses Cre or Flp recombinase. Controlled expression of the transgene or controlled excision of the marker allows expression of the transgene.

The exogenous nucleic acid can encode a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG™tag (Kodak, New Haven, Conn.).

Nucleic acid constructs can be methylated using an SssI CpG methylase (New England Biolabs, Ipswich, Mass.). In general, the nucleic acid construct can be incubated with S-adenosylmethionine and SssI CpG-methylase in buffer at 37° C. Hypermethylation can be confirmed by incubating the construct with one unit of HinPlI endonuclease for 1 hour at 37° C. and assaying by agarose gel electrophoresis.

Nucleic acid constructs can be introduced into embryonic, fetal, or adult animal cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In transposon systems, the transcriptional unit of a nucleic acid construct, i.e., the regulatory region operably linked to an exogenous nucleic acid sequence, is flanked by an inverted repeat of a transposon. Several transposon systems, including, for example, Sleeping Beauty (see, U.S. Pat. No. 6,613,752 and U.S. Publication No. 2005/0003542); Frog Prince (Miskey et al. (2003) Nucleic Acids Res. 31:6873); Tol2 (Kawakami (2007) Genome Biology 8(Suppl.1):S7; Minos (Pavlopoulos et al. (2007) Genome Biology 8(Suppl.1):S2); Hsmar1 (Miskey et al. (2007)) Mol Cell Biol. 27:4589); and Passport have been developed to introduce nucleic acids into cells, including mice, human, and pig cells. The Sleeping Beauty transposon is particularly useful. A transposase can be delivered as a protein, encoded on the same nucleic acid construct as the exogenous nucleic acid, can be introduced on a separate nucleic acid construct, or provided as an mRNA (e.g., an in vitro-transcribed and capped mRNA).

Insulator elements also can be included in a nucleic acid construct to maintain expression of the exogenous nucleic acid and to inhibit the unwanted transcription of host genes. See, for example, U.S. Publication No. 2004/0203158. Typically, an insulator element flanks each side of the transcriptional unit and is internal to the inverted repeat of the transposon. Non-limiting examples of insulator elements include the matrix attachment region-(MAR) type insulator elements and border-type insulator elements. See, for example, U.S. Pat. Nos. 6,395,549, 5,731,178, 6,100,448, and 5,610,053, and U.S. Publication No. 2004/0203158.

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand).

### Founder Animals, Animal Lines, Traits, and Reproduction

Founder animals may be produced by cloning and other methods described herein. The founders can be homozygous for a genetic alteration, as in the case where a zygote or a primary cell undergoes a homozygous modification. Similarly, founders can also be made that are heterozygous. In the case of the animals comprising at least one modified chromosomal sequence in a gene encoding an ANPEP protein, the founders are preferably heterozygous. The founders may be genomically modified, meaning that all of the cells in their genome have undergone modification. Founders can be mosaic for a modification, as may happen when vectors are introduced into one of a plurality of cells in an embryo, typically at a blastocyst stage. Progeny of mosaic animals may be tested to identify progeny that are genomically modified. An animal line is established when a pool of animals has been created that can be reproduced sexually or by assisted reproductive techniques, with heterogeneous or homozygous progeny consistently expressing the modification.

In livestock, many alleles are known to be linked to various traits such as production traits, type traits, workability traits, and other functional traits. Artisans are accustomed to monitoring and quantifying these traits, e.g., Visscher et al., Livestock Production Science, 40 (1994) 123-137, U.S. Pat. No. 7,709,206, US 2001/0016315, US 2011/0023140, and US 2005/0153317. An animal line may include a trait chosen from a trait in the group consisting of a production trait, a type trait, a workability trait, a fertility trait, a mothering trait, and a disease resistance trait. Further traits include expression of a recombinant gene product.

Animals with a desired trait or traits may be modified to prevent their sexual maturation. Since the animals are sterile until matured, it is possible to regulate sexual maturity as a means of controlling dissemination of the animals. Animals that have been bred or modified to have one or more traits can thus be provided to recipients with a reduced risk that the recipients will breed the animals and appropriate the value of the traits to themselves. For example, the genome of an animal can be modified, wherein the modification comprises inactivation of a sexual maturation gene, wherein the sexual maturation gene in a wild type animal expresses a factor selective for sexual maturation. The animal can be treated by administering a compound to remedy a deficiency caused by the loss of expression of the gene to induce sexual maturation in the animal.

Breeding of animals that require administration of a compound to induce sexual maturity may advantageously be accomplished at a treatment facility. The treatment facility can implement standardized protocols on well-controlled stock to efficiently produce consistent animals. The animal progeny may be distributed to a plurality of locations to be raised. Farms and farmers (a term including a ranch and ranchers) may thus order a desired number of progeny with a specified range of ages and/or weights and/or traits and have them delivered at a desired time and/or location. The recipients, e.g., farmers, may then raise the animals and deliver them to market as they desire.

A genetically edited livestock animal having an inactivated sexual maturation gene can be delivered (e.g., to one or more locations, to a plurality of farms). The animals can have an age of between about 1 day and about 180 days. The animal can have one or more traits (for example one that expresses a desired trait or a high-value trait or a novel trait or a recombinant trait).

Having described the invention in detail, it will be apparent that modifications and variations are possible without departing from the scope of the invention defined in the appended claims.

### EXAMPLES

The following non-limiting examples are provided to further illustrate the present invention.

Examples 1 to 3 describe the generation of pigs having modified chromosomal sequences in their CD163 genes, and the resistance of such pigs to PRRSV infection. Example 4 describes the generation of SIGLEC1 knockout pigs. Examples 5 and 6 describe the generation of pigs having modified chromosomal sequences in their ANPEP genes and the resistance of such pigs to TGEV. Example 7 describes the generation of pigs heterozygous for chromosomal modifications in at least two genes selected from CD163, SIGLEC1, and ANPEP. Example 8 describes how the pigs generated in Example 7 will be used to generate animals homozygous for chromosomal modifications in at least two genes selected from CD163, SIGLEC1, and ANPEP, and how such animals will be tested for resistance to TGEV and PRRSV.

### Example 1: Use of the CRISPR/Cas9 System to Produce Genetically Engineered Pigs from In Vitro-Derived Oocytes and Embryos

Recent reports describing homing endonucleases, such as zinc-finger nucleases (ZFNs), transcription activator-like effector nucleases (TALENs), and components in the clustered regularly interspaced short palindromic repeat (CRISPR)/CRISPR-associated (Cas9) system suggest that genetic engineering (GE) in pigs might now be more efficient. Targeted homing endonucleases can induce double-strand breaks (DSBs) at specific locations in the genome and cause either random mutations through nonhomologous end joining (NHEJ) or stimulation of homologous recombination (HR) if donor DNA is provided. Targeted modification of the genome through HR can be achieved with homing endonucleases if donor DNA is provided along with the targeted nuclease. After introducing specific modifications in somatic cells, these cells were used to produce GE pigs for various purposes via SCNT. Thus, homing endonucleases are a useful tool in generating GE pigs. Among the different homing endonucleases, the CRISPR/Cas9 system, adapted from prokaryotes where it is used as a defense mechanism, appears to be an effective approach. In nature, the Cas9 system requires three components, an RNA (˜20 bases) that contains a region that is complementary to the target sequence (cis-repressed RNA [crRNA]), an RNA that contains a region that is complementary to the crRNA (trans-activating crRNA [tracrRNA]), and Cas9, the enzymatic protein component in this complex. A single guide RNA (gRNA) can be constructed to serve the roles of the base-paired crRNA and tracrRNA. The gRNA/protein complex can scan the genome and catalyze a DSB at regions that are complementary to the crRNA/gRNA. Unlike other designed nucleases, only a short oligomer needs to be designed to construct the reagents required to target a gene of interest whereas a series of cloning steps are required to assemble ZFNs and TALENs.

Unlike current standard methods for gene disruption, the use of designed nucleases offers the opportunity to use zygotes as starting material for GE. Standard methods for gene disruption in livestock involve HR in cultured cells and subsequent reconstruction of embryos by somatic cell nuclear transfer (SCNT). Because cloned animals produced through SCNT sometimes show signs of developmental defects, progeny of the SCNT/GE founders are typically used for research to avoid confounding SCNT anomalies and phenotype that could occur if founder animals are used for experiments. Considering the longer gestation period and higher housing costs of pigs compared to rodents, there are time and cost benefits to the reduced need for breeding. A recent report demonstrated that direct injection of ZFNs and TALENs into porcine zygotes could disrupt an endogenous gene and produce piglets with the desired mutations. However, only about 10% of piglets showed biallelic modification of the target gene, and some presented mosaic genotypes. A recent article demonstrated that CRISPR/Cas9 system could induce mutations in developing embryos and produce GE pigs at a higher efficiency than ZFNs or TALENs. However, GE pigs produced from the CRISPR/Cas9 system also possessed mosaic genotypes. In addition, all the above-mentioned studies used in vivo derived zygotes for the experiments, which require intensive labor and numerous sows to obtain a sufficient number of zygotes.

The present example describes an efficient approach to use the CRISPR/Cas9 system in generating GE pigs via both injection of in vitro derived zygotes and modification of somatic cells followed by SCNT. Two endogenous genes (CD163 and CD1D) and one transgene (eGFP) were targeted, and only in vitro derived oocytes or zygotes were used for SCNT or RNA injections, respectively. CD163 appears to be required for productive infection by porcine reproductive and respiratory syndrome virus, a virus known to cause a significant economic loss to swine industry. CD1D is considered a nonclassical major histocompatibility complex protein and is involved in presentation of lipid antigens to invariant natural killer T cells. Pigs deficient in these genes were designed to be models for agriculture and biomedicine. The eGFP transgene was used as a target for preliminary proof-of-concept experiments and optimizations of methods.

### Materials and Methods

Chemical and Reagents. Unless otherwise stated, all of the chemicals used in this study were purchased from Sigma.

Design of gRNAs to Build Specific CRISPRs

Guide RNAs were designed to regions within exon 7 of CD163 that were unique to the wild type CD163 and not present in the domain swap targeting vector (described below), so that the CRISPR would result in DSB within wild type CD163 but not in the domain swap targeting vector. There were only four locations in which the targeting vector would introduce a single nucleotide polymorphism (SNP) that would alter an S. pyogenes (Spy) protospacer adjacent motif (PAM). All four targets were selected including:

(CRISPR 10)
(SEQ ID NO: 1)
GGAAACCCAGGCTGGTTGGAgGG,
(CRISPR 131)
(SEQ ID NO: 2)
GGAACTACAGTGCGGCACTGtGG,
(CRISPR 256)
(SEQ ID NO: 3)
CAGTAGCACCCCGCCCTGACgGG
and
(CRISPR 282)
(SEQ ID NO: 4)
TGTAGCCACAGCAGGGACGTcGG.

The PAM can be identified by the bold font in each gRNA.

For CD1D mutations, the search for CRISPR targets was arbitrarily limited to the coding strand within the first 1000 bp of the primary transcript. However, RepeatMasker [26] (“Pig” repeat library) identified a repetitive element beginning at base 943 of the primary transcript. The search for CRISPR targets was then limited to the first 942 bp of the primary transcript. The search was further limited to the first 873 bp of the primary transcript since the last Spy PAM is located at base 873. The first target (CRISPR 4800) was selected because it overlapped with the start codon located at base 42 in primary transcript (CCAGCCTCGCCCAGCGACATgGG (SEQ ID NO: 5)). Two additional targets (CRISPRs 5620 and 5626) were selected because they were the most distal to the first selection within the arbitrarily selected region (CTTTCATTTATCTGAACTCAgGG (SEQ ID NO: 6)) and TTATCTGAACTCAGGGTCCCcGG (SEQ ID NO: 7)). These targets overlap. In relation to the start codon, the most proximal Spy PAMs were located in simple sequence that contained extensively homopolymeric sequence as determined by visual appraisal. The fourth target (CRISPR 5350) was selected because, in relation to the first target selection, it was the most proximal target that did not contain extensive homopolymeric regions (CAGCTGCAGCATATATTTAAgGG (SEQ ID NO: 8)). Specificity of the designed crRNAs was confirmed by searching for similar porcine sequences in GenBank. The oligonucleotides (Table 2) were annealed and cloned into the p330X vector which contains two expression cassettes, a human codon-optimized S. pyogenes (hSpy) Cas9 and the chimeric guide RNA. P330X was digested with Bbs1 (New England Biolabs) following the Zhang laboratory protocol (http://www.addgene.org/crispezhang/).

To target eGFP, two specific gRNAs targeting the eGFP coding sequence were designed within the first 60 bp of the eGFP start codon. Both eGFP1 and eGFP2 gRNA were on the antisense strand and eGFP1 directly targeted the start codon. The eGFP1 gRNA sequence was CTCCTCGCCCTTGCTCACCAtGG (SEQ ID NO: 9) and the eGFP2 gRNA sequence was GACCAGGATGGGCACCACCCcGG (SEQ ID NO: 10).

TABLE 2
Designed crRNAs. Primer 1 and primer 2 were
annealed following the Zhang protocol.

| | | SEQ ID |
|---|---|---|
| Primer | Sequence (5′-3′) | NO. |
| CD163 10 1 | CACCGGAAACCCAGGCTGGTTGGA | 48 |
| CD163 10 2 | AAACTCCAACCAGCCTGGGTTTCC | 49 |
| CD163 131 1 | CACCGGAACTACAGTGCGGCACTG | 50 |
| CD163 131 2 | AAACCAGTGCCGCACTGTAGTTCC | 51 |
| CD163 256 1 | CACCGCAGTAGCACCCCGCCCTGAC | 52 |
| CD163 256 2 | AAACGTCAGGGCGGGGTGCTACTGC | 53 |
| CD163 282 1 | CACCGTGTAGCCACAGCAGGGACGT | 54 |
| CD163 282 2 | AAACACGTCCCTGCTGTGGCTACAC | 55 |
| CD1D 4800 1 | CACCGCCAGCCTCGCCCAGCGACAT | 56 |
| CD1D 4800 2 | AAACATGTCGCTGGGCGAGGCTGGC | 57 |
| CD1D 5350 1 | CACCGCAGCTGCAGCATATATTTAA | 58 |
| CD1D 5350 2 | AAACTTAAATATATGCTGCAGCTGC | 59 |
| CD1D 5620 1 | CACCGCTTTCATTTATCTGAACTCA | 60 |
| CD1D 5620 2 | AAACTGAGTTCAGATAAATGAAAGC | 61 |
| CD1D 5626 1 | CACCGTTATCTGAACTCAGGGTCCC | 62 |
| CD1D 5626 2 | AAACGGGACCCTGAGTTCAGATAAC | 63 |
| eGFP 1 1 | CACCGCTCCTCGCCCTTGCTCACCA | 64 |
| eGFP 1 2 | AAACTGGTGAGCAAGGGCGAGGAGC | 65 |
| eGFP 2 1 | CACCGGACCAGGATGGGCACCACCC | 66 |
| eGFP 2 2 | AAACGGGTGGTGCCCATCCTGGTCC | 67 |

### Synthesis of Donor DNA for CD163 and CD1D Genes

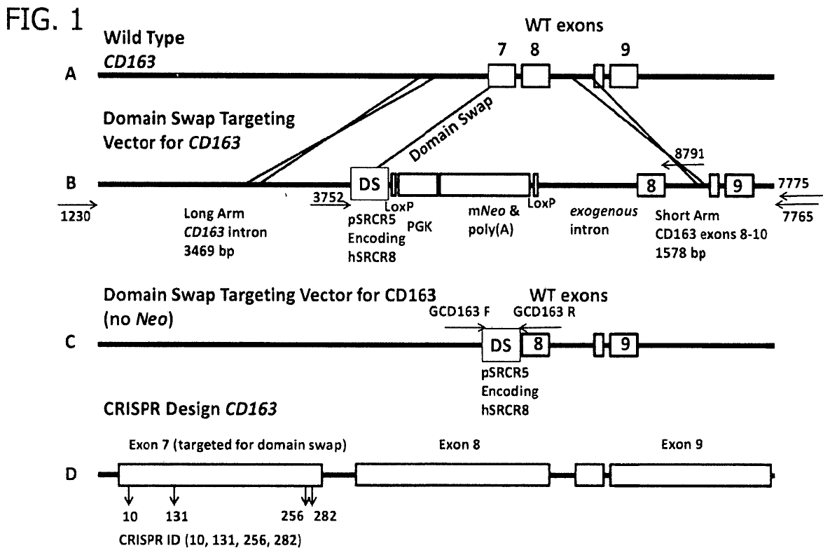

Both porcine CD163 and CD1D were amplified by PCR from DNA isolated from the fetal fibroblasts that would be used for later transfections to ensure an isogenic match between the targeting vector and the transfected cell line. Briefly, LA taq (Clontech) using the forward primer CTCTCCCTCACTCTAACCTACTT (SEQ ID NO: 11), and the reverse primer TATTTCTCTCACATGGCCAGTC (SEQ ID NO: 12) were used to amplify a 9538 bp fragment of CD163. The fragment was DNA sequence validated and used to build the domain-swap targeting vector (FIG. 1). This vector included 33 point mutations within exon 7 so that it would encode the same amino acid sequence as human CD163L from exon 11. The replacement exon was 315 bp. In addition, the subsequent intron was replaced with a modified myostatin intron B that housed a selectable marker gene that could be removed with Cre-recombinase (Cre) and had previously demonstrated normal splicing when harboring the retained loxP site (Wells, unpublished results). The long arm of the construct was 3469 bp and included the domain swap DS exon. The short arm was 1578 bp and included exons 7 and 8 (FIG. 1, panel B). This plasmid was used to attempt to replace the coding region of exon 7 in the first transfection experiments and allowed for selection of targeting events via the selectable marker (G418). If targeting were to occur, the marker could be deleted by Cre-recombinase. The CD163 DS-targeting vector was then modified for use with cell lines that already contained a SIGLEC1 gene disrupted with Neo that could not be Cre deleted. In this targeting vector, the Neo cassette, loxP and myostatin intron B, were removed, and only the DS exon remained with the WT long and short arm (FIG. 1, panel C).

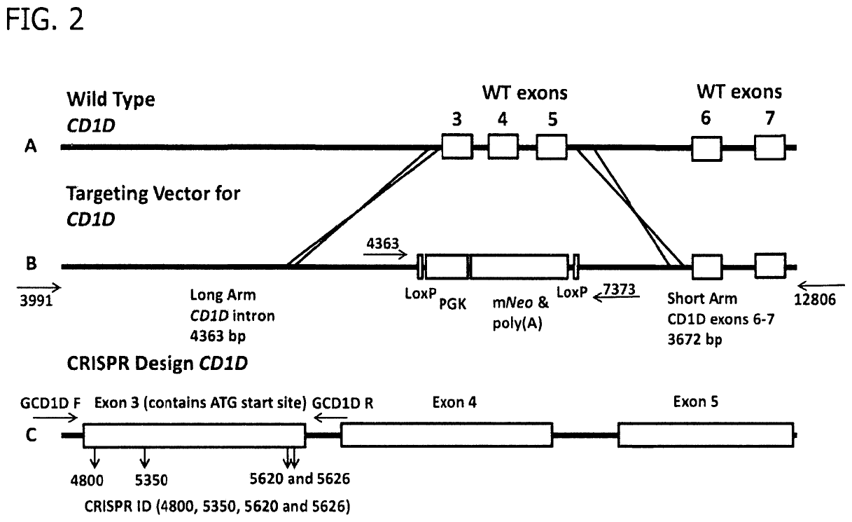

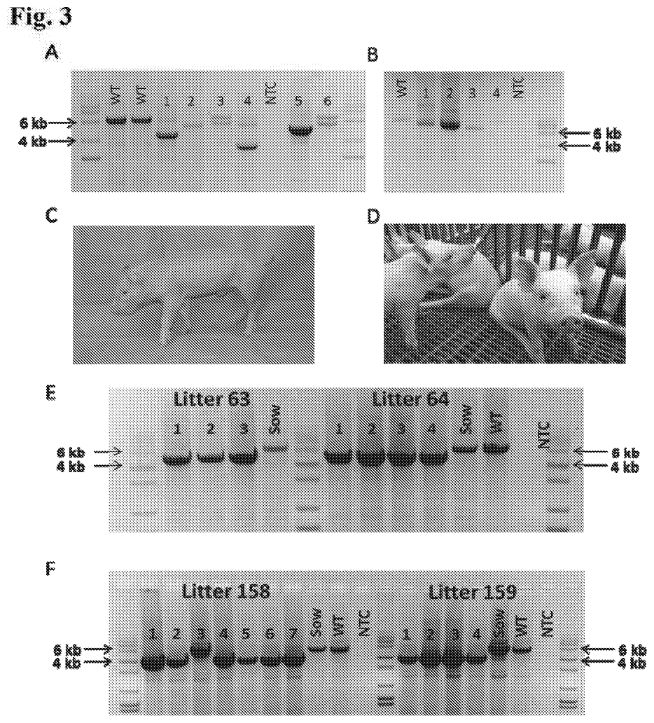

The genomic sequence for porcine CD1D was amplified with LA taq using the forward primer CTCTCCCTCACTCTAACCTACTT (SEQ ID NO: 13) and reverse primer GACTGGCCATGTGAGAGAAATA (SEQ ID NO: 14), resulting in an 8729 bp fragment. The fragment was DNA sequenced and used to build the targeting vector shown in FIG. 2. The Neo cassette is under the control of a phosphoglycerol kinase (PGK) promoter and flanked with loxP sequences, which were introduced for selection. The long arm of the construct was 4832 bp and the short arm was 3563 bp, and included exons 6 and 7. If successful HR occurred, exons 3, 4, and 5 would be removed and replaced with the Neo cassette. If NHEJ repair occurred incorrectly, then exon 3 would be disrupted.

### Fetal Fibroblast Collection

Porcine fetal tissue was collected on Day 35 of gestation to create cell lines. Two wild-type (WT) male and female fetal fibroblast cell lines were established from a large white domestic cross. Male and female fetal fibroblasts that had previously been modified to contain a Neo cassette (SIGLEC1−/− genetics) were also used in these studies. Fetal fibroblasts were collected as described with minor modifications; minced tissue from each fetus was digested in 20 ml of digestion media (Dulbecco-modified Eagle medium [DMEM] containing L-glutamine and 1 g/L D-glucose [Cellgro] supplemented with 200 units/ml collagenase and 25 Kunitz units/ml DNAseI) for 5 hours at 38.5° C. After digestion, fetal fibroblast cells were washed and cultured with DMEM, 15% fetal bovine serum (FBS), and 40 μg/ml gentamicin. After overnight culture, the cells were trypsinized and frozen at −80° C. in aliquots in FBS with 10% dimethyl sulfoxide and stored in liquid nitrogen.

### Cell Transfection and Genotyping

Transfection conditions were essentially as previously reported. The donor DNA was always used at a constant amount of 1 μg with varying amounts of CRISPR/Cas9 plasmid (listed below). Donor DNA was linearized with MLUI (CD163) (NEB) or AFLII (CD1D) (NEB) prior to transfection. The gender of the established cell lines was determined by PCR as described previously prior to transfection. Both male and female cell lines were transfected, and genome modification data was analyzed together between the transfections. Fetal fibroblast cell lines of similar passage number (2-4) were cultured for 2 days and grown to 75%-85% confluency in DMEM containing L-glutamine and 1 g/L D-glucose (Cellgro) supplemented with 15% FBS, 2.5 ng/ml basic fibroblast growth factor, and 10 mg/ml gentamicin. Fibroblast cells were washed with phosphate-buffered saline (PBS) (Life Technologies) and trypsinized. As soon as cells detached, the cells were rinsed with an electroporation medium (75% cytosalts [120 mM KCl, 0.15 mM CaCl₂, 10 mM K₂HPO₄, pH 7.6, 5 Mm MgCl₂]) and 25% Opti-MEM (LifeTechnologies). Cell concentration was quantified by using a hemocytometer. Cells were pelleted at 600×g for 5 minutes and resuspended at a concentration of 1×10⁶ in electroporation medium. Each electroporation used 200 μl of cells in 2 mm gap cuvettes with three (1 msec) square-wave pulses administered through a BTX ECM 2001 at 250 V. After the electroporation, cells were resuspended in DMEM described above. For selection, 600 μg/ml G418 (Life Technologies) was added 24 hours after transfection, and the medium was changed on Day 7. Colonies were picked on Day 14 after transfection. Fetal fibroblasts were plated at 10,000 cells/plate if G418 selection was used and at 50 cells/plate if no G418 selection was used. Fetal fibroblast colonies were collected by applying 10 mm autoclaved cloning cylinders sealed around each colony by autoclaved vacuum grease. Colonies were rinsed with PBS and harvested via trypsin; then resuspended in DMEM culture medium. A part (1/3) of the resuspended colony was transferred to a 96-well PCR plate, and the remaining (2/3) cells were cultured in a well of a 24-well plate. The cell pellets were resuspended in 6 μl of lysis buffer (40 mM Tris, pH 8.9, 0.9% Triton X-100, 0.4 mg/ml proteinase K [NEB]), incubated at 65° C. for 30 minutes for cell lysis, followed by 85° C. for 10 minutes to inactivate the proteinase K.

### PCR Screening for DS and Large and Small Deletions

Detection of HR-directed repair. Long-range PCRs were used to identify mutations on either CD163 or CD1D. Three different PCR assays were used to identify HR events: PCR amplification of regions spanning from the CD163 or CD1D sequences in the donor DNA to the endogenous CD163 or CD1D sequences on either the right or left side and a long-range PCR that amplified large regions of CD163 or CD1D encompassing the designed donor DNAs. An increase in the size of a PCR product, either 1.8 kb (CD1D) or 3.5 kb (CD163), arising from the addition of exogenous Neo sequences, was considered evidence for HR-directed repair of the genes. All the PCR conditions included an initial denaturation of 95° C. for 2 minutes followed by 33 cycles of 30 seconds at 94° C., 30 seconds at 50° C., and 7-10 minutes at 68° C. LA taq was used for all the assays following the manufacturers' recommendations. Primers are shown in Table 3.

TABLE 3
Primers used to identify HR directed repair of CD163
and CD1D

| | | SEQ |
|---|---|---|
| | | ID |
| Primer | Sequence (5′-3′) | NO. |
| CD163 Long Range Assay Primer 1230F | TTGTTGGAAGGCTCACTGTCCTTG | 68 |
| CD163 Long Range Assay Primer 7775 R | ACAACTAAGGTGGGGCAAAG | 69 |
| CD163 Left Arm Assay Primer 1230 F | TTGTTGGAAGGCTCACTGTCCTTG | 70 |
| CD163 Left Arm Assay Primer 8491 R | GGAGCTCAACATTCTTGGGTCCT | 71 |
| CD163 Right Arm Assay Primer 3752 F | GGCAAAATTTTCATGCTGAGGTG | 72 |
| CD163 Right Arm Assay Primer 7765 R | GCACATCACTTCGGGTTACAGTG | 73 |
| CD1D Long Range Assay Primer F 3991 F | CCCAAGTATCTTCAGTTCTGCAG | 74 |
| CD1D Long Range Assay Primer R 12806 R | TACAGGTAGGAGAGCCTGTTTTG | 75 |
| CD1D Left Arm Assay Primer F 3991 F | CCCAAGTATCTTCAGTTCTGCAG | 76 |
| CD1D Left Arm Assay Primer 7373 R | CTCAAAAGGATGTAAACCCTGGA | 77 |
| CD1D Right Arm Assay Primer 4363 F | TGTTGATGTGGTTTGTTTGCCC | 78 |
| CD1D Right Arm Assay Primer 12806 R | TACAGGTAGGAGAGCCTGTTTTG | 79 |

Small deletions assay (NHEJ). Small deletions were determined by PCR amplification of CD163 or CD1D flanking a projected cutting site introduced by the CRISPR/Cas9 system. The size of the amplicons was 435 bp and 1244 bp for CD163 and CD1D, respectively. Lysates from both embryos and fetal fibroblasts were PCR amplified with LA taq. PCR conditions of the assays were an initial denaturation of 95° C. for 2 minutes followed by 33 cycles of 30 seconds at 94° C., 30 seconds at 56° C., and 1 minute at 72° C. For genotyping of the transfected cells, insertions and deletions (INDELs) were identified by separating PCR amplicons by agarose gel electrophoresis. For embryo genotyping, the resulting PCR products were subsequently DNA sequenced to identify small deletions using forward primers used in the PCR. Primer information is shown in Table 4.

TABLE 4
Primers used to identify mutations through
NHEJ on CD163 and CD1D

| Primer | Sequence (5′-3′) | SEQ ID NO. |
|---|---|---|
| GCD163F | GGAGGTCTAGAATCGGCTAAGCC | 80 |
| GCD163R | GGCTACATGTCCCGTCAGGG | 81 |
| GCD1DF | GCAGGCCACTAGGCAGATGAA | 82 |
| GCD1DR | GAGCTGACACCCAAGAAGTTCCT | 83 |
| eGFP1 | GGCTCTAGAGCCTCTGCTAACC | 84 |
| eGFP2 | GGACTTGAAGAAGTCGTGCTGC | 85 |

### Somatic Cell Nuclear Transfer (SCNT)

To produce SCNT embryos, either sow-derived oocytes (ART, Inc.) or gilt-derived oocytes from a local slaughter house were used. The sow-derived oocytes were shipped overnight in maturation medium (TCM-199 with 2.9 mM Hepes, 5 pg/ml insulin, 10 ng/ml epidermal growth factor [EGF], 0.5 pg/ml porcine follicle-stimulating hormone [p-FSH], 0.91 mM pyruvate, 0.5 mM cysteine, 10% porcine follicular fluid, and 25 ng/ml gentamicin) and transferred into fresh medium after 24 hours. After 40-42 hours of maturation, cumulus cells were removed from the oocytes by vortexing in the presence of 0.1% hyaluronidase. The gilt-derived oocytes were matured as described below for in vitro fertilization (IVF). During manipulation, oocytes were placed in the manipulation medium (TCM-199 [Life Technologies] with 0.6 mM NaHCO₃, 2.9 mM Hepes, 30 mM NaCl, 10 ng/ml gentamicin, and 3 mg/ml BSA, with osmolarity of 305 mOsm) supplemented with 7.0 μg/ml cytochalasin B. The polar body along with a portion of the adjacent cytoplasm, presumably containing the metaphase II plate, was removed, and a donor cell was placed in the perivitelline space by using a thin glass capillary. The reconstructed embryos were then fused in a fusion medium (0.3 M mannitol, 0.1 mM CaCl₂, 0.1 mM MgCl₂, and 0.5 mM Hepes) with two DC pulses (1-second interval) at 1.2 kV/cm for 30 seconds using a BTX Electro Cell Manipulator (Harvard Apparatus). After fusion, fused embryos were fully activated with 200 μM thimerosal for 10 minutes in the dark and 8 mM dithiothreitol for 30 minutes. Embryos were then incubated in modified porcine zygote medium PZM3-MU1 with 0.5 μM Scriptaid (S7817; Sigma-Aldrich), a histone deacetylase inhibitor, for 14-16 hours, as described previously.

### In Vitro Fertilization (IVF)

For IVF, ovaries from prepubertal gilts were obtained from an abattoir (Farmland Foods Inc.). Immature oocytes were aspirated from medium size (3-6 mm) follicles using an 18-gauge hypodermic needle attached to a 10 ml syringe. Oocytes with evenly dark cytoplasm and intact surrounding cumulus cells were then selected for maturation. Around 50 cumulus oocyte complexes were place in a well containing 500 μl of maturation medium, TCM-199 (Invitrogen) with 3.05 mM glucose, 0.91 mM sodium pyruvate, 0.57 mM cysteine, 10 ng/ml EGF, 0.5 μg/ml luteinizing hormone (LH), 0.5 μg/ml FSH, 10 ng/ml gentamicin (APP Pharm), and 0.1% polyvinyl alcohol for 42-44 hours at 38.5° C., 5% CO2, in humidified air. At the end of the maturation, the surrounding cumulus cells were removed from the oocytes by vortexing for 3 minutes in the presence of 0.1% hyaluronidase. Then, in vitro matured oocytes were placed in 50 μl droplets of IVF medium (modified Tris-buffered medium containing 113.1 mM NaCl, 3 mM KCl, 7.5 mM CaCl2, 11 mM glucose, 20 mM Tris, 2 mM caffeine, 5 mM sodium pyruvate, and 2 mg/ml bovine serum albumin [BSA]) in groups of 25-30 oocytes. One 100 μl frozen semen pellet was thawed in 3 ml of Dulbecco PBS supplemented with 0.1% BSA. Either frozen WT or fresh eGFP semen was washed in 60% Percoll for 20 minutes at 650 3 g and in modified Tris-buffered medium for 10 minutes by centrifugation. In some cases, freshly collected semen heterozygous for a previously described eGFP transgene was washed three times in PBS. The semen pellet was then resuspended with IVF medium to 0.5×10⁶ cells/ml. Fifty microliters of the semen suspension was introduced into the droplets with oocytes. The gametes were coincubated for 5 hours at 38.5° C. in an atmosphere of 5% CO2 in air. After fertilization, the embryos were incubated in PZM3-MU1 at 38.5° C. and 5% CO2 in air.

### Embryo Transfer

Embryos generated to produce GE CD163 or CD1D pigs were transferred into surrogates either on Day 1 (SCNT) or 6 (zygote injected) after first standing estrus. For Day 6 transfer, zygotes were cultured for five additional days in PZM3-MU1 in the presence of 10 ng/ml ps48 (Stemgent, Inc.). The embryos were surgically transferred into the ampullary-isthmic junction of the oviduct of the surrogate.

### In Vitro Synthesis of RNA for CRISPR/Cas9 System

Template DNA for in vitro transcription was amplified using PCR (Table 5). CRISPR/Cas9 plasmid used for cell transfection experiments served as the template for the PCR. In order to express the Cas9 in the zygotes, the mMESSAGE mMACHINE Ultra Kit (Ambion) was used to produce mRNA of Cas9. Then a poly A signal was added to the Cas9 mRNA using a Poly (A) tailing kit (Ambion). CRISPR guide RNAs were produced by MEGAshortscript (Ambion). The quality of the synthesized RNAs were visualized on a 1.5% agarose gel and then diluted to a final concentration of 10 ng/μl (both gRNA and Cas9) and distributed into 3 μl aliquots.

TABLE 5
Primers used to amplify templates for in vitro transcription.

| | | SEQ ID |
|---|---|---|
| Primers | Sequence (5′-3′) | NO. |
| Cas9 | F: TAATACGACTCACTATAGGGAGAATGGACTATAAGGACCACGAC | 86 |
| | R: GCGAGCTCTAGGAATTCTTAC | 87 |
| eGFP1 | F: TTAATACGACTCACTATAGGCTCCTCGCCCTTGCTCACCA | 88 |
| | R: AAAAGCACCGACTCGGTGCC | 89 |
| CD163 | F: TTAATACGACTCACTATAGGAAACCCAGGCTGGTTGGA | 90 |
| 10 | R: AAAAGCACCGACTCGGTGCC | 91 |
| CD163 | F: TTAATACGACTCACTATAGGAACTACAGTGCGGCACTG | 92 |
| 131 | R: AAAAGCACCGACTCGGTGCC | 93 |
| CD1D | F: TTAATACGACTCACTATAGGCCAGCCTCGCCCAGCGACAT | 94 |
| 4800 | R: AAAAGCACCGACTCGGTGCC | 95 |
| CD1D | F: TTAATACGACTCACTATAGGCAGCTGCAGCATATATTTAA | 96 |
| 5350 | R: AAAAGCACCGACTCGGTGCC | 97 |

### Microinjection of Designed CRISPR/Cas9 System in Zygotes

Messenger RNA coding for Cas9 and gRNA was injected into the cytoplasm of fertilized oocytes at 14 hours post-fertilization (presumptive zygotes) using a FemtoJet microinjector (Eppendorf). Microinjection was performed in manipulation medium on the heated stage of a Nikon inverted microscope (Nikon Corporation; Tokyo, Japan). Injected zygotes were then transferred into the PZM3-MU1 with 10 ng/ml ps48 until further use.

### Statistical Analysis

The number of colonies with a modified genome was classified as 1, and the colonies without a modification of the genome were classified as 0. Differences were determined by using PROC GLM (SAS) with a P-value of 0.05 being considered as significant. Means were calculated as least-square means. Data are presented as numerical means±SEM.

### Results

### CRISPR/Cas9-Mediated Knockout of CD163 and CD1D in Somatic Cells

Efficiency of four different CRISPRs plasmids (guides 10, 131, 256, and 282) targeting CD163 was tested at an amount of 2 μg/μl of donor DNA (Table 6). CRISPR 282 resulted in significantly more average colony formation than CRISPR 10 and 256 treatments (P<0.05). From the long-range PCR assay described above, large deletions were found ranging from 503 bp to as much as 1506 bp instead of a DS through HR as was originally intended (FIG. 3, panel A). This was not expected because previous reports with other DNA-editing systems showed much smaller deletions of 6-333 bp using ZFN in pigs. CRISPR 10 and a mix of all four CRISPRs resulted in a higher number of colonies with a modified genome than CRISPR 256 and 282 (Table 6, P<0.002). Transfection with CRISPR 10 and a plasmid containing Neo but no homology to CD163 resulted in no colonies presenting the large deletion. Interestingly, one monoallelic deletion was also detected when the donor DNA was introduced without any CRISPR. This assay likely represents an underestimation of the mutation rate because any potential small deletions by sequencing which could not be detected on an agarose gel in the transfected somatic cells were not screened for.

TABLE 6
Efficiency of four different CRISPR plasmids (guides 10, 131, 256, and 282) targeting CD163.
Four different CRISPRs were tested at an amount of 2 μg to 1 μg Donor DNA (shown in FIG. 1).

| | | | Average | | | | |
|---|---|---|---|---|---|---|---|
| | Total | Total | No. of | No. of | | Percent Colonies | |
| | No. of | No. of | Colonies/ | Colonies | Colony | with a Modified | |
| Treatment* | Colonies | Plates | plate† | NHEJ | with HR | Genome† | Reps |
| 10 + Donor DNA | 76 | 102 | 0.75^{bc} | 11 | 1‡ | 15.79^{a} | 4 |
| 131 + Donor DNA | 102 | 51 | 2.00^{ab} | 11 | 0 | 10.78^{ab} | 3 |
| 256 + Donor DNA | 43 | 49 | 0.88^{c} | 2 | 0 | 4.65^{bc} | 3 |
| 282 + Donor DNA | 109 | 46 | 2.37^{a} | 3 | 0 | 2.75^{bc} | 3 |
| mix of 4 + Donor DNA | 111 | 55 | 2.02^{ab} | 20 | 0 | 18.02^{a} | 3 |
| Donor DNA | 48 | 52 | 0.92^{bc} | 1 | 0 | 2.08^{bc} | 3 |
| 10 + Neo (no CD163) | 26 | 20 | 1.3^{n/a} | 0 | 0 | 0.00^{c} | 1 |

*Mix of 4 + Donor DNA represents an equal mixing of 0.5 μg of each CRISPR with 1 μg of Donor DNA. The Donor DNA treatment served as the no CRISPR control and the 10 + Neo treatment illustrates that the large deletions observed in the CRISPR treatments were present only when the CD163 Donor DNA was also present.
†ANOVA was performed comparing the average number of colonies/plate to estimate CRISPR toxicity and on the percent colonies with a modified genome. P-values were 0.025 and 0.0002, respectively.
^{n/a} = There were no replicates for this treatment so no statistical analysis was performed.
‡The one colony with HR represents a partial HR event.
^{a-c}Superscript letters indicate a significant difference between treatments for both average number of colonies/plate and percent colonies with a modified genome (P < 0.05).

The initial goal was to obtain a domain swap (DS)-targeting event by HR for CD163, but CRISPRs did not increase the efficiency of targeting CD163. It should be noted that various combinations of this targeting vector had been used to modify CD163 by HR by traditional transfections and resulted in 0 targeting events after screening 3399 colonies (Whitworth and Prather, unpublished results). Two pigs were obtained with a full DS resulting from HR that contained all 33 of the mutations that were attempted to be introduced by transfection with CRISPR 10 and the DS-targeting vector as donor DNA.

Next, the efficiency of CRISPR(Cas9-induced mutations without drug selection was tested; the fetal fibroblast cell line used in this study already had an integration of the Neo resistant cassette and a knockout of SIGLEC1. Whether the ratio of CRISPR/Cas9 and donor DNA would increase genome modification or result in a toxic effect at a high concentration was also tested. CRISPR 131 was selected for this trial because in the previous experiment, it resulted in a high number of total colonies and an increased percentage of colonies possessing a modified genome. Increasing amounts of CRISPR 131 DNA from 3:1 to 20:1 did not have a significant effect on fetal fibroblast survivability. The percent of colonies with a genome modified by NHEJ was not significantly different between the various CRISPR concentrations but had the highest number of NHEJ at a 10:1 ratio (Table 7, P=0.33). Even at the highest ratio of CRISPR DNA to donor DNA (20:1), HR was not observed.

TABLE 7
Efficiency of CRISPR/Cas9-induced mutations without drug selection. Four
different ratios of Donor DNA to CRISPR 131 DNA were compared in a previously
modified cell line without the use of G418 selection.

| | | | | Number | Percent | | | |
|---|---|---|---|---|---|---|---|---|
| | | Number | Mean | of | Colonies | Colony | Percent | |
| Donor DNA: | Number | of | Number of | Colonies | with | with | Colonies | |
| CRISPR Ratio | Plates | Colonies | Colonies/Plate | NHEJ | NHEJ | HR | with HR | Reps |
| 1:0 | 30 | 79 | 2.6 | 1 | 1.3^{a} | 0 | 0.0 | 2 |
| 1:3 | 30 | 84 | 2.8 | 1 | 1.2^{a} | 0 | 0.0 | 2 |
| 1:5 | 27 | 76 | 2.8 | 2 | 2.6^{a} | 0 | 0.0 | 2 |
| 1:10 | 32 | 63 | 2.0 | 5 | 7.9^{a} | 0 | 0.0 | 2 |
| 1:20 | 35 | 77 | 2.2 | 3 | 3.9^{a} | 0 | 0.0 | 2 |

^{a}Significant difference between treatments for percent colonies with NHEJ repair (P > 0.05).
^{b}There was not a significant difference in the number of genome modified colonies with increasing concentration of CRISPR (P > 0.33).

Based on this experience, targeted disruption of CD1D in somatic cells was attempted. Four different CRISPRs were designed and tested in both male and female cells. Modifications of CD1D could be detected from three of the applied CRISPRs, but use of CRISPR 5350 did not result in modification of CD1D with a deletion large enough to detect by agarose gel electrophoresis (Table 8). Interestingly, no genetic changes were obtained through HR although donor DNA was provided. However, large deletions similar to the CD163 knockout experiments were observed (FIG. 3, panel B). No targeted modification of CD1D with a large deletion was detected when CRISPR/Cas9 was not used with the donor DNA. Modification of CD1D from CRISPR/Cas9-guided targeting was 4/121 and 3/28 in male and female colonies of cells, respectively. Only INDELs detectable by agarose gel electrophoresis were included in the transfection data.

TABLE 8
Four different CRISPRS were tested at an amount of 2 μg to
1 μg Donor DNA (shown in FIG. 2). The Donor DNA treatment
served as the no CRISPR control.

| | | Total Number | | |
|---|---|---|---|---|
| Gender | Treatment | of Colonies | INDEL | Efficiency (%) |
| male | 4800 + Donor | 29 | 2 | 6.9 |
| | DNA | | | |
| male | 5350 + Donor | 20 | 0 | 0 |
| | DNA | | | |
| male | 5620 + Donor | 43 | 1 | 2.33 |
| | DNA | | | |
| male | 5626 + Donor | 29 | 2 | 6.9 |
| | DNA | | | |
| male | Donor DNA | 28 | 0 | 0 |
| female | 4800 + Donor | 2 | 0 | 0 |
| | DNA | | | |
| female | 5350 + Donor | 8 | 0 | 0 |
| | DNA | | | |
| female | 5620 + Donor | 10 | 0 | 0 |
| | DNA | | | |
| female | 5626 + Donor | 8 | 3 | 37.5 |
| | DNA | | | |
| female | Donor DNA | 7 | 0 | 0 |

Production of CD163 and CD1D Pigs through SCNT Using the GE Cells

The cells presenting modification of CD163 or CD1D were used for SCNT to produce CD163 and CD1D knockout pigs (FIG. 3). Seven embryo transfers (CD163 Table 9), six embryo transfers (CD163-No Neo), and five embryo transfers (CD1D) into recipient gilts were performed with SCNT embryos from male and female fetal fibroblasts transfected with CRISPR/Cas9 systems. Six (CD163), two (CD163-No Neo), and four (CD1D) (Table 10) of the recipient gilts remained pregnant to term resulting in pregnancy rates of 85.7%. 33.3%, and 80%, respectively. Of the CD163 recipients, five delivered healthy piglets by caesarean section. One (0044) farrowed naturally. Litter size ranged from one to eight. Four pigs were euthanized because of failure to thrive after birth. One piglet was euthanized due to a severe cleft palate. All the remaining piglets appear healthy (FIG. 3, panel C). Two litters of male piglets resulting from fetal fibroblasts transfected with CRISPR 10 and donor DNA described in FIG. 3, panel B had a 30 bp deletion in exon 7 adjacent to CRISPR 10 and an additional 1476 bp deletion of the preceding intron, thus removing the intron 6/exon 7 junction of CD163 (FIG. 3, panel E). The genotypes and predicted translations are summarized in Table 11. One male piglet and one female litter (4 piglets) were obtained from the CD163-No Neo transfection of previously modified SIGLEC1 cells. All five piglets were double knockouts for SIGLEC1 and CD163. The male piglet had a biallelic modification of CD163 with a 28 bp deletion in exon 7 on one allele and a 1387 bp deletion on the other allele that included a partial deletion of exon 7 and complete deletion of exon 8 and the proceeding intron, thus removing the intron exon junction. The female piglets had a biallelic mutation of CD163, including a 1382 bp deletion with a 11 bp insertion on one allele and a 1720 bp deletion of CD163 on the other allele. A summary of the CD163 modifications and the predicted translations can be found in Table 11. A summary of the CD1D modifications and predicted translations by CRISPR modification can be found in Table 12. Briefly, one female and two male litters were born, resulting in 13 piglets. One piglet died immediately after birth. Twelve of the 13 piglets contained either a biallelic or homozygous deletion of CD1D (FIG. 3, panel F). One piglet was WT.

TABLE 9
Embryo Transfer data for CD163.

| | | | # Embryos | Oocyte | Day of | |
|---|---|---|---|---|---|---|
| Pig ID | Line* | Gender | Transferred | Source† | Estrus | Piglet Result |
| O047 | CD163 CRISPR NT | Male | 240 | ART | 2 | 4 live piglets (2 euthanized |
| | | | | | | after birth) |
| O015 | CD163 CRISPR NT | Male | 267 | ART | 1 | 3 live piglets (all healthy) |
| O044 | CD163 CRISPR NT | Male | 206 | ART | 1 | 7 live piglets (1 born dead, |
| | | | | | | 1 euthanized after birth) |
| O053 | CD163 CRISPR NT | Male | 224 | ART | 2 | 1 male piglet (euthanized |
| | | | | | | at day 13) |
| O08 | CD163 CRISPR NT | Male | 226 | ART | 1 | 0 piglets |
| O094 | CD163 CRISPR NT | Female | 193 | MU | 2 | 8 live piglets (1 euthanized |
| | | | | | | due to FTT) |
| O086 | CD163 CRISPR NT | Female | 213 | MU | 1 | 9 live piglets (2 euthanized |
| | | | | | | at day 0, 2 due to FTT) |
| O082 | CRISPR Injected CD 163 | Male/Female | 50 Blast | MU | 5 | 0 piglets |
| | 10/131 | | | | | |
| O083 | CRISPR Injected CD163 | Male | 46 Blast | MU | 5 | 4 live piglets |
| | 10/131 | | | | | |
| O99 | CD163 CRISPR NT-no Neo | Male | 156 | ART | 1 | 1 live piglet, 1 dead piglet |
| O128 | CD163 CRISPR NT-no Neo | Male | 196 | ART | 2 | 0 piglets |
| O100 | CD163 CRISPR NT-no Neo | Male | 261 | MU | 3 | 0 piglets |
| O134 | CD163 CRISPR NT-no Neo | Male/Female | 181 | MU | 1 | 0 piglets |
| 200889 | CD163 CRISPR NT-no Neo | Female | 202 | ART | 1 | 4 live piglets |
| O135 | CD163 CRISPR NT-no Neo | Female | 169 | ART | 2 | 0 piglets |

*The CD163 CRISPR NT line represents embryos created by NT with a fetal fibroblast line modified by transfection. CRISPR injected embryos were IVF embryos injected at the 1 cell stage with CD163 guide RNA with CAS9 RNA. CD163 CRISPR NT-no Neo fetal line represents embryos created by NT with a previously modified fetal fibroblast that was already Neo resistant line modified by transfection without the use of a selectable marker.
†MU refers to gilt oocytes that were aspirated and matured at the University of Missouri as described in the IVF se4ction of the Materials and Methods. ART refers to sow oocytes that were purchased and matured as described in the SCNT section of the Materials and Methods.

TABLE 10
Embryo transfer data for CD1D.

| | | | # Embryos | Oocyte | Day of | |
|---|---|---|---|---|---|---|
| Pig ID | Line* | Gender | Transferred | Source† | Estrus | Result |
| 200888 | CD1D CRISPR NT | Male | 201 | ART | 2 | 7 live piglets |
| O61 | CD1D CRISPR NT | Male | 239 | ART | 0 | 4 live piglets |
| O164 | CD1D CRISPR NT | Female | 199 | MU | 2 | 0 piglets |
| O156 | CD1D CRISPR NT | Female | 204 | MU | 2 | 0 piglets |
| O165 | CD1D Injected | Male/Female | 55 Blast | MU | 6 | 4 piglets (1 female, 3 male) |
| | 4800/5350 | | | | | |
| O127 | CD1D Injected | Male/Female | 55 Blast | MU | 6 | 0 piglets |
| | 4800/5350 | | | | | |
| O121 | CD1D CRISPR NT | Female | 212 | ART | 1 | 2 live piglets |

*CD1D CRISPR NT line represents embryos created by NT with a fetal fibroblast line modified by transfection. CRISPR injected embryos were IVF embryos injected at the 1 cell stage with CD1D guide RNA with CAS9 RNA.
†MU refers to gilt oocytes that were aspirated and matured at the University of Missouri as described in the IVF se4ction of the Materials and Methods. ART refers to sow oocytes that were purchased and matured as described in the SCNT section of the Materials and Methods.

TABLE 11
Genotype and Translational Prediction for CD163 modified pigs. Some pigs contain a
biallelic type of modification, but only have one allele described and another modified
allele that was not amplified by PCR.

| | | | | | | Protein | Premature | | SEQ |
|---|---|---|---|---|---|---|---|---|---|
| | No. of | Repair | | Size of | | trans- | stop | In reference | ID |
| Litter | Piglets | mechanism | Type | INDELS | Description | lation* | codon | SEQ ID NO: 47 | NO^{†} |
| 63 & | 7 | NHEJ | biallelic | 1506 bp deletion | 30 bp deletion in exon 7 | KO or | No | Deletion from nt 1,525 to nt | 98 |
| 64 | | | | Other allele | Uncharacterized, | CD163^{Δ422-527} | | 3,030 | |
| | | | | | unamplifiable | | | | |
| 65 | 3 | NHEJ | Biallelic | 7 bp insertion | Insertion into exon 7 | KO | Yes (491) | Insertion between nt 3,148 & | 99 |
| | | | | | | | | 3.149^{a} | |
| 65 | 2 | NHEJ | Biallelic | 503 bp deletion | Partial deletion of | KO | Yes (491) | ** | ** |
| | | | | | exons 7 and 8 | | | | |
| | | | | Other allele | Uncharacterized | | | | |
| 65 | 2 | NHEJ | Biallelic | 1280 bp deletion | Complete deletion of | CD163^{Δ422-631} | No | Deletion from nt 2,818 to nt | 100 |
| | | | | | exons 7 and 8 | | | 4,097 | |
| | | | | 1373 bp deletion | Complete deletion of | CD163^{Δ422-631} | NO | Deletion from nt 2,724 to nt | 101 |
| | | | | | exons 7 and 8 | | | 4,096 | |
| 66 | 1 | NHEJ | Homo- | 2015 bp | Insertion of targeting | | | ** | ** |
| | | | zygous | insertion | vector backbone into | | | | |
| | | | | | exon 7 | | | | |
| 67-1 | 1 | NHEJ | Biallelic | 11 bp deletion | Deletion in exon 7 | KO | Yes (485) | Deletion from nt 3,137 to nt | 102 |
| | | | | | | | | 3,147 | |
| | | | | 2 bp insertion, | Insertion in exon 7 | | | 2 bp insertion between nt | 103 |
| | | | | 377 bp deletion | | | | 3,149 & nt 3,150^{b} with a 377 | |
| | | | | in intron 6 | | | | bp deletion from nt 2,573 to | |
| | | | | | | | | nt 2,949 | |
| 67-2 | 1 | NHEJ | Biallelic | 124 bp deletion | Deletion in exon 7 | KO | Yes (464) | Deletion from nt 3,024 nt | 104 |
| | | | | | | | | 3,147 | |
| | | | | 123 bp deletion | Deletion in exon 7 | CD163^{Δ429-470} | No | Deletion from nt 3,024 nt | 105 |
| | | | | | | | | 3,146 | |
| 67-3 | 1 | NHEJ | Biallelic | 1 bp insertion | Insertion into exon 7 | KO | Yes (489) | Insertion between nt 3,147 | 106 |
| | | | | | | | | & 3,148^{c} | |
| | | | | other allele | Uncharacterized, | | | | |
| | | | | | unamplifialbe | | | | |
| 67-4 | 1 | NHEJ | Biallelic | 130 bp deletion | Deletion in exon 7 | KO | Yes (462) | Deletion from nt 3,030 to nt | 107 |
| | | | | | | | | 3,159 | |
| | | | | 132 bp deletion | Deletion in exon 7 | CD163^{Δ430-474} | No | Deletion from nt 3,030 to nt | 108 |
| | | | | | | | | 3,161 | |
| 68 & | 6 | NHEJ | Biallelic | 1467 bp deletion | Cpmplete deletion of | CD163^{Δ422-631} | No | Deletion from nt 2,431 to nt | 109 |
| 69 | | | | | exons 7 and 8 | | | 3,897 | |
| | | | | Other allele | Uncharacterized, | | | | |
| | | | | | unamplifiable | | | | |
| 68 & | 2 | NHEJ | Biallelic | 129 bp deletion, | Deletion in exon 7 | CD163^{Δ435-478} | No | Deletion from nt 488 to nt | 110 |
| 69 | | | | 1930 bp intron | Uncharacterized, | | | 2,417 in exon 6, deleted | |
| | | | | 6 deletion | unamplifiable | | | sequence is replaced with a | |
| | | | | other allele | | | | 12 bp insertion^{d} starting at | |
| | | | | | | | | nt 488, & an additional 129 | |
| | | | | | | | | bp deletion from nt 3,044 to | |
| | | | | | | | | nt 3,172 | |
| 65 & | 3 | WT | | | Wild type pigs created | | | SEQ ID NO: 47 | 47 |
| 69 | | | | | from a mixed colony | | | | |
| 70 | 2 | NHEJ | On | 28 bp deletion | Deletion in exon 7 | KO | YES (528) | Deletion from nt 3,145 to nt | 111 |
| | | | SIGLEC^{1-/-} | | | | | 3,172 | |
| | | | Biallelic | 1387 deletion in | Partial deletion in exon | KO | No | Deletion from nt 3,145 to nt | |
| | | | | exon 7 and all | 7 and all of exon 8 | | | 4,531 | |
| | | | | of exon 8 | | | | | |
| 73 | 4 | NHEJ | On | 1382 bp deletion | Partial deletion in exon | KO | No | Deletion from nt 3,113 to nt | 113 |
| | | | SIGLEC^{1-/-} | +11 bp insertion | 7 and all of exon 8 | | | 4,494, deleted sequence | |
| | | | Biallelic | | | | | replaced with an 11 bp | |
| | | | | | | | | insertion^{e} starting at nt | |
| | | | | | | | | 3,113 | |
| | | | | 1720 bp deletion | Complete deletions of | CD163^{Δ422-631} | | Deletion from nt 2,440 to nt | 114 |
| | | | | | exons 7 and 8 | | | 4,160 | |

*KO, knock-out
**Not included because piglets were euthanized
^{†}SEQ ID NOs. in this column refer to the SEQ ID NOs. for the sequences that show the INDELs in relation to SEQ ID NO: 47.
^{a}The inserted sequence was TACTACT (SEQ ID NO: 115)
^{b}The inserted sequence was AG.
^{c}The inserted sequence was a single adenine (A) residue.
^{d}The inserted sequence was TGTGGAGAATTC (SEQ ID NO: 116).
^{e}The inserted sequence was AGCCAGCGTGC (SEQ ID NO: 117).

TABLE 12
Genotype and Translational Prediction for CD1D modified pigs

| | Number | | | | | |
|---|---|---|---|---|---|---|
| | of | Repair | | | | Protein |
| Litter | Piglets | Mechanism | Type | Size of INDEL | Description | Translation |
| 158, | 11 | NHEJ | homozygous | 1653 bp deletion | Deletion of exon 3, 4 and 5 | KO* |
| 159 | | | | | | |
| 167 | 2 | NHEJ | homozygous | 1265 bp deletion | Deletion of exon 5 and 72 | KO |
| | | | | | bp of exon 6 | |
| 166-1 | 1 | NHEJ | biallelic | 24 bp deletion | Removal of start codon in exon 3 | KO |
| | | | | 27 bp deletion | Disruption of start codon in exon 3 | |
| | | | | 362 bp deletion + 5 bp | Deletion of exon 3 | |
| 166-2 | 1 | NHEJ | biallelic | 6 bp insertion + 2 bp | Addition of 6 bp before start codon in | CD1D^{ko/+} |
| | | | | mismatch | exon 3 | |
| | | | | 1598 bp deletion | Removal of start codon in exon 3 | |
| | | | | | and deletion of exons 4, 5 | |
| 166-3 | 1 | NHEJ | biallelic | 1 bp insertion | Addition of G/T in exon 3 before | CD1D^{+/+} |
| | | | | | start codon in exon 3 | |
| 166-4 | 1 | NHEJ | homozygous | 1 bp insertion | Addition of A in exon 3 before start | CD1D^{+/+} |
| | | | | | codon in exon 3 | |

*KO, knock-out

### Efficiency of CRISPR/Cas9 System in Porcine Zygotes

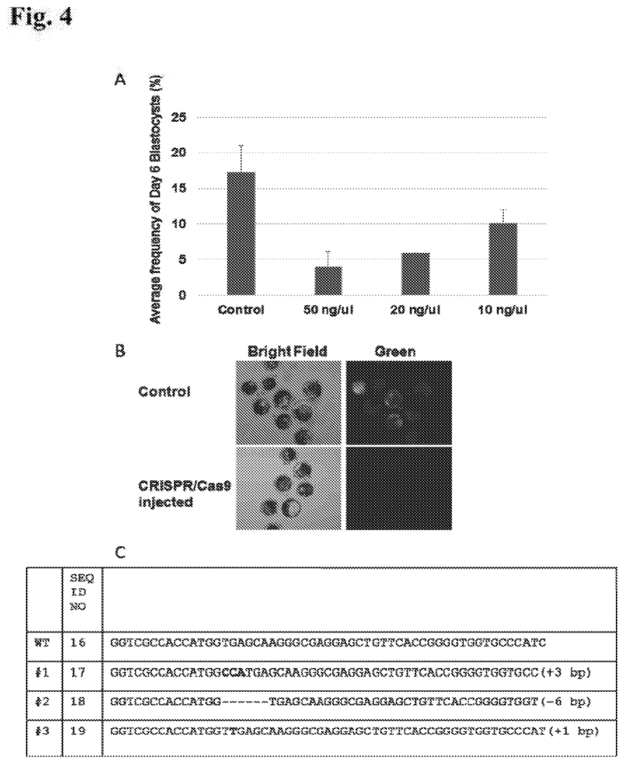

Based on targeted disruption of CD163 and CD1D in somatic cells using the CRISPR/Cas9 system, this approach was applied to porcine embryogenesis. First, the effectiveness of the CRISPR/Cas9 system in developing embryos was tested. CRISPR/Cas9 system targeting eGFP was introduced into zygotes fertilized with semen from a boar heterozygous for the eGFP transgene. After the injection, subsequent embryos expressing eGFP were monitored. Various concentrations of the CRISPR/Cas9 system were tested and cytotoxicity of the delivered CRISPR/Cas9 system was observed (FIG. 4, panel A); embryo development after CRISPR/Cas9 injection was lower compared to control. However, all the concentrations of CRISPR/Cas9 that were examined were effective in generating modification of eGFP because no embryos with eGFP expression were found in the CRISPR/Cas9-injected group (FIG. 4, panel B); of the noninjected control embryos 67.7% were green, indicating expression of eGFP. When individual blastocysts were genotyped, it was possible to identify small mutations near the CRISPR binding sites (FIG. 4, panel C). Based on the toxicity and effectiveness, 10 ng/μl of gRNA and Cas9 mRNA were used for the following experiments.

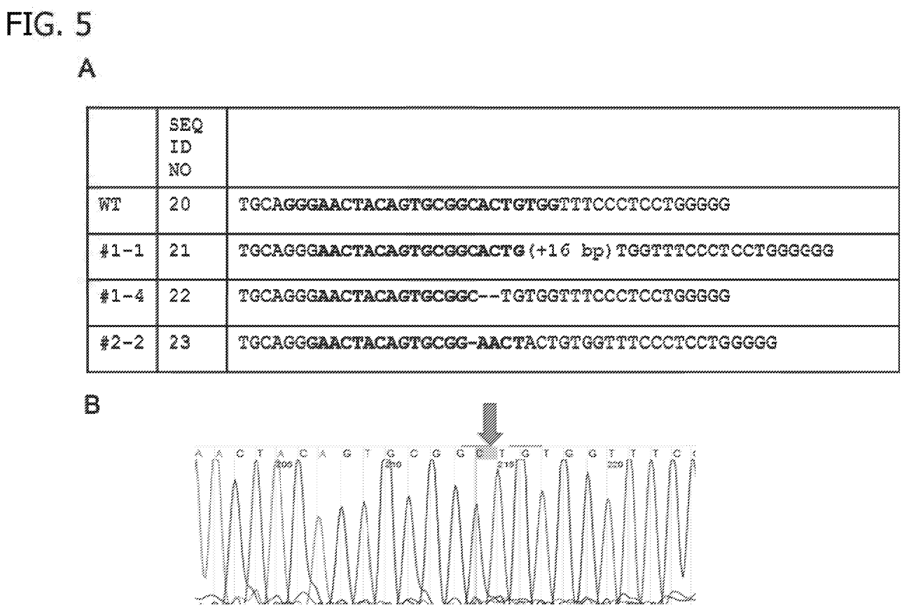

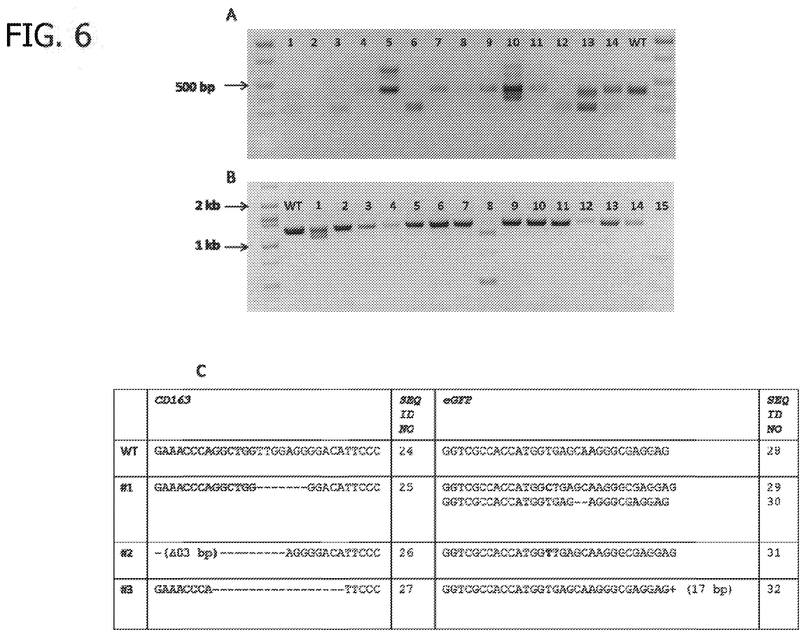

When CRISPR/Cas9 components designed to target CD163 were introduced into presumptive zygotes, targeted editing of the genes in the subsequent blastocysts was observed. When individual blastocysts were genotyped for mutation of CD163, specific mutations were found in all the embryos (100% GE efficiency). More importantly, while embryos could be found with homozygous or biallelic modifications (8/18 and 3/18, respectively) (FIG. 5), mosaic (monoallelic modifications) genotypes were also detected (4/18 embryos). Some embryos (8/10) from the pool were injected with 2 ng/μl Cas9 and 10 ng/μl CRISPR and no difference was found in the efficiency of mutagenesis. Next, based on the in vitro results, two CRISPRs representing different gRNA were introduced to disrupt CD163 or CD1D during embryogenesis to induce a specific deletion of the target genes. As a result, it was possible to successfully induce a designed deletion of CD163 and CD1D by introducing two guides. A designed deletion is defined as a deletion that removes the genomic sequence between the two guides introduced. Among the embryos that received two CRISPRs targeting CD163, all but one embryo resulted in a targeted modification of CD163. In addition, 5/13 embryos were found to have a designed deletion on CD163 (FIG. 6, panel A) and 10/13 embryos appeared to have modification of CD163 in either homozygous or biallelic fashion. Targeting CD1D with two CRISPRs was also effective because all the embryos (23/23) showed a modification of CD1D. However, the designed deletion of CD1D could only be found in two embryos (2/23) (FIG. 6, panel B). Five of twenty-three embryos possessing mosaic genotypes were also found, but the rest of embryos had either homozygous or biallelic modification of CD1D. Finally, whether multiple genes can be targeted by the CRISPR/Cas9 system within the same embryo was tested. For this purpose, targeting both CD163 and eGFP was performed in the zygotes that were fertilized with heterozygous eGFP semen. When blastocysts from the injected embryos were genotyped for CD163 and eGFP, it was found that found that CD163 and eGFP were successfully targeted during embryogenesis. Sequencing results demonstrated that multiple genes can be targeted by introducing multiple CRISPRs with Cas9 (FIG. 6, panel C).

Production of CD163 and CD1D Mutants from CRISPR/Cas9-Injected Zygotes

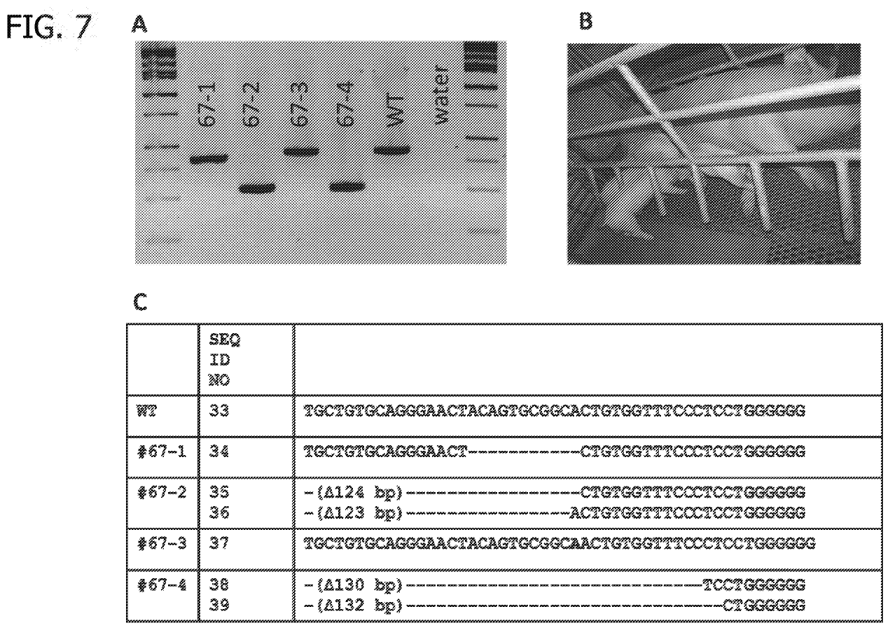

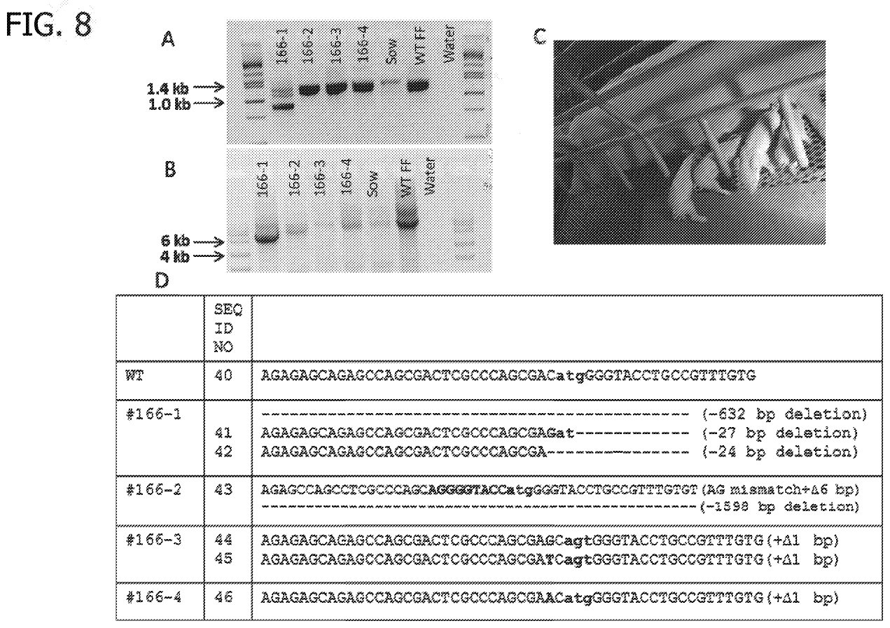

Based on the success from the previous in vitro study, some CRISPR/Cas9-injected zygotes were produced and 46-55 blastocysts were transferred per recipient (because this number has been shown to be effective in producing pigs from the in vitro derived embryos). Four embryo transfers were performed, two each for CD163 and CD1D, and a pregnancy for each modification was obtained. Four healthy piglets were produced carrying modifications on CD163 (Table 9). All the piglets, litter 67 from recipient sow ID 0083 showed either homozygous or biallelic modification of CD163 (FIG. 7). Two piglets showed the designed deletion of CD163 by the two CRISPRs delivered. All the piglets were healthy. For CD1D, one pregnancy also produced four piglets (litter 166 from recipient sow identification no. 0165): one female and three males (Table 10). One piglet (166-1) did carry a mosaic mutation of CD1D, including a 362 bp deletion that completely removed exon 3 that contains the start codon (FIG. 8). One piglet contained a 6 bp insertion with a 2 bp mismatch on one allele with a large deletion on the other allele. Two additional piglets had a biallelic single bp insertion. There were no mosaic mutations detected for CD163.

### Discussion

An increase in efficiency of GE pig production can have a wide impact by providing more GE pigs for agriculture and biomedicine. The data described above show that by using the CRISPR/Cas9 system, GE pigs with specific mutations can be produced at a high efficiency. The CRISPR/Cas9 system was successfully applied to edit genes in both somatic cells and in preimplantation embryos.

When the CRISPR/Cas9 system was introduced into somatic cells, it successfully induced targeted disruption of the target genes by NHEJ but did not increase the ability to target by HR. Targeting efficiency of individual CRISPR/Cas9 in somatic cells was variable, which indicated that the design of the guide can affect the targeting efficiency. Specifically, it was not possible to find targeted modification of CD1D when CRISPR 5350 and Cas9 were introduced into somatic cells. This suggests that it could be beneficial to design multiple gRNAs and validate their efficiencies prior to producing pigs. A reason for the lack of HR-directed repair with the presence of donor DNA is still unclear. After screening 886 colonies (both CD163 and CD1D) transfected with CRISPR and donor DNA, only one colony had evidence for a partial HR event. The results demonstrated that the CRISPR/Cas9 system worked with introduced donor DNA to cause unexpected large deletions on the target genes but did not increase HR efficiency for these two particular targeting vectors. However, a specific mechanism for the large deletion observation is not known. Previous reports from our group suggested that a donor DNA can be effectively used with a ZFN to induce HR-directed repair. Similarly, an increase in the targeting efficiency was seen when donor DNA was used with CRISPR/Cas9 system, but complete HR directed repair was not observed. In a previous study using ZFN, it was observed that targeted modification can occur through a combination of HR and NHEJ because a partial recombination was found of the introduced donor DNA after induced DSBs by the ZFN. One explanation might be that HR and NHEJ pathways are not independent but can act together to complete the repair process after DSBs induced by homing endonucleases. Higher concentrations of CRISPRs might improve targeting efficiency in somatic cells although no statistical difference was found in these experimental results. This may suggest that CRISPR is a limiting factor in CRISPR/Cas9 system, but further validation is needed. Targeted cells were successfully used to produce GE pigs through SCNT, indicating the application of CRISPR/Cas9 does not affect the ability of the cells to be cloned. A few piglets were euthanized because of health issues; however, this is not uncommon in SCNT-derived piglets.

When the CRISPR/Cas9 system was introduced into developing embryos by zygote injection, nearly 100% of embryos and pigs contained an INDEL in the targeted gene, demonstrating that the technology is very effective during embryogenesis. The efficiency observed during this study surpasses frequencies reported in other studies utilizing homing endonucleases during embryogenesis. A decrease in the number of embryos reaching the blastocyst stage suggested that the concentration of CRISPR/Cas9 introduced in this study may be toxic to embryos. Further optimization of the delivery system may increase survivability of embryos and thus improve the overall efficiency of the process. The nearly 100% mutagenesis rate observed here was different from a previous report in CRISPR/Cas9-mediated knockout in pigs; however, the difference in efficiency between the studies could be a combination of the guide and target that was selected. In the present study, lower concentrations of CRISPR/Cas9 (10 ng/μl each) were effective in generating mutations in developing embryos and producing GE pigs. The concentration is lower than previously reported in pig zygotes (125 ng/μl of Cas9 and 12.5 ng/μl of CRISPR). The lower concentration of CRISPR/Cas9 components could be beneficial to developing embryos because introducing excess amounts of nucleic acid into developing embryos can be toxic. Some mosaic genotypes were seen in CRISPR/Cas9-injected embryos from the in vitro assays; however, only one piglet produced through the approach had a mosaic genotype. Potentially, an injection with CRISPR/Cas9 components may be more effective than introduction of other homing endonucleases because the mosaic genotype was considered to be a main hurdle of using the CRISPR/Cas9 system in zygotes. Another benefit of using the CRISPR/Cas9 system demonstrated by the present results is that no CD163 knockout pigs produced from IVF-derived zygotes injected with CRISPR/Cas9 system were lost, whereas a few piglets resulting from SCNT were euthanized after a few days. This suggests that the technology could not only bypass the need of SCNT in generating knockout pigs but could also overcome the common health issues associated with SCNT. Now that injection of CRISPR/Cas9 mRNA into zygotes has been optimized, future experiments will include coinjection of donor DNA as well.

The present study demonstrates that introducing two CRISPRs with Cas9 in zygotes can induce chromosomal deletions in developing embryos and produce pigs with an intended deletion, that is, specific deletion between the two CRISPR guides. This designed deletion can be beneficial because it is possible to specify the size of the deletion rather than relying on random events caused by NHEJ. Specifically, if there is insertion/deletion of nucleotides in a multiple of three caused by a homing endonuclease, the mutation may rather result in a hypomorphic mutation because no frame shift would occur. However, by introducing two CRISPRs, it is possible to cause larger deletions that will have a higher chance of generating non-functional protein. Interestingly, CD1D CRISPRs were designed across a greater area in the genome than CD163; there was a 124 bp distance between CD163 CRISPR 10 and 131 while there was a distance of 550 bp between CRISPR 4800 and 5350 for CD1D. The longer distance between CRISPRs was not very effective in generating a deletion as shown in the study. However, because the present study included only limited number of observations and there is a need to consider the efficacy of individual CRISPRs, which is not addressed here, further study is need to verify the relationship between the distance between CRISPRs and probability of causing intended deletions.

The CRISPR/Cas9 system was also effective in targeting two genes simultaneously within the same embryo with the only extra step being the introduction of one additional CRISPR with crRNA. This illustrates the ease of disrupting multiples genes compared to other homing endonucleases. These results suggest that this technology may be used to target gene clusters or gene families that may have a compensatory effect, thus proving difficult to determine the role of individual genes unless all the genes are disrupted. The results demonstrate that CRISPR/Cas9 technology can be applied in generating GE pigs by increasing the efficiency of gene targeting in somatic cells and by direct zygote injection.

### Example 2: Increased Resistance to PRRSV in Swine Having a Modified Chromosomal Sequence in a Gene Encoding a CD163 Protein

Porcine Reproductive and Respiratory Syndrome Virus (PRRSV) has ravaged the swine industry over the last quarter of a century. It is shown in the present example that CD163 null animals show no clinical signs of infection, lung pathology, viremia or antibody production that are all hallmarks of PRRSV infection. Not only has a PRRSV entry mediator been confirmed; but if similarly created animals were allowed to enter the food supply, then a strategy to prevent significant economic losses and animal suffering has been described.

### Materials and Methods

### Genotyping

Genotyping was based on both DNA sequencing and mRNA sequencing. The sire's genotype had an 11 bp deletion in one allele that when translated predicted 45 amino acids into domain 5, resulting in a premature stop codon at amino acid 64. In the other allele there was a 2 bp addition in exon 7 and 377 bp deletion in intron before exon 7, that when translated predicted the first 49 amino acids of domain 5, resulting in a premature stop code at amino acid 85. One sow had a 7 bp addition in one allele that when translated predicted the first 48 amino acids of domain 5, resulting in a premature stop codon at amino acid 70. The other allele was uncharacterized (A), as there was no band from exon 7 by either PCR or long range 6.3 kb PCR. The other 3 sows were clones and had a 129 bp deletion in exon 7 that is predicted to result in a deletion of 43 amino acids from domain 5. The other allele was uncharacterized (B).

### Growth of PRRSV in Culture and Production of Virus Inoculum for the Infection of Pigs are Covered Under Approved IBC Application 973

A type strain of PRRSV, isolate NVSL 97-7895 (GenBank=AF325691 2001-02-11), was grown as described in approved IBC protocol 973. This laboratory isolate has been used in experimental studies for about 20 years (Ladinig et al., 2015).A second isolate was used for the 2^{nd} trial, KS06-72109 as described previously (Prather et al., 2013).

Infection of Pigs with PRRSV

A standardized infection protocol for PRRSV was used for the infection of pigs. Three week old piglets were inoculated with approximately 10⁴ TCID50 of PRRS virus which was administered by intramuscular (IM) and intranasal (IN) routes. Pigs were monitored daily and those exhibiting symptoms of illness are treated according to the recommendations of the CMG veterinarians. Pigs that show severe distress and are in danger of succumbing to infection are humanely euthanized and samples collected. Staff and veterinarians were blind to the genetic status of the pigs to eliminate bias in evaluation or treatment. PRRSV is present in body fluids during infection; therefore, blood samples were collected and stored at −80° C. until measured to determine the amount or degree of viremia in each pig. At the end of the experiment, pigs were weighed and humanely euthanized, and tissues collected and fixed in 10% buffered formalin, embedded in paraffin, and processed for histopathology by a board-certified pathologist.

### Phenotype Scoring of the Challenged Pigs

The phenotype of the pigs was blindly scored daily as follows: What is the attitude of the pig? Attitude Score: 0: BAR, 1: QAR, 2: Slightly depressed, 3: Depressed, 4: Moribund. What is the body condition of the pig? Body Condition Score: 1: Emaciated, 2: Thin, 3: Ideal, 4: Fat, 5: Overfat/Obese. What is the rectal temperature of the pig? Normal Body Temperature 101.6-103.6° F. (Fever considered≥104° F.). Is there any lameness (grade)? What limb? Evaluate limbs for joint swelling and hoof lesions (check bottom and sides of hoof). Lameness Score: 1: No lameness, 2: Slightly uneven when walking, appears stiff in some joints but no lameness, 3: Mild lameness, slight limp while walking, 4: Moderate lameness, obvious limp including toe touching lame, 5: Severe lameness, non-weight bearing on limb, needs encouragement to stand/walk. Is there any respiratory difficulty (grade)? Is there open mouth breathing? Is there any nasal discharge (discharge color, discharge amount: mild/moderate/severe)? Have you noticed the animal coughing? Is there any ocular discharge? Respiratory Score: 0: Normal, 1: mild dyspnea and/or tachypnea when stressed (when handled), 2: mild dyspnea and/or tachypnea when at rest, 3: moderate dyspnea and/or tachypnea when stressed (when handled), 4: moderate dyspnea and/or tachypnea when at rest, 5: severe dyspnea and/or tachypnea when stressed (when handled), 6: severe dyspnea and/or tachypnea when at rest. Is there evidence of diarrhea (grade) or vomiting? Is there any blood or mucus? Diarrhea Score: 0: no feces noted, 1: normal stool, 2: soft stool but formed (soft serve yogurt consistency, creates cow patty), 3: liquid diarrhea of brown/tan coloration with particulate fecal material, 4: liquid diarrhea of brown/tan coloration without particulate fecal material, 5: liquid diarrhea appearing similar to water.

This scoring system was developed by Dr. Megan Niederwerder at KSU and is based on the following publications (Halbur et al., 1995; Merck; Miao et al., 2009; Patience and Thacker, 1989; Winckler and Willen, 2001). Scores and temperatures were analyzed by using ANOVA separated based on genotypes as treatments.

### Measurement of PRRSV Viremia

Viremia was determined via two approaches. Virus titration was performed by adding serial 1:10 dilutions of serum to confluent MARC-145 cells in a 96 well-plate, Serum was diluted in Eagle's minimum essential medium supplemented with 8% fetal bovine serum, penicillin, streptomycin, and amphotericin B as previously described (Prather et al., 2013). The cells were examined after 4 days of incubation for the presence of a cytopathic effect by using microscope. The highest dilution showing a cytopathic effect was scored as the titration endpoint. Total RNA was isolated from serum by using the Life Technologies MagMAX-96 viral RNA isolation kit for measuring viral nucleic acid. The reverse transcription polymerase chain reaction was performed by using the EZ-PRRSV MPX 4.0 kit from Tetracore on a CFX-96 real-time PCR system (Bio-Rad) according to the manufacturer's instructions. Each reaction (25 μl) contained RNA from 5.8 μl of serum. The standard curve was constructed by preparing serial dilutions of an RNA control supplied in the kit (Tetracore). The number of templates per PCR are reported.

### SIGLEC1 and CD163 Staining of PAM Cells

Porcine alveolar macrophages (PAMs) were collected by excising the lungs and filling them with ˜100 ml cold phosphate buffered saline. After recovering the phosphate buffered saline wash cells were pelleted and resuspended in 5 ml cold phosphate buffered saline and stored on ice. Approximately 10⁷PAMs were incubated in 5 ml of the various antibodies (anti-porcine CD169 (clone 3B11/11; AbD Serotec); anti-porcine CD163 (clone 2A10/11; AbD Serotec)) diluted in phosphate buffered saline with 5% fetal bovine serum and 0.1% sodium azide for 30 minutes on ice. Cells were washed and resuspended in 1/100 dilution of fluorescein isothiocyanate (FITC)-conjugated to goat anti-mouse IgG (life Technologies) diluted in staining buffer and incubated for 30 minutes on ice. At least 10⁴ cells were analyzed by using a FACSCalibur flow cytometer and Cell Quest software (Becton Dickinson).

### Measurement of PRRSV Specific Ig

To measure PRRSV-specific Ig recombinant PRRSV N protein was expressed in bacteria (Trible et al., 2012) and conjugated to magnetic Luminex beads by using a kit (Luminex Corporation). The N protein-coupled beads were diluted in phosphate buffered saline containing 10% goat serum to 2,500 beads/50 μl and placed into the wells of a 96-well round-bottomed polystyrene plate. Serum was diluted 1:400 in phosphate buffered saline containing 10% goat serum and 50 μl was added in duplicate wells and incubated for 30 minutes with gentle shaking at room temperature. Next the plate was washed (3×) with phosphate buffered saline containing 10% goat serum and 50 μl of biotin-SP-conjugated affinity-purified goat anti-swine secondary antibody (IgG, Jackson ImmunoResearch) or biotin-labeled affinity purified goat anti-swine IgM (KPL) diluted to 2 μg/ml in phosphate buffered saline containing 10% goat serum was added. The plates were washed (3×) after 30 minutes of incubation and then 50 μl of streptavidin-conjugated phycoerythrin (2 μg/ml (Moss, Inc.) in phosphate buffered saline containing 10% goat serum) was added. The plates were washed 30 minutes later and microspheres were resuspended in 100 μl of phosphate buffered saline containing 10% goat serum an analyzed by using the MAGPIX and the Luminex xPONENT 4.2 software. Mean fluorescence intensity (MFI) is reported,

### Results

Mutations in CD163 were created by using the CRISPR/Cas9 technology as described above in Example 1. Several founder animals were produced from zygote injection and from somatic cell nuclear transfer. Some of these founders were mated creating offspring to study. A single founder male was mated to females with two genotypes. The founder male (67-1) possessed an 11 bp deletion in exon 7 on one allele and a 2 bp addition in exon 7 (and 377 bp deletion in the preceding intron) of the other allele and was predicted to be a null animal (CD163^{-/-}). One founder female (65-1) had a 7 bp addition in exon 7 in one allele and an uncharacterized corresponding allele and was thus predicted to be heterozygous for the knockout (CD/63^{-/-}). A second founder female genotype (3 animals that were clones) contained an as yet uncharacterized allele and an allele with a 129 bp deletion in exon 7. This deletion is predicted to result in a deletion of 43 amino acids in domain 5. Matings between these animals resulted in all piglets inheriting a null allele from the boar and either the 43 amino acid deletion or one of the uncharacterized alleles from the sows. In addition to the wild type piglets that served as positive controls for the viral challenge, this produced 4 additional genotypes (Table 13).

TABLE 13
Genotypes tested for resistance to
PRRSV challenge (NVSL and KS06 strains)

| | | Resistance to PRRSV Challenge | |
|---|---|---|---|
| Alleles | | as Measured by Viremia | |
| Paternal | Maternal | NVSL | KS06 |
| Null | Null | Resistant | N/A |
| Null | Δ43 Amino Acids | N/A | Resistant |
| Null | Uncharacterized A | Susceptible | N/A |
| Null | Uncharacterized B | Susceptible | Susceptible |
| Wild Type | Wild Type | Susceptible | Susceptible |

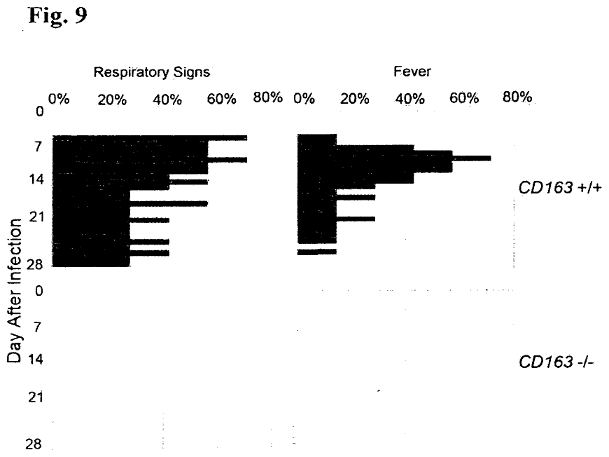

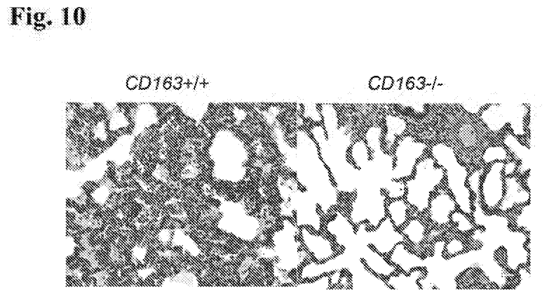

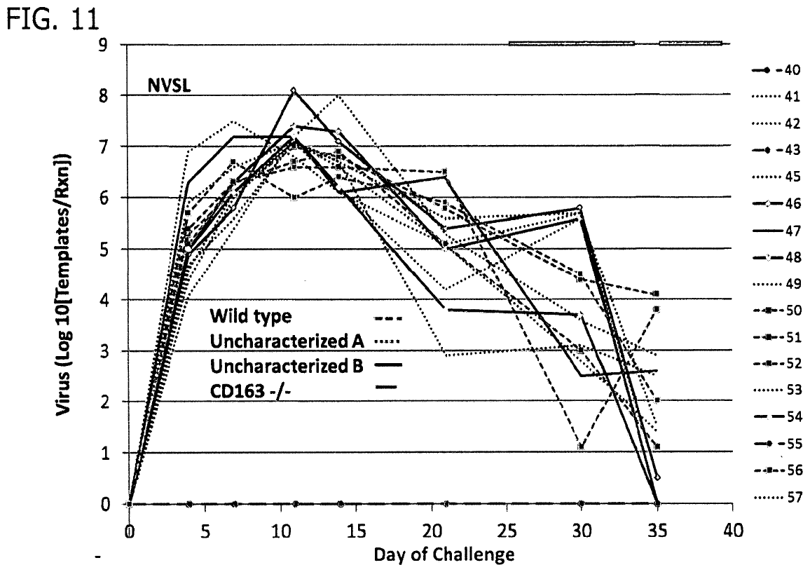

At weaning, gene edited piglets and wild type age-matched piglets were transported to Kansas State University for a PRRSV challenge. A PRRSV challenge was conducted as previously described (Prather et al., 2013). Piglets, at three weeks of age, were brought into the challenge facility and maintained as a single group. All experiments were initiated after approval of institutional animal use and biosafety committees. After acclimation, the pigs were challenged with a PRRSV isolate, NVSL 97-7895 (Ladinig et al., 2015), propagated on MARC-145 cells (Kim et al., 1993). Pigs were challenged with approximately 10⁵ TCID₅₀ of virus. One-half of the inoculum was delivered intramuscularly and the remaining delivered intranasally. All infected pigs were maintained as a single group, which allowed the continuous exposure of virus from infected pen mates. Blood samples were collected at various days up to 35 days after infection and at termination, day 35. Pigs were necropsied and tissues fixed in 10% buffered formalin, embedded in paraffin and processed for histopathology. PRRSV associated clinical signs recorded during the course of the infection included respiratory distress, inappetence, lethargy and fever. The results for clinical signs over the study period are summarized in FIG. 9. As expected, the wild-type Wild Type (CD163+/+) pigs showed early signs of PRRSV infection, which peaked at between days 5 and 14 and persisted in the group during the remainder of the study. The percentage of febrile pigs peaked on about day 10. In contrast, Null (CD163−/−) piglets showed no evidence of clinical signs over the entire study period. The respiratory signs during acute PRRSV infection are reflected in significant histopathological changes in the lung (Table 14). The infection of the wild type pigs showed histopathology consistent with PRRS including interstitial edema with the infiltration of mononuclear cells (FIG. 10). In contrast there was no evidence for pulmonary changes in the Null (CD163−/−) pigs. The sample size for the various genotypes is small; nevertheless the mean scores were 3.85 (n=7) for the wild type, 1.75 (n=4) for the uncharacterized A, 1.33 (n=3) for the uncharacterized B, and 0 (n=3) and for the null (CD163-14

TABLE 14
Microscopic Lung evaluation

| Pig | Genotype | Description | Score |
|---|---|---|---|
| 41 | Wild Type | 100% congestion. Multifocal areas of edema. Infiltration of | 3 |
| | | moderate numbers of lymphocytes and macrophages | |
| 42 | Wild Type | 100% congestion. Multifocal areas of edema. Infiltration of | 3 |
| | | moderate numbers of lymphocytes and macrophages | |
| 47 | Wild Type | 75% multifocal infiltration with mononuclear cells and | 2 |
| | | mild edema | |
| 50 | Wild Type | 75% multifocal infiltration of mononuclear cells within | 3 |
| | | alveolar spaces and around small blood vessels perivascular | |
| | | edema | |
| 51 | Wild Type | 25% atelectasis with moderate infiltration of mononuclear | 1 |
| | | cells | |
| 52 | Wild Type | 10% of alveolar spaces collapsed with infiltration of small | 1 |
| | | numbers of mononuclear cells | |
| 56 | Wild Type | 100% diffuse moderate interstitial infiltration of | 4 |
| | | mononuclear cells. Interalveolar septae moderately | |
| | | thickened by hemorrhage and edema. | |
| 45 | Uncharacterized A | 75% multifocal infiltrates of mononuclear cells, especially | 3 |
| | | around bronchi, blood vessels, subpleural spaces, and | |
| | | interalveolar septae. | |
| 49 | Uncharacterized A | 75% multifocal moderate to large infiltration of | 2 |
| | | mononuclear cells. Some vessels with mild edema. | |
| 53 | Uncharacterized A | 10% multifocal small infiltration of mononuclear cells | 1 |
| 57 | Uncharacterized A | 15% infiltration of mononuclear cells | 1 |
| 46 | Uncharacterized B | Moderate interstitial pneumonia | 2 |
| 48 | Uncharacterized B | Perivascular edema and infiltration of mononuclear cells | 2 |
| | | around small and medium sized vessels and around | |
| | | interalveolar septae | |
| 54 | Uncharacterized B | No changes | 0 |
| 40 | Null | No changes | 0 |
| 43 | Null | No changes | 0 |
| 55 | Null | No changes | 0 |

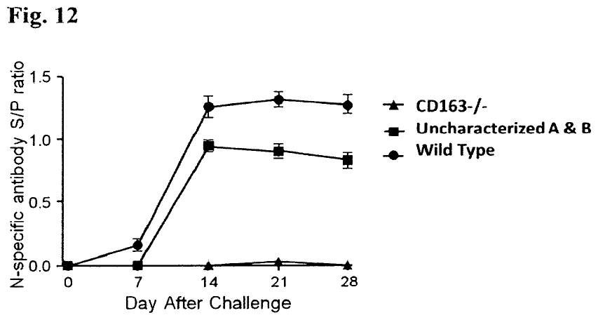

Peak clinical signs correlated with the levels of PRRSV in the blood. The measurement of viral nucleic acid was performed by isolation of total RNA from serum followed by amplification of PRRSV RNA by using a commercial reverse transcriptase real-time PRRSV PCR test (Tetracore, Rockville, Md.). A standard curve was generated by preparing serial dilutions of a PRRSV RNA control, supplied in the RT-PCR kit and results were standardized as the number templates per 50 μl PCR reaction. The PRRSV isolate followed the course for PRRSV viremia in the wild type CD163^{+/+} pigs (FIG. 11). Viremia was apparent at day four, reached a peak at day 11 and declined until the end of the study. In contrast viral RNA was not detected in the CD163^{-/-} pigs at any time point during the study period. Consistent with the viremia, antibody production by the null and uncharacterized allele pigs was detectable by 14 and increased to day 28. There was no antibody production in the null animals (FIG. 12). Together, these data show that wild type pigs support PRRSV replication with the production of clinical signs consistent with PRRS. In contrast, the knockout pigs produced no viremia and no clinical signs, even though pigs were inoculated and constantly exposed to infected pen mates.

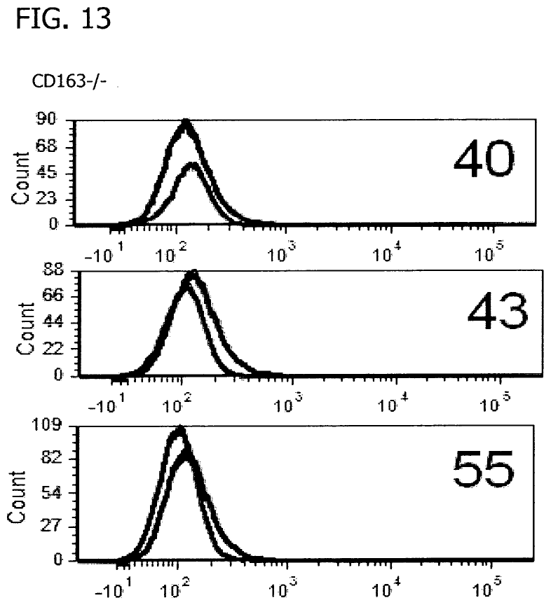

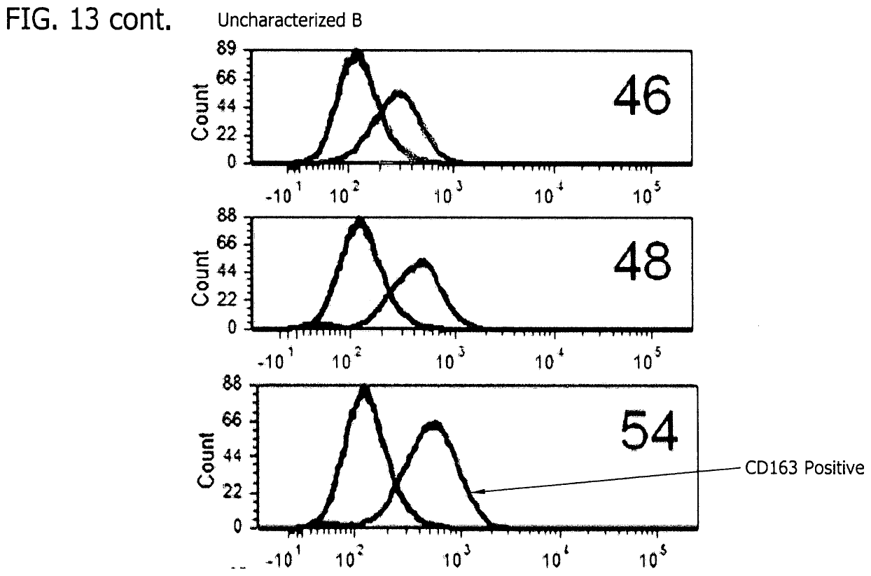

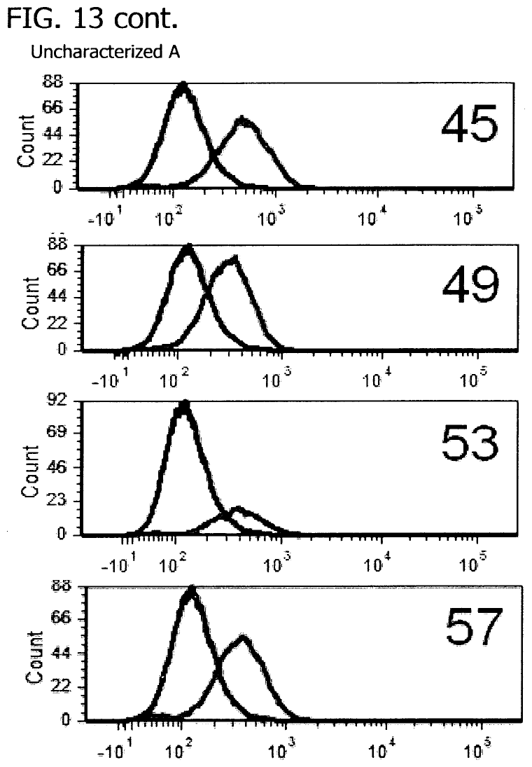

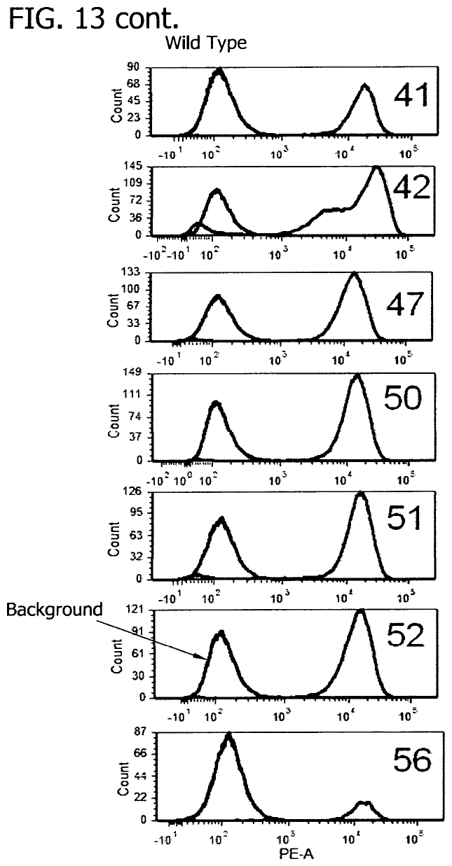

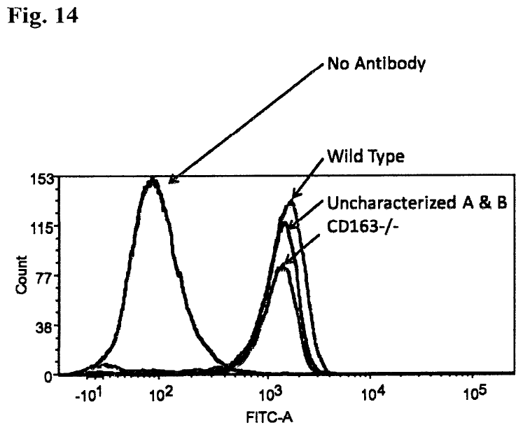

At the end of the study, porcine alveolar macrophages were removed by lung lavage and stained for surface expression of SIGLEC1 (CD169, clone 3B11/11) and CD163 (clone 2A10/11), as described previously (Prather et al., 2013). Relatively high levels of CD163 expression were detected on CD163+/+ wild type animals (FIG. 13). In contrast, CD163−/− pigs showed only background levels of anti-CD163 staining, thus confirming the knockout phenotype. Expression levels for another macrophage marker CD169 were similar for both wild type and knockout pigs (FIG. 14). Other macrophage surface markers, including MHC II and CD172 were the same for both genotypes (data not shown).

While the sample size was small the wild type pigs tended to gain less weight over the course of the experiment (average daily gain 0.81 kg±0.33, n=7) versus the pigs of the other three genotypes (uncharacterized A 1.32 kg±0.17, n=4; uncharacterized B 1.20 kg±0.16, n=3; null 1.21 kg±0.16, n=3).

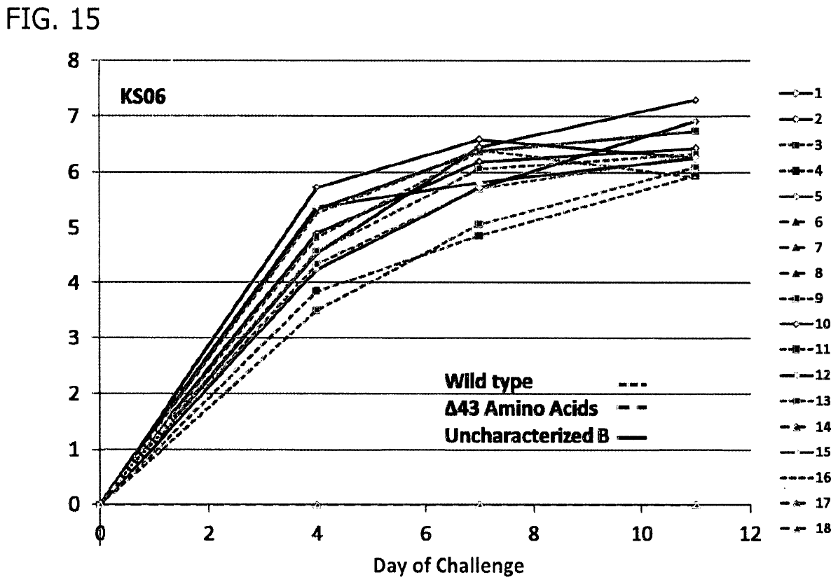

In a second trial 6 wild type, 6 443 amino acids, and 6 pigs with an uncharacterized allele (B) were challenged as described above, except KS06-72109 was used to inoculate the piglets. Similar to the NVSL data the wild type and uncharacterized B piglets developed viremia. However, in the 443 amino acid pigs the KS06 did not result in viremia (FIG. 15; Table 8).

### Implications and Conclusion

The most clinically relevant disease to the swine industry is PRRS. While vaccination programs have been successful to prevent or ameliorate most swine pathogens, the PRRSV has proven to be more of a challenge. Here CD163 is identified as an entry mediator for this viral strain. The founder boar was created by injection of CRISPR/Cas9 into zygotes (Whitworth et al., 2014) and thus there is no transgene. Additionally one of the alleles from the sow (also created by using CRISPR/Cas9) does not contain a transgene. Thus piglet #40 carries a 7 bp addition in one allele and a 11 bp deletion in the other allele, but no transgene. These virus-resistance alleles of CD163 represent minor genome edits considering that the swine genome is about 2.8 billion bp (Groenen et al., 2012). If similarly created animals were introduced into the food supply, significant economic losses could be prevented.

### Example 3: Increased Resistance to Genotype 1 Porcine Reproductive and PRRS Viruses in Swine with CD163 SRCR Domain 5 Replaced with Human CD163-Like Homology SRCR Domain 8

CD163 is considered the principal receptor for porcine reproductive and respiratory syndrome virus (PRRSV). In this study, pigs were genetically edited (GE) to possess one of the following genotypes: complete knock out (KO) of CD163, deletions within CD163 scavenger receptor cysteine-rich (SRCR) domain 5, or replacement (domain swap) of SRCR domain 5 with a synthesized exon encoding a homolog of human CD163-like (hCD163L1) SRCR 8 domain. Immunophenotyping of porcine alveolar macrophages (PAMs) showed that pigs with the KO or SRCR domain 5 deletions did not express CD163 and PAMs did not support PRRSV infection. PAMs from pigs that possessed the hCD163L1 domain 8 homolog expressed CD163 and supported the replication of Type 2, but not Type 1 genotype viruses. Infection of CD163-modified pigs with representative Type 1 and Type 2 viruses produced similar results. Even though Type 1 and Type 2 viruses are considered genetically and phenotypically similar at several levels, including the requirement of CD163 as a receptor, the results demonstrate a distinct difference between PRRSV genotypes in the recognition of the CD163 molecule.

### Materials and Methods

### Genomic Modifications of the Porcine CD163 Gene

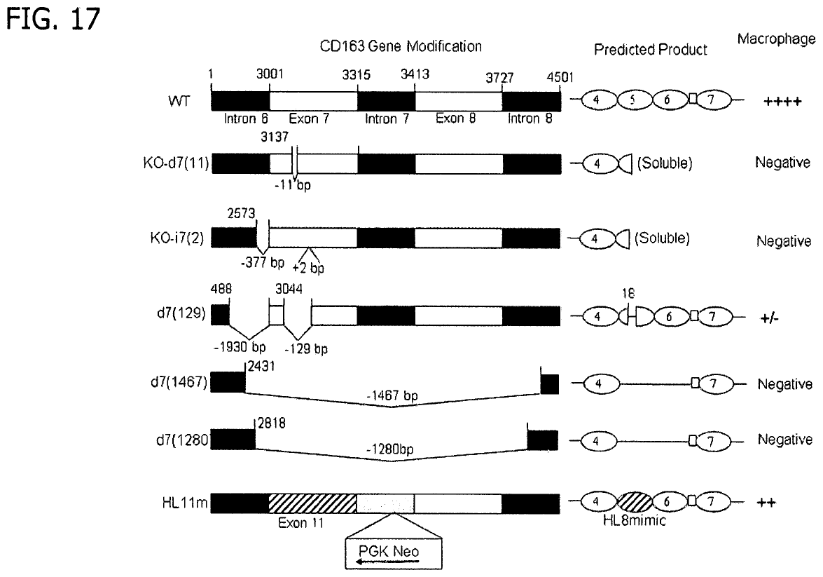

Experiments involving animals and viruses were performed in accordance with the Federation of Animal Science Societies Guide for the Care and Use of Agricultural Animals in Research and Teaching, the USDA Animal Welfare Act and Animal Welfare Regulations, and were approved by the Kansas State University and University of Missouri Institutional Animal Care and Use Committees and Institutional Biosafety Committees. Mutations in CD163 used in this study were created using the CRISPR/Cas9 technology as described hereinabove in the preceding examples. The mutations are diagrammed in FIG. 17. The diagrammed genomic region shown in FIG. 17 covers the sequence from intron 6 to intron 8 of the porcine CD163 gene. The introns and exons diagrammed in FIG. 17 are not drawn to scale. The predicted protein product is illustrated to the right of each genomic structure. Relative macrophage expression, as measured by the level of surface CD163 on PAMs, is shown on the far right of FIG. 17. The black regions indicate introns; the white regions indicate exons; the hatched region indicates hCD163L1 exon 11 mimic, the homolog of porcine exon 7; and the gray region indicates a synthesized intron with the PGK Neo construct as shown in FIG. 17.

The CD163 gene construct KO-d7(11) shown in FIG. 17 possesses an 11 base pair deletion in exon 7 from nucleotide 3,137 to nucleotide 3,147. The CD163 gene construct KO-17(2), possesses a 2 base pair insertion in exon 7 between nucleotides 3,149 and 3,150 as well as a 377 base pair deletion in the intron upstream of exon 7, from nucleotide 2,573 to nucleotide 2,949. These edits are predicted to cause frameshift mutations and premature stop codons, resulting in only partial translation of SRCR 5 and the KO phenotype. Three other mutations produced deletions in exon 7. The first, d7(129), has a 129 base pair deletion in exon 7 from nucleotide 3,044 to nucleotide 3,172. The d7(129) construct also has a deletion from nucleotide 488 to nucleotide 2,417 in exon 6, wherein the deleted sequence is replaced with a 12 bp insertion. The other two deletion constructs, d7(1467) and d7(1280), have complete deletions of exons 7 and 8 as illustrated in FIG. 17. d7(1467) has a 1467 base pair deletion from nucleotide 2,431 to nucleotide 3,897, and d7(1280) has a 1280 base pair deletion from nucleotide 2,818 to nucleotide 4,097. For these deletion constructs the other CD163 exons remained intact.

The last construct shown in FIG. 17, HL11m, was produced using a targeting event that deleted exon 7 and replaced it with a synthesized exon that encoded a homolog of SRCR 8 of the human CD163-like 1 protein (hCD163L1 domain 8 is encoded by hCD163L1 exon 11). The SRCR 8 peptide sequence was created by making 33 nucleotide changes in the porcine exon 7 sequence. A neomycin cassette was included in the synthesized exon to enable screening for the modification. SEQ ID NO: 118 provides the nucleotide sequence for the HL11m construct in the region corresponding to the same region in reference sequence SEQ ID NO: 47.

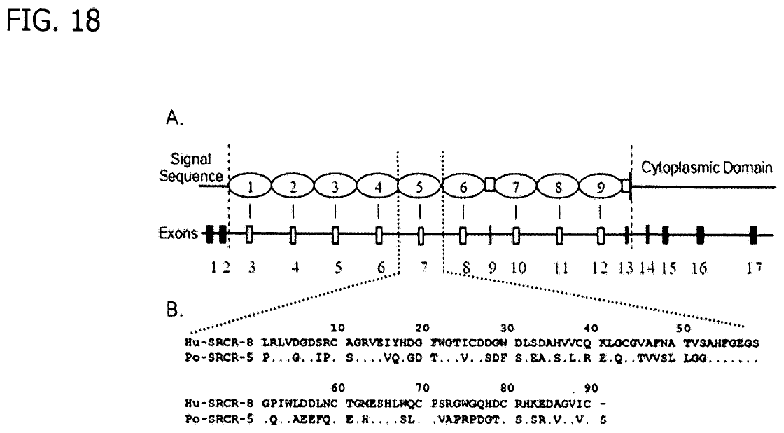

A diagram of the porcine CD163 protein and gene is provided FIG. 18. The CD163 protein SCRC (ovals) and PST (squares) domains along with the corresponding gene exons are shown in panel A of FIG. 18. A peptide sequence comparison for porcine CD163 SRCR 5 (SEQ ID NO: 120) and human CD163 SRCR 8 homolog (SEQ ID NO: 121) is shown in panel B of FIG. 18. The figure is based on GenBank accession numbers AJ311716 (pig CD163) and GQ397482 (hCD163-L1).

### Viruses

The panel of viruses used in this example is listed in Table 15. Isolates were propagated and titrated on MARC-145 cells (Kim et al., 1993). For titration, each virus was serially diluted 1:10 in MEM supplemented with 7% FBS, Pen-Strep (80 Units/ml and 80 μg/ml, respectively), 3 μg/ml FUNGIZONE (amphotericin B), and 25 mM HEPES. Diluted samples were added in quadruplicate to confluent MARC-145 cells in a 96 well plate to a final volume of 200 μl per well and incubated for four days at 37° C. in 5% CO2. The titration endpoint was identified as the last well with a cytopathic effect (CPE). The 50% tissue culture infectious dose (TCID₅₀/ml) was calculated using a method as previously described (Reed and Muench 1938).

TABLE 15
PRRSV isolates.

| | | Year | GenBank |
|---|---|---|---|
| Virus | Genotype | Isolated | No. |
| NVSL 97-7895 | 2 | 1997 | AY545985 |
| KS06-72109 | 2 | 2006 | KM252867 |
| P129 | 2 | 1995 | AF494042 |
| VR2332 | 2 | 1992 | AY150564 |
| CO90 | 2 | 2010 | KM035799 |
| AZ25 | 2 | 2010 | KM035800 |
| MLV-ResPRRS | 2 | NA* | AF066183 |
| KS62-06274 | 2 | 2006 | KM035798 |
| KS483 (SD23983) | 2 | 1992 | JX258843 |
| CO84 | 2 | 2010 | KM035802 |
| SD13-15 | 1 | 2013 | NA |
| Lelystad | 1 | 1991 | M96262 |
| 03-1059 | 1 | 2003 | NA |
| 03-1060 | 1 | 2003 | NA |
| SD01-08 | 1 | 2001 | DQ489311 |
| 4353PZ | 1 | 2003 | NA |

*NA, Not available

### In of Alveolar Macrophages

The preparation and infection of macrophages were performed as previously described (Gaudreault, et al., 2009 and Patton, et al., 2008). Lungs were removed from euthanized pigs and lavaged by pouring 100 ml of cold phosphate buffered saline (PBS) into the trachea. The tracheas were clamped and the lungs gently massaged. The alveolar contents were poured into 50 ml centrifuge tubes and stored on ice. Porcine alveolar macrophages (PAMs) were sedimented by centrifugation at 1200×g for 10 minutes at 4° C. The pellets were re-suspended and washed once in cold sterile PBS. The cell pellets were re-suspended in freezing medium containing 45% RPMI 1640, 45% fetal bovine serum (FBS), and 10% dimethylsulfoxide (DMSO) and stored in liquid nitrogen until use. Frozen cells were thawed on ice, counted and adjusted to 5×10⁵ cells/ml in media (RPMI 1640 supplemented with 10% FBS, PenStrep, and FUNGIZONE; RPMI-FBS). Approximately 10³ PAMs per well were added to 96 well plates and incubated overnight at 37° C. in 5% CO₂. The cells were gently washed to remove non-adherent cells. Serial 1:10 dilutions of virus were added to triplicate wells. After incubation overnight, the cells were washed with PBS and fixed for 10 minutes with 80% acetone. After drying, wells were stained with PRRSV N-protein specific SDOW-17 mAb (Rural Technologies Inc.) diluted 1:1000 in PBS with 1% fish gelatin (PBS-FG; Sigma Aldrich). After a 30 minute incubation at 37° C., the cells were washed with PBS and stained with ALEXAFLUOR 488-labeled anti-mouse IgG (Thermofisher Scientific) diluted 1:200 in PBS-FG. Plates were incubated for 30 minutes in the dark at 37° C., washed with PBS, and viewed under a fluorescence microscope. The 50% tissue culture infectious dose (TCID₅₀)/ml was calculated according to a method as previously described (Reed and Muench 1938).

### Measurement of CD169 and CD163 Surface Expression on PAMs

Staining for surface expression of CD169 and CD163 was performed as described previously (Prather et al., 2013). Approximately 1×10⁶ PAMs were placed in 12 mm×75 mm polystyrene flow cytometry (FACS) tubes and incubated for 15 minutes at room temp in 1 ml of PBS with 10% normal mouse serum to block Fc receptors. Cells were pelleted by centrifugation and re-suspended in 5 μl of FITC-conjugated mouse anti-porcine CD169 mAb (clone 3B11/11; AbD Serotec) and 5 μl of PE-conjugated mouse anti-porcine CD163 mAb (Clone: 2A10/11, AbD Serotec). After 30 minutes incubation the cells were washed twice with PBS containing 1% bovine serum albumin (BSA Fraction V; Hyclone) and immediately analyzed on a BD LSR Fortessa flow cytometer (BD Biosciences) with FCS Express 5 software (De Novo Software). A minimum of 10,000 cells were analyzed for each sample.

### Measurement of PRRS Viremia

RNA was isolated from 50 μl of serum using Ambion's MagMAX 96 Viral Isolation Kit (Applied Biosystems) according to the manufacturer's instructions. PRRSV RNA was quantified using EZ-PRRSV MPX 4.0 Real Time RT-PCR Target-Specific Reagents (Tetracore) performed according to the manufacturer's instructions. Each plate contained Tetracore Quantification Standards and Control Sets designed for use with the RT-PCR reagents. PCR was carried out on a CFX96 Touch Real-Time PCR Detection System (Bio-Rad) in a 96-well format using the recommended cycling parameters. The PCR assay results were reported as log₁₀ PRRSV RNA copy number per 50 μl reaction volume, which approximates the number of copies per ml of serum. The area under the curve (AUC) for viremia over time was calculated using GraphPad Prism version 6.00 for Windows.

### Measurement of PRRSV Antibody

The microsphere fluorescent immunoassay (FMIA) for the detection of antibodies against the PRRSV nucleocapsid (N) protein was performed as described previously (Stephenson et al., 2015). Recombinant PRRSV N protein was coupled to carboxylated Luminex MAGPLEX polystyrene microsphere beads according to the manufacturer's directions. For FMIA, approximately 2500 antigen-coated beads, suspended in 50 μL PBS with 10% goat serum (PBS-GS), were placed in each well of a 96-well polystyrene round bottom plate. Sera were diluted 1:400 in PBS-GS and 50 μl added to each well. The plate was wrapped in foil and incubated for 30 minutes at room temperature with gentle shaking. The plate was placed on a magnet and beads were washed three times with 190 μl of PBS-GS. For the detection of IgG, 50 μl of biotin-SP-conjugated affinity purified goat anti-swine secondary antibody (IgG, Jackson ImmunoResearch) was diluted to 2 μg/ml in PBS-GS and 100 μl added to each well. The plate was incubated at room temperature for 30 minutes and washed three times followed by the addition of 50 μl of streptavidin-conjugated phycoerythrin (2 μg/ml in PBS-GS; SAPE). After 30 minutes, the microspheres were washed, resuspended in 100 μl of PBS-GS, and analyzed using a MAGPIX instrument (LUMINEX) and LUMINEX xPONENT 4.2 software. The mean fluorescence intensity (MFI) was calculated on a minimum of 100 microsphere beads.

### Measurement of Haptoglobin (HP)

The amount of Hp in serum was measured using a porcine-specific Hp ELISA kit (Genway Biotech Inc.) and steps performed according to the manufacturer's instructions. Serum samples were diluted 1:10,000 in 1× diluent solution and pipetted in duplicate on a pre-coated anti-pig Hp 96 well ELISA plate, incubated at room temperature for 15 minutes, then washed three times. Anti-Hp-horseradish peroxidase (HRP) conjugate was added to each well and incubated in the dark at room temperature for 15 minutes. The plate was washed and 100 μl chromogen-substrate solution added to each well. After incubating in the dark for 10 minutes, 100₁1.1 of stop solution was added to each well. The plate was read at 450 nm on a Fluostar Omega filter-based microplate reader (BMG Labtech).

### Results

Phenotypic Properties of PAMs from CD163-Modified Pigs

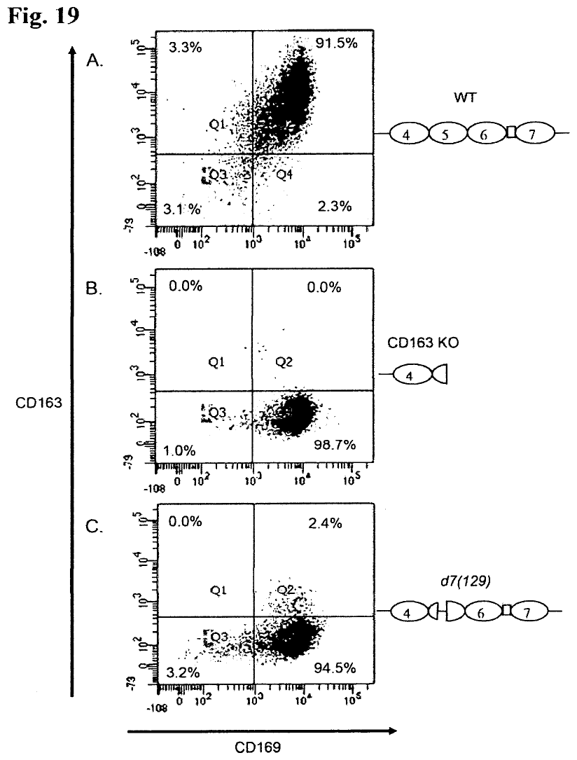

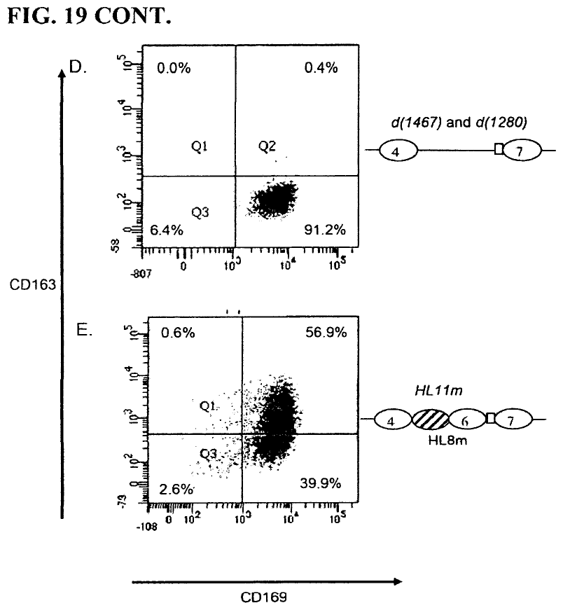

The forward and side scatter properties of cells in the lung lavage material were used to gate on the mononuclear subpopulation of cells. Representative CD169 and CD163 staining results for the different chromosomal modifications shown in FIG. 17 are presented in FIG. 19. In the representative example presented in panel A of FIG. 19, greater than 91% of PAMs from the WT pigs were positive for both CD169 and CD163. Results for 12 WT pigs used in this study showed a mean of 85+/−8% of double-positive cells. As shown in panel B of FIG. 19, PAMs from the CD163 KO pigs showed no evidence of CD163, but retained normal surface levels of CD169. Although it was predicted that the CD163 polypeptides derived from the d7(1467) and d7(1280) deletion genotypes should produce modified CD163 polypeptides anchored to the PAM surface, immunostaining results showed no surface expression of CD163 (see FIG. 19, panel D). Since MAb 2A10 recognizes an epitope located in the first three SRCR domains, the absence of detection was not the result of the deletion of an immunoreactive epitope. The d7(129) genotype was predicted to possess a 43 amino acid deletion in SRCR 5 (see FIG. 17). In the example presented in panel C of FIG. 19, only 2.4% of cells fell in the double-positive quadrant. The analysis of PAMs from nine d7(129) pigs used in this study showed percentages of double-positive cells ranging from 0% to 3.6% (mean=0.9%). The surface expression of CD169 remained similar to WT PAMs. For the purpose of this study, pigs possessing the KO, d7 (1467), d7 (1280), and d7 (129) genotypes were all categorized as possessing a CD163-null phenotype.

The CD163 modification containing the hCD163L1 domain 8 peptide sequence HL11m, showed dual expression of CD163⁺ and CD169⁺ on PAMs (panel E of FIG. 19). However, in all of the HL11m pigs analyzed in this study, the surface expression of CD163 was markedly reduced compared to the WT PAMs. The levels of CD163 fell on a continuum of expression, ranging from no detectable CD163 to pigs possessing moderate levels of CD163. In the example shown in panel E of FIG. 19, approximately 60% of cells were in the double-positive quadrant while 40% of cells stained for only CD169. The analysis of PAMs from a total 24 HL11m pigs showed 38+/−12% of PAM cells were positive only for CD169 and 54+/−14% were double-positive (CD169⁺CD163⁺).

### Circulating Haptoglobin Levels in WT and CD163-Modified Pigs

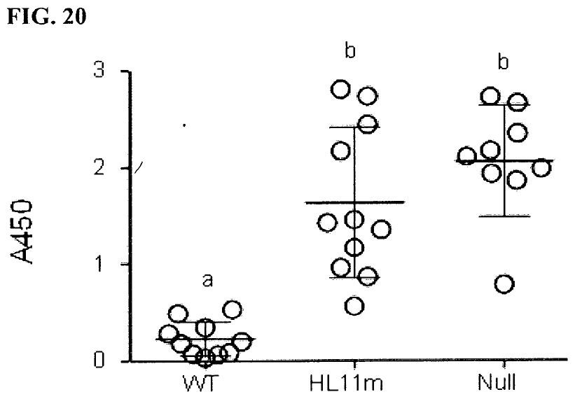

As a scavenging molecule, CD163 is responsible for removing HbHp complexes from the blood (Fabriek, et al., 2005; Kristiansen et al., 2001; and Madsen et al., 2004). The level of Hp in serum provides a convenient method for determining the overall functional properties of CD163-expressing macrophages. Hp levels in sera from WT, HL11m and CD163-null pigs were measured at three to four weeks of age, just prior to infection with PRRSV. The results, presented in FIG. 20, showed that sera from WT pigs had the lowest amounts of Hb (mean A450=23+/−0.18, n=10). The mean and standard deviation for each group were WT, 0.23+/−0.18, n=10; HL11m, 1.63+/−0.8, n=11; and 2.06+/−0.57, n=9, for the null group. The null group was composed of genotypes that did not express CD163 (CD163 null phenotype pigs). Hp measurements were made on a single ELISA plate. Groups with the same letter were not significantly different (p>0.05, Kruskal-Wallis one-way ANOVA with Dunnett's post-test). The mean A450 value was for WT pigs was significantly different from that of the HL11m and CD163-null pigs (p<0.05). Although the mean A450 value was lower for the HL11m group compared to the CD163-null group (A450=1.6+/−0.8 versus 2.1+/−0.6), the difference was not statistically significant. Since the interaction between HbHp and CD163 occurs through SRCR 3 (Madsen et al., 2004), increased circulating Hp in the HL11m pigs compared to WT pigs was likely not a consequence of a reduced affinity of CD163 for Hb/Hp, but the result of reduced numbers of CD163^{k} macrophages along with reduced CD163 expression on the remaining macrophages (see panel E of FIG. 19).

Infection of PAMs with Type 1 and Type 2 Viruses

The permissiveness of the CD163-modified pigs for PRRSV was initially evaluated by infecting PAM cells in vitro with a panel of six Type 1 and nine Type 2 PRRSV isolates (see Table 15 for the list of viruses). The viruses in the panel represent different genotypes, as well as differences in nucleotide and peptide sequences, pathogenesis, and years of isolation. The data presented in Table 16 show the results form experiments using PAMs from three pigs for each CD163 genotype group. The viruses listed correspond to the PRRSV isolates listed in Table 15. The results are shown as mean+/− standard deviation of the percent of PAMs infected. The CD163-null PAMs were from pigs expressing the d7(129) allele (see FIGS. 17 and 19 for CD163 gene constructs and CD163 expression on PAMs, respectively).

TABLE 16
Infection of PAMs from wild-type and GE pigs with
different PRRSV isolates

| | Genotype/Phenotype (% Infection) | | |
|---|---|---|---|
| | WT (%) | HL11m | Null |
| Type 1 | | | |
| 13-15 | 56 +/− 9 | 0 | 0 |
| Lelystad | 62 +/− 15 | 0 | 0 |
| 03-1059 | 50 +/− 18 | 0 | 0 |
| 03-1060 | 61 +/− 12 | 0 | 0 |
| 01-08 | 64 +/− 20 | 0 | 0 |
| 4353-PZ | 62 +/− 15 | 0 | 0 |
| Type 2 | | | |
| NVSL 97 | 59 +/− 15 | 8 +/− 08 | 0 |
| KS-06 | 56 +/− 20 | 12 +/− 09 | 0 |
| P129 | 64 +/− 11 | 8 +/− 06 | 0 |
| VR2332 | 54 +/− 05 | 6 +/− 03 | 0 |
| CO 10-90 | 43 +/− 18 | 8 +/− 08 | 0 |
| CO 10-84 | 51 +/− 22 | 7 +/− 04 | 0 |
| MLV-ResP | 55 +/− 12 | 3 +/− 01 | 0 |
| KS62 | 49 +/− 03 | 10 +/− 11 | 0 |
| KS483 | 55 +/− 23 | 6 +/− 03 | 0 |

As expected, the WT PAMs were infected by all viruses. In contrast, the CD163-null phenotype pigs were negative for infection by all viruses. A marked difference was observed in the response of PAMs from the HL11m pigs. None of the Type 1 viruses were able to infect the HL11m PAMs; whereas, all viruses in the Type 2 panel infected the HL11m PAMs, albeit at much lower percentages compared to the WT PAMs.

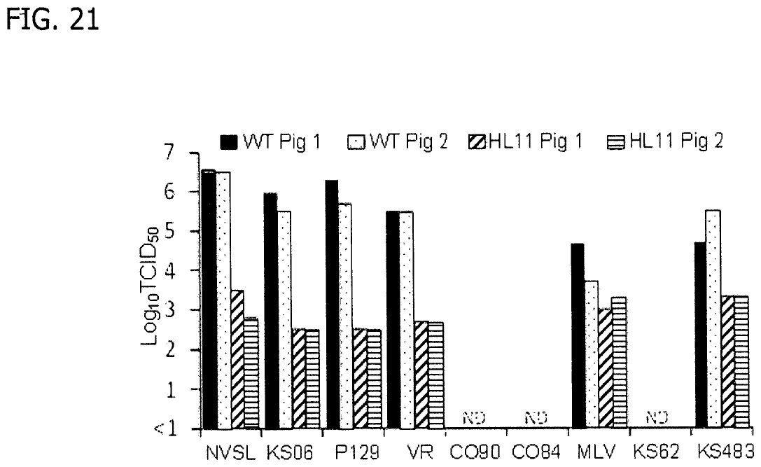

Permissiveness was also evaluated by comparing virus titration endpoints between WT and HL11m PAMs for the same Type 2 viruses. Results are shown for two WT and two HL11m pigs (FIG. 21). The log₁₀ TCID₅₀ values were calculated based on the infection of macrophage cultures with the same virus sample. Infection results represent two different pigs from each genotype. Viruses used for infection are listed in Table 15. The log₁₀ TCID₅₀ values for PAMs from the HL11m pigs were 1-3 logs lower compared to WT PAMs infected with the same virus. The only exception was infection with a modified-live virus vaccine strain. When taken altogether, the results suggest that PAMs from HL11m pigs possess a reduced susceptibility or permissiveness to infection with Type 2 viruses.

Infection of CD163-Modified Pigs with Type 1 and Type 2 Viruses

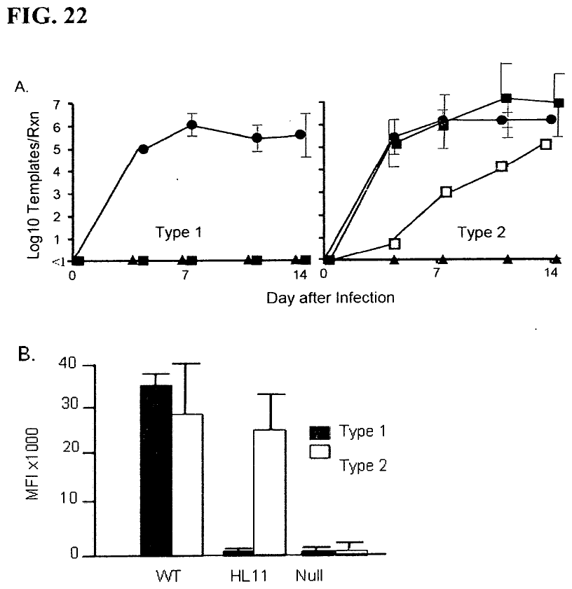

WT (circles), HL11m (squares), and CD163-null (triangles) pigs were infected with representative Type 1 (SD13-15) (FIG. 22, panel A, left graph) and Type 2 (NVSL 97-7895) (FIG. 22, panel A, right graph) viruses. The null phenotype pigs were derived from the KO and d(1567) alleles (see FIG. 17). Pigs from the three genotypes inoculated with the same virus were co-mingled in one pen, which allowed for the continuous exposure of CD163-modified pigs to virus shed from WT pen mates. The number of pigs infected with representative Type 1 virus were: WT (n=4), HL11m (n=5), and Null (n=3); and Type 2 virus: WT (n=4), HL11m (n=4), and Null (n=3). As shown in FIG. 22, the CD163-null pigs infected with either the Type 1 or Type 2 virus were negative for viremia at all time points and did not seroconvert. As expected, the WT pigs were productively infected possessing mean viremia levels approaching 10⁶ templates per 50 μl PCR reaction at 7 days after infection for both viruses. By 14 days, all WT pigs had seroconverted (see FIG. 22, panel B). Consistent with the PAM infection results (Table 16), the five HL11m pigs infected with the Type 1 virus showed no evidence of viremia or PRRSV antibody. All HL11m pigs infected with the Type 2 isolate, NVSL, supported infection and seroconverted (FIG. 22, panel B). The presence of a reduced permissive of the HL11m pigs was unclear. Mean viremia for three of the four HL11m pigs were similar to the WT pigs. However, for one HL11m pig, #101 (open squares in FIG. 22, panel A right graph), viremia was greatly reduced compared to the other pigs in HL11m genotype group. An explanation for the 3 to 4 log reduction in viremia for Pig #101 was not clear, but suggested that some HL11m pigs may be less permissive for PRRSV, an observation supporting the in vitro PAM infection results (Table 16). Since all pigs were inoculated with the same amount of virus and remained co-mingled with the WT pigs, the lower viremia in Pig #101 was not the result of receiving a lower amount of virus or less exposure to virus. Flow cytometry of macrophages showed that CD163 expression for Pig #101 was comparable to the other HL11m pigs (data not shown). There was no difference in the sequence in the exon 11 mimic sequence.

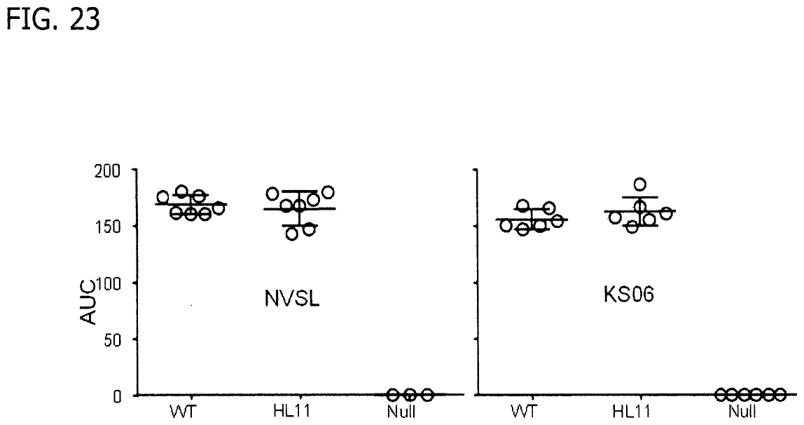

Additional virus infection trials were conducted using two viruses, NVSL 97-7895 and KS06-72109. Results are shown in FIG. 23. Pigs were followed for 35 days after infection and data reported as the area under the curve (AUC) for viremia measurements taken at 3, 7, 11, 14, 21, 28 and 35 days after infection. As shown in FIG. 23, for NVSL, the mean AUC value for the seven WT pigs infected with NVSL was 168+/−8 versus 165+/−15 for the seven HL11m pigs. For KS06, the mean AUC values for the six WT and six HL11m pigs were 156+/−9 and 163+/−13, respectively. For both viruses, there was no statistically significant difference between the WT and HL11m pigs (p>0.05). When taken altogether, the results showed that the HL11m pigs failed to support infection with Type 1 PRRSV, but retained permissiveness for infection with Type 2 viruses. Even though there was a reduction in the PRRSV permissiveness of PAMs from HL11m pigs infected in vitro with the Type 2 isolates, this difference did not translate to the pig. For the results shown in FIG. 23, virus load was determined by calculating the area under the curve (AUC) for each pig over a 35 day infection period. The AUC calculation was performed using log₁₀ PCR viremia measurements taken at 0, 4, 7, 10, 14, 21, 28 and 35 days after infection. The horizontal lines show mean and standard deviation. Key: WT=wild-type pigs, HL11=HL11m genotype pigs; Null=CD163-null genotype.

### Discussion

CD163 is a macrophage surface protein important for scavenging excess Hb from the blood and modulating inflammation in response to tissue damage. It also functions as a virus receptor. CD163 participates in both pro- and anti-inflammatory responses (Van Gorp et al., 2010). CD163-positive macrophages are placed within the alternatively activated M2 group of macrophages, which are generally described as highly phagocytic and anti-inflammatory. M2 macrophages participate in the cleanup and repair after mechanical tissue damage or infection (Stein et al., 1992). In an anti-inflammatory capacity, CD163 expression is upregulated by anti-inflammatory proteins, such as IL-10 (Sulahian, et al., 2002). During inflammation, CD163 decreases inflammation by reducing oxidative through the removal of circulating heme from the blood. Heme degradation products, such as bilverdin, bilirubin, and carbon monoxide are potent anti-inflammatory molecules (Soares and Bach, 2009 and Jeney et al., 2002). In a pro-inflammatory capacity, the crosslinking of CD163 on the macrophage surface by anti-CD163 antibody or bacteria results in the localized release of pro-inflammatory cytokines, including IL-6, GM-CSF, TNFα and IL-1β (Van den Heuvel et al., 1999 and Fabriek et al., 2009).

GE pigs that lack CD163 fail to support the replication of a Type 2 PRRSV isolate (Whitworth et al., 2016). In this study, in vitro infection trials demonstrate the resistance of CD163 null phenotype macrophages to an extensive panel of Type 1 and Type 2 PRRSV isolates, further extending resistance to potentially include all PRRSV isolates (Table 16). Resistance of the CD163-null phenotype macrophages to Type 1 and Type 2 viruses was confirmed in vivo (FIG. 22 and FIG. 23). Based on these results, the contribution of other PRRSV receptors previously described in the literature (Zhang and Yoo, 2015) can be ruled out. For example, Shanmukhappa et al. (2007) showed that non-permissive BHK cells transfected with a CD151 plasmid acquired the ability to support PRRSV replication, and incubation with a polyclonal anti-CD151 antibody was shown to significantly reduce the infection of MARC-145 cells. In addition, a simian cell line, SJPL, originally developed for use in propagating swine influenza viruses, was previously shown to support PRRSV replication (Provost, et al., 2012). Important properties of the SJPL cell line included the presence of CD151 and the absence of sialoadhesin and CD163. When taken together, these data provided convincing evidence that the presence of CD151 alone is sufficient to support PRRSV replication. The results from this study showing the absence of PRRSV infection in macrophages and pigs possessing a CD163 null phenotype indicates that CD151 as an alternative receptor for PRRSV is not biologically relevant.

The viral proteins GP2a and GP4, which form part of the GP2a, GP3, GP4 heterotrimer complex on the PRRSV surface, can be co-precipitated with CD163 in pull-down assays from cells transfected with GP2 and GP4 plasmids (Das, et al., 2009). Presumably, GP2 and GP4 form an interaction with one or more of the CD163 SRCR domains. In vitro infectivity assays incorporating a porcine CD163 cDNA backbone containing a domain swap between porcine SRCR 5 and the homolog from hCD163-L1 SRCR 8 further localized the region utilized by Type 1 viruses to SRCR 5 (Van Gorp, et al., 2010). It is interesting to speculate that the stable interaction between GP2/GP4 and CD163 occurs through SRCR 5. Additional viral glycoproteins, such as GP3 and GP5, may further stabilize the virus-receptor complex or may function as co-receptor molecules. The requirement for SRCR 5 was investigated in this study by infecting macrophages and pigs possessing the HL11m allele, which recreated the CD163L1 SRCR 8 domain swap by making 33 bp substitutions in porcine exon 7. The HL11m allele also included a neomycin cassette for selection of cells positive for the genetic alteration (FIG. 17). The HL11m pigs expressed CD163 on PAMs, albeit at reduced levels compared to WT PAMs (FIG. 19, compare panels A and E). Reduced expression was likely due to the presence of the neomycin cassette, which was located between the exon 11 mimic and the following intron. HL11m pigs were not permissive for infection with a Type 1 virus, confirming the importance of SRCR 5. However, HL11m macrophages and HL11m pigs did support infection with Type 2 viruses. Based on virus titration and percent infection results, the PAMs from the HL11m pigs showed an overall decrease in permissiveness for virus compared to the WT macrophages (Table 16 and FIG. 17). Decreased permissiveness may be due to reduced levels of CD163 on the HL11m macrophages, combined with a reduced affinity of virus for the modified CD163 protein. Assuming that Type 2 viruses possesses a requirement of SRCR 5 and that L1 SRCR 8 can function as a suitable substitute, the lower affinity may be explained by the difference in peptide sequences between human SRCR 8 and porcine SRCR 5 (see FIG. 18, panel B). However, the reduced permissiveness of PAMs did not translate to the pig. Mean viremia for the HL11m pigs was not significantly different when compared to WT pigs (FIG. 23). In addition to PAMs, PRRSV infection of intravascular, septal and lymphoid tissue macrophages contribute to viremia (Lawson et al., 1997 and Morgan et al., 2014). The potential contributions of these and other CD163-positive cells populations in maintaining the overall virus load in HL11m pigs deserves further study.

Even though CD163 plasmids possessing deletions of SRCR domains are stably expressed in HEK cells (Van Gorp et al., 2010), the deletion of exons 7 and 8 in d7(1467) and d7(1280) resulted in a lack of detectable surface expression of CD163 (FIG. 19, panel D). Since the 2A10 mAB used for flow cytometry recognizes the three N-terminal SRCR domains (Van Gorp et al., 2010), and possibly the 7th and 8th domains (Sanchez, et al., 1999), the absence of detection was not due to the removal of a 2A10 epitope in the mutated proteins. While a small amount of CD163 expression could be detected on PAMs from some of the d7(129) pigs (see FIG. 19, panel C), the quantity of expressed protein was not sufficient to support PRRSV infection in PAMs or pigs. The absence of CD163 expression in the exon 7 and 8 deletion mutants is not fully understood, but is likely the result of mRNA and/or protein degradation.

In 2003, CD163 was identified as a receptor for African swine fever virus (ASFV; Sánchez-Torres et al., 2003). This conclusion was based on the observation that infected macrophages possess a mature CD163-positive phenotype, and anti-CD163 antibodies, such as 2A10, block ASFV infection of macrophages in vitro. It remains to be determined if CD163-null pigs are resistant to ASFV infection.

Cell culture models incorporating modifications to the PRRSV receptor have provided valuable insight into the mechanisms of PRRSV entry, replication and pathogenesis. One unique aspect of this study was the conduct of parallel experiment in vivo using receptor-modified pigs. This research has important impacts on the feasibility of developing preventative cures for one of the most serious diseases to ever face the global swine industry.

### Example 4: Generation of SIGLEC Knockout Pigs

The following example describes the generation of SIGLEC1 knockout pigs.

### Materials and Methods

Unless otherwise stated, all of the chemicals used in this study were from Sigma, St. Louis, Mo.

### Targeted Disruption of Porcine SIGLEC1 Gene

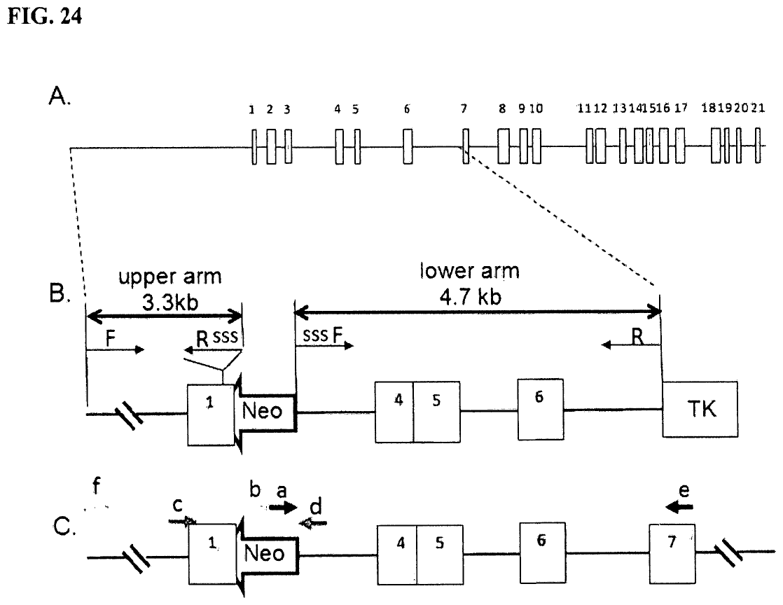

The use of animals and virus was approved by university animal care and institutional biosafety committees at the University of Missouri and/or Kansas State University. Homologous recombination was incorporated to remove protein coding exons 2 and 3 from SIGLEC1 and introduce premature stop codons to eliminate the expression of the remaining coding sequence (FIG. 24). Porcine SIGLEC1 cDNA (GenBank accession no. NM214346) encodes a 210-kDa protein from an mRNAtranscript of 5,193 bases (Vanderheijden et al., 2003). Genomic sequence from the region around SIGLEC1 (GenBank accession no. CU467609) was used to prepare oligonucleotide primers to amplify genomic fragments by high-fidelity PCR (AccuTaq; Invitrogen) for the generation of a targeting construct. On the basis of comparisons with the mouse and human genomic sequences, porcine SIGLEC1 was predicted to possess 21 exons. In addition, exon 2 is conserved among pigs, mice, and humans. Peptide sequence alignments revealed that the six amino acids in the exon 2 coding region in mouse SIGLEC1, known to be involved with sialic acid binding, are conserved in pig SIGLEC1. One fragment, the “upper arm” represented part of the first coding exon and 3,304 bp upstream from the start codon. The second fragment, or “lower arm,” was 4,753 bp in length and represented most of the intron downstream of the third coding exon and extended into the sixth intron (including the fourth, fifth, and sixth coding exons). Between the lower and upper arms was a neo cassette inserted in the opposite direction and placed under the control of a phosphoglycerol kinase (PGK) promoter.

For ease of reference, a partial wild-type SIGLEC1 sequence is provided herein as SEQ ID NO: 122. The reference sequences starts 4,236 nucleotides upstream of exon 1 and includes all introns and exons through exon 7 and 1,008 nucleotides following the end of exon 7. SEQ ID NO: 123 provides a partial SIGLEC1 sequence containing the modification described herein, as illustrated in panel C of FIG. 24. As compared to the partial wild-type sequence (SEQ ID NO: 122), in SEQ ID NO: 123 there is a 1,247 base pair deletion from nucleotide 4,279 to 5,525 and the deleted sequence is replaced with a 1,855 base pair neomycin selectable cassette oriented in the opposite direction as compared to SEQ ID NO: 122. This insertion/deletion results in the loss of part of exon 1 and all of exon 2 and 3 of the SIGLEC1 gene.

Male and female fetal fibroblast primary cell lines, from day 35 of gestation, were isolated from large commercial white pigs (Landrace). The cells were cultured and grown for 48 hours to 80% confluence in Dulbecco's modified Eagles medium (DMEM) containing 5 mM glutamine, sodium bicarbonate (3.7 g/liter), penicillin-streptomycin, and 1 g/liter D-glucose, which was further supplemented with 15% fetal bovine serum (FBS; Hy-Clone), 10 g/ml gentamicin, and 2.5 ng/ml basic fibroblast growth factor. Medium was removed and replaced 4 hours prior to transfection. Fibroblast cells were washed with 10 ml of phosphate-buffered saline (PBS) and lifted off the 75-cm² flask with 1 ml of 0.05% trypsin-EDTA (Invitrogen).

The cells were resuspended in DMEM, collected by centrifugation at 600×g for 10 minutes, washed with Opti-MEM (Invitrogen), and centrifuged again at 600×g for 10 minutes. Cytosalts (75% cytosalts [120 mM KCl, 0.15 mMCaCl₂, 10 mM K₂HPO₄, pH 7.6, 5 mM MgCl₂] and 25% Opti-MEM [Invitrogen]) were used to resuspend the pellet (van den Hoff et al., 1992). The cells were counted with a hemocytometer and adjusted to 1×10⁶/ml. Electroporation of cells was performed with 0.75 to 10 g of double- or single-stranded targeting DNA (achieved by heat denaturation) in 200 μl of transfection medium containing 1×10⁶ cells/ml. The cells were electroporated in a BTX ECM2001 Electro Cell Manipulator by using three 1-ms pulses of 250 V. The electroporated cells were diluted in DMEM-FBS-basic fibroblast growth factor at 10,000/13-cm plate and cultured overnight without selective pressure. The following day, the medium was replaced with culture medium containing G418 (GENTICIN, 0.6 mg/ml). After 10 days of selection, G418-resistant colonies were isolated and transferred to 24-well plates for expansion. PCR was used to determine if targeting of SIGLEC1 was successful. PCR primers “f” and “b” and PCR primers “a” and “e” (Table 17; FIG. 24) were used to determine the successful targeting of both arms. Primers “f” and “e” annealed outside the region of each targeting arm. PCR primers “c” and “d” were used to determine the insertion of an intact neo gene.

### Somatic Cell Nuclear Transfer

Pig oocytes were purchased from AR Inc. (Madison, Wis.) and matured according to the supplier's instructions. After 42 to 44 hours of in vitro maturation, the oocytes were stripped of cumulus cells by gentle vortexing in 0.5 mg/ml hyaluronidase. Oocytes with good morphology and a visible polar body (metaphase II) were selected and kept in manipulation medium (TCM-199 [Life Technologies] with 0.6 mM NaHCO₃, 2.9 mM Hepes, 30 mM NaCl, 10 ng/ml gentamicin, and 3 mg/ml BSA, with osmolarity of 305 mOsm) at 38.5° C. until nuclear transfer.

Using an inverted microscope, a cumulus-free oocyte was held with a holding micropipette in drops of manipulation medium supplemented with 7.5 g/ml cytochalasin B and covered with mineral oil. The zona pellucida was penetrated with a fine glass injecting micropipette near the first polar body, and the first polar body and adjacent cytoplasm, containing the metaphase II chromosomes, were aspirated into the pipette. The pipette was withdrawn, and the contents were discarded. A single round and bright donor cell with a smooth surface was selected and transferred into the perivitelline space adjacent to the oocyte membrane (Lai et al., 2006 and Lai et al., 2002). The nuclear transfer complex (oocyte plus fibroblast) was fused in fusion medium with a low calcium concentration (0.3M mannitol, 0.1 mM CaCl₂.2H₂O, 0.1 mM MgCl₂.6H₂O, 0.5 mM HEPES). The fused oocytes were then activated by treatment with 200 M thimerosal for 10 minutes in the dark, rinsed, and treated with 8 mM dithiothreitol (DTT) for 30 minutes; the oocytes were rinsed again to remove the remaining DTT (Machaty et al., 2001; Machaty et al., 1997). Following fusion and activation, the oocytes were washed three times with Porcine Zygote Culture Medium 3 supplemented with 4 mg/ml of bovine serum albumin (Im et al., 2004) and cultured at 38.5° C. in a humidified atmosphere of 5% O₂, 90% N₂, and 5% CO₂ for 30 minutes. Those complexes that had successfully fused were cultured for 15 to 21 hours until surgical embryo transfer.

### Embryo Transfer

The surrogate gilts were synchronized by administering 18 to 20 mg REGU-MATE (altrenogest, 2.2 mg/mL; Intervet, Millsboro, Del.) mixed into the feed for 14 days according to a scheme dependent on the stage of the estrous cycle. After the last REGU-MATE treatment (105 hours), an intramuscular injection of 1,000 units of human chorionic gonadotropin was given to induce estrus. Surrogate pigs on the day of standing estrus (day 0) or on the first day after standing estrus were used (Lai et al., 2002). The surrogates were aseptically prepared, and a caudal ventral incision was made to expose the reproductive tract. Embryos were transferred into one oviduct through the ovarian fimbria. Pigs were checked for pregnancy by abdominal ultrasound examination around day 30 and then checked once a week through gestation until parturition at 114 days of gestation.

### PCR and Southern Blot Confirmation in Transgenic Piglets

For PCR and Southern blot assays, genomic DNA was isolated from tail tissue Briefly, the tissues were digested overnight at 55° C. with 0.1 mg/ml of proteinase K (Sigma, St. Louis, Mo.) in 100 mM NaCl, 10 mM Tris (pH 8.0), 25 mM EDTA (pH 8.0) and 0.5% SDS. The material was extracted sequentially with neutralized phenol and chloroform, and the DNA was precipitated with ethanol (Green et al., 2012). Detection of both wild-type and targeted SIGLEC1 alleles was performed by PCR with primers that annealed to DNA flanking the targeted region of SIGLEC1. The primers are listed in Table 17 below. Three pairs of primers were used to amplify, respectively, the thymidine kinase (TK) lower-arm region (“a” forward and “e” reverse, black arrows in FIG. 24), the upper-arm Neo region (“f” forward, and “b” reverse, light grey arrows in FIG. 24), and exon 1 and the neo gene (“c” forward and “d” reverse, dark grey arrows in FIG. 24). The incorporation of primers “c” and “d” (dark grey arrows in FIG. 24) was designed to yield 2,400 and 2,900 bp of the wild-type and targeted alleles, respectively.

TABLE 17
PCR primers for amplifying SIGLEC1 modifications

| Primer Name (Target) | Sequence (5′ to 3′) | SEQ ID NO. |
|---|---|---|
| ″a″ forward (TK) | AGAGGCCACTTGTGTAGCGC | 124 |
| ″e″ reverse (TK) | CAGGTACCAGGAAAAACGGGT | 125 |
| ″f″ forward (upper-arm Neo) | GGAACAGGCTGAGCCAATAA | 126 |
| ″b″ reverse (upper-arm Neo) | GGTTCTAAGTACTGTGGTTTCC | 127 |
| ″c″ forward (exon 1 and neo) | GCATTCCTAGGCACAGC | 128 |
| ″d″ reverse (exon 1 and neo) | CTCCTTGCCATGTCCAG | 129 |

For Southern blot assays, the genomic DNA was digested at 37° C. with ScaI and MfeI (New England BioLabs). Sites for MfeI reside in the genomic regions upstream of the translation start site and in intron 6. A ScaI site is present in the neo cassette. Digested DNA was separated on an agarose gel, transferred to a nylon membrane (Immobilon NY+; EMD Millipore) by capillary action, and immobilized by UV cross-linking (Green et al., 2012). A genomic fragment containing intron 4 and portions of exons 4 and 5 was amplified by PCR using the oligonucleotides listed in Table 18 below, and labeled with digoxigenin according to the manufacturer's protocol (Roche). Hybridization, washing, and signal detection were performed in accordance with the manufacturer's recommendations (Roche). The predicted sizes of the wild-type and targeted SIGLEC1 genes were 7,892 and 7,204 bp, respectively.

TABLE 18
Oligonucleotides for Southern Blot Assays

| Oligonucleotide | Sequence (5′ to 3′) | SEQ ID NO. |
|---|---|---|
| 2789 F | GATCTGGTCACCCTCAGCT | 130 |
| 3368R | GCGCTTCCTTAGGTGTATTG | 131 |

### SIGLEC1 (CD169) and CD163 Surface Staining of PAM Cells

PAM cells (porcine alveolar macrophages) were collected by lung lavage. Briefly, excised lungs were filled with approximately 100 ml of cold PBS. After a single wash, the pellet was resuspended in approximately 5 ml of cold PBS and stored on ice. Approximately 10⁷ PAM cells were incubated in 5 ml of 20 μg/ml anti-porcine CD169 (clone 3B11/11; AbD Serotec) or anti-porcine CD163 (clone 2A10/11; AbD Serotec) antibody diluted in PBS with 5% FBS and 0.1% sodium azide (PBS-FBS) for 30 minutes on ice. Cells were centrifuged, washed, and resuspended in 1/100 fluorescein isothiocyanate (FITC)-conjugated goat anti-mouse IgG (Life Technologies) diluted in staining buffer and incubated for 30 minutes on ice. At least 10⁴ cells were analyzed with a FACSCalibur flow cytometer and Cell Quest software (Becton, Dickinson).

### Results

### Creation of SIGLEC1 Knockout Pigs

The knockout strategy used, diagrammed in FIG. 24, focused on creating drastic alterations of SIGLEC1 such that exons 2 and 3 were eliminated and no functional protein was expected to be obtained from the mutated gene. In addition, further disruption of the gene was accomplished by replacing part of exon 1 and all of exons 2 and 3 with a neomycin-selectable cassette oriented in the opposite direction (Mansour et al., 1988). Thirty-four transfections were conducted with a variety of plasmid preparations (0.75 to 10 μg/μl, both single- and double-stranded constructs, and both medium- and large-size constructs). Also included were male and female cells representing five different porcine fetal cell lines. Over 2,000 colonies were screened for the presence of the targeted insertion of the neo cassette. The PCR primers pairs “f” plus “b” and “a” plus “e” (FIG. 24, panels B and C) were used to check for the successful targeting of the upper and lower arms of the construct. Two colonies tested positive for the presence of the correct insertion, one male and one female (data not shown).

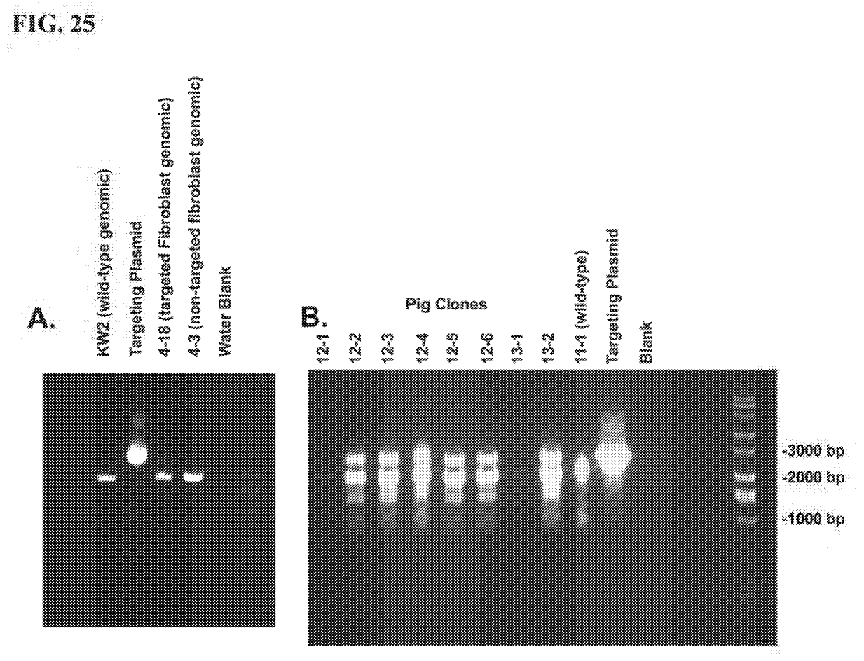

Cells from the male clone, 4-18, were used for somatic cell nuclear transfer and the transfer of 666 embryos into surrogates. The transfer of cloned embryos into two surrogates produced a total of eight piglets. One surrogate delivered six normal male piglets on day 115 of gestation. A C-section was performed on the second surrogate on day 117 of gestation, resulting in two normal male piglets. Three embryo transfers were also conducted with the female cells (658 embryos), but none established a pregnancy. FIG. 25 shows the results for PCRs performed with genomic DNA extracted from the eight male piglet clones (F₀) generated from the 4-18 targeted fetal fibroblast line. To detect both alleles, a PCR was performed with primers “c” and “d” (FIG. 24, panel C). The predicted PCR product sizes were 2,400 bp for the wild-type allele and 2,900 bp for the targeted allele. The results of the PCR with primers “c” and “d” are shown in FIG. 25. All of the pigs tested positive for the presence of the wild-type 2,400-bp and targeted 2,900-bp alleles (FIG. 25, panel B). Control PCRs incorporating DNA from the cell line used for cloning, the targeted 4-18 fibroblast cell line, and the non-targeted 4-18 cell line produced the predicted products (FIG. 25, panel A). The presence of the targeted mutation was further confirmed by amplifying regions with primer pairs identified by the light grey and black arrows in FIG. 24, panel C, which were predicted to yield products of ˜4,500 and ˜5,000 bp, respectively. Results showed the presence of both products in the eight founder pigs (data not shown).

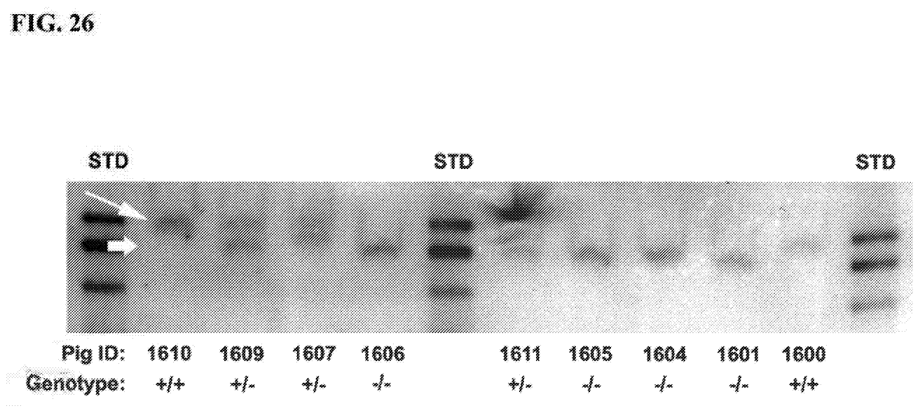

Five of the F₀ males were used for mating to wild-type females that resulted in 67 F₁ offspring (40 males and 27 females), 39 (58%) of which were SIGLEC1^{+/-}. One of the F₁ males was mated to one of the F₁ females (litter 52) to yield a litter of 12 pigs, 11 of which remained viable until weaning. Identification of wild-type and targeted alleles in the offspring was done by Southern blotting of genomic DNA. The results in FIG. 26 show four SIGLEC1^{+/+}, three SIGLEC1^{+/-}, and four SIGLEC1^{-/-} F2 animals.

### Expression of CD169 (SIGLEC1) and CD163 on PAM Cells.

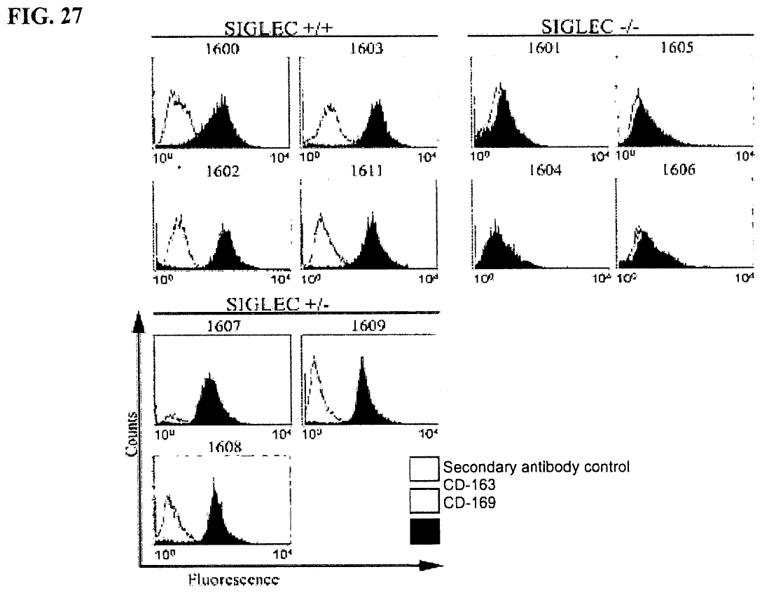

Cells for antibody staining were obtained from pigs at the end of the study. As shown in FIG. 27, greater than 90% of the PAM cells from SIGLEC1^{+//+} and SIGLEC1^{+/-} pigs were doubly positive for CD169 and CD163. In contrast, all of the SIGLEC1^{-/-} pigs were negative for surface expression of CD169 but remained positive for CD163. The results showed the absence of CD169 expression on cells from all of the SIGLEC1^{-/-} pigs. The absence of CD169 surface expression did not alter the expression the PRRSV co-receptor, CD163.

### Example 5: Use of a CRISPR/Cas9 System to Produce Pigs Having Chromosomal Modifications in ANPEP from In-Vitro-Derived Oocytes and Embryos

### Materials and Methods

### Chemicals and Reagents

Unless otherwise stated, all of the chemicals used in this study were purchased from Sigma, St. Louis, Mo.

Design of gRNAs to Build ANPEP Specific CRISPRs

The full-length genomic sequence of ANPEP (SEQ ID NO: 132) was used to design CRISPR guide RNAs. This transcript has 30,000 base pairs and three splice variants (X1, X2, and X3). X1 has 20 exons and encodes a 1017 amino acid protein product (SEQ ID NO: 133). X2 and X3 differ in a splice site occurring before the start codon in exon 2 and both encode the same 963 amino acid product (SEQ ID NO: 134).

Guide RNAs (gRNA) were designed to regions within exon 2 of the ANPEP gene because the start codon lies within exon 2. For ease of reference, a reference sequence comprising a portion of the full-length ANPEP sequence is provided herein (SEQ ID NO: 135). Reference sequence SEQ ID NO: 135 comprises a portion of intron 2, exon 2, intron 3, exon 3, intron 4, exon 4 and a portion of intron 4. This reference sequence (SEQ ID NO: 135) comprises 1000 nucleotides preceding the start codon within exon 2, the coding region of exon 2, and 1000 nucleotides after the end of exon 2. An annotated version of this sequence appears in FIG. 28. In FIG. 28, exons 2, 3 and 4 are underlined. Exon 2 begins at nucleotide 775 in FIG. 28, consistent with variants X1 and X2 (variant X3 starts exon 2 at nucleotide 778). This difference has no effect on the protein product since it occurs prior to the start codon (the start codon is at nucleotides 1001-1003 of SEQ ID NO: 135 and is shown in lowercase bold text in FIG. 28). Therefore, exons 2, 3, and 4 in SEQ ID NO: 135 encode the first 294 amino acids in the two protein products encoded between the three variants (SEQ ID NO: 133 or SEQ ID NO: 134). For ease of reference, each of the INDELs described below in this Example and in Example 6 are described in reference to reference sequence SEQ ID NO: 135. When referring to amino acid sequences, references are made to the 963 amino acid protein encoded by splice variants X2 and X3 variants (SEQ ID NO: 134). However, the person of ordinary skill in the art will readily be able to determine where the insertions or deletions occur in the amino acid sequence encoded by splice variant X1 (SEQ ID NO: 133). A list of the nucleotides corresponding to the introns and exons included in reference SEQ ID NO: 135 appearing in FIG. 28 is provided in Table 19 below.

TABLE 19
Locations of Introns/Exons in FIG. 28

| Nucleotide range | Location/Qualifier |
|---|---|
| 1 . . . 774 | End of Intron 2 |
| 775 . . . 1599 | Exon 2 (start codon (atg) at nt 1001) |
| 1600 . . . 2109 | Intron 3 |
| 2110 . . . 2251 | Exon 3 |
| 2252 . . . 2378 | Intron 4 |
| 2379 . . . 2518 | Exon 4 |
| 2519 . . . 2599 | Beginning of Intron 5 |

All guide RNAs were designed after the start codon so that INDELs would be more likely to result in a frame-shift and premature start codon. The six targets selected were adjacent to an S. pyogenes (Spy) protospacer adjacent motif (PAM) (Ran et al. 2015) and are listed in Table 20 below. The PAM is identified by the parentheses in each gRNA. Guides 2 and 3 are also identified in bold and double underlined in SEQ ID NO: 135 in FIG. 28. Specificity of the designed crRNAs was confirmed by searching for similar porcine sequences in GenBank.

TABLE 20
ANPEP CRISPR Guides

| | | SEQ ID |
|---|---|---|
| Target | Sequence | NO. |
| ANPEP guide 1 | CTTCTACCGCAGCGAGTACA(TGG) | 136 |
| ANPEP guide 2 | TACCGCAGCGAGTACATGGA(GGG) | 137 |
| ANPEP guide 3 | CCTCCTCGGCGTGGCGGCCG(TGG) | 138 |
| ANPEP guide 4 | CACCATCATCGCTCTGTCTG(TGG) | 139 |
| ANPEP guide 5 | TACCTCACTCCCAACGCGGA(TGG) | 140 |
| ANPEP guide 6 | AGCTCAACTACACCACCCAG(GGG). | 141 |

Forward (F) and reverse (R) oligonucleotides corresponding to each ANPEP target, listed in Table 21 below, were annealed and cloned into the p330X vector which contains two expression cassettes, a human codon-optimized S. pyogenes (hSpy) Cas9 and the chimeric guide RNA. P330X was digested with BbsI (New England Biolabs) following the Zhang laboratory protocol (available at http://www.addgene.org/crispr/zhang/; see also Cong et al., 2013 and Hsu et al., 2013). Cloning success of each guide was confirmed by Sanger sequencing by the University of Missouri DNA core facility. Plasmids that were successfully cloned were propagated in TOP10 electrocompetent cells (Invitrogen, Carlsbad, Calif.) and large scale plasmid preps were performed with a Qiagen Plasmid Maxi kits (Qiagen, Germantown, Md.). Plasmids were frozen at −20° C. until use for in vitro transcription template or for transfection.

TABLE 21
Designed crRNAs for ANPEP editing.

| | | SEQ ID |
|---|---|---|
| Primer Name | Sequence (5′-3′) | NO. |
| ANPEP Guide 1 Primer 1 (For) | CACCGCTTCTACCGCAGCGAGTACA | 142 |
| ANPEP Guide 1 Primer 2 (Rev) | AAACTGTACTCGCTGCGGTAGAAGC | 143 |
| ANPEP Guide 2 Primer 1 (For) | CACCGTACCGCAGCGAGTACATGGA | 144 |
| ANPEP Guide 2 Primer 2 (Rev) | AAACTCCATGTACTCGCTGCGGTAC | 145 |
| ANPEP Guide 3 Primer 1 (For) | CACCGCCTCCTCGGCGTGGCGGCCG | 146 |
| ANPEP Guide 3 Primer 2 (Rev) | AAACCGGCCGCCACGCCGAGGAGGC | 147 |
| ANPEP Guide 4 Primer 1 (For) | CACCGCACCATCATCGCTCTGTCTG | 148 |
| ANPEP Guide 4 Primer 2 (Rev) | AAACCAGACAGAGCGATGATGGTGC | 149 |
| ANPEP Guide 5 Primer 1 (For) | CACCGTACCTCACTCCCAACGCGGA | 150 |
| ANPEP Guide 5 Primer 2 (Rev) | AAACTCCGCGTTGGGAGTGAGGTAC | 151 |
| ANPEP Guide 6 Primer 1 (For) | CACCGAGCTCAACTACACCACCCAG | 152 |
| ANPEP Guide 6 Primer 2 (Rev) | AAACCTGGGTGGTGTAGTTGAGCTC | 153 |

### Fetal Fibroblast Collection

Porcine fetuses were collected on day 35 of gestation to create cell lines for transfection. One wild-type male and one wild-type female fetal fibroblast cell line were established from a large white domestic cross. Fetal fibroblasts were collected as described previously with minor modifications (Lai and Prather., 2003a); minced tissue from the back of each fetus was digested in 20 mL of digestion media (Dulbecco's Modified Eagles Medium containing L-glutamine, 1 g/L D-glucose (Cellgro, Manassas, Va.) and 200 units/mL collagenase and 25 Kunitz units/mL DNaseI) for 5 hours at 38.5° C. After digestion, fetal fibroblast cells were washed and cultured with DMEM containing 15% fetal bovine serum (FBS) and 40 μg/mL gentamicin. After overnight culture, cells were trypsinized and slow frozen to −80° C. in aliquots in FBS with 10% dimethyl sulfoxide (DMSO) and stored long term in liquid nitrogen.

Transfection with ANPEP CRISPR gRNAs

Transfection conditions were similar to previously reported protocols (Ross et al., 2010; Whitworth et al., 2014). Briefly, six ANPEP guides were tested in different combinations over 17 transfections. The total CRISPR guide concentration was 2 μg/transfection. Fetal fibroblast cell lines of similar passage number (2-4) were cultured for two days and grown to 75-85% confluency in Dulbecco's Modified Eagles Medium containing L-glutamine and 1 g/L D-glucose (Cellgro, Manassas, Va.; DMEM) supplemented with 15% fetal bovine serum (FBS), 2.5 ng/ml basic fibroblast growth factor (Sigma), 10 mg/ml gentamicin, and 25 μg/ml of FUNGIZONE (amphotericin B). Fibroblast cells were washed with phosphate buffered saline (PBS; Life Technologies, Austin, Tex.) and trypsinized. As soon as cells detached, the cells were rinsed with an electroporation medium (75% cytosalts (120 mM KCl, 0.15 mM CaCl₂, 10 mM K₂HPO₄; pH 7.6, 5 mM MgCl₂)) (Yanez et al., 2016) and 25% OPTI-MEM (Life Technologies). Cells were counted by using a hemocytometer. Cells were pelleted at 600×g for 5 minutes and resuspended at a concentration of 1×10⁶/ml in electroporation medium. Each electroporation used 200 μL (0.2×10⁶ total cells) of cells in 2 mm gap cuvettes with three (1 msec) square-wave pulses administered through a BTX ECM 2001 electroporation system at 250 volts. After the electroporation, cells were resuspended in DMEM medium described above. Colonies were picked on day 14 after transfection. Fetal fibroblasts were plated at 50 cells/plate (Beaton and Wells 2014). Fetal fibroblast colonies were collected by sealing 10 mm autoclaved cloning cylinders around each colony. Colonies were rinsed with PBS and harvested via trypsin and then resuspended in DMEM culture medium. A part (1/3) of the resuspended colony was transferred to a 96-well PCR plate for genotyping and the remaining (2/3) of the cells were cultured in a well of a 24 well plate for cell propagation and subsequent somatic cell nuclear transfer (SCNT). The cell pellets were resuspended in 6 μL of lysis buffer (40 mM Tris, pH 8.9, 0.9% Triton X-100, 0.4 mg/mL proteinase K; New England Biolabs), incubated at 65° C. for 30 minutes for cell lysis followed by 85° C. for 10 minutes to inactivate the proteinase K. Cell lysates were then used for genotyping via PCR.

### Somatic Cell Nuclear Transfer (SCNT)

To produce SCNT embryos, sow-derived oocytes were purchased from Desoto Biosciences LLC (Seymour, NT). The sow derived oocytes were shipped overnight in maturation medium (TCM199 with 2.9 mM HEPES, 5 pg/mL insulin, 10 ng/mL EGF, 0.5 pg/mL p-FSH, 0.91 mM pyruvate, 0.5 mM cysteine, 10% porcine follicular fluid, 25 ng/mL gentamicin) and transferred into fresh medium after 24 hours. After 40-42 hours of maturation, cumulus cells were removed from the oocytes by vortexing in the presence of 0.1% hyaluronidase. During SCNT, oocytes were placed in manipulation medium (TCM199 with 0.6 mM NaHCO₃, 2.9 mM HEPES, 30 mM NaCl, 10 ng/mL gentamicin, and 3 mg/mL BSA; and osmolarity of 305) supplemented with 7.0 μg/mL cytochalasin B. The polar body along with a portion of the adjacent cytoplasm, presumably containing the metaphase II plate, was removed and a donor cell was placed in the perivitelline space by using a thin glass capillary (Lai and Prather., 2003b). The reconstructed embryos were then fused in a fusion medium (0.3 M mannitol, 0.1 mM CaCl₂, 0.1 mM MgCl₂, 0.5 mM HEPES) with two DC pulses (1-second interval) at 1.2 kV/cm for 30 μsec using a BTX Electro Cell Manipulator (Harvard Apparatus). After fusion, fused embryos were chemically activated with 200 μM thimerosal for 10 minutes in the dark and 8 mM dithiothreitol for 30 minutes (Machaty et al., 1997). Embryos were then incubated in modified Porcine Zygote Medium PZM3-MU1 (Bauer et al., 2010; Yoshioka et al., 2002) with 0.5 μM Scriptaid (Sigma-Aldrich, S7817), a histone deacetylase inhibitor, for 14-16 hours, as described previously (Whitworth et al., 2011; Zhao et al., 2010; Zhao et al., 2009).

### In Vitro Fertilization (IVF)

For IVF, ovaries from pre-pubertal gilts were obtained from an abattoir (Farmland Foods Inc., Milan, Mo.). Immature oocytes were aspirated from medium size (3-6 mm) follicles using an 18-gauge hypodermic needle attached to a 10 ml syringe. Oocytes with homogenous cytoplasm and intact plasma membrane and surrounding cumulus cells were then selected for maturation. Around 50 cumulus oocyte complexes were placed in a well containing 500 μL of maturation medium (TCM 199 (Invitrogen, Grand Island, N.Y.) with 3.05 mM glucose, 0.91 mM sodium pyruvate, 0.57 mM cysteine, 10 ng/mL epidermal growth factor (EGF), 0.5 μg/mL luteinizing hormone (LH), 0.5 μg/mL follicle stimulating hormone (FSH), 10 ng/mL gentamicin (APP Pharm, Schaumburg, Ill.), and 0.1% polyvinyl alcohol (PVA)) for 42-44 hours at 38.5° C., 5% CO2, in humidified air. Following maturation, the surrounding cumulus cells were removed from the oocytes by vortexing for 3 minutes in the presence of 0.1% hyaluronidase. In vitro-matured oocytes were placed in 50 μL droplets of IVF medium (modified Tris-buffered medium (mTBM) containing 113.1 mM NaCl, 3 mM KCl, 7.5 mM CaCl₂, 11 mM glucose, 20 mM Tris, 2 mM caffeine, 5 mM sodium pyruvate, and 2 mg/mL BSA) in groups of 25-30 oocytes. One 100 μL frozen pellet of wild type semen was thawed in 3 mL of Dulbecco's phosphate-buffered saline (DPBS) supplemented with 0.1% BSA. Semen was washed in 60% percoll for 20 minutes at 650×g and in mTBM for 10 minutes by centrifugation. The semen pellet was then re-suspended with IVF medium to 0.5×10⁶ cells/mL. Fifty μL of the semen suspension was introduced into the droplets with the oocytes. The gametes were co-incubated for 5 hours at 38.5° C. in an atmosphere of 5% CO2 in air. After fertilization, the embryos were incubated in PZM3-MU1 (Bauer et al. 2010; Yoshioka et al. 2002) at 38.5° C., 5% CO2 in air atmosphere.

### In Vitro Synthesis of RNA for CRISPR/Cas9 System

gRNA for zygote injection was prepared as previously described (Whitworth et al., 2017). Template guide DNA was first synthesized by Integrated DNA Technologies in the form of a gBlock. A T7 promoter sequence was added upstream of the guide for in vitro transcription (underlined in Table 22). Each gBlock was diluted to final concentration 0.1 ng/μl and PCR amplified with the in vitro transcription (IVT) forward primers (unique for each CRISPR guide) and the same reverse primer (gRNA Rev1) listed in Table 22. PCR conditions included an initial denaturation of 98° C. for 1 minutes followed by 35 cycles of 98° C. (10 seconds), 68° C. (30 seconds) and 72° C. (30 seconds). Each PCR-amplified gBlock was purified by using a QIAGEN PCR purification kit. Purified gBlock amplicons were then used as templates for in vitro transcription using the MEGASHORTSCRIPT transcription kit (Ambion). RNA quality was visualized on a 2.0% RNA-free agarose gel. Concentrations and 260:280 ratios were determined via NANODROP spectrophotometry. Capped and polyadenylated Cas9 mRNA was purchased from Sigma. RNA was diluted to a final concentration of 20 ng/μL (both gRNA and Cas9), distributed into 3 μL aliquots, and stored at −80° C. until injection.

TABLE 22
Primers used to amplify template DNA for in vitro transcription.

| | | SEQ |
|---|---|---|
| IVT Guide ID | Sequence (5′-3′) | ID NO. |
| ANPEP Guide 1 (For) | TTAATACGACTCACTATAGGCTTCTACCGCAGCGAGTACA | 154 |
| ANPEP Guide 2 (For) | TTAATACGACTCACTATAGGTACCGCAGCGAGTACATGGA | 155 |
| ANPEP Guide 3 (For) | TTAATACGACTCACTATAGGCCTCCTCGGCGTGGCGGCCG | 156 |
| ANPEP Guide 4 (For) | TTAATACGACTCACTATAGGCACCATCATCGCTCTGTCTG | 157 |
| ANPEP Guide 5 (For) | TTAATACGACTCACTATAGGCACCATCATCGCTCTGTCTG | 158 |
| ANPEP Guide 6 (For) | TTAATACGACTCACTATAGGAGCTCAACTACACCACCCAG | 159 |
| gRNA Rev1 | AAA AGC ACC GAC TCG GTG CC | 160 |

### Zygote Injection of ANPEP CRISPR/Cas9 System in Zygotes

Cas9 mRNA was purchased from Sigma Aldrich (St. Louis, Mo.) and was mixed with ANPEP gRNA 2 and 3 (Table 20). gRNA 2 was chosen because it had the highest editing efficiency after fetal fibroblast transfection. gRNA 3 was chosen as a negative control because it had no editing ability after fetal fibroblast transfection. This design was chosen to see if a similar editing rate would be observed between the two methods, fetal fibroblast transfection and zygote injection. The mix of gRNA 2 and gRNA 3 (20 ng/μl) and Cas9 mRNA (20 ng/μl) was coinjected into the cytoplasm of fertilized oocytes at 14 hours post-fertilization (presumptive zygotes) by using a FEMTOJET microinjector (Eppendorf; Hamburg, Germany). Microinjection was performed in manipulation medium (TCM199 with 0.6 mM NaHCO₃, 2.9 mM HEPES, 30 mM NaCl, 10 ng/mL gentamicin, and 3 mg/mL BSA; and osmolarity of 305) on the heated stage of a Nikon inverted microscope (Nikon Corporation; Tokyo, Japan). Injected zygotes were then transferred into the PZM3-MU1 with 10 ng/mL ps48 until embryo transfer or allowed to develop to the blastocyst stage for genotype confirmation.

### Genotyping Assays

Genomic DNA was used to assess genotype by PCR, agarose gel electrophoresis, and subsequent Sanger DNA sequencing. PCR was performed with the ANPEP-specific primers listed in Table 23 below using a standard protocol and LA Taq (Takara, Mountain View, Calif.). PCR conditions consisted of 96° C. for 2 minutes and 35 cycles of 95° C. for 30 seconds, 50° C. for 40 seconds, and 72° C. for 1 minute, followed by an extension of 72° C. for 2 minutes. A 965 bp amplicon was then separated on a 2.0% agarose gel to determine obvious insertions or deletions. Amplicons were also subjected to Sanger sequencing to determine the exact location of the modification. Amplicons from live pigs were TOPO cloned and DNA sequenced to determine the exact modification of both alleles.

TABLE 23
ANPEP Specific Primers for PCR

| Primer | Sequence | SEQ ID NO. |
|---|---|---|
| ANPEP Forward | ACGCTGTTCCTGAATCT | 161 |
| ANPEP Reverse | GGGAAAGGGCTGATTGTCTA | 162 |

### Embryo Transfer

Embryos generated to produce ANPEP edited pigs were transferred into recipient gilts for term birth. For SCNT and IVF zygote injected embryos, embryos were either cultured overnight and transferred into the oviduct of a gilt on day 1 of the estrous cycle (SCNT) or cultured for five days and then transferred to the oviduct of a gilt on day 4, 5, or 6 of the estrous cycle (IVF and SCNT). All embryos were transported to the surgical site in PZM3-MU1 (Bauer et al. 2010) in the presence of 10 ng/mL ps48 (5-(4-Chloro-phenyl)-3-phenyl-pent-2-enoic acid; Stemgent, Inc., Cambridge, Mass.). Regardless of stage of development, all embryos were surgically transferred into the ampullary-isthmic junction of the oviduct of the recipient gilt (Lee et al. 2013). There were a total of four embryo transfers (ETs) performed with SCNT embryos and six ETs performed with zygote injected embryos. The first two embryo transfers for SCNT were performed using donor cells from the original colonies isolated after transfection. The donor cells for the second two ETs for SCNT were isolated from day 35 fetuses collected from the first two ETs.

### Immunohistochemistry

Immunohistochemistry to detect the presence of ANPEP in the ileum of modified pigs was performed using standard procedures. Upon collection, intestinal tissues were immediately placed in 10% buffered formalin. After processing, the paraffin-embedded sections were mounted on slides. Sections were dewaxed with Leica BOND Dewax Solution (a solvent-based deparaffinization solution) and antigen retrieval performed using Leica BOND Epitope Retrieval Solution 1 (a citrate-based pH 6.0 epitope retrieval solution for the heat-induced epitope retrieval of formalin-fixed, paraffin-embedded tissue) for 20 minutes at 100° C. Slides were incubated with 3% hydrogen peroxide for 5 minutes at room temperature and visualized by using an automated procedure on a NexES IHC Staining Module (Ventana Medical). A rabbit anti-CD13 (APN) polyclonal antibody (Abcam) prepared against a peptide covering amino acids 400 to 500 of human CD13 was used for the detection of APN antigen. The antibody was diluted 1:3200 in Leica BOND Primary Antibody Diluent (containing Tris-buffered saline, surfactant, protein stabilizer, and 0.35% PROCLIN 950 (2-Methyl-4-isothiazolin-3-one solution)) and incubated on slides for 15 minutes at room temperature. Slides were washed and bound antibody detected with anti-Rabbit IgG horseradish peroxidase (HRP). HRP activity was visualized with 3,3′-diaminobenzidine (DAB) and slides were counterstained with hematoxylin.

### Results

Transfections with ANPEP CRISPR Guide Plasmid

A total of 17 transfections were performed to determine which CRISPR guide would efficiently edit the ANPEP gene as well as to isolate primary cell lines with CRISPR induced ANPEP edits for use in SCNT. The transfection efficiency in each experiment is summarized in Table 24 below. The ANPEP guide 2 resulted in the highest number of edited colonies when transfected alone. There were a total of four transfections with ANPEP guide 2 and the editing efficiency ranged from 0-23.3%. A colony was considered edited if there was an observable size difference of the PCR amplicon after DNA electrophoresis. Only the resulting pigs and fetuses were sequenced to determine the precise location and size of the edits. ANPEP guide 1 was the second most efficient guide with an editing rate ranging from 0-7.1% across four transfections. Interestingly, when ANPEP guide 1 and 2 were mixed and cotransfected, the editing rate was 0% across three transfections. ANPEP guides 3 and 4 did not result in edits (two transfections each) and ANPEP guides 5 and 6 resulted in 2.9% and 4.2% editing, but only a single transfection was performed for each guide. Colonies E9, F7, D11 transfected with ANPEP guide 2 and colony A10 transfected with ANPEP guide 1 were selected for SCNT.

TABLE 24
Transfection Efficiency of CRISPR Guides

| | | | Num- | Num- | | Number | Percent |
|---|---|---|---|---|---|---|---|
| | | | ber | ber | Average | of | Edited |
| Treat- | Trans- | | Colo- | of | Colonies/ | Edited | Colo- |
| ment | fection | Sex | nies | Plates | plate | Colonies | nies |
| ANPEP 1 | 1 | male | 42 | 17 | 2.47 | 3 | 7.1 |
| ANPEP 2 | 2 | male | 31 | 12 | 2.58 | 1 | 3.2 |
| ANPEP | 3 | male | 23 | 19 | 1.21 | 0 | 0.0 |
| 1 + 2 | | | | | | | |
| mix | | | | | | | |
| ANPEP | 4 | female | 27 | 10 | 2.70 | 1 | 3.7 |
| ANPEP 2 | 5 | female | 30 | 10 | 3.00 | 7 | 23.3^{a} |
| ANPEP | 6 | female | 14 | 10 | 1.40 | 0 | 0.0 |
| 1 + 2 | | | | | | | |
| mix | | | | | | | |
| ANPEP 1 | 7 | male | 46 | 10 | 4.60 | 0 | 0.0 |
| ANPEP 2 | 8 | male | 36 | 10 | 3.60 | 0 | 0.0 |
| ANPEP 3 | 9 | male | 40 | 10 | 4.00 | 0 | 0.0 |
| ANPEP 4 | 10 | male | 35 | 10 | 3.50 | 0 | 0.0 |
| ANPEP 1 | 11 | male | 41 | 10 | 4.10 | 1 | 2.4^{b} |
| ANPEP 2 | 12 | male | 21 | 10 | 2.10 | 3 | 14.3^{c} |
| ANPEP | 13 | male | 34 | 10 | 3.40 | 0 | 0.0 |
| 1 + 2 | | | | | | | |
| mix | | | | | | | |
| ANPEP 3 | 14 | female | 28 | 10 | 2.80 | 0 | 0.0 |
| ANPEP 4 | 15 | female | 33 | 10 | 3.30 | 0 | 0.0 |
| ANPEP 5 | 16 | female | 35 | 10 | 3.50 | 1 | 2.9 |
| ANPEP 6 | 17 | female | 24 | 10 | 2.40 | 1 | 4.2 |

^{a}Cells used for SCNT (E9, F7);
^{b}Cells used for SCNT (A10);
^{c}Cells used for SCNT (D11)

### Somatic Cell Nuclear Transfer of ANPEP Edited Cells

Cells from colony E9, F7, D11 and A10 were used for SCNT. An equal number of embryos were reconstructed from each group of cells, but the embryos were mixed in a single pig during the ET. Two embryo transfers were performed with these primary colony cells and both pigs resulted in pregnancies. The pregnancies were terminated at day 35 for fetus collection. Ten fetuses were collected from pig O279, of which three contained biallelic edits in the ANPEP gene. Five fetuses were collected from pig O307, of which three contained biallelic edits in the ANPEP gene. Each fetus was genotyped and the resulting genotypes are listed in Table 25 below.

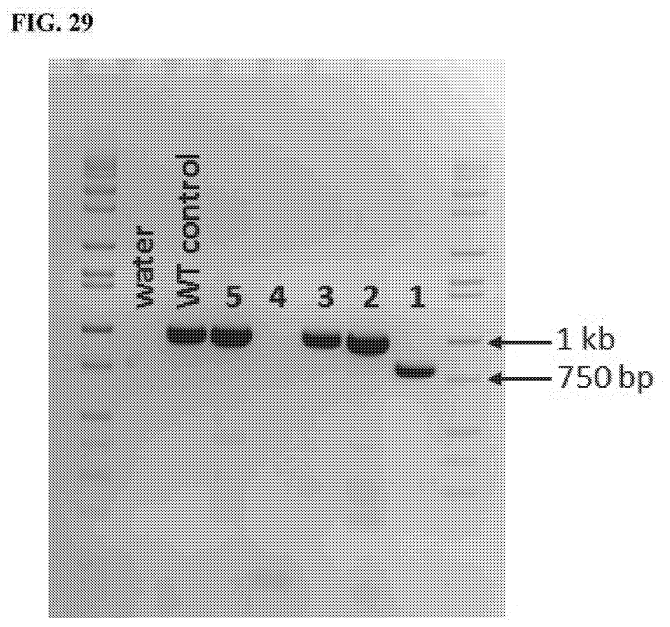

FIG. 29 shows a representative agarose gel showing the amplicons from PCR across the four genotypes observed from ET into recipients O279 and O307. Lane 1 is a 182 bp deletion/no WT, Lane 2 is a 9 bp deletion/no WT, Lane 3 is wild-type, Lane 4 is a867 bp deletion (light band towards bottom of gel)/no WT, and Lane 5 is wild-type. Many of the fetuses were biallelic (i.e., had two modified alleles). In each case, they had a characterized allele (e.g., 182 bp deletion) and a non-wild-type allele. The second non-wildtype allele was not sequenced or identified. Wild-type nucleic acid and water were used as the positive and negative controls, respectively.

Fetal fibroblast cell lines were created from each fetus and three fetal lines were then used for SCNT for two additional SCNT and ET. Neither recipient pig became pregnant from the newly established fetal cell lines (Table 25).

TABLE 25
Embryo Transfer data from somatic cell nuclear transfer of ANPEP edited embryos.

| | | | | Days | # Fetuses | |
|---|---|---|---|---|---|---|
| Pig | | Fusion | # Embryos | Post | Collected | |
| ID | Line* | rate (%) | Transferred | Estrus | (day 35) | Genotype Outcome |
| O279 | ANPEP E9, | 82.8 | 213 | 1 | 10 | 182 bp deletion, no WT (biallelic) |
| | F7, A11, | | (1-cell stage) | | | 9 bp deletion, no WT (biallelic) |
| | D10 | | | | | 867 bp deletion, no WT (biallelic) |
| | Primary | | | | | WT |
| | lines from | | | | | WT |
| | transfections | | | | | |
| O307 | ANPEP E9, | 86.5 | 213 | 2 | 5 | 182 bp deletion, no WT (biallelic) |
| | F7, A11, | | (1-cell stage) | | | 9 bp deletion, no WT (biallelic) |
| | D10 | | | | | 867 bp deletion, no WT (biallelic) |
| | Primary | | | | | |
| | lines from | | | | | |
| | transfections | | | | | |
| O380 | ANPEP FF | 75 | 53 (morula/ | 5 | N/A | Cycled back 20 days after ET |
| | from O279, | | blastocyst | | | |
| | O307 | | stage) | | | |
| O394 | ANPEP FF | 75.5 | 50 (morula/ | 6 | N/A | Cycled back 15 days after ET |
| | from O279, | | blastocyst | | | |
| | O307 | | stage) | | | |

*The primary cell lines were derived directly from the transfections. The ANPEP FF lines were derived from the fetuses collected from Pig O279 and O307.

### Zygote Injection

Six ETs were attempted with IVF zygotes injected with ANPEP gRNA. Embryo transfer data is summarized in Table 26 below. The first three ETs resulted in two pregnancies. One pig did not become pregnant. One recipient (pig O345) was euthanized on day 35 and six fetuses were collected. Of the six fetuses, four contained an edit of the ANPEP gene as summarized in Table 26. FIG. 30 provides representative PCR results depicting the alleles present in these six fetuses. The lane marked “water” is a negative no template control. The lane marked “WT control” is a wild-type positive control containing DNA from non-transfected fetal fibroblasts. Lanes 1-6 provide PCR results for the six fetuses, which were found to have the following genetic edits: (1) lane 1: 1 base pair insertion/no wild-type; (2) lane 2: 2 base pair insertion/wild-type; (3) lane 3: wild-type; (4) lane 4: wild-type; (5) lane 5: 3 base pair insertion, 9 base pair deletion, and 267 base pair deletion (mosaic); and (6) lane 6: 9 base pair deletion/wild-type. When a genotypic description includes the phrase “no WT” or “no wild-type” this means the fetus had an uncharacterized and not sequenced (but not wild-type) second allele. The named modified alleles were sequenced and are described hereinbelow.

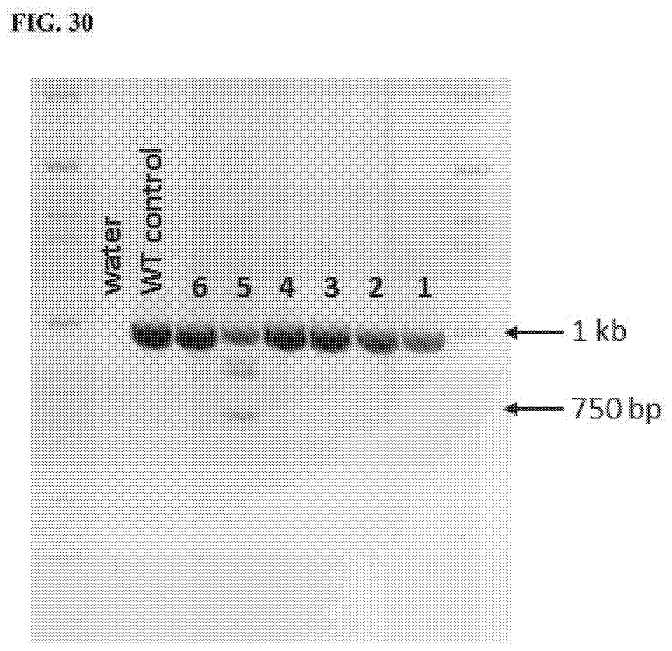

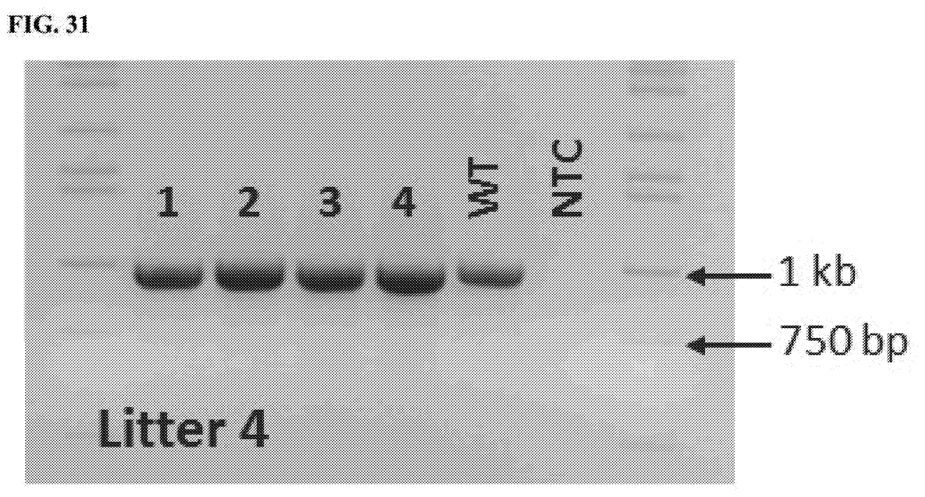

A third pig farrowed four piglets, of which three were edited. Genotypes of this litter (“litter 4”) were determined using TOPO cloning and Sanger sequencing and are summarized in Table 27. Representative PCR results showing each ANPEP allele from these four piglets as compared to wild-type (WT) or no template control (NTC) are shown in FIG. 4. Lanes 1-4 in FIG. 31 correspond to: (1) piglet 4-1, having a 9 base pair deletion in exon 2 in allele 1 and wild-type sequence in allele 2; (2) piglet 4-2, having a 1 base pair insertion in exon 2 in allele 1 and a 2 base pair insertion in exon 2 in allele 2; (3) piglet 4-3, having wild-type sequence in both alleles; and (4) piglet 4-4, having a 9 base pair deletion in exon 2 in allele 1 and wild-type sequence in allele 2. Modified alleles were sequenced and are described herein below (Tables 29 and 30). One female from this litter (4-2) was used as a founder animal for creating piglets for the PEDV and TGEV challenges described in Example 6 below.

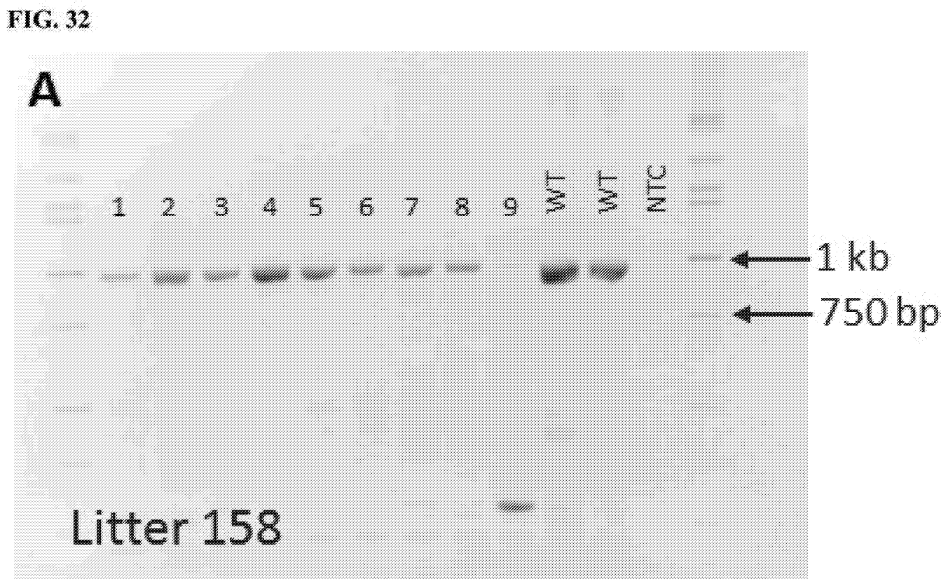

The remaining three ETs were performed with oocytes that were matured in media containing fibroblast growth factor 2 (FGF2), leukemia inhibitory factor (LIF) and insulin-like growth factor 1 (IGF1) (at 40 ng/ml, 20 ng/ml, and 20 ng/ml, respectively). These growth factors (collectively called FLI) were shown by Yuan and colleagues to improve the quality of oocyte maturation (Yuan et al., 2017). Of these three FLI embryo transfers, two recipients did not become pregnant and one recipient farrowed nine piglets. Of the nine piglets, seven contained edits in ANPEP and two were wild-type. Genotypes of this litter (“litter 158”) were determined using TOPO cloning and Sanger sequencing and are summarized in Table 28 below. Representative PCR results depicting each ANPEP allele from these piglets as compared to wild-type (WT) or no template control (NTC) are shown in FIG. 32. One female from this litter (158-1) and one male from this litter (158-9) were used as founder animals to create piglets for the PEDV and TGEV challenges described in Example 6 below.

TABLE 26
Embryo Transfer Data from In-Vitro Fertilization Derived Zygotes Directly
Injected with ANPEP Guide RNA.

| | | | | # Fetuses/Pigs | |
|---|---|---|---|---|---|
| | | # Embryos | Days Post | Collected/ | |
| Pig ID | Line | Transferred | Estrus | Farrowed | Genotype Outcome |
| O345 | ANPEP | 52 (morula/ | 5 | 6 (day 35 | 1 bp deletion, no WT (biallelic) |
| | Injected | blastocyst | | fetuses) | 2 bp deletion, WT (monoallelic) |
| | | stage) | | | 2 bp deletion, 9 bp deletion, WT |
| | | | | | (mosaic) |
| | | | | | 9 bp deletion, WT (monoallelic) |
| | | | | | WT |
| O432 | ANPEP | 68 (morula/ | 4 | — | Cycled back 30 days after ET |
| | Injected | blastocyst | | | |
| | | stage) | | | |
| O448 | ANPEP | 60 (morula/ | 5 | 4 live piglets | 9 bp deletion, WT (monoallelic), |
| | Injected | blastocyst | | | 2 pigs |
| | | stage) | | | 1 bp insertion, 2 bp insertion |
| | | | | | (biallelic) |
| | | | | | WT |
| O606 | ANPEP | 63 (morula/ | 5 | — | Cycled back 30 days after ET |
| | Injected* | blastocyst | | | |
| | | stage) | | | |
| O642 | ANPEP | 60 (morula/ | 5 | 9 live piglets | 1 bp insertion, 12 bp insertion, 9 |
| | Injected* | blastocyst | | | bp deletion, WT (mosaic) |
| | | stage) | | | 1 bp insertion, 1 bp deletion, 25 |
| | | | | | bp deletion, 2 bp mismatch (mosaic) |
| | | | | | 8 bp deletion, 2 bp mismatch |
| | | | | | (biallelic) |
| | | | | | 1 bp insertion, 2 bp insertion |
| | | | | | (biallelic) |
| | | | | | 9 bp deletion, 1 bp mismatch |
| | | | | | (biallelic) |
| | | | | | 1 bp insertion, 2 bp insertion |
| | | | | | (biallelic) |
| | | | | | 661 bp deletion + 8 bp insertion, |
| | | | | | 7 bp deletion + 3 bp addition |
| | | | | | (biallelic) |
| | | | | | WT, 2 pigs |
| O533 | ANPEP | 70 morula/ | 4 | — | Never cycled back, not pregnant |
| | Injected* | (blastocyst | | | |
| | | stage) | | | |

*Indicates that oocytes were cultured in FLI treated medium (Yuan et al., 2017)

TABLE 27
Genotypes of Litter “4”

| Pig ID | Sex | Allele 1 | Allele 2 | Genotype |
|---|---|---|---|---|
| 4-1 | F | 9 bp deletion | WT | ANPEP^{+/-9bp} |
| | | in exon 2 | | |
| 4-2 | F | 1 bp insertion | 2 bp insertion | ANPEP^{-/-} |
| | | in exon 2 | in exon 2 | |
| 4-3 | M | WT | WT | ANPEP^{+/+} |
| 4-4 | M | 9 bp deletion | WT | ANPEP^{+/-9bp} |
| | | in exon 2 | | |

TABLE 28
Genotypes of Litter “158”

| Pig ID | Sex | Allele 1 | Allele 2 | Genotype |
|---|---|---|---|---|
| 158-1* | F | 1 bp deletion | 12 bp deletion | ANPEP^{-/-mosaic} |
| | | in exon 2 | in exon 2 | |
| 158-2^{#} | F | 1 bp insertion | 25 bp deletion | ANPEP^{-/-mosaic} |
| | | in exon 2 | in exon 2 | |
| 158-3 | F | WT | WT | ANPEP^{+/+} |
| 158-4 | F | 8 bp deletion | GT/CA mismatch | ANPEP^{-/+} |
| | | in exon 2 | in exon 2 | |
| 158-5 | F | 1 bp insertion | 2 bp insertion | ANPEP^{-/-} |
| | | in exon 2 | in exon 2 | |
| 158-6 | F | WT | WT | ANPEP^{+/+} |
| 158-7 | M | 9 bp deletion | C/T mismatch | ANPEP^{+/-9bp} |
| | | in exon 2 | in exon 2 | |
| 158-8 | M | 1 bp insertion | 2 bp addition | ANPEP^{-/-} |
| | | in exon 2 | in exon 2 | |
| 158-9 | M | 661 bp deletion + | 7 bp deletion, 3 bp | ANPEP^{-/-} |
| | | 8 bp addition | insertion in exon 2 | |
| | | in exon 2 | | |

*158-1 was mosaic for allele 1, allele 2, a 1 bp insertion in exon 2, a wildtype allele, and a 9 bp deletion in exon 2.
^{#}158-2 was mosaic for allele 1, allele 2, a 1 bp deletion in exon 2, a 2 bp mismatch in exon 2, and a 26 bp deletion in exon 2.

### Genotyping and Phenotypic Characterization of Insertion-Deletions (INDELs)

Each of the modified alleles identified in Tables 25-28 was identified based on sequencing of PCR products amplified from genomic DNA flanking exon 2. The expected effect of these alleles on protein translation and phenotype was determined by translating representative RNA from modified animals to amino acid sequences. Each allele is summarized in detail in Tables 29 and 30 below.

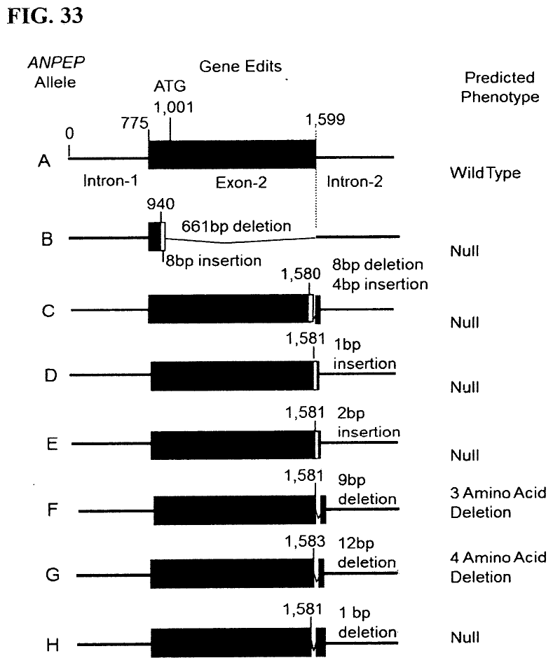

Three pigs from the two live litters (158-1, 158-9, and 4-2) were chosen as founder animals for disease studies described in Example 6 below. Each allele was assigned a letter designation, A-H, with allele A being the wild-type. Each modified allele and the wild-type ANPEP allele is diagrammed in FIG. 33, together with the predicted phenotype. In FIG. 33, the black rectangles represent the coding region and the grey areas represent insertions. The numbers indicate the locations where the insertion and/or deletions occurred.

The ANPEP modified boar (158-9) and one modified dam possessed bi-allelic null edits, consisting of the B and C alleles (boar) or D and E alleles (dam). The second modified dam (158-1) was mosaic for a combination of wild-type (A), null (H), null (D) and other edited alleles (F and G). The B allele has a 661 base pair deletion that includes deletion of the start codon and the deleted sequence is replaced with an 8 base pair insertion. Thus, the B allele results in a complete loss of protein. The C allele results from an 8 base pair deletion, wherein the deleted sequence is replaced by a and 3 base pair insertion, causing a frame shift edit with miscoding starting at amino acid 194 and a premature stop codon at amino acid 223. The two null alleles, D and E also contained frame shift edits, the result of 1 or 2 base pair insertions, respectively. Specifically, the 1 and 2 bp insertions in exon 2 resulted in miscoding at amino acid 194 for both alleles and a premature stop codon at amino acid 220 for the 1 base pair insertion and at amino acid 225 for the 2 base pair insertion. Allele H contained a single base pair deletion that also resulted in miscoding at amino acid 194 and a premature stop codon at amino acid 224. The F and G alleles possessed 9 and 12 base pair deletions, respectively which did not cause a frame shift edit; rather these resulted in the removal of the peptide sequences, 194-M-E-G and 194-M-E-G-N, respectively, as compared to the wild-type amino acid sequence (SEQ ID NO: 134). Allele G also had a single amino acid substitution of V1981 as compared to the wild-type amino acid sequence (SEQ ID NO: 134).

For ease of reference, Table 29 below describes each edit identified in the fetuses collected from the SCNT and IVF experiments (FIGS. 29 and 30). Table 30 below describes each edit found in the live pigs from litters 4 (FIG. 31) and 158 (FIG. 32), including those in the founder animals (alleles A-H). When applicable, alleles identical to alleles A-H found in the non-founder animals (or fetuses) are identified.

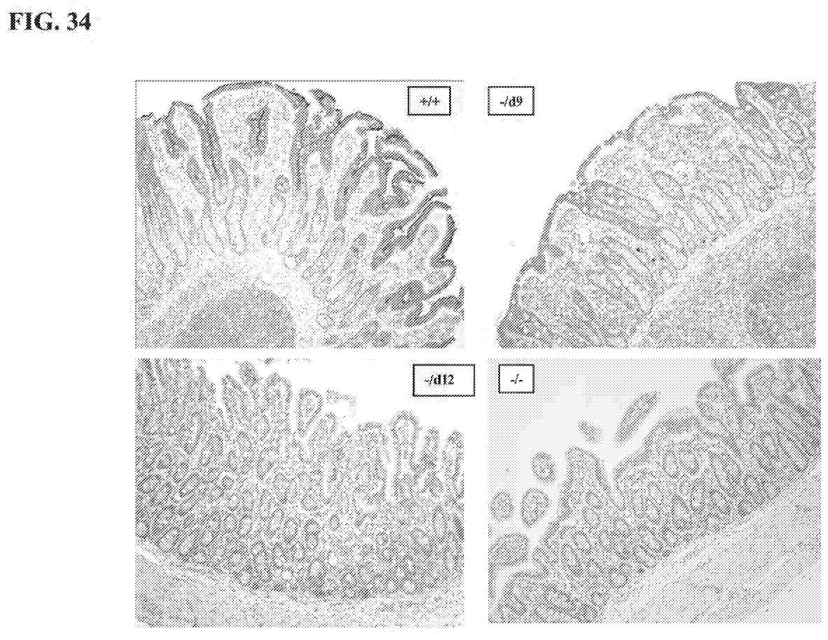

The phenotype of each edit in the founder animals (alleles A-H) was confirmed by immunohistochemistry (IHC) for the expression of ANPEP (CD13) in the ileum of modified pigs (FIG. 34). FIG. 34 shows representative IHC images of ileum of pigs having two A alleles (wild-type, +/+), two null alleles (E/B; −/−), or a null allele in combination with allele F (9 base pair/3 amino acid deletion; B/F; −/d9) or G (12 base pair/4 amino acid deletion; B/G or C/G; −/d12). The image labeled as −/d12 in FIG. 34 is representative of the results obtained with either the B/G or C/G genotype.

To generate the results shown in FIG. 34, paraffin-embedded tissue sections were stained with a 1:32,000 dilution of rabbit anti-CD13 polyclonal antibody (Abcam) prepared against a peptide covering amino acids 400-500 of human CD13. Bound antibody was detected with a horseradish peroxidase-labeled anti-rabbit IgG, and HRP activity was visualized with 3,3′-diaminobenzidine (DAB). Strong immunoreactivity for ANPEP was observed in ileum from animals having two wild-type alleles, while no ANPEP immunoreactivity was observed in the animals having two null alleles. Phenotypically, pigs possessing either the F or G allele showed immunoreactivity for ANPEP, except for weaker immunoreactivity in the four amino acid deletion edit.

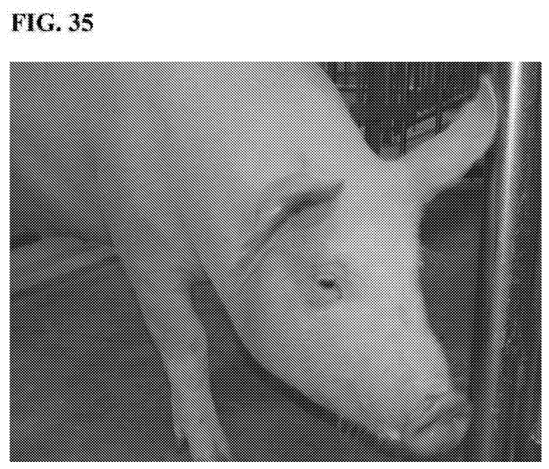

The founder animals (158-1, 158-9 and 4-2) were observed on a daily basis for any phenotypic effects of the mutations. FIG. 35 shows a photograph of pig 158-1 at sexual maturity. The animals all appeared to be healthy and no adverse observations were noted for any of the animals. In particular, no notable problems with lactation were observed. Founder sow 158-1 (a mosaic animal with wild-type alleles) milked normally. Founder sow 4-2 did not milk well with her first litter, but this is not unusual for first parity sows. Founder sow 4-2 milked normally with her second litter. Thus, mammogenesis and lactation appeared to be normal. At the time of filing, pigs 158-1 and 158-9 were approximately 2.5 years old and pig 4-2 was about 3 years old, and no adverse observations had been noted.

All animals used in the virus challenge studies described in Example 6 below were also monitored daily for any phenotypic effects of the mutations. The animals containing the modified ANPEP alleles did not show any signs of TGEV infection and appeared to be healthy.

TABLE 29
Edits in fetuses from SCNT and IVF experiments (FIGS. 29 AND 30)

| | | | | Description as compared to wild-type | | |
|---|---|---|---|---|---|---|
| INDEL | | | Protein Translation as compared to | nucleic acid sequence (reference | | SEQ ID |
| # | FETUS/PIG | Description | wild-type ANPEP (SEQ ID: 134) | sequence SEQ ID NO: 135) | NOTES | NO. |
| 1 | Fetus (FIG. 29) | 182 bp | In-frame deletion of AA129-167 and 174- | 182 bp deletion from nt 1,397 to nt | | 163 |
| | Genotype 1 | deletion, 5 | 193 and Y171V, E172P, and M173S | 1,578; deleted sequence is replaced with | | |
| | | bp insertion | substitutions. No premature stop codon. | a 5 bp insertiona beginning at nt 1,397 | | |
| 2 | Fetus (FIG. 29) | 9 bp | In-frame deletion of AA192, 193, 194 | 9 bp deletion from nt 1,574 to nt 1,582 | | 164 |
| | Genotype 2 | deletion | (E-Y-M). | | | |
| | | | No premature stop codon. | | | |
| 3 | Fetus (FIG. 29) | 867 bp | No translated protein. Deletion removes | 867 bp deletion from nt 819 to nt 1,685. | | 165 |
| | Genotype 4 | deletion | the start codon. | | | |
| 4 | Fetus (FIG. 30) | 1 bp | Miscoding starts at AA194 (M→I) with | 1 bp insertion between nt. 1,581 and nt | SAME AS | 166 |
| | Genotype I | insertion | premature stop codon at AA220. | 1,582.^{b} | ALLELE D | |
| | | | | | (Table 30) | |
| 5 | Fetus (FIG. 30) | 2 bp | Miscoding starts at AA194 (M→I) with | 2 bp insertion between nt. 1,581 and nt | SAME AS | 167 |
| | Genotype 2 | insertion | premature stop codon at AA225. | 1,582.^{c} | ALLELE E | |
| | | | | | (Table 30) | |
| 6 | Fetus (FIG. 30) | 2 bp | Miscoding starts at AA194 (M→I) with | 2 bp insertion between nt. 1,581 and nt | SAME AS | 167 |
| | Genotype 5a | insertion | premature stop codon at AA225. | 1,582.^{c} | ALLELE E | |
| | | | | | (and INDEL 5) | |
| 7 | Fetus (FIG. 30) | 9 bp | In-frame deletion of AA192-194 (E-Y-M). | 9 bp deletion from nt 1,574 to nt 1,582. | SAME AS | 164 |
| | Genotype 5b | deletion | No premature stop codons | | INDEL 2 | |
| 8 | Fetus (FIG.30) | 267 bp | In-frame deletion of AA108-196. No | 267 bp deletion from nt 1,321 to nt | | 168 |
| | Genotype 5c | deletion | premature stop codons | 1,587. | | |
| 9 | Fetus (FIG. 30) | 9 bp | In-frame deletion of AA192-194 (E-Y-M). | 9 bp deletion from nt 1,574 to nt 1,582. | SAME AS | 164 |
| | Genotype 6 | deletion | No premature stop codons. | | INDEL 2 | |

^{a}Insertion is CCCTC (SEQ ID NO: 169)
^{b}Insertion is a single thymine (T) residue.
^{c}The inserted sequence is AT.

Table 30: Edits in Live Pigs from Litters 4 and 158(FIGS. 31 and 32)

TABLE 30
Edits in live pigs from litters 4 and 158 (FIGs. 31 and 32)

| | | | | Description as compared | | |
|---|---|---|---|---|---|---|
| | | | Protein Translation | to wild-type nucleic | | |
| | | | as compared to | acid sequence | | SEQ |
| INDEL | FETUS/ | | wild-type ANPEP | (reference sequence | | ID |
| # | PIG | Description | (SEQ ID NO: 134) | SEQ ID NO: 135) | NOTES | NO: |
| 10 | 4-1 | 9 bp deletion | In-frame deletion of | 9 bp deletion from nt | ALLELE F | 170 |
| | | | AA194-196 (M-E-G). | 1,581 to nt 1,589. | | |
| | | | No premature stop codons. | | | |
| 11 | 4-2* | 1 bp insertion | Miscoding at AA194 (M->I). | 1 bp insertion^{a} between | ALLELE D | 166 |
| | | | Premature stop codon at AA220 | nt 1,581 and nt 1,582. | | |
| 12 | | 2 bp insertion | Miscoding at AA194 (M-> I). | 2 bp insertion^{b} between | ALLELE E | 167 |
| | | | Premature stop codon at AA 225 | nt1,581 and nt 1,582. | | |
| 13 | 4-4 | 9 bp deletion | In-frame deletion of AA194-196 | 9 bp deletion from | ALLELE F | 170 |
| | | | (M-E-G). No premature stop | n, 1,581 to nt 1,589. | | |
| | | | codon. | | | |
| 14 | 158-* | 1 bp deletion | Miscoding at AA194 (M-->R). | 1 bp deletion of nt 1,581. | ALLELE H | 171 |
| | | | Premature stop codon at 224. | | | |
| 15 | | 1 bp insertion | Miscoding at AA194 (M->I). | 1 bp insertion^{a} between | ALLELE D | 166 |
| | | | Premature stop codon at | nt 1,581 and nt 1,589. | | |
| | | | AA220 | | | |
| 16 | | 9 bp deletion | In-frame deletion of AA194- | 9 bp deletion from nt | ALLELE F | 170 |
| | | | 196. (M-E-G). No premature | 1,581 to nt 1,589. | | |
| | | | stop codon. | | | |
| 17 | | 12 bp deletion | In-frame deletion of AA194- | 12 bp deletion from nt | ALLELE G | 172 |
| | | | 197 (M-E-G-N) and V198I amino | 1,582 to nt 1,593 | | |
| | | | acid substitution. | | | |
| 18 | 158-2 | 1 bp insertion | Miscoding at AA194 (M->I). | 1 bp insertion^{a} between | ALLELE D | 166 |
| | | | Premature stop codon at AA220. | nt 1,581 and nt 1,582 | | |
| 19 | | 25 bp deletion | Miscoding at AA188 (F->A). | 25 bp deletion from nt | | 173 |
| | | | Premature stop codon at | 1,561 to nt 1,585. | | |
| | | | AA 216 | | | |
| 20 | 158-4 | 8 bp deletion | Miscoding at AA192 (E->G). | 8 bp deletion from nt | | 174 |
| | | | Premature stop codon at AA217. | 1,575 to nt 1,582. | | |
| 21 | | 2 bp mismatch | M194N substitution that | 2 bp substitution from | | 175 |
| | | | likely does not confer disease | TG to AC at nt 1,581 and | | |
| | | | resistance but is not wild- | nt 1,582. | | |
| | | | type. | | | |
| 22 | 158-5 | 1 bp insertion | Miscoding at AA194 (M-->N). | 1 bp insertion^{c} between | ALLELE E | 176 |
| | | | Premature stop codon at 220. | nt 1,579 and nt 1,580. | | |
| 23 | | 2 bp insertion | Miscoding at AA194 (M-->I). | 2 bp insertion^{b} between | | 167 |
| | | | Premature stop codon at | nt 1,581 and nt 1,582. | | |
| | | | AA 225. | | | |
| 24 | 158-7# | 9 bp deletion | In-frame deletion of AA194- | 9 bp deletion from nt | ALLELE F | 170 |
| | | | 196. (M-E-G). No premature | 1,581 to nt 1,589. | | |
| | | | stop codon. | | | |
| 25 | 158-8 | 1 bp insertion | Miscoding at AA194 (M->I). | 1 bp insertion^{a} between | ALLELE D | 166 |
| | | | Premature stop codon at AA220. | nt 1,581 and nt 1,582. | | |
| 26 | | 2 bp insertion | Miscoding at AA194 (M-> I). | 2 bp insertion^{b} between | ALLELE E | 167 |
| | | | Premature stop codon at | 1,581 and nt 1,582. | | |
| | | | AA 225 | | | |
| 27 | 158-9* | 661 bp deletion, | No translation (start codon | 661 bp deletion from nt | ALLELE B | 177 |
| | | 8 bp insertion | is deleted)/ | 940 to nt 1600; deleted | | |
| | | | | sequence is replaced with | | |
| | | | | an 8 bp insertion^{d} start- | | |
| | | | | ing at nt 940. | | |
| 28 | | 7 bp deletion | Miscoding at AA194 (M->S). | 8 bp deletion from nt | ALLELE C | 178 |
| | | and 3 bp | Premature Stop codon at | 1,580 to nt 1,587; deleted | | |
| | | insertion | AA223. | sequence is replaced with | | |
| | | | | a 4 bp insertion^{e} begin- | | |
| | | | | ning at nt 1,580. | | |

^{a}Insertion is a single thymine (T) residue.
^{b}The inserted sequence is AT.
^{c}Insertion is a single adenine (A) residue.
^{d}THe inserted sequence is GGGGCTTA (SEQ ID NO: 179)
^{e}The inserted sequence is TCGT (SEQ ID NO: 180)
#This pig also had a 1 bp mismatch (C->T) that was identified as a polymorphism unrelated to the CRISPR modifications
*Founder pigs

### Example 6: Increased Resistance to TGEV in Swine Having a Modified Chromosomal Sequence in a Gene Encoding an ANPEP Protein

In the present example, pigs having a modified chromosomal sequence in ANPEP were challenged with porcine epidemic diarrhea virus (PEDV) or transmissible gastroenteritis virus (TGEV) and monitored to assess their resistance to infection. Lack of ANPEP resulted in an increased resistance to TGEV, but not PEDV, as measured by viremia titers and other markers.

### Materials and Methods

### Breeding Pigs for PEDV Studies

For PEDV studies, two gilts (4-2 and 158-1) were synchronized by feeding 6.8 mL containing 15 mg of altrenogest product, MATRIX (Intervet Inc. Millsboro, Del.) each day for 14 days. Gilts 4-2 and 158-1 came into heat within five days after the altrenogest was stopped and were bred by artificial insemination (AI) with semen collected from boar 158-9. Gilt 4-2 did not become pregnant. After 117 days of gestation, sow 158-1 farrowed 8 healthy piglets. One piglet was crushed by the sow.

### Breeding Pigs for TGEV Challenge

ANPEP-edited F1 pigs were again bred to create litters of ANPEP-edited pigs for the TGEV challenge. The same two gilts (4-2 and 158-1) were synchronized by the same method described above and were bred by artificial insemination (AI) with semen collected from boar 158-9. Both sows 158-1 and 4-2 became pregnant. Sow 158-1 farrowed four piglets (litter 127). Three piglets were healthy and one piglet had poor rear leg structure and was euthanized. Sow 4-2 farrowed 13 piglets (litter 20); 11 were healthy. One piglet would not nurse and died and another piglet had poor rear leg structure. Two of the other piglets were later crushed by the sow.

### Viruses

PEDV KS13-09 was propagated on VERO76 cells maintained in MEM supplemented with 10% fetal bovine serum (FBS; Sigma), 1% Pen-Strep (Gibco) and 0.25 μg/ml FUNGIZONE (amphotericin B). Cells were infected in medium containing 2% Tryptose Phosphate Broth (Sigma) and 1 μg/ml L-1-Tosylamide-2-phenylethyl chloromethyl ketone (TPCK; Sigma). For virus titration, VERO76 cells in 96-well plates were infected with serial 1:10 dilutions of virus in octuplicate at 37° C. with 5% CO₂. After 3 hours, the cell culture medium was replaced with fresh infection medium. At 18 hours, the cells were fixed with an acetone:methanol mixture (at 3:2 ratio) for 30 minutes at 4° C. and reacted with a 1:500 dilution of rabbit polyclonal antibody directed against the PEDV M protein (Genscript). After washing with PBS, FITC-conjugated goat-anti-rabbit IgG (Jackson ImmunoResearch) was added as the secondary antibody. Virus concentration was calculated as the TCID₅₀/ml using Reed and Muench method (Reed and Muench, 1938).

TGEV Purdue strain was cultivated on swine testicular (ST) cells maintained in MEM-FBS media 10%, the same as described for PEDV. For titration, the virus was serially diluted 1:10 in quadruplicate on confluent ST cells in a 96-well tissue culture plate (BD Falcon). Following 3 days of incubation at 37° C. and 5% CO2, wells were examined for the presence of cytopathic effect (CPE). The last well showing CPE was used as the titration endpoint and the 50% tissue culture infectious dose (TCID₅₀) per ml was calculated as described in (Reed and Muench, 1938.

Infection with PEDV/TGEV

Experiments involving animals and viruses were performed in accordance with the Federation of Animal Science Societies Guide for the Care and Use of Agricultural Animals in Research and Teaching, the USDA Animal Welfare Act and Animal Welfare Regulations, and were approved by the Kansas State University and University of Missouri institutional animal care and institutional biosafety committees. During the challenges, all infected WT and ANPEP-modified pigs were housed together in a single room in the large animal resource center. Therefore, all ANPEP-edited pigs received continuous exposure to viruses shed by the infected wild-type littermates. For infection, pigs received an initial dose of PEDV prepared from a PCR-positive intestinal tissue homogenate from experimentally infected pigs (Niederwerder et al., 2016). Four days later, the pigs were infected a second time with a tissue culture-derived isolate, PEDV KS13-09, which was orally administered as a single 10 ml dose containing 10⁶ TCID₅₀ of virus. For TGEV, pigs received the same amount of virus administered orally.

Fecal swabs were collected daily from each animal beginning one day prior to challenge with PEDV until the termination of the study. Each swab was placed in a 15 ml conical tube containing 1 ml of MEM with 1% Pen-Strep and 1% FUNGIZONE. The tube was vortexed briefly to mix the swab contents, aliquoted into 1.5 ml cryovials and then stored at −80° C.

Sera were collected on days 3, 7, and 9 after initial exposure. Both feces and sera were and examined using RT-PCR to detect PEDV or TGEV nucleic acid. After nine days, the animals were sacrificed and immunohistochemistry (IHC) was performed on paraffin-embedded intestine (ileum) to detect PEDV or TGEV antigen.

RT-PCR for the detection of viral nucleic acid

Total RNA was extracted from fecal and serum samples using a MAGMAXTM-96 Total RNA Isolation Kit (Invitrogen) according to the manufacturer's instructions on a KINGFISHER instrument (Thermo Scientific). PEDV nucleic acid was amplified using a SUPERSCRIPT III one-step RT-PCR kit with PLATINUM Taq DNA polymerase and the primers listed in Table 31 in a total volume of 50 μl. PCR was performed as follows: initial reverse transcription at 58° C. for 30 minutes followed by denaturation at 94° C. for 2 minutes; and then 40 cycles of 94° C. for 15 seconds, 48° C. for 30 seconds, and 68° C. for 90 seconds. PCR products were visualized on a 1% agarose gel. The results were recorded based on the intensity of ethidium bromide staining.

TGEV nucleic acid was amplified using a real time procedure (Vemulapalli R., 2016). Forward and reverse primers and a TAQMAN probe (BHQ-1) included in the TAQMAN Fast Virus 1-Step Master Mix (Thermo Fisher) are listed in Table 31. RT-PCR included reverse transcription at 50° C. for 30 minutes, reverse transcription at 95° C. for 15 minutes followed by 45 cycles of 95° C. for 15 seconds, 56° C. for 30 seconds and 72° C. for 15 seconds. PCR was performed on a CFX-96 real-time PCR system (Bio-Rad) in a 96-well format and the result for each sample is reported as a Ct value.

TABLE 31
Primers for RT-PCR of Viral Nucleic Acid

| Primer | Sequence (5′-3′) | SEQ ID NO. |
|---|---|---|
| PEDV (F) | ATGGCTTCTGTCAGTTTTCAG | 181 |
| PEDV (R) | TTAATTTCCTGTGTCGAAGAT | 182 |
| TGEV (F) | TCTGCTGAAGGTGCTATTATATGC | 183 |
| TGEV (R) | CACAATTTGCCTCTGAATTAGAAG | 184 |
| BHQ1 probe | YAAGGGCTCACCACCTACTACCACCA | 185 |

### Immunohistochemistry (IHC) for Detection of Viral Antigen in Tissues

Immunohistochemistry to detect the levels of PEDV and TGEV antigen in the intestine (ileum) of infected animals were performed as a routine diagnostic test by the Kansas State University and University of Missouri veterinary diagnostic laboratories using similar methods as described above in Example 5 for the detection of ANPEP antigen in modified pig. Anti-spike protein monoclonal antibody was used to detect PEDV antigen (Cao et al., 2013). TGEV antigen was detected using anti-feline infectious peritonitis coronavirus antibody.

### Detection of TGEV-Specific Antibody in Serum

Blocking ELISA and indirect immunofluorescence antibody (IFA) were used to detect TGEV-specific antibodies in serum. For IFA, confluent ST cells on 96 well plates were infected with 200 TCID₅₀/ml of TGEV Purdue. After 3 days incubation at 37° C. and 5% CO2, cells were fixed with 80% acetone. Serum from each pig was serially diluted in PBS with 5% goat serum (PBS-GS). A serum sample obtained from each pig prior to infection served as a negative control. After incubation for 1 hour at 37° C., plates were washed and secondary antibody added to each well. Alexa-Fluor-488 AffiPure goat anti-swine IgG (Cat #114-545-003, Jackson ImmunoResearch) was diluted 1:400 dilution in PBS-GS. Plates were incubated for 1 hour at 37° C., washed with PBS, and viewed under a fluorescence microscope. Blocking assays were performed using a kit, SVANOVIR TGEV/PRCV, from Sanova. Assays were performed according to the kit instructions and results reported as percent inhibition of binding of labeled TGEV-specific antibody.

### Results

Breeding of Pigs and Infection with PEDV

The genotypic classification of each offspring piglet from the litter used for the PEDV challenge is summarized in Table 32 below. Piglets that were challenged included three pigs heterozygous for the wildtype ANPEP allele, two pigs possessing the four amino acid deletion, a single pig with the three amino acid deletion, and a single knockout pig. Five wildtype pigs from a separate litter were used as unmodified controls and are not included on the table.

TABLE 32
Genotype of each allele from F2 piglets that were
challenged with PEDV

| | | | | | | Geno- |
|---|---|---|---|---|---|---|
| | | | | | | type |
| | Ear | | | Allele | | Classi- |
| ANPEP | Tags | Sex | Genotype | 1 | Allele 2 | fication |
| 121-1 | 126 | Boar | ANPEP^{+/-} | WT | 8 bp deletion, | A/C |
| | | | | | 4 bp | |
| | | | | | addition | |
| 121-2 | crushed | Boar | ANPEP^{+/-} | WT | 8 bp deletion, | A/C |
| | | | | | 4 bp | |
| | | | | | addition | |
| 121-3 | 133 | Boar | ANPEP^{+/-} | 1 bp | 8 bp deletion, | H/C |
| | | | | deletion | 4 bp | |
| | | | | | addition | |
| 121-4 | 125 | Boar | ANPEP^{-12/-} | 12 bp | 661 bp deletion, | G/B |
| | | | | deletion | 8 bp | |
| | | | | | addition | |
| 121-5 | 136 | Gilt | ANPEP^{-12/-} | 12 bp | 8 bp deletion, | G/C |
| | | | | deletion | 4 bp | |
| | | | | | addition | |
| 121-6 | 131 | Gilt | ANPEP^{+/-} | WT | 8 bp deletion, | A/C |
| | | | | | 4 bp | |
| | | | | | addition | |
| 121-7 | 134 | Gilt | ANPEP^{+/-} | WT | 661 bp deletion, | A/B |
| | | | | | 8 bp | |
| | | | | | addition | |
| 121-8 | 130 | Gilt | ANPEP^{+/-} | 9 bp | 661 bp deletion, | F/B |
| | | | | deletion | 8 bp | |
| | | | | | addition | |

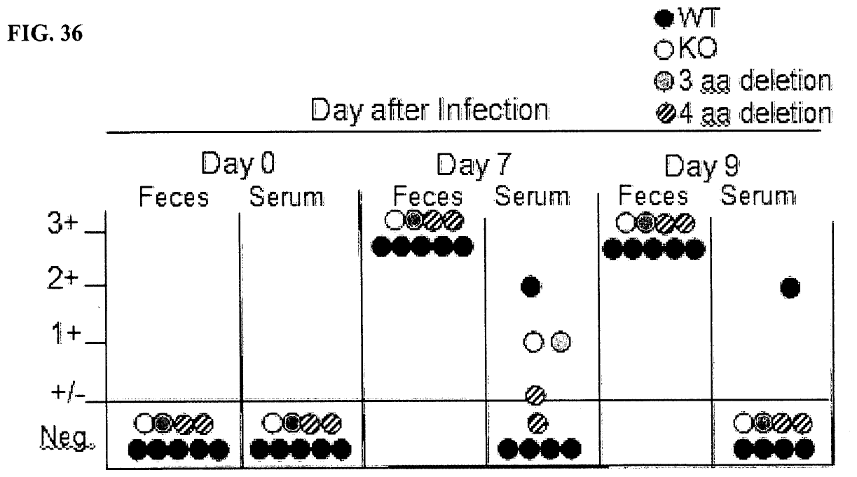

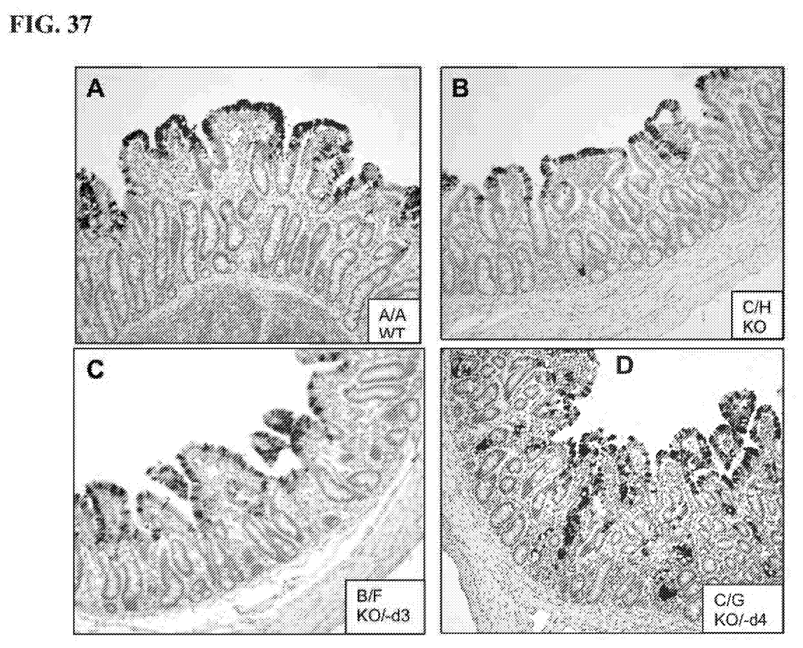

At three weeks, the piglets were exposed to PEDV as described above and feces and sera were collected for characterization with RT-PCR. FIG. 36 shows the normalized amount of PEDV nucleic acid in feces and serum of each infected pig, except those heterozygous for the WT allele, at day 0, 7 and 9. Each pig was classified based on its genotype: wildtype (black), knockout/null (white), 3 aa deletion (9 bp deletion, grey), and 4 aa deletion (12 bp deletion, striped). The results for PEDV quantification by PCR and IHC for all pigs are depicted in Table 33. PEDV quantification in terms of the RT-PCR product is depicted in FIG. 36 as a measure of ethidium bromide staining; from (3+) for intense staining to (Neg.) for no detectable PCR product. All pigs were strongly positive for PEDV nucleic acid in feces beginning at seven days after infection (Table 33, FIG. 36). At least one pig from each of the groups (null, three amino acid deletion, four amino acid deletion, and WT) were also positive in serum at day 7 (Table 33; FIG. 36). In addition, IHC confirmed that all pigs possessed antigen in enterocytes (Table 33, FIG. 37). FIG. 37 shows representative images of the ileum in wildtype (panel A), knockout (panel B), 3 aa deletion (panel C), and 4 aa deletion (panel D) pigs stained for PEDV antigen (black). Thus, the absence of ANPEP did not prevent PEDV infection.

TABLE 33
Summary of PEDV PCR and IHC results*¹

| | | Day after Infection | | | | | | | | | | | | |
|---|---|---|---|---|---|---|---|---|---|---|---|---|---|---|
| | Geno- | 1 | 2 | 3 | | 4 | 5 | 6 | 7 | | 8 | 9 | | |
| Pig No. | type | F | F | F | S | F | F | F | F | S | F | F | S | IHC |

Wild type pigs

| | | | | | | | | | | | | | | |
|---|---|---|---|---|---|---|---|---|---|---|---|---|---|---|
| 127 | +/ + | − | − | − | − | − | − | +++ | +++ | − | − | +++ | − | +++ |
| 128 | +/ + | − | − | − | − | − | − | +++ | +++ | ++ | +++ | +++ | ++ | +++ |
| 129 | +/ + | − | − | − | − | − | + | +++ | +++ | − | +++ | +++ | − | +++ |
| 132 | +/ + | − | − | − | − | − | − | ++ | +++ | − | +++ | +++ | − | +++ |
| 135 | +/ + | − | − | − | − | − | − | ++ | +++ | − | +++ | +++ | − | +++ |

Genetically-modified pigs

| | | | | | | | | | | | | | | |
|---|---|---|---|---|---|---|---|---|---|---|---|---|---|---|
| 126 | −/+ | − | − | − | − | − | + | +++ | +++ | − | +++ | +++ | − | +++ |
| 131 | −/+ | − | − | − | − | − | +++ | +++ | +++ | − | +++ | +++ | − | +++ |
| 134 | −/+ | − | − | − | − | − | +++ | +++ | +++ | − | +++ | +++ | − | +++ |
| 125 | −/d12*² | − | − | − | − | − | +++ | +++ | +++ | − | +++ | +++ | − | +++ |
| 136 | −/d12 | − | − | − | − | − | +++ | +++ | +++ | +/− | +++ | +++ | − | +++ |
| 130 | −/d9*² | − | − | − | − | − | ++ | +++ | +++ | + | − | +++ | − | +++ |
| 133 | −/− | − | − | − | − | − | +++ | +++ | +++ | + | +++ | +++ | − | +++ |

*¹Pigs were infected with virus on days 1 and 4. Samples for PCR include feces (F) and serum (S). Immunohistochemistry (IHC) was performed in paraffin-embedded intestine (ileum). PCR and IHC results are presented as: −, negative; +, weakly positive; ++, positive; +++, strongly positive.
*²The mutated ANPEP gene possessed deletions of 9 or 12 bp in exon 2, which did not alter the reading frame.

Breeding of Pigs for Infection with TGEV

The genotypic classification of each offspring piglet used for the TGEV challenge is summarized in Table 34 (for litter 20) and 35 (for litter 127) below. In all, six piglets from litter 20 and two piglets from litter 127 were challenged with TGEV. Of these, seven were null for ANPEP and one had a three amino acid deletion. Seven wild-type pigs from a separate litter were used as positive controls.

TABLE 34
Genotypes of Litter 20 piglets from sow 4-2 that were
challenged with TGEV

| | | | | | | Geno- |
|---|---|---|---|---|---|---|
| | | | | | | type |
| Ear | Study | | | | | Classi- |
| Tag | ID^{#} | Sex* | Genotype* | Allele 1 | Allele 2 | fication |
| 20-1 | 144 | Gilt | ANPEP^{-/-} | 2 bp | 661 bp | B/E |
| | | | | insertion | deletion + 8 bp | |
| | | | | | addition | |
| 20-2 | 147 | Gilt | ANPEP^{-/-} | 1 bp | 8 bp deletion, | C/D |
| | | | | insertion | 4 bp | |
| | | | | | addition | |
| 20-3 | 142 | Gilt | ANPEP^{-/-} | 2 bp | 8 bp deletion, | C/E |
| | | | | insertion | 4 bp | |
| | | | | | addition | |
| 20-4 | 151 | Boar | ANPEP^{-/-} | 1 bp | 8 bp deletion, | C/D |
| | | | | insertion | 4 bp | |
| | | | | | addition | |
| 20-5 | 146 | Boar | ANPEP^{-/-} | 2 bp | 661 bp | B/E |
| | | | | insertion | deletion + 8 bp | |
| | | | | | addition | |
| 20-6 | 149 | Boar | ANPEP^{-/-} | 1 bp | 8 bp deletion, | C/D |
| | | | | insertion | 4 bp | |
| | | | | | addition | |
| 20-7 | NC | Boar | ANPEP^{-/-} | 2 bp | 8 bp deletion, | C/E |
| (dead) | | | | insertion | 4 bp | |
| | | | | | addition | |
| 20-8 | NC | Boar | ANPEP^{-/-} | 1 bp | 8 bp deletion, | C/D |
| | | | | insertion | 4 bp | |
| | | | | | addition | |
| 20-9 | NC | Boar | ANPEP^{-/-} | 2 bp | 8 bp deletion, | C/E |
| | | | | insertion | 4 bp | |
| | | | | | addition | |
| 20-10 | NC | Boar | ANPEP^{-/-} | 2 bp | 661 bp | B/E |
| | | | | insertion | deletion + 8 bp | |
| | | | | | addition | |
| 20-11 | NC | ND | ND | not | 661 bp | |
| (dead) | | | | genotyped | deletion + 8 bp | |
| | | | | | addition | |
| 20-12 | NC | ND | ND | not | 661 bp | |
| (dead) | | | | genotyped | deletion + 8 bp | |
| | | | | | addition | |
| 20-13 | NC | ND | ND | not | 8 bp deletion, | |
| (dead) | | | | genotyped | 4 bp | |
| | | | | | addition | |

^{#}NC: Not challenged;
*ND: Not determined

TABLE 35
Genotypes of Litter 127 piglets from sow 158-1 that
were challenged with TGEV

| | | | | | | Genotype |
|---|---|---|---|---|---|---|
| Ear | Study | | | | | Classi- |
| Tag | ID^{#} | Sex | Genotype | Allele 1 | Allele 2 | fication |
| 127-1 | NC | Boar | ANPEP^{-/-} | 1 bp insertion | 8 bp deletion, | C/D |
| | | | | | 4 bp | |
| | | | | | addition | |
| 127-2 | 140 | Gilt | ANPEP^{-9/-} | 9 bp deletion | 661 bp | B/F |
| | | | | | deletion + 8 bp | |
| | | | | | addition | |
| 127-3 | 153 | Gilt | ANPEP^{-/-} | 1 bp deletion | 8 bp deletion, | C/H |
| | | | | | 4 bp | |
| | | | | | addition | |
| 127-4 | NC | Gilt | ANPEP^{+/-} | WT | 661 bp | A/B |
| | | | | | deletion + 8 bp | |
| | | | | | addition | |

^{#}NC: Not challenged

Outcome from TGEV Challenge

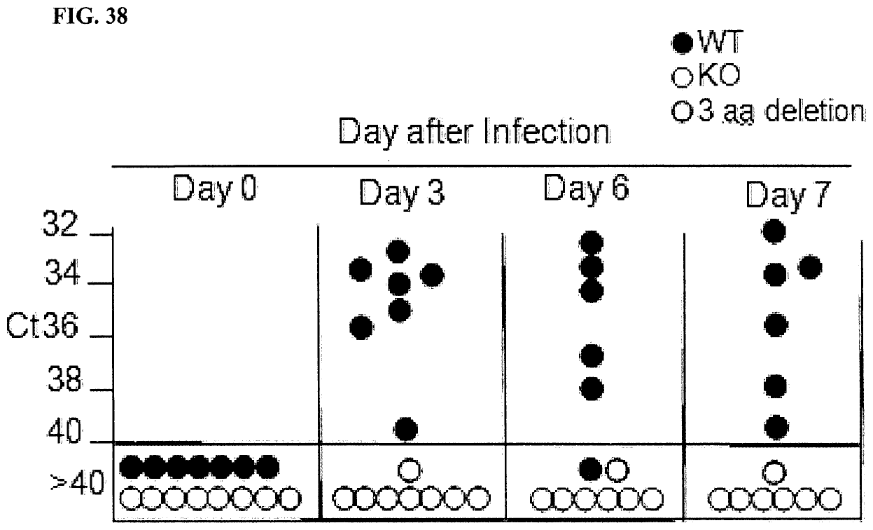

When the piglets were three weeks old, they were challenged with TGEV Purdue as described above, using the same route, dose, and housing conditions as for the PEDV challenge. A wild-type (WT) and a knockout (KO) pig were each removed from the study and euthanized at day 4 for testing. A commercial RT-PCR assay was used to detect the presence of virus in feces and sera, and IHC was used to detect TGEV antigen in ileum. PCR results for virus in feces at days 0, 3, 6, and 7 after initial exposure to TGEV are provided in FIG. 38. Results are shown as Ct values. The black circles represent WT pigs, which were positive for the presence of TGEV nucleic acid in feces starting on day 3. Viral nucleic acid was not detected in feces of the single pig possessing the F allele (three amino acid deletion, grey circle) or in any of the seven knockout (KO) pigs (white circles) during the first week of infection (FIG. 38). Note that only 6 WT and KO animals are plotted for day 6 and 7 because one WT and one KO pig were removed from the study at day 4 for immunohistochemistry (below). All pigs were RT-PCR negative by the end of the 13 day study (data not shown).

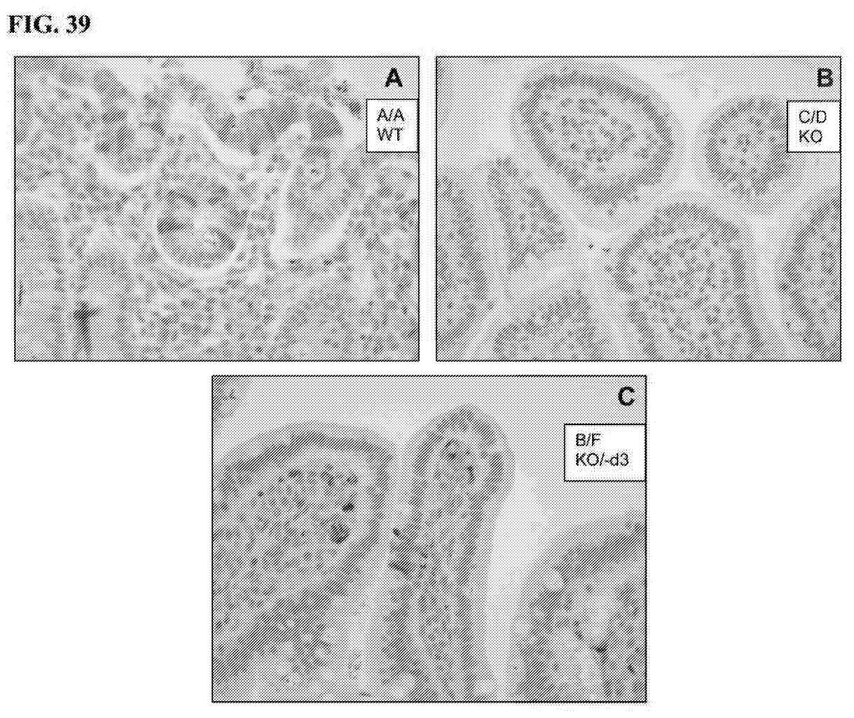

FIG. 39 shows representative immunohistochemistry images of ileum stained for TGEV antigen from wild-type pigs (WT, Panel A), knockout pigs (KO, Panel B) or pigs having a null allele and an allele containing the three aa deletion (KO/−d3; Panel C). TGEV antigen staining on intestinal tissues was performed on a single WT and KO pig removed from the study 4 days after infection, during a period of time when the greatest amount of viral nucleic acid was present in feces. The WT pig was positive for the presence of TGEV antigen in ileum (FIG. 39, Panel A), while the ANPEP KO pig was negative (FIG. 39, Panel B). The intestinal tissue from the pig possessing the three amino acid deletion (the F allele) was stained for TGEV antigen at 13 days after infection. The results showed positive antibody staining for viral antigen in ileum (FIG. 39, panel C).

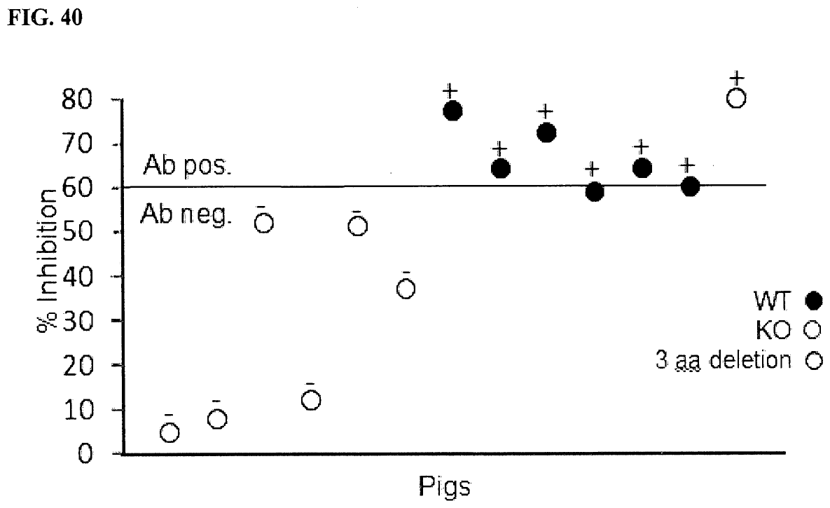

Sera obtained at the end of the study were tested for the presence of the TGEV-specific antibody using immunofluorescent (IFA) and blocking ELISA assays. Both the immunofluorescent assay (IFA) and the blocking ELISA assay showed that the WT and F allele pigs were positive for the presence of TGEV-specific antibody; whereas, no TGEV specific antibody was detected in the ANPEP KO pigs (FIG. 40). In FIG. 40, the horizontal line shows the cutoff for a positive/negative result. The plus and minus symbols show the results for antibody measurements using indirect IFA. Even though the pig possessing the three amino acid deletion was negative for TGEV nucleic acid in feces, positive staining for TGEV antigen in ileum and a positive antibody response confirmed that this pig was productively infected. Note that the number of pigs in FIG. 40 reflects the number of pigs remaining after the removal of a WT and KO pig for IHC at day 4.

These data establish that the presence of ANPEP is required for the infection of pigs with TGEV. They also suggest that reducing ANPEP function (e.g., as in the case of the F allele) may provide a beneficial outcome as measured by reduced viral levels in the feces.

### Example 7: Generation of Animals Heterozygous for Chromosomal Modifications in at Least Two Genes Selected from ANPEP, SIGLEC1 and CD163

### Materials and Methods

### Breeding

An outcross gilt (14-1) that carried one allele with an ANPEP edit (a 1 bp insertion, allele D, SEQ ID NO: 166), and a wild type (WT) allele was bred by artificial insemination with an outcross gilt that was heterozygous for edits in both the CD163 gene and the SIGLEC1 gene (Table 36). The edit in the CD163 gene was the 1387 base pair deletion from nucleotide 3,145 to nucleotide 4,531 as compared to reference sequence SEQ ID NO: 4, such that the CD163 gene comprised SEQ ID NO: 112. The edit in the SIGLEC1 gene was a 1,247 base pair deletion from nucleotide 4,279 to nucleotide 5,525 as compared to reference sequence SEQ ID NO: 122, wherein the deleted sequence was replaced with a neomycin gene cassette, such that the SIGLEC1 gene comprised SEQ ID NO: 123.

The sow farrowed 10 healthy piglets with no mummies or still born fetuses. The piglets all appeared to be healthy at birth. Two piglets were euthanized because only one allele was edited. The remaining piglets continue to be healthy and as of filing, were almost 2 months old.

TABLE 36
Breeding combination that produced Litter 144

| Pig | Sex | Genotype | Allele 1 | | Allele 2 |
|---|---|---|---|---|---|
| 14-1 | Gilt | ANPEP^{+/-} | 1 bp insertion | SEQ ID NO: 166 | WT (outcross) |
| 193-2 (P156) | Boar | CD163^{+/-} | 1387 bp deletion | SEQ ID NO: 112 | WT (outcross) |
| 193-2 (P156) | Boar | SIGLEC^{+/-} | Neo inserted | SEQ ID NO: 123 | WT (outcross) |

### Genotyping

DNA isolation: Genomic DNA lysates were prepared by digesting a small piece of the cropped tail in 250 μL of lysis buffer (40 mM Tris, pH 8.9, 0.9% Triton X-100, 0.4 mg/mL proteinase K, (NEB)) and incubating at 56° C. for 12 hours for cell lysis followed by incubation at 85° C. for 10 minutes to inactivate the proteinase K. Tail lysate genomic DNA was used directly as template for PCR.

CD163: Genomic DNA was used to assess genotype by PCR and agarose gel electrophoresis. PCR was performed with the CD163 specific forward primer “TTGTTGGAAGGCTCACTGTCCTTG” (SEQ ID NO: 68, Table 3) and reverse primer “ACAACTAAGGTGGGGCAAAG” (SEQ ID NO: 69, Table 3) by using standard protocol and LA Taq (Takara, Mountain View, Calif.). PCR conditions were 95° C. for 2 minutes and 33 cycles of 94° C. for 30 seconds, 50° C. for 30 seconds and 68° C. for 7 minutes followed by a final extension of 72° C. for 2 minutes. A 6358 bp amplicon was then separated on a 1.25% agarose gel. The 1387 bp deletion was visible after electrophoresis and was not sequenced. The exact sequence was known from the founder animals.

ANPEP: Genomic DNA was used to assess genotype by PCR agarose gel electrophoresis and subsequent Sanger DNA sequencing. PCR was performed with the ANPEP specific forward primer “ACGCTGTTCCTGAATCT” (SEQ ID NO: 161, Table 23) and reverse primer “GGGAAAGGGCTGATTGTCTA” (SEQ ID NO: 162, Table 23) by using standard protocol and LA Taq (Takara, Mountain View, Calif.). PCR conditions were 96° C. for 2 minutes and 35 cycles of 95° C. for 30 seconds, 50° C. for 40 seconds and 72° C. for 1 minute followed by an extension of 72° C. for 2 minutes. A 965 bp amplicon was then separated on a 2.0% agarose gel. Amplicons were PCR purified and sequenced by Sanger sequencing at the University of Missouri DNA Core. If the 1 bp insertion was present, the allele was classified as ANPEP edited.

SIGLEC1: Genomic DNA was used to assess genotype by PCR and agarose gel electrophoresis. PCR was performed with the following SIGLEC1 specific forward primer “GCATTCCTAGGCACAGC” (SEQ ID NO: 128, Table 17) and reverse primer “CTCCTTGCCATGTCCAG” (SEQ ID NO: 129, Table 17) by using standard protocol and LA Taq (Takara, Mountain View, Calif.). PCR conditions were 94° C. for 2 minutes and 35 cycles of 94° C. for 30 seconds, 50° C. for 10 seconds and 72° C. for 2.5 minutes followed by a final extension of 72° C. for 5 minutes. The primers flanked the Neo insert. A wildtype SIGLEC amplicon is 2000 bp. If Neo is inserted the amplicon is 2600 bp. SIGLEC1+/− from litter 144 would have two amplicons on the gel, 2000 bp and 2600 bp.

### Results

Genotyping of litter 144 piglets resulted in 1 female piglet (144-7) that had all three modifications (Table 37). Two male piglets (144-3, 144-4) carried both ANPEP and CD163 edits, but not the SIGLEC1 edit. The pigs were genotyped by PCR and results are shown in FIG. 41. The 1387 bp deletion in CD163 was illustrated by a smaller amplicon in addition to the wild type (FIG. 41, panel A). The 1 bp insertion in the ANPEP gene was not visible after gel electrophoresis and the amplicon was sequenced to determine the presence of the 1 bp insertion (FIG. 41 panels B and D). SIGLEC1 knockout was achieved by the insertion of a neomycin cassette (Neo) and therefore, an increased size in the amplicon indicates a knock-out (FIG. 41 panel C).

TABLE 37
Genotypes of Litter 144

| | | ANPEP Allele | ANPEP | | CD163 | SIGLEC1 | SIGLEC |
|---|---|---|---|---|---|---|---|
| Piglet | Sex | 1 | Allele 2 | CD163 Allele 1 | Allele 2 | Allele 1 | Allele 2 |
| 144-1 | M | 1 bp insertion | Wild Type | Wild Type | Wild Type | SIGLEC- | Wild Type |
| | | | | | | (Neo) | |
| 144-2 | M | Wild Type | Wild Type | Wild Type | Wild Type | SIGLEC- | Wild Type |
| | | | | | | (Neo) | |
| 144-3 | M | 1 bp insertion | Wild Type | 1387 bp deletion | Wild Type | Wild Type | Wild Type |
| 144-4 | M | 1 bp insertion | Wild Type | 1387 bp deletion | Wild Type | Wild Type | Wild Type |
| 144-5 | M | 1 bp insertion | Wild Type | Wild Type | Wild Type | SIGLEC- | Wild Type |
| | | | | | | (Neo) | |
| 144-6 | M | 1 bp insertion | Wild Type | Wild Type | Wild Type | Wild Type | Wild Type |
| 144-7 | F | 1 bp insertion | Wild Type | 1387 bp deletion | Wild Type | SIGLEC- | Wild Type |
| | | | | | | (Neo) | |
| 144-8 | F | Wild Type | Wild Type | 1387 bp deletion | Wild Type | Wild Type | Wild Type |
| 144-9 | F | 1 bp insertion | Wild Type | Wild Type | Wild Type | SIGLEC- | Wild Type |
| | | | | | | (Neo) | |
| 144-10 | F | Wild Type | Wild Type | 1387 bp deletion | Wild Type | Wild Type | Wild Type |

### Example 8: Generation of Pigs Homozygous for Chromosomal Modifications in Two or More Genes Selected from ANPEP, SIGLEC1 and CD163, and Testing of Such Pigs for Resistance to TGEV and PRRSV

Once they reach sexual maturity, the pigs generated as described above in Example 7 will be used to create pigs that are homozygous for the chromosomal modifications both ANPEP and CD163, or all three of ANPEP, CD163, and SIGLEC1. This will be done by breeding the female containing all three modifications (144-7) with the two males having modifications for ANPEP and CD163 (144-3, 144-4). This cross should result in offspring that are homozygous for ANPEP (−/−) and CD163 (−/−), but are only heterozygous for SIGLEC1 (+/−). To generate animals containing homozygous knockouts of all three alleles (ANPEP, CD163, and SIGLEC), these offspring (F1 generation) will be back-crossed with additional triple heterozygous offspring generated as in Example 7. Alternatively, or in conjunction, the breeding described in Example 7 will be repeated to create male and female triple heterozygous lines which will be crossed to generate triple homozygous offspring. Thus, generation of homozygous triple knockout animals will take at minimum two generations but will likely require additional generations to establish male and female triple heterozygous lines.

Once the homozygous double (ANPEP^{-/-}/CD163^{-/-}) and triple (ANPEP^{-/-}/CD163^{-/-}/SIGLEC1^{-/-}) knockout animals are made, they will be tested for resistance to TGEV using the methods described above in Example 6 and for resistance to PRRSV using the methods described above in Example 2. It is expected that both the double and triple knockout animals will be resistant to both TGEV and PRRSV.

In view of the above, it will be seen that the several objects of the invention are achieved and other advantageous results attained.

As various changes could be made in the above products and methods without departing from the scope of the invention, it is intended that all matter contained in the above description and shown in the accompanying drawings shall be interpreted as illustrative and not in a limiting sense.

### Example 9: In Vitro Infection of ANPEP KO and WT Cells with TGEV, PRCV and PEDV

Porcine alveolar macrophages (PAMs) were collected from an ANPEP KO pig (pig 20-10, Table 34) and a WI pig by excising the lungs and performing a lung lavage with ˜1.00 ml cold phosphate buffered saline. After culturing for two weeks in MEM supplemented with 7% fetal bovine serum (FBS) and antibiotics, a population of fibroblast cells emerged. The fibroblast-like cells were infected at a multiplicity of infection (moi)=1 with TGEV, PRCV, and PEDV isolates. Preparation of TGEV and PEDV isolates are described in Example 6. The PRCV isolate was prepared by growing the virus on ST cells. After incubating for 24 hours, the cells were fixed with 80% acetone and dried. Virus-infected cells were detected using FITC-labeled coronavirus anti-N protein antibodies. TGEV and PRCV were detected with anti-FIPV3-70 mAb. PEDV was detected by a monoclonal antibody prepared in house. Nuclei were stained using propidium iodide. Cells were viewed under a fluorescence microscope.

FIG. 42 shows representative fluorescent images of cells infected with the three different viruses. ANPEP KO cells showed clear resistance to TGEV and PCRV infection, but were susceptible to PEDV infection (FIG. 42, panel A). All WT cells showed clear infection with all three viruses (FIG. 42, panel B). Thus, the loss of the ANPEP protein may confer resistance to PRCV as well as TGEV in susceptible populations.

### REFERENCES

Allende R., et al., North American and European Porcine Reproductive and Respiratory Syndrome Viruses Differ in Non-Structural Protein Coding Regions, The Journal of General Virology, 1999, Pages 307-315, Volume 80, Part 2.
An, T. Q., et al., Porcine Reproductive and Respiratory Syndrome Virus Attachment is Mediated by the N-Terminal Domain of the Sialoadhesin Receptor, Veterinary Microbiology, 2010, Pages 371-378, Volume 143, Numbers 2-4.
Bauer, B. K., et al., 1 Arginine Supplementation In Vitro Increases Porcine Embryo Development and Affects mRNA Transcript Expression, Reproduction, Fertility and Development, 2010, Pages 107-107, Volume 23, Number 1.
Beaton B. P., et al., Compound Transgenics: Recombinase-Mediated Gene Stacking, Transgenic Animal Technology (Third Edition), A Laboratory Handbook, Elsevier, 2014, Pages 565-578.
Benfield D. A., et al., Characterization of Swine Infertility and Respiratory Syndrome (SIRS) Virus (Isolate ATCC VR-2332), Journal of Veterinary Diagnostic Investigation, 1992, Pages 127-133, Volume 4, Number 2.
Bhagwat, S. V., et al., CD13/APN is Activated by Angiogenic Signals and is Essential for Capillary Tube Formation, Blood, 2001, Pages 652-659, Volume 97.
Boddicker, N. J., et al., Genome-Wide Association and Genomic Prediction for Host Response to Porcine Reproductive and Respiratory Syndrome Virus Infection, Genetics, Selection, evolution: GSE, 2014, Page 18, Volume 46.
Borg, N. A., et al., CD1d-lipid-antigen Recognition by the Semi-Invariant NKT T-Cell Receptor, Nature, 2007, Pages 44-49, Volume 448, Number 7149.
Brinster, R. L., et al., Factors Affecting the Efficiency of Introducing Foreign DNA into Mice by Microinjecting Eggs, Proceedings of the National Academy of Sciences of the United States of America, 1985, Pages 4438-4442, Volume 82, Number 13.
Cafruny, W. A., et al, Trojan Horse Macrophages: Studies with the Murine Lactate Dehydrogenase-Elevating Virus and Implications for Sexually Transmitted Virus Infection, The Journal of General Virology, 1996, Pages 3005-3012, Volume 77, Part 12.
Calvert, J. G., et al., CD163 Expression Confers Susceptibility to Porcine Reproductive and Respiratory Syndrome Viruses, Journal of Virology, 2007, Pages 7371-7379, Volume 81, Number 14.
Cao, L., et al., Generation of a Monoclonal Antibody to 51 Protein of Porcine Epidemic Diarrhea Virus, Monoclonal Antibodies in Immunodiagnosis and Immunotherapy, 2013, Pages 371-374, Volume 32, Issue 5.
Carter, D. B., et al., Phenotyping of Transgenic Cloned Piglets, Cloning and Stem Cells, 2002, Pages 131-145, Volume 4, Number 2.
Chen, L., et al., Structural Basis for Multifunctional Roles of Mammalian Aminopeptidase N, Proceedings of the National Academy of Sciences of the United States of America, October 2012, Pages 17966-17971, Volume 109, Number 44.
Christianson, W. T., et al., Experimental Reproduction of Swine Infertility and Respiratory Syndrome in Pregnant Sows, American Journal of Veterinary Research, 1992, Pages 485-488, Volume 53, Number 4.
Christopher-Hennings, J., et al., Effects of A Modified-Live Virus Vaccine Against Porcine Reproductive and Respiratory Syndrome in Boars, American Journal of Veterinary Research, 1997, Pages 40-45, Volume 58, Number 1.
Cong L., et al., Multiplex Genome Engineering Using CRISPR/Cas Systems, Science, 2013, Pages 819-823, Volume 339.
Dai, Y., et al., Targeted Disruption of the Alpha1,3-Galactosyltransferase Gene in Cloned Pigs, Nature Biotechnology, 2002, Pages 251-255, Volume 20, Number 3.
Das, P. B., et al., The Minor Envelope Glycoproteins GP2a and GP4 of Porcine Reproductive and Respiratory Syndrome Virus Interact with the Receptor CD163, Journal of Virology, 2010, Pages 1731-1740, Volume 84, Number 4.
Dee, S. A., et al., Elimination of Porcine Reproductive and Respiratory Syndrome Virus Using A Test and Removal Process, The Veterinary Record, 1998, Pages 474-476, Volume 143, Number 17.
Delmas, B., et al., Aminopeptidase N is a Major Receptor for the Entero-Pathogenic Coronavirus TGEV, Nature, 1992, Pages 417-420, Volume 357.
Delmas, B., et al., Determinants Essential for the Transmissible Gastroenteritis Virus-Receptor Interaction Reside within a Domain of Aminopeptidase-N that is Distinct from the Enzymatic Site, Journal of Virology, August 1994, Pages 5216-5224, Volume 68, Number 8.
Delputte, P. L., et al., Effect of Virus-Specific Antibodies on Attachment, Internalization and Infection of Porcine Reproductive and Respiratory Syndrome Virus in Primary Macrophages, Veterinary Immunology and Immunopathology, 2004, Pages 179-188, Volume 102, Number 3.
Delputte, P. L., et al., Involvement of the Matrix Protein in Attachment of Porcine Reproductive and Respiratory Syndrome Virus to A Heparin-Like Receptor on Porcine Alveolar Macrophages, Journal of Virology, 2002, Pages 4312-4320, Volume 76, Number 9.
Delputte, P. L., et al., Porcine Arterivirus Attachment to the Macrophage-Specific Receptor Sialoadhesin is Dependent on the Sialic Acid-Binding Activity of the N-Terminal Immunoglobulin Domain of Sialoadhesin, Journal of Virology, 2007, Pages 9546-9550, Volume 81, Number 17.
Delputte, P. L., et al., Porcine Arterivirus Infection of Alveolar Macrophages is Mediated by Sialic Acid on the Virus, Journal of Virology, 2004, Pages 8094-8101, Volume 78, Number 15
Doyle, L. P., et al., A Transmissible Gastroenteritis in Pigs, Journal of the American Veterinary Medical Association, 1946, Pages 257-259, Volume 108.
Etzerodt, A., et al., Plasma Clearance of Hemoglobin and Haptoglobin in Mice and Effect of CD163 Gene Targeting Disruption, Antioxidants & Redox Signaling, 2013, Pages 2254-2263, Volume 18, Number 17.
Etzerodt, A., et al., CD163 and Inflammation: Biological, Diagnostic, and Therapeutic Aspects, Antioxidants & Redox Signaling, 2013, Pages 2352-2363, Volume 18, Number 17.
Fabriek, B. O., et al., The Macrophage Scavenger Receptor CD163, Immunobiology, 2005, Pages 153-160, Volume 210, Numbers 2-4.
Fabriek, B. O., et al. The Macrophage Scavenger Receptor CD163 Functions as an Innate Immune Sensor for Bacteria, Blood, 2009, Pages 887-892, Volume 113, Number 4.
Gaj, T., et al., ZFN, TALEN, and CRISPR/Cas-Based Methods for Genome Engineering, Trends in Biotechnology, 2013, Pages 397-405, Volume 31, Number 7.
Gaudreault, N., et al., Factors Affecting the Permissiveness of Porcine Alveolar Macrophages for Porcine Reproductive and Respiratory Syndrome Virus, Archives of Virology, 2009, Pages 133-136, Volume 154, Number 1.
Graversen, J. H., et al., Targeting the Hemoglobin Scavenger Receptor CD163 in Macrophages Highly Increases the Anti-Inflammatory Potency of Dexamethasone, Molecular Therapy: The Journal of the American Society of Gene Therapy, 2012, Pages 1550-1558, Volume 20, Number 8.
Green, M. R., et al., Molecular Cloning: A Laboratory Manual, 4th Edition, Cold Spring Harbor Laboratory Press, New York.
Groenen, M. A., et al., Analyses of Pig Genomes Provide Insight into Porcine Demography and Evolution, Nature, 2012, Pages 393-398, Volume 491, Number 7424.
Guzman-Rojas, L., et al., Cooperative Effects of Aminopeptidase N (CD13) Expressed by Nonmalignant and Cancer Cells Within the Tumor Microenvironment, Proceedings of the National Academy of Sciences of the United States of America, 2012, Pages 1637-1642, Volume 109, Number 5.
Hai T., et al., One-Step Generation of Knockout Pigs by Zygote Injection of CRISPR/Cas System, Cell Research, 2014, Pages 372-375, Volume 24, Number 3.
Halbur, P. G., et al., Comparison of the Pathogenicity of Two US Porcine Reproductive and Respiratory Syndrome Virus Isolates with that of the Lelystad Virus, Veterinary Pathology, 1995, Pages 648-660, Volume 32, Number 6.
Halbur, P. G., et al., Update on Abortion Storms and Sow Mortality, Journal of Swine Health and Production, 1997, Page 73, Volume 5, Number 2.
Hammer, R. E., et al., Production of Transgenic Rabbits, Sheep and Pigs by Microinjection, Nature, 1985, Pages 680-683, Volume 315, Number 6021.
Hauschild, J., et al., Efficient Generation of A Biallelic Knockout in Pigs Using Zinc-Finger Nucleases, Proceedings of the National Academy of Sciences of the United States of America, 2011, Pages 12013-12017, Volume 108, Number 29.
Holtkamp, D. J., et al., Assessment of the Economic Impact of Porcine Reproductive and Respiratory Syndrome Virus on United States Pork Producers, Journal of Swine Health and Production, 2013, Pages 72-84, Volume 21, Number 2.
Hsu P. D., et al., DNA Targeting Specificity of RNA-Guided Cas9 Nucleases, Nature Biotechnology, 2013, Pages 827-832, Volume 31, Number 9.
Hwang, W. Y., et al., Efficient Genome Editing in Zebrafish Using A CRISPR-Cas System, Nature Biotechnology, 2013, Pages 227-229, Volume 31.
Im, G.-S., et al., In vitro Development of Preimplantation Porcine Nuclear Transfer Embryos Cultured in Different Media and Gas Atmospheres, Theriogenology, 2004, Pages 1125-1135, Volume 61, Number 6.
Jeney, V., et al., Pro-Oxidant and Cytotoxic Effects of Circulating Heme, Blood, 2002, Pages 879-887, Volume 100, Number 3.
Kamau, A. N., et al., Porcine Amino Peptidase N Domain VII has Critical Role in Binding and Entry of Porcine Epidemic Diarrhea Virus, Virus Research, 2017, Pages 150-157, Volume 227.
Keffaber, K. K., Reproductive Failure of Unknown Etiology, American Association of Swine Practitioners Newsletter, 1989, Pages 1-10, Volume 1.
Key, K. F. et al., Genetic Variation and Phylogenetic Analyses of the ORF5 Gene of Acute Porcine Reproductive and Respiratory Syndrome Virus Isolates, Veterinary Microbiology, 2001, Pages 249-263, Volume 83, Number 3.
Killoren, K. E. et al., Porcine Respiratory Coronavirus, Swine Health Information Center and Center for Food Security and Public Health, 2016. http://www.cfsph.iastate.edu/pdf/shic-factsheet-porcine-respiratory-coronavirus.
Kim, H. S., et al, Enhanced Replication of Porcine Reproductive and Respiratory Syndrome (PRRS) Virus in A Homogeneous Subpopulation of MA-104 Cell Line, Archives of Virology, 1993, Pages 477-483, Volume 133, Numbers 3-4.
Kolb, A. F., et al., Mammary Gland Development is Delayed in Mice Deficient for Aminopeptidase N, Transgenic Research, 2013, Pages 425-434, Volume 22, Issue 2.
Kristiansen, M., et al., Identification of the Haemoglobin Scavenger Receptor, Nature, 2001, Pages 198-201, Volume 409, Number 6817.
Kwon, D. N., et al., Production of Biallelic CMP-Neu5Ac Hydroxylase Knock-Out Pigs, Scientific Reports, 2013, Page 1981, Volume 3.
Ladinig, A., et al., Pathogenicity of Three Type 2 Porcine Reproductive and Respiratory Syndrome Virus Strains in Experimentally Inoculated Pregnant Gilts, Virus Research, 2015, Pages 24-35, Volume 203.
Lai, L., et al., Production of Alpha-1,3-Galactosyltransferase Knockout Pigs by Nuclear Transfer Cloning, Science, 2002, Pages 1089-1092, Volume 295, Number 5557.
Lai L., et al., Creating Genetically Modified Pigs by Using Nuclear Transfer, Reproductive Biology and Endocrinology, 2003, Page 82, Volume 1, Number 1. (Lai et al., 2003a).
Lai L., et al., Production of Cloned Pigs by Using Somatic Cells as Donors, Cloning and Stem Cells, 2003, Pages 233-241, Volume 5, Number 4. (Lai et al., 2003b).
Lai, L., et al., Generation of Cloned Transgenic Pigs Rich in Omega-3 Fatty Acids, Nature Biotechnology, 2006, Pages 435-436, Volume 24, Number 4.
Lawson, S. R., et al., Porcine Reproductive and Respiratory Syndrome Virus Infection of Gnotobiotic Pigs: Sites of Virus Replication and Co-Localization with MAC-387 Staining at 21 Days Post-Infection, Virus Research, 1997, Pages 105-113, Volume 51, Number 2.
Lee, K., et al., Engraftment of Human iPS Cells and Allogeneic Porcine Cells into Pigs With Inactivated RAG2 and Accompanying Severe Combined Immunodeficiency, Proceedings of the National Academy of Sciences of the United States of America, 2014, Pages 7260-7265, Volume 111, Number 20.
Lee K., et al., Piglets Produced from Cloned Blastocysts Cultured in vitro with GM-CSF, Molecular Reproduction and Development, 2013, Pages 145-154, Volume 80, Issue 2.
Li, B. X., et al., Porcine Aminopeptidase N is a Functional Receptor for the PEDV Coronavirus, Virology, 2007, Pages 166-172, Volume 365, Issue 1.
Li, D., et al., Heritable Gene Targeting in the Mouse and Rat Using a CRISPR-Cas System, Nature Biotechnology, 2013, Pages 681-683, Volume 31, Number 8.
Li, F., Receptor Recognition Mechanisms of Coronaviruses: A Decade of Structural Studies, Journal of Virology, 2015, Pages 1954-1964, Volume 89, Number 4.
Li, W., et al., Aminopeptidase N is not Required for Porcine Epidemic Diarrhea Virus Cell Entry, Virus Research, 2017, Pages 6-13, Volume 235.
Li, W., et al., Angiotensin-Converting Enzyme 2 is a Functional Receptor for the SARS Coronavirus, Nature, 2003, Pages 450-454, Volume 426.
Lillico, S. G., et al., Live Pigs Produced From Genome Edited Zygotes, Scientific Reports, 2013, Page 2847, Volume 3.
Lin, C.-M., et al., Antigenic Relationships among Porcine Epidemic Diarrhea Virus and Transmissible Gastroenteritis Virus Strains, Journal of Virology, 2015, Pages 3332-3342, Volume 89, Number 6.
Liu, C., et al., Receptor Usage and Cell Entry of Porcine Epidemic Diarrhea Coronavirus, Journal of Virology, June 2015, Pages 6121-6125, Volume 89, Number 11.
Machaty Z., et al., Complete Activation of Porcine Oocytes Induced by the Sulfhydryl Reagent, Thimerosal, Biology of Reproduction, 1997, Pages 1123-1127, Volume 57, Number 5.
Madsen, M., et al., Molecular Characterization of the Haptoglobin-Hemoglobin Receptor CD163. Ligand Binding Properties of the Scavenger Receptor Cysteine-Rich Domain Region, The Journal of Biological Chemistry, 2004, Pages 51561-51567, Volume 279, Number 49.
Madson, D. M., et al., Characterization of Porcine Epidemic Diarrhea Virus Isolate US/Iowa/18984/2013 Infection in 1-Day-Old Cesarean-Derived Colostrum-Deprived Piglets, Veterinary Pathology, 2016, Pages 44-52, Volume 53, Number 1.
Martinez-Pomares, L., et al., CD169+ Macrophages at the Crossroads of Antigen Presentation, Trends in Immunology, 2012, Pages 66-70, Volume 33, Number 2.
Meng, F., et al., A Phage-Displayed Peptide Recognizing Porcine Aminopeptidase N is a Potent Small Molecule Inhibitor of PEDV Entry, Virology, 2014, Pages 20-27, Volumes 456-457.
Mengeling, W. L., et al., Clinical Consequences of Exposing Pregnant Gilts to Strains of Porcine Reproductive and Respiratory Syndrome (PRRS) Virus Isolated from Field Cases of “Atypical” PRRS, American Journal of Veterinary Research, 1998, Pages 1540-1544, Volume 59, Number 12. Merck, The Merck Veterinary Manual, http://www.merckmanuals.com/vet/appendixes/referenceguides/normal rectal temperature ranges html.
Miao, Y. L., et al., Centrosome Abnormalities During Porcine Oocyte Aging, Environmental and Molecular Mutagenesis, 2009, Pages 666-671, Volume 50, Number 8.
Morgan, S. B., et al., Pathology and Virus Distribution in the Lung and Lymphoid Tissues of Pigs Experimentally Inoculated with Three Distinct Type 1 PRRS Virus Isolates of Varying Pathogenicity, Transbound and Emerging Diseases, 2016, Pages 285-295, Volume 63, Number 3.
Nelsen, C. J., et al., Porcine Reproductive and Respiratory Syndrome Virus Comparison: Divergent Evolution on Two Continents, Journal of Virology, 1999, Pages 270-280, Volume 73, Number 1.
Neumann, E. J., et al., Assessment of the Economic Impact of Porcine Reproductive and Respiratory Syndrome on Swine Production in the United States, Journal of the American Veterinary Medical Association, 2005, Pages 385-392, Volume 227, Number 3.
Niederwerder, M. C., et al., Tissue Localization, Shedding, Virus Carriage, Antibody Response, and Aerosol Transmission of Porcine epidemic diarrhea virus Following Inoculation of 4-week-old Feeder Pigs, Journal of Veterinary Diagnostic Investigation, 2016, Pages 671-678, Volume 28, Issue 6.
Nielsen, M. J., et al., The Macrophage Scavenger Receptor CD163: Endocytic Properties of Cytoplasmic Tail Variants, Journal of Leukocyte Biology, 2006, Pages 837-845, Volume 79.
Niu, Y., et al., Generation of Gene-Modified Cynomolgus Monkey Via Cas9/RNA-Mediated Gene Targeting in One-Cell Embryos, Cell, 2014, Pages 836-843, Volume 156, Number 4.
Oetke, C., et al., Sialoadhesin-Deficient Mice Exhibit Subtle Changes in B- and T-Cell Populations and Reduced Immunoglobulin M Levels, Molecular and Cellular Biology, 2006, Pages 1549-1557, Volume 26, Number 4.
Oh, J. S., et al., Identification of a Putative Cellular Receptor 150 kDa Polypeptide for Porcine Epidemic Diarrhea Virus in Porcine Enterocytes, Journal of Veterinary Science, 2003, Pages 269-275, Volume 4, Number 3.
O'Neill, A. S., et al., Sialoadhesin—A Macrophase-Restricted Marker of Immunoregulation and Inflammation, Immunology, 2013, Pages 198-207, Volume 138, Number 3.
Park, J. E., et al., Clathrin- and Serine Proteases-Dependent Uptake of Porcine Epidemic Diarrhea Virus into Vero Cells, Virus Research, 2014, Pages 21-29, Volume 191.
Park, J. E., et al., Porcine Epidemic Diarrhea Virus Infects and Replicates in Porcine Alveolar Macrophages, Virus Research, 2014, Pages 143-152, Volume 191.
Park, J. E., Development of Transgenic Mouse Model Expressing Porcine Aminopeptidase N and Its Susceptibility to Porcine Epidemic Diarrhea Virus, Virus Research, 2015, Pages 108-115, Volume 197.
Patience, J. F., et al., Swine Nutrition Guide, 1989, Pages 149-171, Saskatoon, SK: Prairie Swine Centre, University of Saskatchewan, Saskatoon, Calif.
Patton, J. P., et al., Modulation of CD163 Receptor Expression and Replication of Porcine Reproductive and Respiratory Syndrome Virus in Porcine Macrophages, Virus Research, 2009, Pages 161-171, Volume 140, Issues 1-2.
Plagemann, P. G. W., Lactate Dehydrogenase-Elevating Virus and Related Viruses, Fields Virology, 1996, Pages 1105-1120, Volume 3.
Prather, R. S., et al., An Intact Sialoadhesin (Sn/SIGLEC1/CD169) is not Required for Attachment/Internalization of the Porcine Reproductive and Respiratory Syndrome Virus, Journal of Virology, 2013, Pages 9538-9546, Volume 87, Number 17.
Provost, C., et al., Identification of A New Cell Line Permissive to Porcine Reproductive and Respiratory Syndrome Virus Infection and Replication which is Phenotypically Distinct from MARC-145 Cell Line, Virology Journal, 2012, Page 267, Volume 9.
Ran, F. A., et al., In vivo Genome Editing Using Staphylococcus aureus Cas9, Nature, 2015, Pages 186-191, Volume 520.
Rangel, R., et al., Impaired Angiogenesis in Aminopeptidase N-Null Mice, Proceedings of the National Academy of Sciences of the United States of America, 2007, Pages 4588-4593, Volume 104, Number 11.
Reed, L. J., et al., A Simple Method of Estimating Fifty Per Cent Endpoints, American Journal of Epidemiology, May 1938, Pages 493-497, Volume 27, Issue 3.
Ren, X., et al., Binding Characterization of Determinants in Porcine Aminopeptidase N, the Cellular Receptor for Transmissible Gastroenteritis Virus, Journal of Biotechnology, 2010, Pages 202-206, Volume 150, Issue 1.
Ritter, M., et al., Genomic Organization and Chromosomal Localization of the Human CD163 (M130) Gene: A Member of the Scavenger Receptor Cysteine-Rich Superfamily, Biochemical and Biophysical Research Communications, 1999, Pages 466-474, Volume 260, Number 2.
Ritter, M., et al., The Scavenger Receptor CD163: Regulation, Promoter Structure and Genomic Organization, Pathobiology, 1999, Pages 257-261, Volume 67, Numbers 5-6.
Ren, X., et al., Phage Displayed Peptides Recognizing Porcine Aminopeptidase N Inhibit Transmissible Gastroenteritis Coronavirus Infection in vitro, Virology, 2011, Pages 299-306, Volume 410, Issue 2.
Ross, J. W., et al., Optimization of Square-Wave Electroporation for Transfection of Porcine Fetal Fibroblasts, Transgenic Research, 2010, Pages 611-620, Volume 19, Issue 4.
Rowland, R. R. R., et al., The Evolution of Porcine Reproductive and Respiratory Syndrome Virus: Quasispecies and Emergence of A Virus Subpopulation During Infection of Pigs with VR-2332, Virology, 1999, Pages 262-266, Volume 259, Number 2.
Rowland, R. R. R., et al., Lymphoid Tissue Tropism of Porcine Reproductive and Respiratory Syndrome Virus Replication During Persistent Infection of Pigs Originally Exposed to Virus In utero, Veterinary Microbiology, 2003, Pages 219-235, Volume 96, Issue 3.
Rowland, R. R., The Interaction Between PRRSV and the Late Gestation Pig Fetus, Virus Research, 2010, Pages 114-122, Volume 154, Numbers 1-2.
Rowland, R. R., et al., Control of Porcine Reproductive and Respiratory Syndrome (PRRS) Through Genetic Improvements in Disease Resistance and Tolerance, Frontiers in Genetics, 2012, Page 260, Volume 3.
Saif, L. J., et al., Coronaviruses, Diseases of Swine, Chapter 35, 10th Edition, 2012, Wiley-Blackwell.
Sanchez, C., et al., The Porcine 2A10 Antigen is Homologous to Human CD163 and Related to Macrophage Differentiation, Journal of Immunology, 1999, Pages 5230-5237, Volume 162, Number 9.
Sánchez-Torres, C., et al., Expression of Porcine CD163 on Monocytes/Macrophages Correlates with Permissiveness to African Swine Fever Infection, Archives of Virology, 2003, Pages 2307-2323, Volume 148, Number 12.
Schaer, C. A., et al., Constitutive Endocytosis of CD163 Mediates Hemoglobin-Heme Uptake and Determines the Noninflammatory and Protective Transcriptional Response of Macrophages to Hemoglobin, Circulation Research, 2006, Pages 943-950, Volume 99, Number r9.
Schaer, D. J., et al., CD163 is the Macrophage Scavenger Receptor for Native and Chemically Modified Hemoglobins in the Absence of Haptoglobin, Blood, 2006, Pages 373-380, Volume 107, Number 1.
Schaer, D. J., et al., Hemophagocytic Macrophages Constitute A Major Compartment of Heme Oxygenase Expression in Sepsis, European Journal of Haematology, 2006, Pages 432-436, Volume 77, Number 5.
Shanmukhappa, K., et al., Role of CD151, A Tetraspanin, in Porcine Reproductive and Respiratory Syndrome Virus Infection, Virology Journal, 2007, Page 62, Volume 4.
Shapiro, L. H., et al., Separate Promoters Control Transcription of the Human Aminopeptidase N Gene in Myeloid and Intestinal Epithelial Cells, The Journal of Biological Chemistry, 1991, Pages 11999-12007, Volume 266, Number 18.
Sheahan, T., et al., Pathways of Cross-Species Transmission of Synthetically Reconstructed Zoonotic Severe Acute Respiratory Syndrome Coronavirus, Journal of Virology, 2008, Pages 8721-8732, Volume 82, Number 17.
Shi, M., et al., Phylogeny-Based Evolutionary, Demographical, and Geographical Dissection of North American Type 2 Porcine Reproductive and Respiratory Syndrome Viruses, Journal of Virology, 2010, Pages 8700-8711, Volume 84, Number 17.
Shimozawa, N., et al., Abnormalities in Cloned Mice are not Transmitted to the Progeny, Genesis, 2002, Pages 203-207, Volume 34, Number 3.
Smit, A. F. A., et al., RepeatMasker Open-3.0. 1996-2010. Current Version: open-4.0.5 (RMLib: 20140131 and Dfam: 1.2). http://www.repeatmasker.org&gt. CD163: accessed Jul. 25, 2014; CD1D: accessed Aug. 27, 2013.
Soares, M. P., et al., Heme Oxygenase-1: From Biology to Therapeutic Potential, Trends in Molecular Medicine, 2009, Pages 50-58, Volume 15, Number 2.
Stein, M., et al., Interleukin 4 Potently Enhances Murine Macrophage Mannose Receptor Activity: A Marker of Alternative Immunologic Macrophage Activation, The Journal of Experimental Medicine, 1992, Pages 287-292, Volume 176, Number 1.
Stephenson, R., et al., Multiplex Serology for Common Viral Infections in Feral Pigs (Sus scrofa) in Hawaii Between 2007 and 2010, Journal of Wildlife Diseases, 2015, Pages 239-243, Volume 51, Number 1.
Stevenson, G. W., et al., Emergence of Porcine Epidemic Diarrhea Virus in the United States: Clinical Signs, Lesions, and Viral Genomic Sequences, Journal of Veterinary Diagnostic Investigation, 2013, Pages 649-654, Volume 25, Number 5.
Sulahian, T. H., et al., Human Monocytes Express CD163, Which is Upregulated by IL-10 and Identical to p155, Cytokine, 2000, Pages 1312-1321, Volume 12, Issue 9.
Sun, D., et al., Virus-Binding Activity of the Truncated C Subunit of Porcine Aminopeptidase N Expressed in Escherichia coli, Biotechnology Letters, 2012, Pages 533-539, Volume 34, Issue 3.
Terns, M. P., et al., CRISPR-Based Adaptive Immune Systems, Current Opinion in Microbiology, 2011, Pages 321-327, Volume 14, Issue 3.
Tian, K., et al., Emergence of Fatal PRRSV Variants: Unparalleled Outbreaks of Atypical PRRS in China and Molecular Dissection of the Unique Hallmark, PLoS One, 2007, e526, Volume 2, Number 6.
Trible, B. R., et al., Recognition of the Different Structural Forms of the Capsid Protein Determines the Outcome Following Infection with Porcine Circovirus Type 2, Journal of Virology, 2012, Pages 13508-13514, Volume 86, Number 24.
Tusell, S. M., et al., Mutational Analysis of Aminopeptidase N, a Receptor for Several Group 1 Coronaviruses, Identifies Key Determinants of Viral Host Range, Journal of Virology, 2007, Pages 1261-1273, Volume 81, Number 3.
U.S. Department of Agriculture and U.S. Department of Health and Human Services, Dietary Guidelines for Americans, 2010, 7th Edition.
Van Breedam, W., et al., Porcine Reproductive and Respiratory Syndrome Virus Entry into the Porcine Macrophage, Journal of General Virology, 2010, Pages 1659-1667, Volume 91.
Van Breedam, W., et al., The M/GP(5) Glycoprotein Complex of Porcine Reproductive and Respiratory Syndrome Virus Binds the Sialoadhesin Receptor in a Sialic Acid-Dependent Manner, PloS Pathogens, 2010, Document e1000730, Volume 6, Number 1.
van den Heuvel, M. M., et al., Regulation of CD163 on Human Macrophages: Cross-Linking of CD163 Induces Signaling and Activation, Journal of Leukocyte Biology, 1999, Pages 858-866, Volume 66, Issue 5.
van den Hoff, M. J., et al., Electroporation in ‘Intracellular’ Buffer Increases Cell Survival, Nucleic Acids Research, 1992, Page 2902, Volume 20, Number 11.
Van Gorp, H., et al., Scavenger Receptor CD163, A Jack-of-all-trades and Potential Target for Cell-Directed Therapy, Molecular Immunology, 2010, Pages 1650-1660, Volume 47, Issues 7-8.
Van Gorp, H., et al., Identification of the CD163 Protein Domains Involved in Infection of the Porcine Reproductive and Respiratory Syndrome Virus, Journal of Virology, 2010, Pages 3101-3105, Volume 84, Number 6.
Van Reeth, Dual Infections of Feeder Pigs with Porcine Reproductive and Respiratory Syndrome Virus Followed by Porcine Respiratory Coronavirus or Swine Influenza Virus: A Clinical and Virological Study, Veterinary Microbiology, 1996, Pages 325-335, Volume 48, Issues 3-4.
Vanderheijden, N., et al., Involvement of Sialoadhesin in Entry of Porcine Reproductive and Respiratory Syndrome Virus into Porcine Alveolar Macrophages, Journal of Virology, 2003, Pages 8207-8215, Volume 77, Number 15.
Vemulapalli, R., Real-Time Reverse Transcription Polymerase Chain Reaction for Rapid Detection of Transmissible Gastroenteritis Virus, Animal Coronaviruses, A Laboratory Handbook, Chapter 10,2016, Pages 115-119.
Vijgen, L., et al., Identification of Six New Polymorphisms in the Human Coronavirus 229E Receptor Gene (Aminopeptidase N/CD13), International Journal of Infectious Diseases, 2004, Pages 217-222, Volume 8, Issue 4.
Walters, E. M., et al., Advancing Swine Models for Human Health and Diseases, Missouri Medicine, 2013, Pages 212-215, Volume 110, Number 3.
Wang, H., et al., One-Step Generation of Mice Carrying Mutations in Multiple Genes by CRISPR/Cas-Mediated Genome Engineering, Cell, 2013, Pages 910-918, Volume 153, Issue 4.
Welch, S. K., et al., A Brief Review of CD163 and its Role in PRRSV Infection, Virus Research, 2010, Pages 98-103, Volume 154, Issues 1-2.
Wells, K. D., et al., Use of the CRISPR/Cas9 System to Produce Genetically Engineered Pigs from In Vitro-Derived Oocytes and Embryos, Biology of Reproduction, 2014, Pages 1-13, Volume 91, Issue 3.
Wells, K. D., et al., Substitution of Porcine CD163 SRCR Domain 5 with a CD163-like Homolog Confers Resistance of Pigs to Genotype 1 but not Genotype 2 Porcine Reproductive and Respiratory Syndrome (PRRS) Viruses, Journal of Virology, 2018, Volume 92, Issue 9.
Wensvoort, G., et al., Mystery Swine Disease in The Netherlands: The Isolation of Lelystad Virus, Veterinary Quarterly, 1991, Pages 121-130, Volume 13, Issue 3.
Wentworth, D. E., et al., Cells of Human Aminopeptidase N (CD13) Transgenic Mice are Infested by Human Coronavirus-229E in vitro, but not in vivo, Virology, 2005, Pages 185-197, Volume 335, Issue 2.
Whitworth, K. M., et al., Gene-Edited Pigs are Protected from Porcine Reproductive and Respiratory Syndrome Virus, Nature Biotechnology, 2016, Pages 20-22, Volume 34.
Whitworth, K. M., et al., Method of Oocyte Activation Affects Cloning Efficiency in Pigs, Molecular Reproduction & Development, 2009, Pages 490-500, Volume 76, Issue 5.
Whitworth, K. M., et al., Scriptaid Corrects Gene Expression of a Few Aberrantly Reprogrammed Transcripts in Nuclear Transfer Pig Blastocyst Stage Embryos, Cellular Reprogramming, 2011, Pages 191-204, Volume 13, Number 3.
Whitworth, K. M., et al., Activation Method does not Alter Abnormal Placental Gene Expression and Development in Cloned Pigs, Molecular Reproduction & Development, 2010, Pages 1016-1030, Volume 77, Issue 12.
Whitworth, K. M., et al., Use of the CRISPR/Cas9 System to Produce Genetically Engineered Pigs from In Vitro-Derived Oocytes and Embryos, Biology of Reproduction, 2014, Pages 1-13, Article 78, Volume 91, Issue 3.
Whitworth, K. M., et al., Zygote Injection of CRISPR/Cas9 RNA Successfully Modifies the Target Gene Without Delaying Blastocyst Development or Altering the Sex Ratio in Pigs, Transgenic Research, 2017, Pages 97-107, Volume 26, Issue 1.
Whyte, J. J., et al., Genetic Modifications of Pigs for Medicine and Agriculture, Molecular Reproduction & Development, 2011, Pages 879-891, Volume 78, Issue 10-11.
Whyte, J. J., et al., Gene Targeting with Zinc Finger Nucleases to Produce Cloned eGFP Knockout Pigs, Molecular Reproduction & Development, 2011, Page 2, Volume 78, Issue 1.
Wiedenheft, B., et al., RNA-Guided Genetic Silencing Systems in Bacteria and Archaea, Nature, 2012, Pages 331-338, Volume 482.
Wilkinson, J. M., et al., Genome-Wide Analysis of the Transcriptional Response to Porcine Reproductive and Respiratory Syndrome Virus Infection at the Maternal/Fetal Interface and in the Fetus, BMC Genomics, 2016, Page 383, Volume 17.
Winckler, C., et al., The Reliability and Repeatability of a Lameness Scoring System for Use as an Indicator of Welfare in Dairy Cattle, Acta Agriculturae Scandinavica Section A—Animal Science, 2001, Pages 103-107, Volume 51.
Wissink, E. H. J., et al., Identification of Porcine Alveolar Macrophage Glycoproteins Involved in Infection of Porcine Respiratory and Reproductive Syndrome Virus, Archives of Virology, 2003, Pages 177-187, Volume 148, Number 1.
Yanez, J. M., et al., Genome-wide Single Nucleotide Polymorphism Discovery in Atlantic Salmon (Salmo salar): Validation in Wild and Farmed American and European Populations, Molecular Ecology Resources, 2016, Pages 1002-1011, Volume 16, Issue 4.
Yang, D., et al., Generation of PPARgamma Mono-Allelic Knockout Pigs via Zinc-Finger Nucleases and Nuclear Transfer Cloning, Cell Research, 2011, Pages 979-982, Volume 21.
Yeager, C. L., et al., Human Aminopeptidase N is a Receptor for Human Coronavirus 229E, Nature, 1992, Pages 420-422, Volume 357.
Yoshioka K., et al., Birth of Piglets Derived from Porcine Zygotes Cultured in a Chemically Defined Medium, Biology of Reproduction, 2002, Pages 112-119, Volume 66, Issue 1.
Yuan Y., et al., Quadrupling Efficiency in Production of Genetically Modified Pigs Through Improved Oocyte Maturation, Proceedings of the National Academy of Sciences of the United States of America, 2017, Pages E5796-E5804, Volume 114, Number 29.
Zhang, Q., et al., PRRS Virus Receptors and Their Role for Pathogenesis, Veternary Microbiology, 2015, Pages 229-241, Volume 177, Issue 3-4.
Zhang, Zhang Lab General Cloning Protocol, http://www.addgene.org/crispr/zhang/.
Zhao J., et al., Histone Deacetylase Inhibitors Improve In Vitro and In Vivo Developmental Competence of Somatic Cell Nuclear Transfer Porcine Embryos, Cellular Reprogramming, 2010, Pages 75-83, Volume 12, Number 1.
Zhao J., et al., Significant Improvement in Cloning Efficiency of an Inbred Miniature Pig by Histone Deacetylase Inhibitor Treatment after Somatic Cell Nuclear Transfer, Biology of Reproduction, 2009, Pages 525-530, Volume 81, Issue 3.

TABLE OF SEQUENCES

| SEQ | TYPE | DESCRIPTION |
|---|---|---|
| SEQ ID NO: 1 | nucleotide | CRISPR 10 |
| SEQ ID NO: 2 | nucleotide | CRISPR 131 |
| SEQ ID NO: 3 | nucleotide | CRISPR 256 |
| SEQ ID NO: 4 | nucleotide | CRISPR 282 |
| SEQ ID NO: 5 | nucleotide | CRISPR 4800 |
| SEQ ID NO: 6 | nucleotide | CRISPR 5620 |
| SEQ ID NO: 7 | nucleotide | CRISPR 5626 |
| SEQ ID NO: 8 | nucleotide | CRISPR 5350 |
| SEQ ID NO: 9 | nucleotide | eGFP1 |
| SEQ ID NO: 10 | nucleotide | eGFP2 |
| SEQ ID NO: 11 | nucleotide | forward primer 9538 fragment |
| SEQ ID NO: 12 | nucleotide | reverse primer 9538 fragment |
| SEQ ID NO: 13 | nucleotide | forward primer 8729 fragment |
| SEQ ID NO: 14 | nucleotide | forward primer 8729 fragment |
| SEQ ID NO: 15 | nucleotide | WILD TYPE CD163 |
| SEQ ID NO: 16 | nucleotide | FIG. 4, panel C WT |
| SEQ ID NO: 17 | nucleotide | FIG. 4, panel C #1 |
| SEQ ID NO: 18 | nucleotide | FIG. 4, panel C #2 |
| SEQ ID NO: 19 | nucleotide | FIG. 4, panel C #3 |
| SEQ ID NO: 20 | nucleotide | FIG. 5, panel A WT |
| SEQ ID NO: 21 | nucleotide | FIG. 5, panel A #1-1 |
| SEQ ID NO: 22 | nucleotide | FIG. 5, panel A #1-4 |
| SEQ ID NO: 23 | nucleotide | FIG. 5, panel A #2-2 |
| SEQ ID NO: 24 | nucleotide | FIG. 6, panel C CD163 WT |
| SEQ ID NO: 25 | nucleotide | FIG. 6, panel C CD163 #1 |
| SEQ ID NO: 26 | nucleotide | FIG. 6, panel C CD163 #2 |
| SEQ ID NO: 27 | nucleotide | FIG. 6, panel C CD163 #3 |
| SEQ ID NO: 28 | nucleotide | FIG. 6, panel C eGFP WT |
| SEQ ID NO: 29 | nucleotide | FIG. 6, panel C eGFP #1-1 |
| SEQ ID NO: 30 | nucleotide | FIG. 6, panel C eGFP #1-2 |
| SEQ ID NO: 31 | nucleotide | FIG. 6, panel C eGFP #2 |
| SEQ ID NO: 32 | nucleotide | FIG.6, panel C eGFP #3 |
| SEQ ID NO: 33 | nucleotide | FIG. 7, panel C WT |
| SEQ ID NO: 34 | nucleotide | FIG. 7, panel C #67-1 |
| SEQ ID NO: 35 | nucleotide | FIG. 7, panel C #67-2 a1 |
| SEQ ID NO: 36 | nucleotide | FIG. 7, panel C #67-2 a2 |
| SEQ ID NO: 37 | nucleotide | FIG. 7, panel C #67-3 |
| SEQ ID NO: 38 | nucleotide | FIG. 7, panel C #67-4 a1 |
| SEQ ID NO: 39 | nucleotide | FIG. 7, panel C #67-4 a2 |
| SEQ ID NO: 40 | nucleotide | FIG. 8, panel D WT |
| SEQ ID NO: 41 | nucleotide | FIG. 8, panel D #166-1.1 |
| SEQ ID NO: 42 | nucleotide | FIG. 8, panel D #166-1.2 |
| SEQ ID NO: 43 | nucleotide | FIG. 8, panel D #166-2 |
| SEQ ID NO: 44 | nucleotide | FIG. 8, panel D #166-3.1 |
| SEQ ID NO: 45 | nucleotide | FIG. 8, panel D #166-3.2 |
| SEQ ID NO: 46 | nucleotide | FIG. 8, panel D #166-4 |
| SEQ ID NO: 47 | nucleotide | FIG. 16 WT CD163 partial |
| SEQ ID NOs. 48-67 | nucleotide | Primer sequences (Table 2) |
| SEQ ID NOs. 68-79 | nucleotide | Primer sequences (Table 3) |
| SEQ ID NOs. 80-85 | nucleotide | Primer sequences (Table 4) |
| SEQ ID NOs. 86-97 | nucleotide | Primer sequences (Table 5) |
| SEQ ID NO: 98 | nucleotide | CD163 Allele with 1506 bp deletion |
| SEQ ID NO: 99 | nucleotide | CD163 Allele with 7 bp insertion |
| SEQ ID NO: 100 | nucleotide | CD163 Allele with 1280 bp deletion |
| SEQ ID NO: 101 | nucleotide | CD163 Allele with 1373 bp deletion |
| SEQ ID NO: 102 | nucleotide | CD163 Allele with 11 bp deletion |
| SEQ ID NO: 103 | nucleotide | CD163 Allele with 2 bp insertion and |
| | | 377 bp deletion |
| SEQ ID NO: 104 | nucleotide | CD163 Allele with 124 bp deletion |
| SEQ ID NO: 105 | nucleotide | CD163 Allele with 123 bp deletion |
| SEQ ID NO: 106 | nucleotide | CD163 Allele with 1 bp insertion |
| SEQ ID NO: 107 | nucleotide | CD163 Allele with 130 bp deletion |
| SEQ ID NO: 108 | nucleotide | CD163 Allele with 132 bp deletion |
| SEQ ID NO: 109 | nucleotide | CD163 Allele with 1467 bp deletion |
| SEQ ID NO: 110 | nucleotide | CD163 Allele with 1930 bp deletion |
| | | in exon 6, 129 bp deletion in exon 7, |
| | | and 12 bp insertion |
| SEQ ID NO: 111 | nucleotide | CD163 Allele with 28 bp deletion |
| SEQ ID NO: 112 | nucleotide | CD163 Allele with 1387 bp deletion |
| SEQ ID NO: 113 | nucleotide | CD163 Allele with 1382 bp deletion |
| | | and 11 bp insertion |
| SEQ ID NO: 114 | nucleotide | CD163 Allele with 1720 bp deletion |
| SEQ ID NO: 115 | nucleotide | Inserted sequence for SEQ ID NO: 99 |
| SEQ ID NO: 116 | nucleotide | Inserted sequence for SEQ ID NO:110 |
| SEQ ID NO: 117 | nucleotide | Inserted sequence for SEQ ID NO:113 |
| SEQ ID NO: 118 | nucleotide | Domain swap sequence |
| SEQ ID NO: 119 | nucleotide | CD163 Allele with 452 bp deletion |
| SEQ ID NO: 120 | peptide | Porcine CD163 SRCR 5 |
| SEQ ID NO: 121 | peptide | Human CD163L1 SRCR 8 homolog |
| SEQ ID NO: 122 | nucleotide | SIGLEC1 partial WT reference |
| | | sequence |
| SEQ ID NO: 123 | nucleotide | SIGLEC1 Allele with 1,247 bp |
| | | deletion and neo insertion |
| SEQ ID NO: 124-129 | nucleotide | Primer sequences (Table 17) |
| SEQ ID NO: 130-131 | nucleotide | Oligonucleotide sequences (Table 18) |
| SEQ ID NO: 132 | nucleotide | Full length ANPEP sequence |
| SEQ ID NO: 133 | peptide | Porcine ANPEP (X1 homolog) |
| SEQ ID NO: 134 | peptide | Porcine ANPEP (X2, X3 homolog) |
| SEQ ID NO: 135 | nucleotide | ANPEP partial WT reference |
| | | sequence (FIG. 28) |
| SEQ ID NO: 136 | nucleotide | CRISPR guide 1 for ANPEP targeting |
| SEQ ID NO: 137 | nucleotide | CRISPR guide 2 for ANPEP targeting |
| SEQ ID NO: 138 | nucleotide | CRISPR guide 3 for ANPEP targeting |
| SEQ ID NO: 139 | nucleotide | CRISPR guide 4 for ANPEP targeting |
| SEQ ID NO: 140 | nucleotide | CRISPR guide 5 for ANPEP targeting |
| SEQ ID NO: 141 | nucleotide | CRISPR guide 6 for ANPEP targeting |
| SEQ ID NO: 142 | nucleotide | ANPEP guide 1 Primer (Forward) |
| SEQ ID NO: 143 | nucleotide | ANPEP guide 1 Primer (Reverse) |
| SEQ ID NO: 144 | nucleotide | ANPEP guide 2 Primer (Forward) |
| SEQ ID NO: 145 | nucleotide | ANPEP guide 2 Primer (Reverse) |
| SEQ ID NO: 146 | nucleotide | ANPEP guide 3 Primer (Forward) |
| SEQ ID NO: 147 | nucleotide | ANPEP guide 3 Primer (Reverse) |
| SEQ ID NO: 148 | nucleotide | ANPEP guide 4 Primer (Forward) |
| SEQ ID NO: 149 | nucleotide | ANPEP guide 4 Primer (Reverse) |
| SEQ ID NO: 150 | nucleotide | ANPEP guide 5 Primer (Forward) |
| SEQ ID NO: 151 | nucleotide | ANPEP guide 5 Primer (Reverse) |
| SEQ ID NO: 152 | nucleotide | ANPEP guide 6 Primer (Forward) |
| SEQ ID NO: 153 | nucleotide | ANPEP guide 6 Primer (Reverse) |
| SEQ ID NO: 154-160 | nucleotide | Primers for RNA amplification |
| | | (Table 22) |
| SEQ ID NO: 161-162 | nucleotide | Primer sequences (Table 23) |
| SEQ ID NO: 163 | nucleotide | ANPEP allele having 182 bp deletion |
| | | and 5 bp insertion |
| SEQ ID NO: 164 | nucleotide | ANPEP allele having 9 bp deletion |
| SEQ ID NO: 165 | nucleotide | ANPEP allele having 867 bp deletion |
| SEQ ID NO: 166 | nucleotide | ANPEP allele having 1 bp insertion |
| | | (allele D) |
| SEQ ID NO: 167 | nucleotide | ANPEP allele having 2 bp insertion |
| | | (allele E) |
| SEQ ID NO: 168 | nucleotide | ANPEP allele having 267 bp deletion |
| SEQ ID NO: 169 | nucleotide | Inserted sequence for SEQ NO: 163 |
| SEQ ID NO: 170 | nucleotide | ANPEP allele having 9 bp deletion |
| | | (allele F) |
| SEQ ID NO: 171 | nucleotide | ANPEP allele having 1 bp deletion |
| | | (allele H) |
| SEQ ID NO: 172 | nucleotide | ANPEP allele having 12 bp deletion |
| | | (allele G) |
| SEQ ID NO: 173 | nucleotide | ANPEP allele having 25 bp deletion |
| SEQ ID NO: 174 | nucleotide | ANPEP allele having 8 bp deletion |
| SEQ ID NO: 175 | nucleotide | ANPEP allele having 2 bp mismatch |
| SEQ ID NO: 176 | nucleotide | ANPEP allele having 1 bp insertion |
| SEQ ID NO: 177 | nucleotide | ANPEP allele having 661 bp deletion |
| | | and 8 bp insertion (allele B) |
| SEQ ID NO: 178 | nucleotide | ANPEP allele having 8 bp deletion |
| | | and 4 bp insertion (allele C) |
| SEQ ID NO: 179 | nucleotide | Inserted sequence for SEQ ID NO: |
| | | 177 |
| SEQ ID NO: 180 | nucleotide | Inserted sequence for SEQ ID NO: |
| | | 178 |
| SEQ ID NO: 181-185 | nucleotide | Primer sequences (Table 31) |
| SEQ ID NO: 186 | nucleotide | Intron consensus sequence |

### Embodiments

For further illustration, additional non-limiting embodiments of the present disclosure are set forth below.

Embodiment 1 is a livestock animal or offspring thereof or an animal cell comprising at least one modified chromosomal sequence in a gene encoding an aminopeptidase N (ANPEP) protein.

Embodiment 2 is the livestock animal, offspring, or cell of embodiment 1, wherein the modified chromosomal sequence in the gene encoding the ANPEP protein reduces the susceptibility of the animal, offspring, or cell to infection by a pathogen, as compared to the susceptibility of a livestock animal, offspring, or cell that does not comprise a modified chromosomal sequence in a gene encoding an ANPEP protein to infection by the pathogen.

Embodiment 3 is the livestock animal, offspring, or cell of embodiment 2, wherein the pathogen comprises a virus.

Embodiment 4 is the livestock animal, offspring, or cell of embodiment 3, wherein the virus comprises a Coronaviridae family virus.

Embodiment 5 is the livestock animal, offspring, or cell of embodiment 4, wherein the virus comprises a Coronavirinae subfamily virus.

Embodiment 6 is the livestock animal, offspring, or cell of embodiment 5, wherein the virus comprises a coronavirus.

Embodiment 7 is the livestock animal, offspring, or cell of embodiment 6, wherein the coronavirus comprises an Alphacoronavirus genus virus.

Embodiment 8 is the livestock animal, offspring, or cell of embodiment 7, wherein the Alphacoronavirus genus virus comprises a transmissible gastroenteritis virus (TGEV) or a porcine respiratory coronavirus (PRCV).

Embodiment 9 is the livestock animal, offspring, or cell of embodiment 8, wherein the TGEV comprises TGEV Purdue strain.

Embodiment 10 is the livestock animal, offspring, or cell of any one of embodiments 1-9, wherein the livestock animal is selected from the group consisting of an ungulate, an avian animal, and an equine animal; or wherein the cell is derived from an animal selected from the group consisting of an ungulate, an avian animal, and an equine animal.

Embodiment 11 is the livestock animal, offspring, or cell of embodiment 10, wherein the avian animal comprises a chicken, a turkey, a duck, a goose, a guinea fowl, or a squab; or wherein the equine animal comprises a horse or a donkey.

Embodiment 12 is the livestock animal, offspring, or cell of embodiment 10 wherein the ungulate comprises an artiodactyl.

Embodiment 13 is the livestock animal, offspring, or cell of embodiment 11, wherein the artiodactyl comprises a porcine animal, a bovine animal, an ovine animal, a caprine animal, a buffalo, a camel, a llama, an alpaca, or a deer.

Embodiment 14 is the livestock animal, offspring, or cell of embodiment 13, wherein the bovine animal comprises beef cattle or dairy cattle.

Embodiment 15 is the livestock animal, offspring, or cell of embodiment 13, wherein the artiodactyl comprises a porcine animal.

Embodiment 16 is the livestock animal, offspring, or cell of embodiment 15, wherein the porcine animal comprises a pig.

Embodiment 17 is the livestock animal, offspring, or cell of any one of embodiments 1-16, wherein the animal or offspring is an embryo, a juvenile, or an adult, or wherein the cell comprises an embryonic cell, a cell derived from a juvenile animal, or a cell derived from an adult animal.

Embodiment 18 is the livestock animal, offspring, or cell of any one of embodiments 1-17, wherein the animal, offspring, or cell is heterozygous for the modified chromosomal sequence in the gene encoding the ANPEP protein.

Embodiment 19 is the livestock animal, offspring, or cell of any one of embodiments 1-17, wherein the animal, offspring, or cell is homozygous for the modified chromosomal sequence in the gene encoding the ANPEP protein.

Embodiment 20 is the livestock animal, offspring, or cell of any one of embodiments 1-19, wherein the modified chromosomal sequence comprises an insertion in an allele of the gene encoding the ANPEP protein, a deletion in an allele of the gene encoding the ANPEP protein, a substitution in an allele of the gene encoding the ANPEP protein, or a combination of any thereof.

Embodiment 21 is the livestock animal, offspring, or cell of embodiment 20, wherein the modified chromosomal sequence comprises a deletion in an allele of the gene encoding the ANPEP protein.

Embodiment 22 is the livestock animal, offspring, or cell of embodiment 21, wherein the deletion comprises an in-frame deletion.

Embodiment 23 is the livestock animal, offspring, or cell of any one of embodiments 20-22, wherein the modified chromosomal sequence comprises an insertion in an allele of the gene encoding the ANPEP protein.

Embodiment 24 is the livestock animal, offspring, or cell of any one of embodiments 20,21, and 23, wherein the insertion, the deletion, the substitution, or the combination of any thereof results in a miscoding in the allele of the gene encoding the ANPEP protein.

Embodiment 25 is the livestock animal, offspring, or cell of any one of embodiments 20,21,23, and 24, wherein the insertion, the deletion, the substitution, or the miscoding results in a premature stop codon in the allele of the gene encoding the ANPEP protein.

Embodiment 26 is the livestock animal, offspring, or cell of any one of embodiments 20,21, and 23, wherein the deletion comprises a deletion of the start codon of the allele of the gene encoding the ANPEP protein.

Embodiment 27 is the livestock animal, offspring, or cell of any one of embodiments 20,21,23, and 26 wherein the deletion comprises a deletion of the entire coding sequence of the allele of the gene encoding the ANPEP protein.

Embodiment 28 is the livestock animal, offspring, or cell of any one of embodiments 20-26, wherein the modified chromosomal sequence comprises a substitution in an allele of the gene encoding the ANPEP protein.

Embodiment 29 is the livestock animal, offspring, or cell of any one of embodiments 1-28, wherein the modified chromosomal sequence in the gene encoding the ANPEP protein causes ANPEP protein production or activity to be reduced, as compared to ANPEP protein production or activity in an animal, offspring, or cell that lacks the modified chromosomal sequence in the gene encoding the ANPEP protein.

Embodiment 30 is the livestock animal, offspring, or cell of any one of embodiments 1-29, wherein the modified chromosomal sequence in the gene encoding the ANPEP protein results in production of substantially no functional ANPEP protein by the animal, offspring, or cell.

Embodiment 31 is the livestock animal, offspring, or cell of any one of embodiments 1-30, wherein the animal, offspring, or cell does not produce ANPEP protein.

Embodiment 32 is the livestock animal, offspring, or cell of any one of embodiments 1-31, wherein the modified chromosomal sequence comprises a modification in: exon 2 of an allele of the gene encoding the ANPEP protein; exon 4 of an allele of the gene encoding the ANPEP protein; an intron that is contiguous with exon 2 or exon 4 of the allele of the gene encoding the ANPEP protein; or a combination of any thereof.

Embodiment 33 is the livestock animal, offspring, or cell of embodiment 32, wherein the modified chromosomal sequence comprises a modification in exon 2 of the allele of the gene encoding the ANPEP protein, a modification in intron 1 of the allele of the gene encoding the ANPEP protein, or a combination thereof.

Embodiment 34 is the livestock animal, offspring, or cell of embodiment 32 or 33, wherein the modified chromosomal sequence comprises a deletion that begins in intron 1 of the allele of the gene encoding the ANPEP protein and ends in exon 2 of the allele of the gene encoding the ANPEP protein.

Embodiment 35 is the livestock animal, offspring, or cell of embodiment 32 or 33, wherein the modified chromosomal sequence comprises an insertion or a deletion in exon 2 of the allele of the gene encoding the ANPEP protein.

Embodiment 36 is the livestock animal, offspring, or cell of embodiment 35, wherein the insertion or deletion in exon 2 of the allele of the gene encoding the ANPEP protein is downstream of the start codon.

Embodiment 37 is the livestock animal, offspring, or cell of any one of embodiments 32, 33, 36, and 37, wherein the modified chromosomal sequence comprises a deletion in exon 2 of the allele of the gene encoding the ANPEP protein.

Embodiment 38 is the livestock animal, offspring, or cell of embodiment 37, wherein the deletion comprises an in-frame deletion in exon 2.

Embodiment 39 is the livestock animal, offspring, or cell of embodiment 38, wherein the in-frame deletion in exon 2 results in deletion of amino acids 194 through 196 of the ANPEP protein.

Embodiment 40 is the livestock animal, offspring, or cell of embodiment 38, wherein the in-frame deletion in exon 2 results in deletion of amino acids 194 through 197 of the ANPEP protein.

Embodiment 41 is the livestock animal, offspring, or cell of embodiment 40, wherein the in-frame deletion further results in substitution of the valine residue at position 198 of the ANPEP protein with an isoleucine residue.

Embodiment 42 is the livestock animal, offspring, or cell of any one of embodiments 32-41, wherein the modified chromosomal sequence comprises an insertion in exon 2 of the allele of the gene encoding the ANPEP protein.

Embodiment 43 is the livestock animal, offspring, or cell of any one of embodiments 32-42, wherein the modified chromosomal sequence comprises a modification selected from the group consisting of:

a 182 base pair deletion from nucleotide 1,397 to nucleotide 1,578, as compared to reference sequence SEQ ID NO: 135, wherein the deleted sequence is replaced with a 5 base pair insertion beginning at nucleotide 1,397;

a 9 base pair deletion from nucleotide 1,574 to nucleotide 1,582, as compared to reference sequence SEQ ID NO: 135;

a 9 base pair deletion from nucleotide 1,577 to nucleotide 1,585, as compared to reference sequence SEQ ID NO: 135;

a 9 base pair deletion from nucleotide 1,581 to nucleotide 1,589, as compared to reference sequence SEQ ID NO: 135;

an 867 base pair deletion from nucleotide 819 to nucleotide 1,685, as compared to reference sequence SEQ ID NO: 135;

an 867 base pair deletion from nucleotide 882 to nucleotide 1,688, as compared to reference sequence SEQ ID NO: 135;

a 1 base pair insertion between nucleotides 1,581 and 1,582, as compared to reference sequence SEQ ID NO: 135;

a 1 base pair insertion between nucleotides 1,580 and 1,581, as compared to reference sequence SEQ ID NO: 135;

a 1 base pair insertion between nucleotides 1,579 and 1,580, as compared to reference sequence SEQ ID NO: 135;

a 2 base pair insertion between nucleotides 1,581 and 1,582, as compared to reference sequence SEQ ID NO: 135;

a 267 base pair deletion from nucleotide 1,321 to nucleotide 1,587, as compared to reference sequence SEQ ID NO: 135;

a 267 base pair deletion from nucleotide 1,323 to nucleotide 1,589, as compared to reference sequence SEQ ID NO: 135;

a 1 base pair deletion of nucleotide 1,581, as compared to reference sequence SEQ ID NO: 135;

a 12 base pair deletion from nucleotide 1,582 to nucleotide 1,593, as compared to reference sequence SEQ ID NO: 135;

a 25 base pair deletion from nucleotide 1,561 to nucleotide 1,585, as compared to reference sequence SEQ ID NO: 135;

a 25 base pair deletion from nucleotide 1,560 to nucleotide 1,584, as compared to reference sequence SEQ ID NO: 135;

an 8 base pair deletion from nucleotide 1,575 to nucleotide 1,582, as compared to reference sequence SEQ ID NO: 135;

an 8 base pair deletion from nucleotide 1,574 to nucleotide 1,581, as compared to reference sequence SEQ ID NO: 135;

a 661 base pair deletion from nucleotide 940 to nucleotide 1,600, as compared to reference sequence SEQ ID NO: 135, wherein the deleted sequence is replaced with an 8 base pair insertion beginning at nucleotide 940;

an 8 base pair deletion from nucleotide 1,580 to nucleotide 1,587, as compared to reference sequence SEQ ID NO: 135, wherein the deleted sequence is replaced with a 4 base pair insertion beginning at nucleotide 1,580;

and combinations of any thereof.

Embodiment 44 is the livestock animal, offspring, or cell of embodiment 43, wherein:

the modification comprises the 182 base pair deletion from nucleotide 1,397 to nucleotide 1,578, as compared to reference sequence SEQ ID NO: 135, wherein the deleted sequence is replaced with the 5 base pair insertion beginning at nucleotide 1,397, and the 5 base pair insertion comprises the sequence CCCTC (SEQ ID NO: 169);

the modification comprises the 1 base pair insertion between nucleotides 1,581 and 1,582, as compared to reference sequence SEQ ID NO: 135, and the insertion comprises a single thymine (T) residue;

the modification comprises the 1 base pair insertion between nucleotides 1,580 and 1,581, as compared to reference sequence SEQ ID NO: 135, and the insertion comprises a single thymine (T) residue or a single adenine (A) residue;

the modification comprises the 1 base pair insertion between nucleotides 1,579 and 1,580, as compared to reference sequence SEQ ID NO: 135, and the insertion comprises a single adenine (A) residue;

the modification comprises the 2 base pair insertion between nucleotides 1,581 and 1,582, as compared to reference sequence SEQ ID NO: 135, and the insertion comprises an AT dinucleotide;

the modification comprises the 661 base pair deletion from nucleotide 940 to nucleotide 1,600, as compared to reference sequence SEQ ID NO: 135, wherein the deleted sequence is replaced with the 8 base pair insertion beginning at nucleotide 940, and the 8 base pair insertion comprises the sequence GGGGCTTA (SEQ ID NO: 179); or the modification comprises the 8 base pair deletion from nucleotide 1,580 to nucleotide 1,587, as compared to reference sequence SEQ ID NO: 135, wherein the deleted sequence is replaced with the 4 base pair insertion beginning at nucleotide 1,580, and the 4 base pair insertion comprises the sequence TCGT (SEQ ID NO: 180).

Embodiment 45 is the livestock animal, offspring, or cell of embodiment 43 or 44, wherein the modified chromosomal sequence comprises a modification selected from the group consisting of:

the 661 base pair deletion from nucleotide 940 to nucleotide 1,600, as compared to reference sequence SEQ ID NO: 135, wherein the deleted sequence is replaced with the 8 base pair insertion beginning at nucleotide 940;

the 8 base pair deletion from nucleotide 1,580 to nucleotide 1,587, as compared to reference sequence SEQ ID NO: 135, wherein the deleted sequence is replaced with the 4 base pair insertion beginning at nucleotide 1,580;

the 1 base pair insertion between nucleotides 1,581 and 1,582, as compared to reference sequence SEQ ID NO: 135;

the 2 base pair insertion between nucleotides 1,581 and 1,582, as compared to reference sequence SEQ ID NO: 135;

the 9 base pair deletion from nucleotide 1,581 to nucleotide 1,589, as compared to reference sequence SEQ ID NO: 135;

the 12 base pair deletion from nucleotide 1,582 to nucleotide 1,593, as compared to reference sequence SEQ ID NO: 135;

the 1 base pair deletion of nucleotide 1,581, as compared to reference sequence SEQ ID NO: 135;

and combinations of any thereof.

Embodiment 46 is the livestock animal, offspring, or cell of embodiment 45, wherein the modified chromosomal sequence comprises a modification selected from the group consisting of:

the 661 base pair deletion from nucleotide 940 to nucleotide 1,600, as compared to reference sequence SEQ ID NO: 135, wherein the deleted sequence is replaced with the 8 base pair insertion beginning at nucleotide 940;

the 8 base pair deletion from nucleotide 1,580 to nucleotide 1,587, as compared to reference sequence SEQ ID NO: 135, wherein the deleted sequence is replaced with the 4 base pair insertion beginning at nucleotide 1,580;

the 1 base pair insertion between nucleotides 1,581 and 1,582, as compared to reference sequence SEQ ID NO: 135;

the 2 base pair insertion between nucleotides 1,581 and 1,582, as compared to reference sequence SEQ ID NO: 135;

the 1 base pair deletion of nucleotide 1,581, as compared to reference sequence SEQ ID NO: 135;

and combinations of any thereof.

Embodiment 47 is the livestock animal, offspring, or cell of any one of embodiments 43-46, wherein the animal, offspring, or cell comprises:
(a) the 661 base pair deletion from nucleotide 940 to nucleotide 1,600, as compared to reference sequence SEQ ID NO: 135 in one allele of the gene encoding the ANPEP protein, wherein the deleted sequence is replaced with the 8 base pair insertion beginning at nucleotide 940; and
   the 2 base pair insertion between nucleotides 1,581 and 1,582, as compared to reference sequence SEQ ID NO: 135 in the other allele of the gene encoding the ANPEP protein;
(b) the 8 base pair deletion from nucleotide 1,580 to nucleotide 1,587, as compared to reference sequence SEQ ID NO: 135 in one allele of the gene encoding the ANPEP protein, wherein the deleted sequence is replaced with the 4 base pair insertion beginning at nucleotide 1,580; and the 1 base pair insertion between nucleotides 1,581 and 1,582, as compared to reference sequence SEQ ID NO: 135 in the other allele of the gene encoding the ANPEP protein;
(c) the 8 base pair deletion from nucleotide 1,580 to nucleotide 1,587, as compared to reference sequence SEQ ID NO: 135 in one allele of the gene encoding the ANPEP protein, wherein the deleted sequence is replaced with the 4 base pair insertion beginning at nucleotide 1,580; and
   the 1 base pair deletion of nucleotide 1,581, as compared to reference sequence SEQ ID NO: 135 in the other allele of the gene encoding the ANPEP protein;
(d) the 8 base pair deletion from nucleotide 1,580 to nucleotide 1,587, as compared to reference sequence SEQ ID NO: 135 in one allele of the gene encoding the ANPEP protein, wherein the deleted sequence is replaced with the 4 base pair insertion beginning at nucleotide 1,580; and
   the 2 base pair insertion between nucleotides 1,581 and 1,582, as compared to reference sequence SEQ ID NO: 135 in the other allele of the gene encoding the ANPEP protein; or
(e) the 661 base pair deletion from nucleotide 940 to nucleotide 1,600, as compared to reference sequence SEQ ID NO: 135 in one allele of the gene encoding the ANPEP protein, wherein the deleted sequence is replaced with the 8 base pair insertion beginning at nucleotide 940; and
   the 9 base pair deletion from nucleotide 1,581 to nucleotide 1,589, as compared to reference sequence SEQ ID NO: 135 in the other allele of the gene encoding the ANPEP protein.

Embodiment 48 is the livestock animal, offspring, or cell of any one of embodiments 1-31, wherein the modified chromosomal sequence comprises a modification within the region comprising nucleotides 17,235 through 22,422 of reference sequence SEQ ID NO: 132.

Embodiment 49 is the livestock animal, offspring, or cell of embodiment 48, wherein the modified chromosomal sequence comprises a modification within the region comprising nucleotides 17,235 through 22,016 of reference sequence SEQ ID NO: 132.

Embodiment 50 is the livestock animal, offspring, or cell of embodiment 48 or 49, wherein the modified chromosomal sequence comprises a modification within the region comprising nucleotides 21,446 through 21,537 of reference sequence SEQ ID NO: 132.

Embodiment 51 is the livestock animal, offspring, or cell of any one of embodiments 48-50, wherein the modified chromosomal sequence comprises a modification within the region comprising nucleotides 21,479 through 21,529 of reference sequence SEQ ID NO: 132.

Embodiment 52 is the livestock animal, offspring, or cell of any one of embodiments 48-51, wherein the modified chromosomal sequence comprises a modification within the region comprising nucleotides 21,479 through 21,523 of reference sequence SEQ ID NO: 132.

Embodiment 53 is the livestock animal, offspring, or cell of embodiment 52, wherein the modified chromosomal sequence comprises a modification within the region comprising nucleotides 21,538 through 22,422 of reference sequence SEQ ID NO: 132.

Embodiment 54 is the livestock animal, offspring, or cell of embodiment 48 or 53, wherein the modified chromosomal sequence comprises a modification within the region comprising nucleotides 22,017 through 22,422 of reference sequence SEQ ID NO: 132.

Embodiment 55 is the livestock animal, offspring, or cell of any one of embodiments 48, 53, and 54, wherein the modified chromosomal sequence comprises a modification within the region comprising nucleotides 22,054 through 22,256 of reference sequence SEQ ID NO: 132.

Embodiment 56 is the livestock animal, offspring, or cell of any one of embodiments 48 and 53-55, wherein the modified chromosomal sequence comprises a modification within the region comprising nucleotides 22,054 through 22,126 of reference sequence SEQ ID NO: 132.

Embodiment 57 is the livestock animal, offspring, or cell of any one of embodiments 48-56, wherein the modified chromosomal sequence comprises an insertion or a deletion.

Embodiment 58 is the livestock animal, offspring, or cell of embodiment 57, wherein the modified chromosomal sequence comprises a deletion.

Embodiment 59 is the livestock animal, offspring, or cell of embodiment 58, wherein the deletion comprises an in-frame deletion.

Embodiment 60 is the livestock animal, offspring or cell of any one of embodiments 32-59, wherein the modified chromosomal sequence disrupts an intron-exon splice region.

Embodiment 61 is the livestock animal, offspring, or cell of any one of embodiments 48-60, wherein the modified chromosomal sequence comprises a 51 base pair deletion from nucleotide 21,479 to nucleotide 21,529 of reference sequence SEQ ID NO: 132.

Embodiment 62 is the livestock animal, offspring, or cell of any one of embodiments 48-60, wherein the modified chromosomal sequence comprises a 45 base pair deletion from nucleotide 21,479 to nucleotide 21,523 of reference sequence SEQ ID NO: 132.

Embodiment 63 is the livestock animal, offspring, or cell of any one of embodiments 48-60, wherein the modified chromosomal sequence comprises a 3 base pair deletion from nucleotide 21,509 to nucleotide 21,511 of reference sequence SEQ ID NO: 132.

Embodiment 64 is the livestock animal, offspring, or cell of any one of embodiments 48-60, wherein the modified chromosomal sequence comprises a substitution.

Embodiment 65 is the livestock animal, offspring, or cell of embodiment 64, wherein the substitution comprises a substitution of one or more of the nucleotides in the ACC codon at nucleotides 21,509 through 21,511 of SEQ ID NO: 132 with a different nucleotide, to produce a codon that encodes a different amino acid.

Embodiment 66 is the livestock animal, offspring, or cell of embodiment 65, wherein the substitution of the one or more nucleotides results in replacement of the threonine (T) at amino acid 738 of SEQ ID NO: 134 or the threonine (T) at amino acid 792 of SEQ ID NO: 133 with a glycine (G), alanine (A), cysteine (C), valine (V), leucine (L), isoleucine (I), methionine (M), proline (P), phenylalanine (F), tyrosine (Y), tryptophan (W), aspartic acid (D), glutamic acid (E), asparagine (N), glutamine (Q), histidine (H), lysine (K), or arginine (R) residue.

Embodiment 67 is the livestock animal, offspring, or cell of embodiment 65 or 66, wherein the substitution results in replacement of the threonine (T) at amino acid 738 of SEQ ID NO: 134 or the threonine (T) at amino acid 792 of SEQ ID NO: 133 with a glycine (G), alanine (A), cysteine (C), valine (V), leucine (L), isoleucine (I), methionine (M), proline (P), phenylalanine (F), tryptophan (W), asparagine (N), glutamine (Q), histidine (H), lysine (K), or arginine (R) residue.

Embodiment 68 is the livestock animal, offspring, or cell of any one of embodiments 65-67, wherein the substitution results in replacement of the threonine (T) at amino acid 738 of SEQ ID NO: 134 or the threonine (T) at amino acid 792 of SEQ ID NO: 133 with a valine (V) or arginine (R) residue.

Embodiment 69 is the livestock animal, offspring, or cell of any one of embodiments 32-68, wherein the modified chromosomal sequence in the gene encoding the ANPEP protein consists of the deletion, insertion, or substitution.

Embodiment 70 is the livestock animal, offspring, or cell of any one of embodiments 20-69, wherein the animal, offspring or cell comprises a chromosomal sequence in the gene encoding the ANPEP protein having at least 80%, at least 85%, at least 90%, at least 95%, at least 98%, at least 99%, at least 99.9%, or 100% sequence identity to SEQ ID NO: 135 or 132 in the regions of the chromosomal sequence outside of the insertion, the deletion, or the substitution.

Embodiment 71 is the livestock animal, offspring, or cell of any one of embodiments 1-70, wherein the livestock animal, offspring, or cell comprises a chromosomal sequence comprising SEQ ID NO: 163, 164, 165, 166, 167, 168, 170, 171, 172, 173, 174, 176, 177, or 178.

Embodiment 72 is the livestock animal, offspring, or cell of embodiment 71, wherein the livestock animal, offspring, or cell comprises a chromosomal sequence comprising SEQ ID NO: 177, 178, 166, 167, 170, 172, or 171.

Embodiment 73 is the livestock animal, offspring, or cell of embodiment 71, wherein the livestock animal, offspring, or cell comprises a chromosomal sequence comprising SEQ ID NO: 177, 178, 166, 167, or 171.

Embodiment 74 is the livestock animal, offspring, or cell of any one of embodiments 1-73, wherein the livestock animal, offspring, or cell further comprises at least one modified chromosomal sequence in a gene encoding a CD163 protein.

Embodiment 75 is the livestock animal, offspring, or cell of embodiment 74, wherein the modified chromosomal sequence in the gene encoding the CD163 protein reduces the susceptibility of the animal, offspring, or cell to infection by a pathogen, as compared to the susceptibility of an animal, offspring, or cell that does not comprise a modified chromosomal sequence in a gene encoding a CD163 protein to infection by the pathogen.

Embodiment 76 is the livestock animal, offspring, or cell of embodiment 75, wherein the pathogen comprises a virus.

Embodiment 77 is the livestock animal, offspring, or cell of embodiment 76, wherein the virus comprises a porcine reproductive and respiratory syndrome virus (PRRSV).

Embodiment 78 is the livestock animal, offspring, or cell of embodiment 77, wherein the modified chromosomal sequence in the gene encoding the CD163 protein reduces the susceptibility of the animal, offspring, or cell to a Type 1 PRRSV virus, a Type 2 PRRSV, or to both Type 1 and Type 2 PRRSV viruses.

Embodiment 79 is the livestock animal, offspring, or cell of embodiment 78, wherein the modified chromosomal sequence in the gene encoding the CD163 protein reduces the susceptibility of the animal, offspring, or cell to a PRRSV isolate selected from the group consisting of NVSL 97-7895, KS06-72109, P129, VR2332, C090, AZ25, MLV-ResPRRS, KS62-06274, KS483 (SD23983), C084, SD13-15, Lelystad, 03-1059, 03-1060, SD01-08, 4353PZ, and combinations of any thereof.

Embodiment 80 is the livestock animal, offspring, or cell of any one of embodiments 74-79, wherein the animal, offspring, or cell is heterozygous for the modified chromosomal sequence in the gene encoding the CD163 protein.

Embodiment 81 is the livestock animal, offspring, or cell of any one of embodiments 74-79, wherein the animal, offspring, or cell is homozygous for the modified chromosomal sequence in the gene encoding the CD163 protein.

Embodiment 82 is the livestock animal, offspring, or cell of any one of embodiments 74-81, wherein the modified chromosomal sequence in the gene encoding the CD163 protein comprises an insertion in an allele of the gene encoding the CD163 protein, a deletion in an allele of the gene encoding the CD163 protein, a substitution in an allele of the gene encoding the CD163 protein, or a combination of any thereof.

Embodiment 83 is the livestock animal, offspring, or cell of embodiment 82, wherein the modified chromosomal sequence in the gene encoding the CD163 protein comprises a deletion in an allele of the gene encoding the CD163 protein.

Embodiment 84 is the livestock animal, offspring, or cell of embodiment 82 or 83, wherein the modified chromosomal sequence in the gene encoding the CD163 protein comprises an insertion in an allele of the gene encoding the CD163 protein.

Embodiment 85 is the livestock animal, offspring, or cell of any one of embodiments 82-84, wherein the insertion, the deletion, the substitution, or the combination of any thereof results in a miscoding in the allele of the gene encoding the CD163 protein.

Embodiment 86 is the livestock animal, offspring, or cell of any one of embodiments 82-85, wherein the insertion, the deletion, the substitution, or the miscoding results in a premature stop codon in the allele of the gene encoding the CD163 protein.

Embodiment 87 is the livestock animal, offspring, or cell of any one of embodiments 74-86, wherein the modified chromosomal sequence in the gene encoding the CD163 protein causes CD163 protein production or activity to be reduced, as compared to CD163 protein production or activity in an animal, offspring, or cell that lacks the modified chromosomal sequence in the gene encoding the CD163 protein.

Embodiment 88 is the livestock animal, offspring, or cell of any one of embodiments 74-87, wherein the modified chromosomal sequence in the gene encoding the CD163 protein results in production of substantially no functional CD163 protein by the animal, offspring, or cell.

Embodiment 89 is the livestock animal, offspring, or cell of any one of embodiments 74-80, wherein the animal, offspring, or cell does not produce CD163 protein.

Embodiment 90 is the livestock animal, offspring, or cell of any one of embodiments 74-89, wherein the livestock animal or offspring comprises a porcine animal or wherein the cell comprises a porcine cell.

Embodiment 91 is the livestock animal, offspring, or cell of embodiment 90, wherein the modified chromosomal sequence in the gene encoding the CD163 protein comprises a modification in: exon 7 of an allele of the gene encoding the CD163 protein; exon 8 of an allele of the gene encoding the CD163 protein; an intron that is contiguous with exon 7 or exon 8 of the allele of the gene encoding the CD163 protein; or a combination of any thereof.

Embodiment 92 is the livestock animal, offspring, or cell of embodiment 91, wherein the modified chromosomal sequence in the gene encoding the CD163 protein comprises a modification in exon 7 of the allele of the gene encoding the CD163 protein.

Embodiment 93 is the livestock animal, offspring, or cell of embodiment 92, wherein the modification in exon 7 of the allele of the gene encoding the CD163 protein comprises a deletion.

Embodiment 94 is the livestock animal, offspring, or cell of any one of embodiments 82-93, wherein the deletion comprises an in-frame deletion.

Embodiment 95 is the livestock animal, offspring, or cell of any one of embodiments 92-94, wherein the modification in exon 7 of the allele of the gene encoding the CD163 protein comprises an insertion.

Embodiment 96 is the livestock animal, offspring, or cell of any one of embodiments 90-95, wherein the modified chromosomal sequence in the gene encoding the CD163 protein comprises a modification selected from the group consisting of:

an 11 base pair deletion from nucleotide 3,137 to nucleotide 3,147 as compared to reference sequence SEQ ID NO: 47;

a 2 base pair insertion between nucleotides 3,149 and 3,150 as compared to reference sequence SEQ ID NO: 47, with a 377 base pair deletion from nucleotide 2,573 to nucleotide 2,949 as compared to reference sequence SEQ ID NO: 47 on the same allele;

a 124 base pair deletion from nucleotide 3,024 to nucleotide 3,147 as compared to reference sequence SEQ ID NO: 47;

a 123 base pair deletion from nucleotide 3,024 to nucleotide 3,146 as compared to reference sequence SEQ ID NO: 47;

a 1 base pair insertion between nucleotides 3,147 and 3,148 as compared to reference sequence SEQ ID NO: 47;

a 130 base pair deletion from nucleotide 3,030 to nucleotide 3,159 as compared to reference sequence SEQ ID NO: 47;

a 132 base pair deletion from nucleotide 3,030 to nucleotide 3,161 as compared to reference sequence SEQ ID NO: 47;

a 1506 base pair deletion from nucleotide 1,525 to nucleotide 3,030 as compared to reference sequence SEQ ID NO: 47;

a 7 base pair insertion between nucleotide 3,148 and nucleotide 3,149 as compared to reference sequence SEQ ID NO: 47;

a 1280 base pair deletion from nucleotide 2,818 to nucleotide 4,097 as compared to reference sequence SEQ ID NO: 47;

a 1373 base pair deletion from nucleotide 2,724 to nucleotide 4,096 as compared to reference sequence SEQ ID NO: 47;

a 1467 base pair deletion from nucleotide 2,431 to nucleotide 3,897 as compared to reference sequence SEQ ID NO: 47;

a 1930 base pair deletion from nucleotide 488 to nucleotide 2,417 as compared to reference sequence SEQ ID NO: 47, wherein the deleted sequence is replaced with a 12 base pair insertion beginning at nucleotide 488, and wherein there is a further 129 base pair deletion in exon 7 from nucleotide 3,044 to nucleotide 3,172 as compared to reference sequence SEQ ID NO: 47;

a 28 base pair deletion from nucleotide 3,145 to nucleotide 3,172 as compared to reference sequence SEQ ID NO: 47;

a 1387 base pair deletion from nucleotide 3,145 to nucleotide 4,531 as compared to reference sequence SEQ ID NO: 47;

a 1382 base pair deletion from nucleotide 3,113 to nucleotide 4,494 as compared to reference sequence SEQ ID NO: 47, wherein the deleted sequence is replaced with an 11 base pair insertion beginning at nucleotide 3,113;

a 1720 base pair deletion from nucleotide 2,440 to nucleotide 4,160 as compared to reference sequence SEQ ID NO: 47;

a 452 base pair deletion from nucleotide 3,015 to nucleotide 3,466 as compared to reference sequence SEQ ID NO: 47;

and combinations of any thereof.

Embodiment 97 is the livestock animal, offspring, or cell of embodiment 96, wherein:

the modification comprises the 2 base pair insertion between nucleotides 3,149 and 3,150 as compared to reference sequence SEQ ID NO: 47, with the 377 base pair deletion from nucleotide 2,573 to nucleotide 2,949 as compared to reference sequence SEQ ID NO: 47 on the same allele, and the 2 base pair insertion comprises the dinucleotide AG;

the modification comprises the 1 base pair insertion between nucleotides 3,147 and 3,148 as compared to reference sequence SEQ ID NO: 47, and the 1 base pair insertion comprises a single adenine residue;

the modification comprises the 7 base pair insertion between nucleotide 3,148 and nucleotide 3,149 as compared to reference sequence SEQ ID NO: 47, and the 7 base pair insertion comprises the sequence TACTACT (SEQ ID NO: 115);

the modification comprises the 1930 base pair deletion from nucleotide 488 to nucleotide 2,417 as compared to reference sequence SEQ ID NO: 47, wherein the deleted sequence is replaced with a 12 base pair insertion beginning at nucleotide 488, and wherein there is a further 129 base pair deletion in exon 7 from nucleotide 3,044 to nucleotide 3,172 as compared to reference sequence SEQ ID NO: 47, and wherein the 12 base pair insertion comprises the sequence TGTGGAGAATTC (SEQ ID NO: 116); or the modification comprises the 1382 base pair deletion from nucleotide 3,113 to nucleotide 4,494 as compared to reference sequence SEQ ID NO: 47, wherein the deleted sequence is replaced with an 11 base pair insertion beginning at nucleotide 3,113, and the 11 base pair insertion comprises the sequence AGCCAGCGTGC (SEQ ID NO: 117).

Embodiment 98 is the livestock animal, offspring, or cell of embodiment 96, wherein the modified chromosomal sequence in the gene encoding the CD163 protein comprises a modification selected from the group consisting of:

the 7 base pair insertion between nucleotide 3,148 and nucleotide 3,149 as compared to reference sequence SEQ ID NO: 47;

the 2 base pair insertion between nucleotides 3,149 and 3,150 as compared to reference sequence SEQ ID NO: 47, with the 377 base pair deletion from nucleotide 2,573 to nucleotide 2,949 as compared to reference sequence SEQ ID NO: 47 on the same allele;

the 11 base pair deletion from nucleotide 3,137 to nucleotide 3,147 as compared to reference sequence SEQ ID NO: 47;

the 1382 base pair deletion from nucleotide 3,113 to nucleotide 4,494 as compared to reference sequence SEQ ID NO: 47, wherein the deleted sequence is replaced with the 11 base pair insertion beginning at nucleotide 3,113;

the 1387 base pair deletion from nucleotide 3,145 to nucleotide 4,531 as compared to reference sequence SEQ ID NO: 47;

and combinations of any thereof.

Embodiment 99 is the livestock animal, offspring, or cell of any one of embodiments 96-98, wherein the animal, offspring, or cell comprises:
(a) the 7 base pair insertion between nucleotide 3,148 and nucleotide 3,149 as compared to reference sequence SEQ ID NO: 47 in one allele of the gene encoding the CD163 protein; and the 11 base pair deletion from nucleotide 3,137 to nucleotide 3,147 as compared to reference sequence SEQ ID NO: 47 in the other allele of the gene encoding the CD163 protein;
(b) the 7 base pair insertion between nucleotide 3,148 and nucleotide 3,149 as compared to reference sequence SEQ ID NO: 47 in one allele of the gene encoding the CD163 protein; and
   the 1382 base pair deletion from nucleotide 3,113 to nucleotide 4,494 as compared to reference sequence SEQ ID NO: 47, wherein the deleted sequence is replaced with an 11 base pair insertion beginning at nucleotide 3,113 in the other allele of the gene encoding the CD163 protein;
(c) the 1280 base pair deletion from nucleotide 2,818 to nucleotide 4,097 as compared to reference sequence SEQ ID NO: 47 in one allele of the gene encoding the CD163 protein; and
   the 11 base pair deletion from nucleotide 3,137 to nucleotide 3,147 as compared to reference sequence SEQ ID NO: 47 in the other allele of the gene encoding the CD163 protein;
(d) the 1280 base pair deletion from nucleotide 2,818 to nucleotide 4,097 as compared to reference sequence SEQ ID NO: 47 in one allele of the gene encoding the CD163 protein; and
   the 2 base pair insertion between nucleotides 3,149 and 3,150 as compared to reference sequence SEQ ID NO: 47, with the 377 base pair deletion from nucleotide 2,573 to nucleotide 2,949 as compared to reference sequence SEQ ID NO: 47 in the other allele of the gene encoding the CD163 protein;
(e) the 1930 base pair deletion from nucleotide 488 to nucleotide 2,417 as compared to reference sequence SEQ ID NO: 47, wherein the deleted sequence is replaced with a 12 base pair insertion beginning at nucleotide 488, and wherein there is a further 129 base pair deletion in exon 7 from nucleotide 3,044 to nucleotide 3,172 as compared to reference sequence SEQ ID NO: 47 in one allele of the gene encoding the CD163 protein; and
   the 2 base pair insertion between nucleotides 3,149 and 3,150 as compared to reference sequence SEQ ID NO: 47, with the 377 base pair deletion from nucleotide 2,573 to nucleotide 2,949 as compared to reference sequence SEQ ID NO: 47 in the other allele of the gene encoding the CD163 protein;
(f) the 1930 base pair deletion from nucleotide 488 to nucleotide 2,417 as compared to reference sequence SEQ ID NO: 47, wherein the deleted sequence is replaced with a 12 base pair insertion beginning at nucleotide 488, and wherein there is a further 129 base pair deletion in exon 7 from nucleotide 3,044 to nucleotide 3,172 as compared to reference sequence SEQ ID NO: 47 in one allele of the gene encoding the CD163 protein; and
   the 11 base pair deletion from nucleotide 3,137 to nucleotide 3,147 as compared to reference sequence SEQ ID NO: 47 in the other allele of the gene encoding the CD163 protein;
(g) the 1467 base pair deletion from nucleotide 2,431 to nucleotide 3,897 as compared to reference sequence SEQ ID NO: 47 in one allele of the gene encoding the CD163 protein; and
   the 2 base pair insertion between nucleotides 3,149 and 3,150 as compared to reference sequence SEQ ID NO: 47, with the 377 base pair deletion from nucleotide 2,573 to nucleotide 2,949 as compared to reference sequence SEQ ID NO: 47 in the other allele of the gene encoding the CD163 protein;
(h) the 1467 base pair deletion from nucleotide 2,431 to nucleotide 3,897 as compared to reference sequence SEQ ID NO: 47 in one allele of the gene encoding the CD163 protein; and
   the 11 base pair deletion from nucleotide 3,137 to nucleotide 3,147 as compared to reference sequence SEQ ID NO: 47 in the other allele of the gene encoding the CD163 protein;
(i) the 11 base pair deletion from nucleotide 2,431 to nucleotide 3,897 as compared to reference sequence SEQ ID NO: 47 in one allele of the gene encoding the CD163 protein; and
   the 2 base pair insertion between nucleotides 3,149 and 3,150 as compared to reference sequence SEQ ID NO: 47, with the 377 base pair deletion from nucleotide 2,573 to nucleotide 2,949 as compared to reference sequence SEQ ID NO: 47 in the other allele of the gene encoding the CD163 protein;
(j) the 124 base pair deletion from nucleotide 3,024 to nucleotide 3,147 as compared to reference sequence SEQ ID NO: 47 in one allele of the gene encoding the CD163 protein; and
   the 123 base pair deletion from nucleotide 3,024 to nucleotide 3,146 as compared to reference sequence SEQ ID NO: 47 in the other allele of the gene encoding the CD163 protein;
(k) the 130 base pair deletion from nucleotide 3,030 to nucleotide 3,159 as compared to reference sequence SEQ ID NO: 47 in one allele of the gene encoding the CD163 protein; and
   the 132 base pair deletion from nucleotide 3,030 to nucleotide 3,161 as compared to reference sequence SEQ ID NO: 47 in the other allele of the gene encoding the CD163 protein;
(l) the 1280 base pair deletion from nucleotide 2,818 to nucleotide 4,097 as compared to reference sequence SEQ ID NO: 47 in one allele of the gene encoding the CD163 protein; and
   the 1373 base pair deletion from nucleotide 2,724 to nucleotide 4,096 as compared to reference sequence SEQ ID NO: 47 in the other allele of the gene encoding the CD163 protein;
(m) the 28 base pair deletion from nucleotide 3,145 to nucleotide 3,172 as compared to reference sequence SEQ ID NO: 47 in one allele of the gene encoding the CD163 protein; and
   the 1387 base pair deletion from nucleotide 3,145 to nucleotide 4,531 as compared to reference sequence SEQ ID NO: 47 in the other allele of the gene encoding the CD163 protein;
(n) the 1382 base pair deletion from nucleotide 3,113 to nucleotide 4,494 as compared to reference sequence SEQ ID NO: 47, wherein the deleted sequence is replaced with an 11 base pair insertion beginning at nucleotide 3,113, in one allele of the gene encoding the CD163 protein; and
   the 1720 base pair deletion from nucleotide 2,440 to nucleotide 4,160 as compared to reference sequence SEQ ID NO: 47 in the other allele of the gene encoding the CD163 protein;
(o) the 7 base pair insertion between nucleotide 3,148 and nucleotide 3,149 as compared to reference sequence SEQ ID NO: 47 in one allele of the CD163 gene; and the 2 base pair insertion between nucleotides 3,149 and 3,150 as compared to reference sequence SEQ ID NO: 47, with the 377 base pair deletion from nucleotide 2,573 to nucleotide 2,949 as compared to reference sequence SEQ ID NO: 47, in the other allele of the CD163 gene;
(p) the 1382 base pair deletion from nucleotide 3,113 to nucleotide 4,494 as compared to reference sequence SEQ ID NO: 47, wherein the deleted sequence is replaced with the 11 base pair insertion beginning at nucleotide 3,113, in one allele of the CD163 gene; and
   the 2 base pair insertion between nucleotides 3,149 and 3,150 as compared to reference sequence SEQ ID NO: 47, with the 377 base pair deletion from nucleotide 2,573 to nucleotide 2,949 as compared to reference sequence SEQ ID NO: 47, in the other allele of the CD163 gene; or
(q) the 1382 base pair deletion from nucleotide 3,113 to nucleotide 4,494 as compared to reference sequence SEQ ID NO: 47, wherein the deleted sequence is replaced with the 11 base pair insertion beginning at nucleotide 3,113, in one allele of the CD163 gene; and
   the 11 base pair deletion from nucleotide 3,137 to nucleotide 3,147 as compared to reference sequence SEQ ID NO: 47 in the other allele of the CD163 gene.

Embodiment 100 is the livestock animal, offspring, or cell of any one of embodiments 82-99, wherein the modified chromosomal sequence in the gene encoding the CD163 protein consists of the deletion insertion, or substitution.

Embodiment 101 is the livestock animal, offspring, or cell of any one of embodiments 82-100, wherein the animal, offspring, or cell comprises a chromosomal sequence in the gene encoding the CD163 protein having at least 80%, at least 85%, at least 90%, at least 95%, at least 98%, at least 99%, at least 99.9%, or 100% sequence identity to SEQ ID NO: 47 in the regions of the chromosomal sequence outside of the insertion, the deletion, or the substitution.

Embodiment 102 is the livestock animal, offspring, or cell of any one of embodiments 74-101, wherein the animal, offspring, or cell comprises a chromosomal sequence comprising SEQ ID NO: 98, 99, 100, 101, 102, 103, 104, 105, 106, 107, 108, 109, 110, 111, 112, 113, 114, or 119.

Embodiment 103 is the livestock animal, offspring, or cell of any one of embodiments 74-102, wherein:

the modified chromosomal sequence in the gene encoding the ANPEP protein comprises the 1 base pair insertion between nucleotides 1,581 and 1,582, as compared to reference sequence SEQ ID NO: 135; and the modified chromosomal sequence in the gene encoding the CD163 protein comprises the 1387 base pair deletion from nucleotide 3,145 to nucleotide 4,531 as compared to reference sequence SEQ ID NO: 47.

Embodiment 104 is the livestock animal, offspring, or cell of any one of embodiments 1-103, wherein the livestock animal, offspring, or cell further comprises a modified chromosomal sequence in a gene encoding a SIGLEC1 protein.

Embodiment 105 is the livestock animal, offspring, or cell of embodiment 104, wherein the animal, offspring, or cell is heterozygous for the modified chromosomal sequence in the gene encoding the SIGLEC1 protein.

Embodiment 106 is the livestock animal, offspring, or cell of embodiment 104, wherein the animal, offspring, or cell is homozygous for the modified chromosomal sequence in the gene encoding the SIGLEC1 protein.

Embodiment 107 is the livestock animal, offspring, or cell of any one of embodiments 104-106, wherein the modified chromosomal sequence in the gene encoding the SIGLEC1 protein comprises an insertion in an allele of the gene encoding the SIGLEC1 protein, a deletion in an allele of the gene encoding the SIGLEC1 protein, a substitution in an allele of the gene encoding the SIGLEC1 protein, or a combination of any thereof.

Embodiment 108 is the livestock animal, offspring, or cell of embodiment 107, wherein the modified chromosomal sequence in the gene encoding the SIGLEC1 protein comprises a deletion in an allele of the gene encoding the SIGLEC1 protein.

Embodiment 109 is the livestock animal, offspring, or cell of embodiment 108, wherein the deletion comprises an in-frame deletion.

Embodiment 110 is the livestock animal, offspring, or cell of any one of embodiments 107-109, wherein the modified chromosomal sequence in the gene encoding the SIGLEC1 protein comprises an insertion in an allele of the gene encoding the SIGLEC1 protein.

Embodiment 111 is the livestock animal, offspring, or cell of any one of embodiments 107-110, wherein the modified chromosomal sequence in the gene encoding the SIGLEC1 protein comprises a substitution in an allele of the gene encoding the SIGLEC1 protein.

Embodiment 112 is the livestock animal, offspring, or cell of any one of embodiments 107,108,110, and 111, wherein the insertion, the deletion, the substitution, or the combination of any thereof results in a miscoding in the allele of the gene encoding the SIGLEC1 protein.

Embodiment 113 is the livestock animal, offspring, or cell of any one of embodiments 107,108, and 110-112, wherein the insertion, the deletion, the substitution, or the miscoding results in a premature stop codon in the allele of the gene encoding the SIGLEC1 protein.

Embodiment 114 is the livestock animal, offspring, or cell of any one of embodiments 104-113, wherein the modified chromosomal sequence in the gene encoding the SIGLEC1 protein causes SIGLEC1protein production or activity to be reduced, as compared to SIGLEC1 protein production or activity in an animal, offspring, or cell that lacks the modified chromosomal sequence in the gene encoding the SIGLEC1 protein.

Embodiment 115 is the he livestock animal, offspring, or cell of any one of embodiments 104-114, wherein the modified chromosomal sequence in the gene encoding the SIGLEC1 protein results in production of substantially no functional SIGLEC1 protein by the animal, offspring, or cell.

Embodiment 116 is the livestock animal, offspring, or cell of any one of embodiments 104-115, wherein the animal, offspring, or cell does not produce SIGLEC1 protein.

Embodiment 117 is the livestock animal, offspring, or cell of any one of embodiments 104-116, wherein the animal or offspring comprises a porcine animal or wherein the cell comprises a porcine cell.

Embodiment 118 is the livestock animal, offspring, or cell of embodiment 117, wherein the modified chromosomal sequence in the gene encoding the SIGLEC1 protein comprises a modification in: exon 1 of an allele of the gene encoding the SIGLEC1 protein; exon 2 of an allele of the gene encoding the SIGLEC1 protein; exon 3 of an allele of the gene encoding the SIGLEC1 protein; an intron that is contiguous with exon 1, exon 2, or exon 3 of an allele of the gene encoding the SIGLEC1 protein; or a combination of any thereof.

Embodiment 119 is the livestock animal, offspring, or cell of embodiment 118, wherein the modified chromosomal sequence in the gene encoding the SIGLEC1 protein comprises a deletion in exon 1, exon 2, and/or exon 3 of an allele of the gene encoding the SIGLEC1 protein.

Embodiment 120 is the livestock animal, offspring, or cell of embodiment 118 or 119, wherein the modified chromosomal sequence in the gene encoding the SIGLEC1 protein comprises a deletion of part of exon 1 and all of exons 2 and 3 of an allele of the gene encoding the SIGLEC1 protein.

Embodiment 121 is the livestock animal, offspring, or cell of any one of embodiments 118-120, wherein the modified chromosomal sequence comprises a 1,247 base pair deletion from nucleotide 4,279 to nucleotide 5,525 as compared to reference sequence SEQ ID NO: 122.

Embodiment 122 is the livestock animal, offspring, or cell of any one of embodiments 119-121, wherein the deleted sequence is replaced with a neomycin gene cassette.

Embodiment 123 is the livestock animal, offspring, or cell of any one of embodiments 107-122, wherein the modified chromosomal sequence in the gene encoding the SIGLEC1 protein consists of the deletion insertion, or substitution.

Embodiment 124 is the livestock animal, offspring, or cell of any one of embodiments 107-123, wherein the animal, offspring, or cell comprises a chromosomal sequence in the gene encoding the SIGLEC1 protein having at least 80%, at least 85%, at least 90%, at least 95%, at least 98%, at least 99%, at least 99.9%, or 100% sequence identity to SEQ ID NO: 122 in the regions of the chromosomal sequence outside of the insertion, the deletion, or the substitution.

Embodiment 125 is the livestock animal, offspring, or cell of any one of embodiments 104-124, wherein the animal, offspring, or cell comprises a chromosomal sequence comprising SEQ ID NO: 123.

Embodiment 126 is the livestock animal, offspring, or cell of any one of embodiments 121-125, wherein:

the modified chromosomal sequence in the gene encoding the ANPEP protein comprises the 1 base pair insertion between nucleotides 1,581 and 1,582, as compared to reference sequence SEQ ID NO: 135; and the modified chromosomal sequence in the gene encoding the SIGLEC1 protein comprises the 1,247 base pair deletion from nucleotide 4,279 to nucleotide 5,525 as compared to reference sequence SEQ ID NO: 122.

Embodiment 127 is the livestock animal, offspring, or cell of embodiment 126, wherein the animal, offspring, or cell further comprises a modified chromosomal sequence in the gene encoding the CD163 protein, the modified chromosomal sequence in the gene encoding the CD163 protein comprising the 1387 base pair deletion from nucleotide 3,145 to nucleotide 4,531 as compared to reference sequence SEQ ID NO: 47.

Embodiment 128 is the livestock animal, offspring, or cell of any one of embodiments 1-127, wherein the animal or offspring comprises a genetically edited animal or offspring or wherein the cell comprises a genetically edited cell.

Embodiment 129 is the livestock animal, offspring, or cell of embodiment 128, wherein the animal or cell has been genetically edited using a homing endonuclease.

Embodiment 130 is the livestock animal, offspring, or cell of embodiment 129, wherein the homing endonuclease comprises a designed homing endonuclease.

Embodiment 131 is the livestock animal, offspring, or cell of embodiment 129 or 130, wherein the homing endonuclease comprises a Clustered Regularly Interspaced Short Palindromic Repeats (CRISPR) system, a Transcription Activator-Like Effector Nuclease (TALEN), a Zinc Finger Nuclease (ZFN), a recombinase fusion protein, a meganuclease, or a combination of any thereof.

Embodiment 132 is the livestock animal, offspring, or cell of any one of embodiments 128-131, wherein the animal or cell has been genetically edited using a CRISPR system.

Embodiment 133 is the livestock animal of any one of embodiments 1-132.

Embodiment 134 is the offspring of any one of embodiments 1-132.

Embodiment 135 is the cell of any one of embodiments 1-132.

Embodiment 136 is the cell of embodiment 135, wherein the cell comprises a sperm cell.

Embodiment 137 is the cell of embodiment 135, wherein the cell comprises an egg cell.

Embodiment 138 is the cell of embodiment 137, wherein the egg cell comprises a fertilized egg.

Embodiment 139 is the cell of embodiment 135, wherein the cell comprises a somatic cell.

Embodiment 140 is the cell of embodiment 139, wherein the somatic cell comprises a fibroblast.

Embodiment 141 is the cell of embodiment 141, wherein the fibroblast comprises a fetal fibroblast.

Embodiment 142 is the cell of any one of embodiments 135, 139, and 140, wherein the cell comprises an embryonic cell or a cell derived from a juvenile animal.

Embodiment 143 is a method of producing a non-human animal or a lineage of non-human animals having reduced susceptibility to infection by a pathogen, wherein the method comprises:

modifying an oocyte or a sperm cell to introduce a modified chromosomal sequence in a gene encoding an aminopeptidase N (ANPEP) protein into at least one of the oocyte and the sperm cell, and fertilizing the oocyte with the sperm cell to create a fertilized egg containing the modified chromosomal sequence in the gene encoding a ANPEP protein; or

modifying a fertilized egg to introduce a modified chromosomal sequence in a gene encoding an ANPEP protein into the fertilized egg;

transferring the fertilized egg into a surrogate female animal, wherein gestation and term delivery produces a progeny animal;

screening the progeny animal for susceptibility to the pathogen; and

selecting progeny animals that have reduced susceptibility to the pathogen as compared to animals that do not comprise a modified chromosomal sequence in a gene encoding an ANPEP protein.

Embodiment 144 is the method of embodiment 143, wherein the animal comprises a livestock animal.

Embodiment 145 is the method of embodiment 143 or 144, wherein the step of modifying the oocyte, sperm cell, or fertilized egg comprises genetic editing of the oocyte, sperm cell, or fertilized egg.

Embodiment 146 is the method of any one of embodiments 143-145, wherein the oocyte, sperm cell, or fertilized egg is heterozygous for the modified chromosomal sequence.

Embodiment 147 is the method of any one of embodiments 143-145, wherein the oocyte, sperm cell, or fertilized egg is homozygous for the modified chromosomal sequence.

Embodiment 148 is the method of any one of embodiments 143-147, wherein the fertilizing comprises artificial insemination.

Embodiment 149 is a method of increasing a livestock animal's resistance to infection with a pathogen, comprising modifying at least one chromosomal sequence in a gene encoding an aminopeptidase N (ANPEP) protein, so that ANPEP protein production or activity is reduced, as compared to ANPEP protein production or activity in a livestock animal that does not comprise a modified chromosomal sequence in a gene encoding an ANPEP protein.

Embodiment 150 is the method of embodiment 149, wherein the step of modifying the at least one chromosomal sequence in the gene encoding the ANPEP protein comprises genetic editing of the chromosomal sequence.

Embodiment 151 is the method of any one of embodiments 145-148 and 150, wherein the genetic editing comprises use of a homing endonuclease.

Embodiment 152 is the method of embodiment 151, wherein the homing endonuclease comprises a designed homing endonuclease.

Embodiment 153 is the method of embodiment 151 or 152, wherein the homing endonuclease comprises a Clustered Regularly Interspaced Short Palindromic Repeats (CRISPR) system, a Transcription Activator-Like Effector Nuclease (TALEN), a Zinc Finger Nuclease (ZFN), a recombinase fusion protein, a meganuclease, or a combination thereof.

Embodiment 154 is the method of any one of embodiments 145-148 and 150-153, wherein the genetic editing comprises the use of a CRISPR system.

Embodiment 155 is the method of any one of embodiments 143-154, wherein the method produces an animal of any one of embodiments 1-153.

Embodiment 156 is the method of any one of embodiments 143-155, further comprising using the animal as a founder animal.

Embodiment 157 is a population of livestock animals comprising two or more livestock animals and/or offspring thereof of any one of embodiments 1-133.

Embodiment 158 is a population of animals comprising two or more animals made by the method of any one of embodiments 143-156 and/or offspring thereof.

Embodiment 159 is the population of embodiment 157 or 158, wherein the population of animals is resistant to infection by a pathogen.

Embodiment 160 is the population of embodiment 159, wherein the pathogen comprises a virus.

Embodiment 161 is the population of embodiment 160, wherein the virus comprises a transmissible gastroenteritis virus (TGEV) or a porcine respiratory coronavirus (PRCV).

Embodiment 162. is a nucleic acid molecule comprising a nucleotide sequence selected from the group consisting of:

(a) a nucleotide sequence having at least 80% sequence identity to the sequence of SEQ ID NO: 135, wherein the nucleotide sequence comprises at least one substitution, insertion, or deletion relative to SEQ ID NO: 135;

(b) a nucleotide sequence having at least 80% sequence identity to the sequence of SEQ ID NO: 132, wherein the nucleotide sequence comprises at least one substitution, insertion, or deletion relative to SEQ ID NO: 132; and

(c) a cDNA of (a) or (b).

Embodiment 163 is the nucleic acid molecule of embodiment 162, wherein the nucleic acid molecule is an isolated nucleic acid molecule.

Embodiment 164 is the nucleic acid molecule of embodiment 162 or 163, wherein the nucleic acid comprises a nucleotide sequence having at least 80%, at least 85%, at least 87.5%, at least 90%, at least 95%, at least 98%, at least 99%, or at least 99.9% identity to SEQ ID NO: 132 or 135, wherein the nucleotide sequence comprises at least one substitution, insertion, or deletion relative to SEQ ID NO: 132 or 135.

Embodiment 165 is the nucleic acid molecule of any one of embodiments 162-164, wherein the substitution, insertion, or deletion reduces or eliminates ANPEP protein production or activity, as compared to a nucleic acid that does not comprise the substitution, insertion, or deletion.

Embodiment 166 is the nucleic acid molecule of any one of embodiments 162-165, wherein the nucleic acid comprises SEQ ID NO. 163, 164, 165, 166, 167, 168, 170, 171, 172, 173, 174, 176, 177, or 178.

Embodiment 167 is the nucleic acid molecule of embodiment 166, wherein the nucleic acid comprises SEQ ID NO: 177, 178, 166, 167, or 171.

1. A livestock animal or offspring thereof or an animal cell comprising at least one modified chromosomal sequence in a gene encoding an aminopeptidase N (ANPEP) protein.

2. The livestock animal, offspring, or cell of claim 1, wherein the modified chromosomal sequence in the gene encoding the ANPEP protein reduces the susceptibility of the animal, offspring, or cell to infection by a pathogen, as compared to the susceptibility of a livestock animal, offspring, or cell that does not comprise a modified chromosomal sequence in a gene encoding an ANPEP protein to infection by the pathogen.

3. The livestock animal, offspring, or cell of claim 2, wherein the pathogen comprises an Alphacoronavirus genus virus.

4. (canceled)

5. The livestock animal, offspring, or cell of claim 3, wherein the Alphacoronavirus genus virus comprises a transmissible gastroenteritis virus (TGEV) or a porcine respiratory coronavirus (PRCV).

6. The livestock animal, offspring, or cell of claim 1, wherein the livestock animal comprises a porcine animal or wherein the cell is derived from a porcine animal.

7. The livestock animal, offspring, or cell of claim 1, wherein the animal or offspring is an embryo, a juvenile, or an adult, or wherein the cell comprises an embryonic cell, a cell derived from a juvenile animal, or a cell derived from an adult animal.

8. The livestock animal, offspring, or cell of claim 1, wherein the animal, offspring, or cell is heterozygous for the modified chromosomal sequence in the gene encoding the ANPEP protein.

9. The livestock animal, offspring, or cell of claim 1, wherein the animal, offspring, or cell is homozygous for the modified chromosomal sequence in the gene encoding the ANPEP protein.

10. The livestock animal, offspring, or cell of claim 1, wherein the modified chromosomal sequence comprises an insertion in an allele of the gene encoding the ANPEP protein, a deletion in an allele of the gene encoding the ANPEP protein, a substitution in an allele of the gene encoding the ANPEP protein, or a combination of any thereof.

11. The livestock animal, offspring, or cell of claim 10, wherein the insertion, the deletion, the substitution, or the combination of any thereof results in a miscoding in the allele of the gene encoding the ANPEP protein.

12. The livestock animal, offspring, or cell of claim 10, wherein the deletion comprises:
a deletion of the start codon of the allele of the gene encoding the ANPEP protein; or
a deletion of the entire coding sequence of the allele of the gene encoding the ANPEP protein.

13. The livestock animal, offspring, or cell of claim 1, wherein the modified chromosomal sequence in the gene encoding the ANPEP protein causes ANPEP protein production or activity to be reduced, as compared to ANPEP protein production or activity in an animal, offspring, or cell that lacks the modified chromosomal sequence in the gene encoding the ANPEP protein.

14. The livestock animal, offspring, or cell of claim 1, wherein the modified chromosomal sequence in the gene encoding the ANPEP protein results in production of substantially no functional ANPEP protein by the animal, offspring, or cell.

15. The livestock animal, offspring, or cell of claim 1, wherein the modified chromosomal sequence comprises a modification in: exon 2 of an allele of the gene encoding the ANPEP protein; exon 4 of an allele of the gene encoding the ANPEP protein; an intron that is contiguous with exon 2 or exon 4 of the allele of the gene encoding the ANPEP protein; or a combination of any thereof.

16. The livestock animal, offspring, or cell of claim 15, wherein the modified chromosomal sequence comprises a deletion in exon 2 of the allele of the gene encoding the ANPEP protein, the deletion comprising an in-frame deletion in exon 2.

17. The livestock animal, offspring, or cell of claim 16, wherein the in-frame deletion in exon 2:
results in deletion of amino acids 194 through 196 of the ANPEP protein; or
results in deletion of amino acids 194 through 197 of the ANPEP protein, wherein the in-frame deletion optionally further results in substitution of the valine residue at position 198 of the ANPEP protein with an isoleucine residue.

18. The livestock animal, offspring, or cell of claim 15, wherein the modified chromosomal sequence comprises a modification selected from the group consisting of:
a 182 base pair deletion from nucleotide 1,397 to nucleotide 1,578, as compared to reference sequence SEQ ID NO: 135, wherein the deleted sequence is replaced with a 5 base pair insertion beginning at nucleotide 1,397;
a 9 base pair deletion from nucleotide 1,574 to nucleotide 1,582, as compared to reference sequence SEQ ID NO: 135;
a 9 base pair deletion from nucleotide 1,577 to nucleotide 1,585, as compared to reference sequence SEQ ID NO: 135;
a 9 base pair deletion from nucleotide 1,581 to nucleotide 1,589, as compared to reference sequence SEQ ID NO: 135;
an 867 base pair deletion from nucleotide 819 to nucleotide 1,685, as compared to reference sequence SEQ ID NO: 135;
an 867 base pair deletion from nucleotide 882 to nucleotide 1,688, as compared to reference sequence SEQ ID NO: 135;
a 1 base pair insertion between nucleotides 1,581 and 1,582, as compared to reference sequence SEQ ID NO: 135;
a 1 base pair insertion between nucleotides 1,580 and 1,581, as compared to reference sequence SEQ ID NO: 135;
a 1 base pair insertion between nucleotides 1,579 and 1,580, as compared to reference sequence SEQ ID NO: 135;
a 2 base pair insertion between nucleotides 1,581 and 1,582, as compared to reference sequence SEQ ID NO: 135;
a 267 base pair deletion from nucleotide 1,321 to nucleotide 1,587, as compared to reference sequence SEQ ID NO: 135;
a 267 base pair deletion from nucleotide 1,323 to nucleotide 1,589, as compared to reference sequence SEQ ID NO: 135;
a 1 base pair deletion of nucleotide 1,581, as compared to reference sequence SEQ ID NO: 135;
a 12 base pair deletion from nucleotide 1,582 to nucleotide 1,593, as compared to reference sequence SEQ ID NO: 135;
a 25 base pair deletion from nucleotide 1,561 to nucleotide 1,585, as compared to reference sequence SEQ ID NO: 135;
a 25 base pair deletion from nucleotide 1,560 to nucleotide 1,584, as compared to reference sequence SEQ ID NO: 135;
an 8 base pair deletion from nucleotide 1,575 to nucleotide 1,582, as compared to reference sequence SEQ ID NO: 135;
an 8 base pair deletion from nucleotide 1,574 to nucleotide 1,581, as compared to reference sequence SEQ ID NO: 135;
a 661 base pair deletion from nucleotide 940 to nucleotide 1,600, as compared to reference sequence SEQ ID NO: 135, wherein the deleted sequence is replaced with an 8 base pair insertion beginning at nucleotide 940;
an 8 base pair deletion from nucleotide 1,580 to nucleotide 1,587, as compared to reference sequence SEQ ID NO: 135, wherein the deleted sequence is replaced with a 4 base pair insertion beginning at nucleotide 1,580;
and combinations of any thereof.

19. (canceled)

20. The livestock animal, offspring, or cell of claim 1, wherein the modified chromosomal sequence comprises a modification within the region comprising nucleotides 17,235 through 22,422 of reference sequence SEQ ID NO: 132.

21. The livestock animal, offspring, or cell of claim 10, wherein the animal, offspring or cell comprises a chromosomal sequence in the gene encoding the ANPEP protein having at least 80%, at least 85%, at least 90%, at least 95%, at least 98%, at least 99%, at least 99.9%, or 100% sequence identity to SEQ ID NO: 135 or 132 in the regions of the chromosomal sequence outside of the insertion, the deletion, or the substitution.

22. The livestock animal, offspring, or cell of claim 1, wherein the livestock animal, offspring, or cell comprises a chromosomal sequence comprising SEQ ID NO: 163, 164, 165, 166, 167, 168, 170, 171, 172, 173, 174, 176, 177, or 178.

23. The livestock animal, offspring, or cell of claim 1, wherein the livestock animal, offspring, or cell further comprises at least one modified chromosomal sequence in a gene encoding a CD163 protein.

24. The livestock animal, offspring, or cell of claim 23, wherein the modified chromosomal sequence in the gene encoding the CD163 protein:
reduces the susceptibility of the animal, offspring, or cell to infection by a porcine reproductive and respiratory syndrome virus (PRRSV), as compared to the susceptibility of an animal, offspring, or cell that does not comprise a modified chromosomal sequence in a gene encoding a CD163 protein to infection by the porcine reproductive and respiratory syndrome virus; and/or
results in production of substantially no functional CD163 protein by the animal, offspring, or cell.

25-26. (canceled)

27. The livestock animal, offspring, or cell of claim 1, wherein the animal or offspring comprises a genetically edited animal or offspring or wherein the cell comprises a genetically edited cell, wherein the animal or cell has been genetically edited using a homing endonuclease, the homing endonuclease comprising a Clustered Regularly Interspaced Short Palindromic Repeats (CRISPR) system, a Transcription Activator-Like Effector Nuclease (TALEN), a Zinc Finger Nuclease (ZFN), a recombinase fusion protein, a meganuclease, or a combination of any thereof.

28. (canceled)

29. The cell of claim 1, wherein the cell comprises a sperm cell, an egg cell (optionally a fertilized egg), or a somatic cell (optionally a fibroblast).

30. A method of producing a non-human animal or a lineage of non-human animals having reduced susceptibility to infection by a pathogen, wherein the method comprises:
modifying an oocyte or a sperm cell to introduce a modified chromosomal sequence in a gene encoding an aminopeptidase N (ANPEP) protein into at least one of the oocyte and the sperm cell, and fertilizing the oocyte with the sperm cell to create a fertilized egg containing the modified chromosomal sequence in the gene encoding a ANPEP protein; or
modifying a fertilized egg to introduce a modified chromosomal sequence in a gene encoding an ANPEP protein into the fertilized egg;
transferring the fertilized egg into a surrogate female animal, wherein gestation and term delivery produces a progeny animal;
screening the progeny animal for susceptibility to the pathogen; and
selecting progeny animals that have reduced susceptibility to the pathogen as compared to animals that do not comprise a modified chromosomal sequence in a gene encoding an ANPEP protein.

31. (canceled)

32. A population of livestock animals comprising two or more livestock animals and/or offspring thereof of claim 1.

33. A nucleic acid molecule comprising a nucleotide sequence selected from the group consisting of:
(a) a nucleotide sequence having at least 80% sequence identity to the sequence of SEQ ID NO: 135, wherein the nucleotide sequence comprises at least one substitution, insertion, or deletion relative to SEQ ID NO: 135;
(b) a nucleotide sequence having at least 80% sequence identity to the sequence of SEQ ID NO: 132, wherein the nucleotide sequence comprises at least one substitution, insertion, or deletion relative to SEQ ID NO: 132; and
(c) a cDNA of (a) or (b).

34-35. (canceled)
